(19) United States
(12) Patent Application Publication
(10) Pub. No.: US 2024/0382600 A1
(43) Pub. Date: Nov. 21, 2024

(54) BIFUNCTIONAL DEGRADERS COMPRISING A TEAD BINDER

(71) Applicant: Novartis AG, Basel (CH)

(72) Inventors: Emilie CHAPEAU, Huningue (FR); Patrick CHENE, Mulhouse (FR); Pascal FURET, Thann (FR); Laurence KIEFFER, Niffer (FR); Rainer MACHAUER, Ehrenkirchen (DE); Anne PICARD, Kembs (FR); Tobias SCHMELZLE, Riehen (CH); Holger SELLNER, Buchs (CH); Nicolas SOLDERMANN, Village-Neuf (FR); Markus VOEGTLE, Lörrach (DE); Markus WARTMANN, Riehen (CH)

(21) Appl. No.: 18/687,615

(22) PCT Filed: Aug. 30, 2022
(86) PCT No.: PCT/IB2022/058133

§ 371 Date: Feb. 28, 2024

Related U.S. Application Data
(60) Provisional application No. 63/239,510, filed on Sep. 1, 2021.

(51) Int. Cl. A61K 47/54; A61K 47/55; C07D 405/14; C07D 417/12

(52) U.S. Cl. CPC A61K 47/545; A61K 47/55; C07D 405/14; C07D 417/12

(57) ABSTRACT

The present disclosure relates to a conjugate comprising a YAP/TAZ-TEAD Protein Protein Interaction Inhibitor (PPII) linked to a Ligase Binder via a linker, or a pharmaceutically acceptable salt thereof, as well as methods of using such conjugates.

### FIELD OF THE DISCLOSURE

The present disclosure relates to a conjugate comprising a YAP/TAZ-TEAD Protein Protein Interaction Inhibitor (PPII) linked to a Ligase Binder via a linker, or a pharmaceutically acceptable salt thereof, as well as methods of using such conjugates.

### BACKGROUND

### Bifunctional Degraders

Proteins within the body are regulated transcriptionally, translationally and post-translationally. Regulatory failure can result in a number of diseases including cancer. Traditional small molecule therapeutics target the inhibition of enzymes or receptors through binding to the active site or the allosteric site.

An alternative to targeting these sites is the use of bifunctional protein degraders which bind both to the target protein and to an additional protein (typically E3 ubiquitin ligase) that flags the target protein for natural degradation. The principle of induced degradation of protein targets as a potential therapeutic approach has been described by Crews, J. Med, Chem. 61(2): 403-404 (2018) and references cited therein.

### Hippo Pathway

Normal tissue growth, as well as tissue repair and remodeling, requires specific control and regulated balance of transcriptional activity. Transcriptional output is coordinated through a number of key signaling modules, one of which is the Hippo pathway. Genetic studies in Drosophila and mammals have defined a conserved core signaling cassette, composed of Mst1/2 and Lats1/2 kinases which inhibit the transcriptional co-activators YAP and TAZ (official gene name: WWTR1).

An activated Hippo pathway translates to YAP and TAZ being phosphorylated and sequestered/degraded in the cytoplasm. Upon inactivation of the Hippo pathway, YAP and TAZ translocate to the nucleus and associate with transcription factors, namely members of the TEAD family (TEAD1-4). The YAP/TAZ-TEAD complexes in turn promote transcription of downstream genes involved in cellular proliferation, death and differentiation. While YAP and TAZ can also interact with a number of other factors, TEADs are commonly accepted to be the key mediators of the growth-promoting and tumorigenic potential of YAP and TAZ (pathway reviewed in Yu et al., 2015; Holden and Cunningham, 2018).

Accordingly, a hyperactivation of YAP and/or TAZ (and subsequent hyperactivity of the YAP/TAZ-TEAD transcriptional complex) is commonly observed in several human cancers. This is evidenced by the levels and nuclear localization of YAP/TAZ being elevated in many tumors, including breast, lung (e.g., non-small cell; NSCLC), ovarian, colorectal, pancreas, prostate, gastric, esophagus, liver and bone (sarcoma) (Steinhardt et al., 2008; Harvey et al., 2013; Moroishi et al., 2015; extensively reviewed in Zanconato et al., 2016 and references therein). While genetic alterations of the core Hippo pathway components have thus far been detected with limited frequency in primary samples, the most prominent cancer malignancy associated with inactivating mutations in NF2 or Lats1/2 and associated YAP/TEAD hyperactivity is malignant pleural mesothelioma (MPM) (reviewed in Sekido, 2018). Similarly, a number of human tumors are characterized by amplification of YAP at the 11q22.1 locus (e.g., hepatocellular carcinomas, medulloblastomas, esophageal squamous cell carcinomas), TAZ (WWTR1) at the 3q25.1 locus (e.g., rhabdomyosarcomas, triple negative breast cancer) or gene fusions involving YAP or TAZ (epithelioid hemangioendotheliomas, ependymal tumors) (reviewed in Yu et al., 2015 and references therein). As is the case for MPM, such tumors are also anticipated to depend on their elevated YAP/TAZ-TEAD activity.

Disruption of the YAP/TAZ-TEAD PPI as the most distal effector node of the Hippo pathway is anticipated to abolish the oncogenic potential of this complex.

The bifunctional degraders of this invention are designed and optimized to bind to both TEADs and E3 ubiquitin ligase, and thus flag TEAD for natural degradation, which is believed to result in drugs useful in the treatment of above-mentioned cancers. In particular, such cancers may be characterized by (but not restricted to) some of the described aberrations.

Notably, tumor cells with activated YAP/TAZ-TEAD display resistance to chemotherapeutic drugs, possibly related to YAP/TAZ conferring cancer stem cell-like characteristics. Moreover, YAP/TAZ-TEAD activation also confers resistance to molecularly targeted therapies, such as BRAF, MEK or EGFR inhibitors, as reported from the outcome of various genetic and pharmacological screens (Kapoor et al., 2014; Shao et al., 2014; Lin et al., 2015). This in turn suggests that degrading TEAD and thus retarding YAP/TAZ-TEAD activity—either in parallel or sequentially to other cancer treatments—may provide a beneficial therapeutic impact by reducing growth of tumors resistant to other treatments.

The retardation of YAP/TAZ-TEAD activity achieved through targeted TEAD degradation may also blunt the tumor's escape from immune surveillance. This is, for instance, evidenced by reported data on YAP promoting the expression of chemokine CXCL5 which results in the recruitment of myeloid cells that suppress T-cells (Wang et al., 2016). YAP in Tregs (regulatory T-cells) has also been demonstrated to support FOXP3 expression via activin signaling and Treg function. Accordingly, YAP deficiency results in dysfunctional Tregs which are no longer able to suppress antitumor immunity. Retardation of YAP/TEAD activity may therefore contribute to bolster antitumor immunity by preventing Treg function (Ni et al., 2018). Recent literature also suggests that YAP upregulates PD-L1 expression and by this mechanism directly mediates evasion of cytotoxic T-cell immune responses, for instance in BRAF inhibitor-resistant melanoma cells (Kim et al., 2018). For treatment purposes, conjugates of the invention may be used in combination with cancer immunotherapy drugs, such as immune checkpoint inhibitors (e.g., anti-PD-1 antibodies).

### See for Example:

Yu, F-X., Zhao, B. and Guan, K.-L. (2015). Hippo pathway in organ size control, tissue homeostasis, and cancer. Cell, 163, 811-828.
Holden, J. K. and Cunningham, C. N. (2018). Targeting the Hippo pathway and cancer through the TEAD family of transcription factors. Cancers (Basel), 10, E81.
Steinhardt, A. A., Gayyed, M. F., Klein, A. P., Dong, J., Maitra, A., Pan, D., Montgomery, E. A., Anders, R. A. (2008). Expression of Yes-associated protein in common solid tumors. Hum. Pathol., 39, 1582-1589.
Harvey, K. F., Zhang, X., and Thomas, D. M. (2013). The Hippo pathway and human cancer. Nat. Rev. Cancer, 13, 246-257.
Moroishi, T., Hansen, C. G., and Guan, K.-L. (2015). Nat. Rev. Cancer, 15, 73-79.
Zanconato, F., Cordenonsi, M., and Piccolo, S. (2016). YAP/TAZ at the roots of cancer. Cancer Cell, 29, 783-803.
Sekido, Y. (2018). Cancers (Basel), 10, E90.
Kapoor, A., Yao, W., Ying, H., Hua, S., Liewen, A., Wang, Q., Zhong, Y., Wu, C. J., Sadanandam, A., Hu, B. et al. (2014). Yap1 activation enables bypass of oncogenic Kras addiction in pancreatic cancer. Cell, 158, 185-197.
Shao, D. D., Xue, W., Krall, E. B., Bhutkar, A., Piccioni, F., Wang, X., Schinzel, A. C., Sood, S., Rosenbluh, J., Kim, J. W., et al. (2014). KRAS and YAP1 converge to regulate EMT and tumor survival. Cell, 158, 171-184.
Lin, L., Sabnis, A. J., Chan, E., Olivas, V., Cade, L., Pazarentzos, E., Asthana, S., Neel, D., Yan, J. J., Lu, X. et al. (2015). The Hippo effector YAP promotes resistance to RAF- and MEK-targeted cancer therapies. Nat. Genet., 47, 250-256.
Wang, G., Lu, X., Dey, P., Deng, P., Wu, C. C., Jiang, S., Fang, Z., Zhao, K., Konaprathi, R., Hua, S., et al. (2016). Cancer Discov., 6, 80-95.
Ni, X., Tao, J., Barbi, J., Chen, Q., Park B. V., Li, Z., Zhang, N., Lebid, A., Ramaswamy, A., Wei, P., et al. (2018). YAP is essential for Treg-mediated suppression of antitumor immunity. Cancer Discov., 8, 1026-1043.
Kim, M. H., Kim, C. G., Kim, S. K., Shin, S. J., Choe, E. A., Park, S. H., Shin, E. C., and Kim, J. (2018). Cancer Immunol Res., 6, 255-266.

### SUMMARY

There remains a need in the art for new treatments and therapies for diseases mediated by YAP overexpression and/or YAP amplification and/or YAP/TAZ-TEAD protein-protein interaction (PPI). The disclosure provides compounds that recruit a targeted protein, such as TEAD, to E3 Ubiquitin ligase for degradation.

Therefore, according to a first aspect of the invention, there is hereby provided a conjugate of Formula (I):

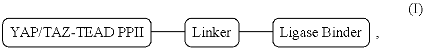

or a pharmaceutically acceptable salt thereof, wherein:
the YAP/TAZ-TEAD PPI inhibitor is a group that is capable of binding to TEAD;
the Linker is a group that covalently links the YAP/TAZ-TEAD PPI inhibitor to the Ligase Binder; and
the Ligase Binder is a group that is capable of binding to a ligase (e.g., Cereblon E3 Ubiquitin ligase).

In another aspect, the invention provides a pharmaceutical composition comprising the conjugate or pharmaceutically acceptable salt thereof according to the first aspect of the invention, and one or more pharmaceutically acceptable carriers.

In another aspect, the invention provides a combination comprising the conjugate or pharmaceutically acceptable salt thereof according to the first aspect of the invention, and one or more therapeutically active agents.

In another aspect, the invention provides the conjugate or pharmaceutically acceptable salt thereof according to the first aspect of the invention for use as a medicament.

In another aspect, the invention provides the conjugate or pharmaceutically acceptable salt thereof according to the first aspect of the invention for use in the treatment of cancer, preferably wherein the cancer is selected from breast cancer, lung cancer, ovarian cancer, colorectal cancer, malignant pleural mesothelioma, pancreatic cancer, prostate cancer, gastric cancer, esophageal cancer, liver cancer and bone cancer.

In another aspect, the invention provides the conjugate or pharmaceutically acceptable salt thereof according to the first aspect of the invention for use in the treatment of a disease or condition mediated by YAP overexpression and/or YAP amplification and/or YAP/TAZ-TEAD interaction.

### DETAILED DESCRIPTION

The invention provides a conjugate of Formula (I):

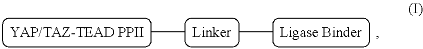

or a pharmaceutically acceptable salt thereof, wherein:
the YAP/TAZ-TEAD PPI inhibitor is a group that is capable of binding to TEAD;
the Linker is a group that covalently links the YAP/TAZ-TEAD PPI inhibitor to the Ligase Binder; and
the Ligase Binder is a group that is capable of binding to a ligase (e.g., Cereblon E3 Ubiquitin ligase).

In an embodiment, the YAP/TAZ-TEAD PPI inhibitor has a formula (Ia)

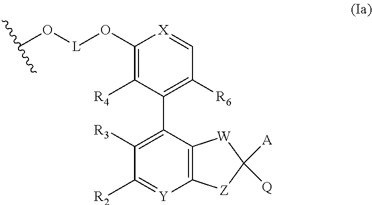

wherein:
L is C₁-C₆alkylene;
W is selected from O; and CH—R_{w};
X is selected from CH; and N;
Y is selected from CH; and N;
Z is selected from CH₂; O; and NH;
wherein when Y is N, W is CH—R_{w}, and Z is O;
A is selected from
(i) phenyl, which phenyl is optionally substituted with halo or haloC₁-C₃alkoxy;
(ii) a 5- or 6-membered aromatic heterocyclic ring comprising at least one heteroatom selected from N, O, and S, preferably from N and S, which aromatic heterocyclic ring is optionally substituted with hydroxy; C₁-C₃alkoxy; or oxo; and
(iii)

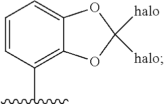

R_{w} is selected from (i) hydrogen; (ii) hydroxy; (iii) C₁-C₃alkoxy; (iv) hydroxyC₁-C₃alkyl; (v) C₁-C₃alkyl; and (vi) C₁-C₃alkoxy-C₁-C₃alkyl;
Q is selected from
   (i) —C(R⁷)₂—N(R⁸)—R₁;
   (ii) 9- or 10-membered partially saturated heteroaryl comprising at least one N heteroatom; and
   (iii) 4-, 5- or 6-membered saturated heterocyclic ring comprising at least one heteroatom or heteroatom group selected from N, O, S, —S(═O) and —S(═O)₂, with the proviso that at least one N heteroatom is present, wherein the heterocyclic ring is unsubstituted or substituted with one or more substituents independently selected from hydroxy, C₁-C₃alkyl, C₁-C₃alkoxy, halo and C₁-C₃alkylene forming a bridge between two ring atoms of the saturated heterocyclic ring, thus forming a bridged bicyclic structure;
R₁ is selected from (i) hydrogen, (ii) C₁-C₆alkyl (wherein the alkyl is in one embodiment optionally deuterated, e.g. perdeuterated), and (iii) (CH₂)₀₋₂R₁ₐ;
R₁ₐ is selected from
   (i) hydroxyC₁-C₄alkyl,
   (ii) C₁-C₃alkoxy,
   (iii) a 5- or 6-membered saturated heterocyclic ring comprising at least one heteroatom selected from N and O, which saturated heterocyclic ring is optionally substituted once or more than once independently with C₁-C₃alkyl, (CH₂)₀₋₁C(O)di(C₁-C₃alkyl)amino, SO₂C₁-C₃alkyl, C(O)C₁-C₃alkyl, or oxo,
   (iv) C₃-C₆cycloalkyl optionally substituted once or more than once independently with hydroxy, hydroxyC₁-C₄alkyl, C₁-C₆alkoxy (preferably C₁-C₄alkoxy), C(O)OC₁-C₃alkyl, CO₂H, SO₂C₁-C₃alkyl, haloC₁-C₃alkyl, NHR^{1b}, (CH₂)₀₋₁C(O)NR^{1c}R^{1d} (preferably C(O)NR^{1c}R^{1d}), C₁-C₆alkyl, haloC₁-C₃alkoxy-C₁-C₃alkyl, halo, a 5- or 6-membered aromatic heterocyclic ring comprising at least one heteroatom selected from N, O, and S, or with two R^{1e} groups,
      wherein the two R^{1e} attached at the same carbon atom form together with the carbon atom to which they are attached a 5-membered saturated heterocyclic ring comprising at least one heteroatom selected from N (which is preferred) and O, or a C₃-C₆cycloalkyl, which saturated heterocyclic ring or cycloalkyl are optionally substituted with hydroxy or oxo;
R^{1b} is selected from (i) C(O)C₁-C₃alkyl, and (ii) SO₂C₁-C₃alkyl,
R^{1c} and R^{1d} are each independently selected from (i) hydrogen, (ii) C₁-C₃alkyl, and (iii) hydroxyC₁-C₄alkyl, preferably from (i) hydrogen and (ii) C₁-C₃alkyl,
R₂ is selected from (i) hydrogen, and (ii) halo,
R₃ is selected from (i) halo, (ii) haloC₁-C₃alkyl, especially from halo and mono-, di- or preferably tri-halomethyl, and (iii) cyano,
R₄ is selected from (i) hydrogen, (ii) halo, and (iii) C₁-C₃alkyl, especially from hydrogen, halo and methyl;
R₆ is selected from (i) hydrogen, (ii) cyano, (iii) C(O)NHR^{6a}, (iv) NHR^{6b}, and (v) C₁-C₃alkoxy substituted with NH₂ or hydroxy;
R^{6a} is selected from (i) hydrogen, (ii) C₁-C₃alkyl, (iii) C₃-C₆cycloalkyl, (iv) a 5- or 6-membered aromatic heterocyclic ring comprising at least one heteroatom selected from N, O, and S, preferably at least one N heteroatom, which aromatic heterocyclic ring is optionally substituted with C₁-C₃alkyl;
R^{6b} is C₁-C₃alkyl substituted with NH₂ or hydroxy;
R⁷ is each independently selected from hydrogen and C₁-C₃alkyl; and
R⁸ is hydrogen or C₁-C₃-alkyl, especially hydrogen or methyl;
and wherein [symbol] indicates the point of attachment to the linker.

In an embodiment, the YAP/TAZ-TEAD PPI inhibitor has a formula (Ib)

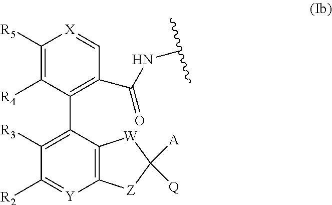

wherein:
W is selected from O; and CH—R_{w};
X is selected from CH; and N;
Y is selected from CH; and N;
Z is selected from CH₂; O; and NH;
wherein when Y is N, W is CH—R_{w}, and Z is O;
A is selected from
(i) phenyl, which phenyl is optionally substituted with halo or haloC₁-C₃alkoxy;
(ii) a 5- or 6-membered aromatic heterocyclic ring comprising at least one heteroatom selected from N, O, and S, preferably from N and S, which aromatic heterocyclic ring is optionally substituted with hydroxy; C₁-C₃alkoxy; or oxo; and
(iii)

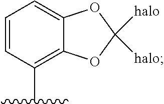

R_{w} is selected from (i) hydrogen; (ii) hydroxy; (iii) C₁-C₃alkoxy; (iv) hydroxyC₁-C₃alkyl; (v) C₁-C₃alkyl; and (vi) C₁-C₃alkoxy-C₁-C₃alkyl;
Q is selected from
   (i) —C(R⁷)₂—N(R⁸)—R₁;
   (ii) 9- or 10-membered partially saturated heteroaryl comprising at least one N heteroatom; and
   (iii) 4-, 5- or 6-membered saturated heterocyclic ring comprising at least one heteroatom or heteroatom group selected from N, O, S, —S(═O) and —S(═O)₂, with the proviso that at least one N heteroatom is present, wherein the heterocyclic ring is unsubstituted or substituted with one or more substituents independently selected from hydroxy, C₁-C₃alkyl, C₁-C₃alkoxy, halo and C₁-C₃alkylene forming a bridge between two ring atoms of the saturated heterocyclic ring, thus forming a bridged bicyclic structure;
R₁ is selected from (i) hydrogen, (ii) C₁-C₆alkyl (wherein the alkyl is in one embodiment optionally deuterated, e.g. perdeuterated), and (iii) (CH₂)₀₋₂R₁ₐ;
R₁ₐ is selected from
   (i) hydroxyC₁-C₄alkyl,
   (ii) C₁-C₃alkoxy,
   (iii) a 5- or 6-membered saturated heterocyclic ring comprising at least one heteroatom selected from N and O, which saturated heterocyclic ring is optionally substituted once or more than once independently with C₁-C₃alkyl, (CH₂)₀₋₁C(O)di(C₁-C₃alkyl)amino, SO₂C₁-C₃alkyl, C(O)C₁-C₃alkyl, or oxo,
   (iv) C₃-C₆cycloalkyl optionally substituted once or more than once independently with hydroxy, hydroxyC₁-C₄alkyl, C₁-C₃alkoxy (preferably C₁-C₄alkoxy), C(O)OC₁-C₃alkyl, CO₂H, SO₂C₁-C₃alkyl, haloC₁-C₃alkyl, NHR^{1b}, (CH₂)₀₋₁C(O)NR^{1c}R^{1d} (preferably C(O)NR^{1c}R^{1d}), C₁-C₆alkyl, haloC₁-C₃alkoxy-C₁-C₃alkyl, halo, a 5- or 6-membered aromatic heterocyclic ring comprising at least one heteroatom selected from N, O, and S, or with two R^{1e} groups,
      wherein the two R^{1e} attached at the same carbon atom form together with the carbon atom to which they are attached a 5-membered saturated heterocyclic ring comprising at least one heteroatom selected from N (which is preferred) and O, or a C₃-C₆cycloalkyl, which saturated heterocyclic ring or cycloalkyl are optionally substituted with hydroxy or oxo;
R^{1b} is selected from (i) C(O)C₁-C₃alkyl, and (ii) SO₂C₁-C₃alkyl,
R^{1c} and R^{1d} are each independently selected from (i) hydrogen, (ii) C₁-C₃alkyl, and (iii) hydroxyC₁-C₄alkyl, preferably from (i) hydrogen and (ii) C₁-C₃alkyl,
R₂ is selected from (i) hydrogen, and (ii) halo,
R₃ is selected from (i) halo, (ii) haloC₁-C₃alkyl, especially from halo and mono-, di- or preferably tri-halomethyl, and (iii) cyano,
R₄ is selected from (i) hydrogen, (ii) halo, and (iii) C₁-C₃alkyl, especially from hydrogen, halo and methyl;
R₅ is selected from
   (i) hydrogen,
   (ii) C₁-C₃alkoxy optionally substituted with C₃-C₆cycloalkyl, CO₂H, SO₂C₁-C₃alkyl, a 5- or 6-membered aromatic heterocyclic ring comprising at least one heteroatom selected from N, O, and S, preferably at least one N heteroatom, or a 5- or 6-membered saturated heterocyclic ring comprising at least one heteroatom selected from N and O, which ring is optionally substituted with C(O)C₁-C₃alkyl, halo, (iii)
   (iv) hydroxyC₁-C₆alkoxy (where the alkoxy part is in one embodiment optionally deuterated, e.g. perdeuterated),
   (v) haloC₁-C₃alkoxy optionally substituted with hydroxy,
   (vi) S-haloC₁-C₃alkyl optionally substituted with hydroxy,
   (vii) C₁-C₃alkoxyC₁-C₃alkoxy,
   (viii) NR^{5a}R^{5b}
   (ix) C₁-C₃alkyl,
   (x) a 5- or 6-membered aromatic heterocyclic ring comprising at least one heteroatom selected from N, O, and S, preferably at least one N heteroatom, and
   (xi) hydroxy;
R^{5a} and R^{5b} are each independently selected from (i) hydrogen, and (ii) C₁-C₃alkyl,
or
R^{5a} and R^{5b} together with the nitrogen atom to which they are attached form a 5- or 6-membered saturated heterocyclic ring, which saturated heterocyclic ring optionally in addition carries a hydroxy group;
R⁷ is each independently selected from hydrogen and C₁-C₃alkyl, and
R⁸ is hydrogen or C₁-C₃-alkyl, especially hydrogen or methyl;
and wherein [symbol] indicates the point of attachment to the linker.

In an embodiment, the YAP/TAZ-TEAD PPI inhibitor has a formula (Ic)

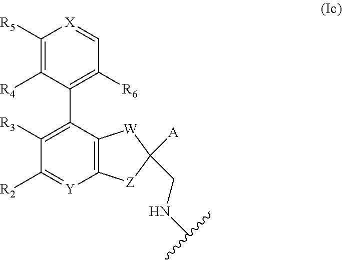

wherein:
W is selected from O; and CH—R_{w};
X is selected from CH; and N;
Y is selected from CH; and N;
Z is selected from CH₂; O; and NH;
wherein when Y is N, W is CH—R_{w}, and Z is O;
A is selected from
(i) phenyl, which phenyl is optionally substituted with halo or haloC₁-C₃alkoxy;
(ii) a 5- or 6-membered aromatic heterocyclic ring comprising at least one heteroatom selected from N, O, and S, preferably from N and S, which aromatic heterocyclic ring is optionally substituted with hydroxy; C₁-C₃alkoxy; or oxo; and
(iii)

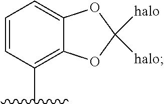

R_{w} is selected from (i) hydrogen; (ii) hydroxy; (iii) C₁-C₃alkoxy; (iv) hydroxyC₁-C₃alkyl; (v) C₁-C₃alkyl; and (vi) C₁-C₃alkoxy-C₁-C₃alkyl;
R₂ is selected from (i) hydrogen, and (ii) halo,
R₃ is selected from (i) halo, (ii) haloC₁-C₃alkyl, especially from halo and mono-, di- or preferably tri-halomethyl, and (iii) cyano,
R₄ is selected from (i) hydrogen, (ii) halo, and (iii) C₁-C₃alkyl, especially from hydrogen, halo and methyl;
R₅ is selected from
   (i) hydrogen,
   (ii) C₁-C₃alkoxy optionally substituted with C₃-C₆cycloalkyl, CO₂H, SO₂C₁-C₃alkyl, a 5- or 6-membered aromatic heterocyclic ring comprising at least one heteroatom selected from N, O, and S, preferably at least one N heteroatom, or a 5- or 6-membered saturated heterocyclic ring comprising at least one heteroatom selected from N and O, which ring is optionally substituted with C(O)C₁-C₃alkyl,
   (iii) halo,
   (iv) hydroxyC₁-C₃alkoxy (where the alkoxy part is in one embodiment optionally deuterated, e.g. perdeuterated),
   (v) haloC₁-C₃alkoxy optionally substituted with hydroxy,
   (vi) S-haloC₁-C₃alkyl optionally substituted with hydroxy,
   (vii) C₁-C₃alkoxyC₁-C₃alkoxy,
   (viii) NR^{5a}R^{5b},
   (ix) C₁-C₃alkyl,
   (x) a 5- or 6-membered aromatic heterocyclic ring comprising at least one heteroatom selected from N, O, and S, preferably at least one N heteroatom, and
   (xi) hydroxy;
R^{5a} and R^{5b} are each independently selected from (i) hydrogen, and (ii) C₁-C₃alkyl, or
R^{5a} and R^{5b} together with the nitrogen atom to which they are attached form a 5- or 6-membered saturated heterocyclic ring, which saturated heterocyclic ring optionally in addition carries a hydroxy group;
R₆ is selected from (i) hydrogen, (ii) cyano, (iii) C(O)NHR^{6a}, (iv) NHR^{6b}, and (v) C₁-C₃alkoxy substituted with NH₂ or hydroxy,
R^{6a} is selected from (i) hydrogen, (ii) C₁-C₃alkyl, (iii) C₃-C₆cycloalkyl, (iv) a 5- or 6-membered aromatic heterocyclic ring comprising at least one heteroatom selected from N, O, and S, preferably at least one N heteroatom, which aromatic heterocyclic ring is optionally substituted with C₁-C₃alkyl;
R^{6b} is C₁-C₃alkyl substituted with NH₂ or hydroxy;
and wherein [symbol] indicates the point of attachment to the linker.

In an embodiment, the YAP/TAZ-TEAD PPI inhibitor has a formula YTT-1, YTT-2 or YTT-3:

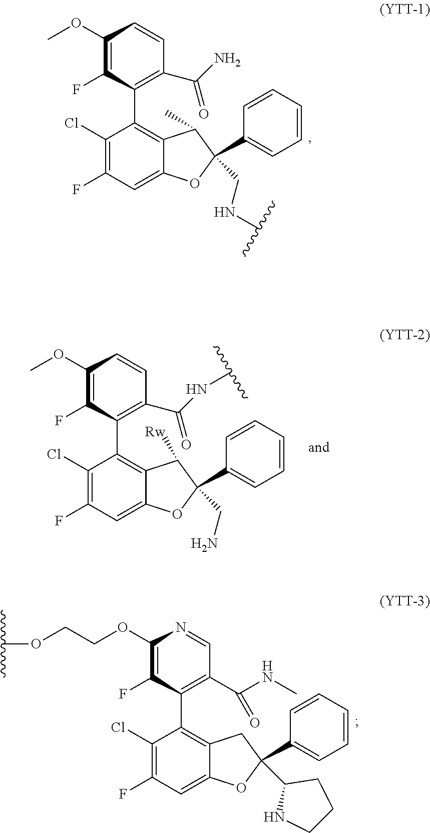

wherein R_{w} is H or CH₃ (preferably wherein is R_{w} is H) and wherein [symbol] indicates the point of attachment to the linker.

In an embodiment, the YAP/TAZ-TEAD PPI inhibitor has a formula YTT-1:

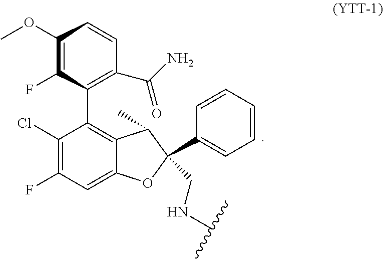

In an embodiment, the linker has a formula LIN-1: -L¹-X¹-L²-X²-L³-(LIN-1), and the conjugate has a formula (II):

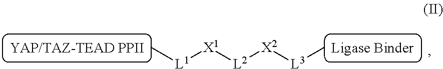

wherein:
L¹ is selected from the group consisting of a bond, —O—, —NR′—, —C(O)—, C₁-C₉alkylene, C₁-C₉heteroalkylene, *C(O)—C₁-C₆alkylene, *C(O)—C₁-C₆heteroalkylene, *C₁-C₃alkylene-C(O), *C₁-C₆ heteroalkylene-C(O), and *L^{1a}-C₄-C₇cycloalkylene, wherein * denotes the point of attachment of L¹ to X¹;
wherein L^{1a} is selected from the group consisting of C(O), *NH—C(O) and *C₁-C₃alkylene-NH—C(O) wherein * denotes the point of attachment of L^{1a} to X¹;
X¹ and X² are each independently selected from the group consisting of a bond, C₄-C₇cycloalkylene, 4 to 7 membered heterocyclylene comprising 1 to 3 heteroatoms independently selected from the group consisting of N, O and S, and 5 or 6 membered heteroarylene comprising 1 to 3 heteroatoms independently selected from the group consisting of N, O and S;
L² is selected from the group consisting of a bond, —O—, —NR′—, —C(O)—, C₁-C₃alkylene, —NR′—C₁-C₉alkylene-NR′—, *C₁-C₉alkylene-NR′—,*NR′—C₁-C₉alkylene, *C(O)NR′—C₁-C₆alkylene, polyethylene glycol, —NR′-polyethylene glycol-NR′—, polyethylene glycol, *NR′-polyethylene glycol and *polyethylene glycol-NR′— wherein * denotes the point of attachment of L² to X²; or
X¹-L²-X² form a 7 to 13 membered spiroheterocyclylene comprising 1 to 4 heteroatoms independently selected from the group consisting of N, O and S;
L³ is selected from the group consisting of a bond, C₁-C₆alkylene, C₂-C₆alkenylene, C₂-C₆alkynylene, C₁-C₆heteroalkylene, —C(O)—, —S(O)₂—, —O—, *C(O)—C₁-C₉alkylene, *C(O)—C₁-C₆alkylene-O, *C(O)—C₁-C₉heteroalkylene, *C₁-C₆alkylene-NH and *NH—C₁-C₆alkylene wherein * denotes the point of attachment of L³ to X²;
and wherein each R′ is independently hydrogen or C₁-C₆alkyl (preferably wherein each R′ is hydrogen).

In an embodiment, L¹ is *L^{1a}-C₄-C₇cycloalkylene **, wherein L^{1a} is selected from the group consisting of a bond, C(O), *NH—C(O) and *C₁-C₆alkylene-NH—C(O), * denotes the point of attachment to X¹, and ** denotes the point of attachment to the YAP/TAZ-TEAD PPI inhibitor. In an embodiment, L¹ is *L^{1a}-C₆cycloalkylene ** wherein L^{1a} is selected from the group consisting of C(O), *NH—C(O) and *C₁-C₃alkylene-NH—C(O) (e.g. *C₂alkylene-NH—C(O)). In an embodiment, L¹ is

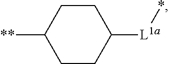

In an embodiment, X¹-L²-X² is selected from the group consisting of:
a)

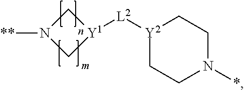

b) 7 to 11 membered spiroheterocyclylene comprising 1 to 3 heteroatoms independently selected from the group consisting of N and O (e.g. 11 membered spiroheterocyclylene comprising 2 heteroatoms which are both N);
c)

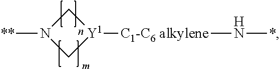

d) **NH—C₁-C₉alkylene-NH—* (e.g. **NH—C₇alkylene-NH—*), e) NH, f) C₁-C₃alkylene (e.g. C₅alkylene), g) **C₁-C₃alkylene-5 or 6 membered heteroarylene comprising 1 to 3 heteroatoms independently selected from the group consisting of N and O—* (e.g. C₅alkylene-5 membered heteroarylene comprising 3 heteroatoms which are all N—*),
h)

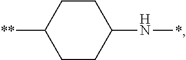

and
i)

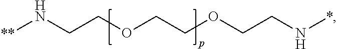

wherein:
Y¹ and Y² are each independently selected from the group consisting of CH and N, n and m are independently 1 or 2, * denotes the point of attachment to L³, ** denotes the point of attachment to L¹, and p is 0 to 23, e.g. p is 0 to 5, e.g. p is 1 to 4, e.g. p is 2 or 3, e.g. p is 2. In an embodiment, n and m are both 1 or both 2, for example both 2.

In an embodiment, L² is selected from the group consisting of: a bond, C₁-C₂alkylene, —O— and —C(O)—.

In an embodiment, L³ is selected from the group consisting of: bond, —C(O)—, *C(O)—C₁-C₃alkylene-O, C₁-C₃alkylene (e.g. C₂-C₅alkylene) and *C(O)—C₁-C₃alkylene wherein * denotes the point of attachment to X².

In an embodiment, the ligase binder is of the formula LIG-1:

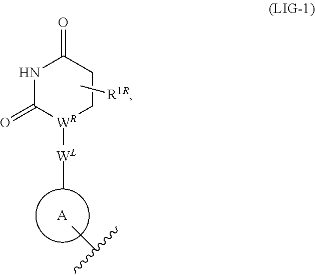

wherein [symbol] indicates the point of attachment to the linker, R^{1R} is selected from the group consisting of H, C₁-C₃alkyl, O—C₁-C₃alkyl, halo (e.g. fluoro or chloro), C₁-C₃haloalkyl, and C₁-C₃hydroxyalkyl, W^{R} is selected from the group consisting of N and CH, W^{L} is selected from the group consisting of bond and CH₂ (preferably W^{L} is a bond), and

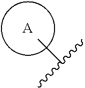

is selected from the group consisting of:

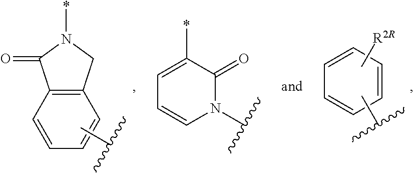

wherein * indicates the point of attachment to W^{L}, R^{2R} is selected from the group consisting of H, C₁-C₃alkyl, O—C₁-C₃alkyl and halo (e.g. fluoro or chloro), and with the caveat that when

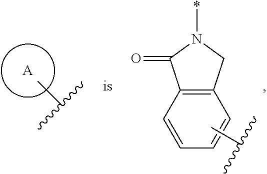

W^{R} is CH. In an embodiment, R^{1R} is H. In an embodiment, the ligase binder is selected from the group consisting of:

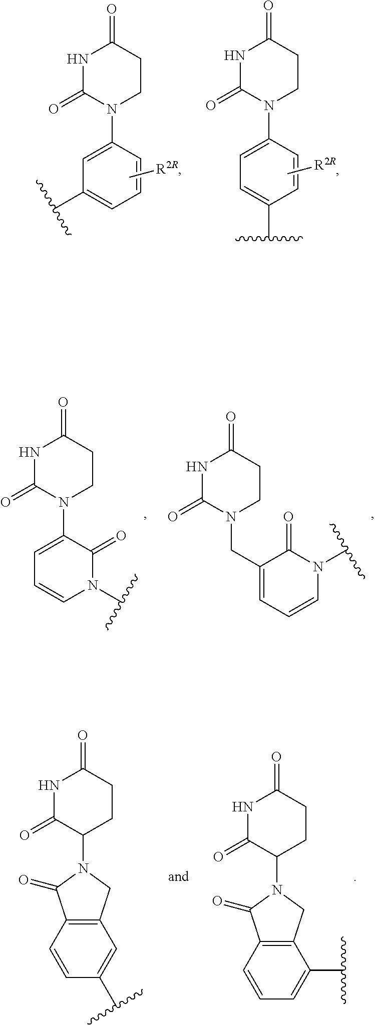

In an embodiment, the ligase binder is selected from the group consisting of

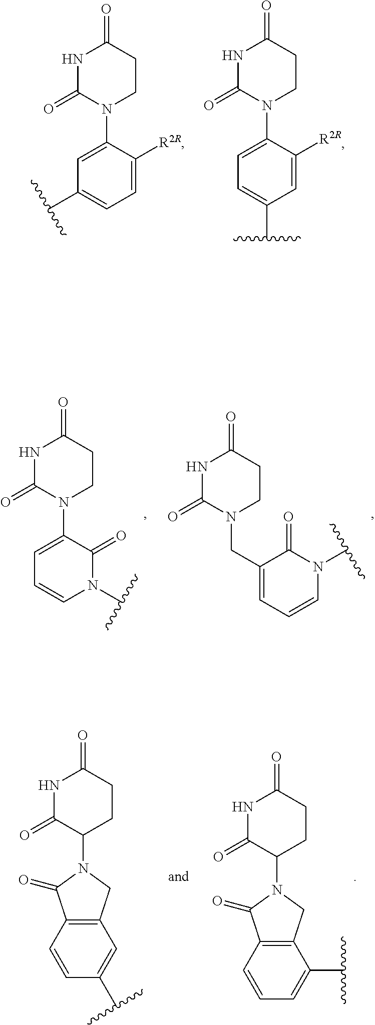

In an embodiment, the ligase binder is selected from the group consisting of:

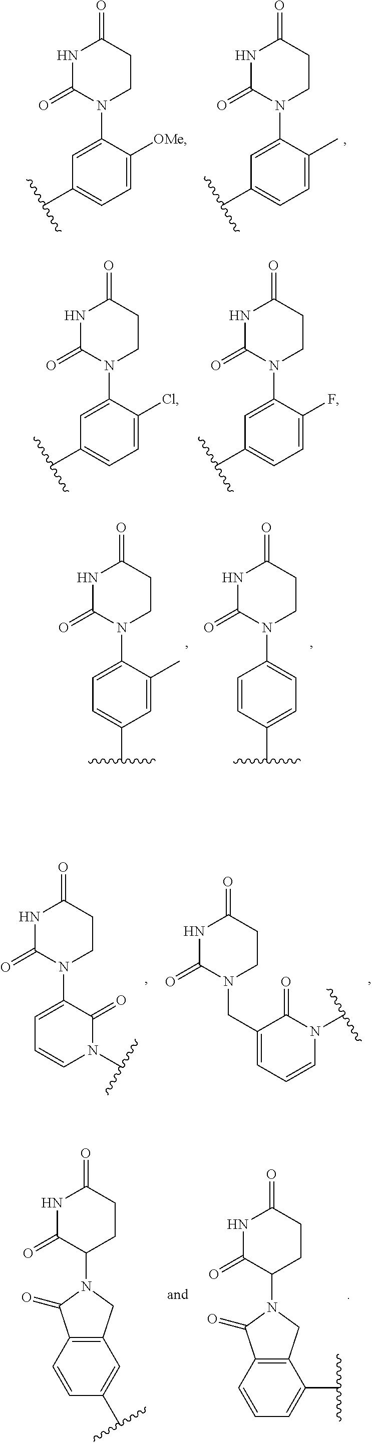

In an embodiment, the ligase binder is selected from the group consisting of:

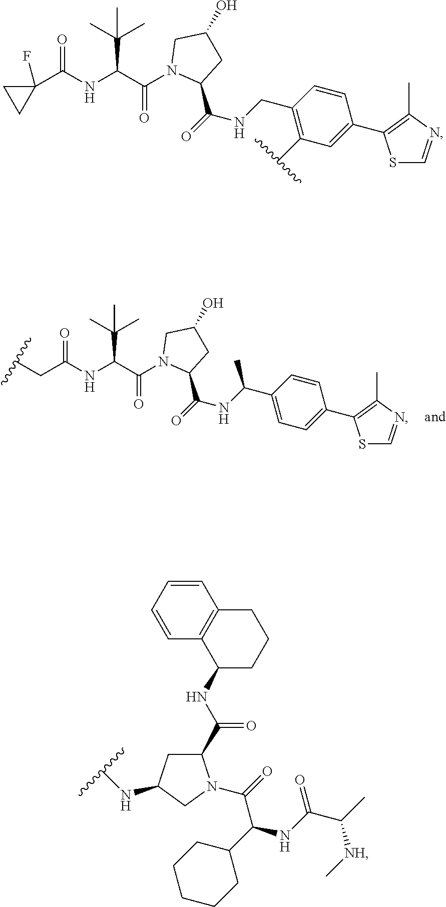 [SMILES: *c1cc(-c2scnc2C)ccc1CNC(=O)[C@@H]1C[C@@H](O)CN1C(=O)[C@@H](NC(=O)C1(F)CC1)C(C)(C)C ; *CC(=O)N[C@H](C(=O)N1C[C@H](O)C[C@H]1C(=O)N[C@@H](C)c1ccc(-c2scnc2C)cc1)C(C)(C)C ; *N[C@H]1C[C@@H](C(=O)N[C@@H]2CCCc3ccccc32)N(C(=O)[C@@H](NC(=O)[C@H](C)NC)C2CCCCC2)C1]

wherein [symbol] indicates the point of attachment to the linker.

In another aspect, the invention provides a pharmaceutical composition comprising the conjugate or pharmaceutically acceptable salt thereof according to the first aspect of the invention, and one or more pharmaceutically acceptable carriers.

In another aspect, the invention provides a combination comprising the conjugate or pharmaceutically acceptable salt thereof according to the first aspect of the invention, and one or more therapeutically active agents.

In another aspect, the invention provides the conjugate or pharmaceutically acceptable salt thereof according to the first aspect of the invention for use as a medicament.

In another aspect, the invention provides the conjugate or pharmaceutically acceptable salt thereof according to the first aspect of the invention for use in the treatment of cancer, preferably wherein the cancer is selected from breast cancer, lung cancer, ovarian cancer, colorectal cancer, malignant pleural mesothelioma, pancreatic cancer, prostate cancer, gastric cancer, esophageal cancer, liver cancer and bone cancer.

In another aspect, the invention provides the conjugate or pharmaceutically acceptable salt thereof according to the first aspect of the invention for use in the treatment of a disease or condition mediated by YAP overexpression and/or YAP amplification and/or YAP/TAZ-TEAD interaction.

Unless specified otherwise, the term “compound(s) of the present invention” or “conjugate(s) of the present invention” refers to the conjugates of formula (I) and subformulae thereof, and exemplified compounds, and salts thereof, as well as all stereoisomers (including diastereoisomers and enantiomers), rotamers, tautomers and isotopically labeled compounds (including deuterium substitutions), as well as inherently formed moieties.

Various (enumerated) embodiments of the invention are described herein. It will be recognized that features specified in each embodiment may be combined with other specified features to provide further embodiments of the present invention.

Embodiment 1. A conjugate of Formula (I):

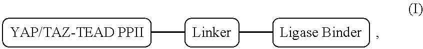

or a pharmaceutically acceptable salt thereof, wherein:
the YAP/TAZ-TEAD PPI inhibitor is a group that is capable of binding to TEAD;
the Linker is a group that covalently links the YAP/TAZ-TEAD PPI inhibitor to the Ligase Binder; and
the Ligase Binder is a group that is capable of binding to a ligase (e.g., Cereblon E3 Ubiquitin ligase).

Embodiment 2. The conjugate or pharmaceutically acceptable salt thereof according to Embodiment 1, wherein the YAP/TAZ-TEAD PPI inhibitor has a formula (Ia)

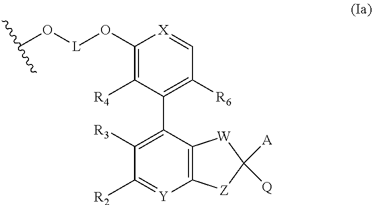

wherein:
L is C₁-C₃alkylene;
W is selected from O; and CH—R_{w};
X is selected from CH; and N;
Y is selected from CH; and N;
Z is selected from CH₂; O; and NH;
wherein when Y is N, W is CH—R_{w}, and Z is O;
A is selected from
(i) phenyl, which phenyl is optionally substituted with halo or haloC₁-C₃alkoxy;
(ii) a 5- or 6-membered aromatic heterocyclic ring comprising at least one heteroatom selected from N, O, and S, preferably from N and S, which aromatic heterocyclic ring is optionally substituted with hydroxy; C₁-C₃alkoxy; or oxo; and
(iii)

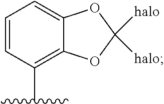

R_{w} is selected from (i) hydrogen; (ii) hydroxy; (iii) C₁-C₃alkoxy; (iv) hydroxyC₁-C₃alkyl; (v) C₁-C₃alkyl; and (vi) C₁-C₃alkoxy-C₁-C₃alkyl;
Q is selected from
   (i) —C(R⁷)₂—N(R⁸)—R₁;
   (ii) 9- or 10-membered partially saturated heteroaryl comprising at least one N heteroatom; and
   (iii) 4-, 5- or 6-membered saturated heterocyclic ring comprising at least one heteroatom or heteroatom group selected from N, O, S, —S(═O) and —S(═O)₂, with the proviso that at least one N heteroatom is present, wherein the heterocyclic ring is unsubstituted or substituted with one or more substituents independently selected from hydroxy, C₁-C₃alkyl, C₁-C₃alkoxy, halo and C₁-C₃alkylene forming a bridge between two ring atoms of the saturated heterocyclic ring, thus forming a bridged bicyclic structure;
R₁ is selected from (i) hydrogen, (ii) C₁-C₃alkyl (wherein the alkyl is in one embodiment optionally deuterated, e.g. perdeuterated), and (iii) (CH₂)₀₋₂R₁ₐ;
R₁ₐ is selected from
   (i) hydroxyC₁-C₄alkyl,
   (ii) C₁-C₃alkoxy,
   (iii) a 5- or 6-membered saturated heterocyclic ring comprising at least one heteroatom selected from N and O, which saturated heterocyclic ring is optionally substituted once or more than once independently with C₁-C₃alkyl, (CH₂)₀₋₁C(O)di(C₁-C₃alkyl)amino, SO₂C₁-C₃alkyl, C(O)C₁-C₃alkyl, or oxo,
   (iv) C₃-C₆cycloalkyl optionally substituted once or more than once independently with hydroxy, hydroxyC₁-C₄alkyl, C₁-C₃alkoxy (preferably C₁-C₄alkoxy), C(O)OC₁-C₃alkyl, CO₂H, SO₂C₁-C₃alkyl, haloC₁-C₃alkyl, NHR^{1b}, (CH₂)₀₋₁C(O)NR^{1c}R^{1d} (preferably C(O)NR^{1c}R^{1d}), C₁-C₆alkyl, haloC₁-C₃alkoxy-C₁-C₃alkyl, halo, a 5- or 6-membered aromatic heterocyclic ring comprising at least one heteroatom selected from N, O, and S, or with two R^{1e} groups,
      wherein the two R^{1e} attached at the same carbon atom form together with the carbon atom to which they are attached a 5-membered saturated heterocyclic ring comprising at least one heteroatom selected from N (which is preferred) and O, or a C₃-C₆cycloalkyl, which saturated heterocyclic ring or cycloalkyl are optionally substituted with hydroxy or oxo;
R^{1b} is selected from (i) C(O)C₁-C₃alkyl, and (ii) SO₂C₁-C₃alkyl,
R^{1c} and R^{1d} are each independently selected from (i) hydrogen, (ii) C₁-C₃alkyl, and (iii) hydroxyC₁-C₄alkyl, preferably from (i) hydrogen and (ii) C₁-C₃alkyl,
R₂ is selected from (i) hydrogen, and (ii) halo,
R₃ is selected from (i) halo, (ii) haloC₁-C₃alkyl, especially from halo and mono-, di- or preferably tri-halomethyl, and (iii) cyano,
R₄ is selected from (i) hydrogen, (ii) halo, and (iii) C₁-C₃alkyl, especially from hydrogen, halo and methyl;
R₆ is selected from (i) hydrogen, (ii) cyano, (iii) C(O)NHR^{6a}, (iv) NHR^{6b}, and (v) C₁-C₃alkoxy substituted with NH₂ or hydroxy;
R^{6a} is selected from (i) hydrogen, (ii) C₁-C₃alkyl, (iii) C₃-C₆cycloalkyl, (iv) a 5- or 6-membered aromatic heterocyclic ring comprising at least one heteroatom selected from N, O, and S, preferably at least one N heteroatom, which aromatic heterocyclic ring is optionally substituted with C₁-C₃alkyl;
R^{6b} is C₁-C₃alkyl substituted with NH₂ or hydroxy;
R⁷ is each independently selected from hydrogen and C₁-C₃alkyl; and
R⁸ is hydrogen or C₁-C₃-alkyl, especially hydrogen or methyl;
and wherein [symbol] indicates the point of attachment to the linker.

Embodiment 3. The conjugate or pharmaceutically acceptable salt thereof according to Embodiment 1, wherein the YAP/TAZ-TEAD PPI inhibitor has a formula (Ib)

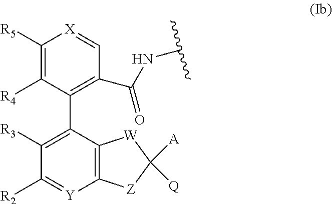

wherein:
W is selected from O; and CH—R_{w};
X is selected from CH; and N;
Y is selected from CH; and N;
Z is selected from CH₂; O; and NH;
wherein when Y is N, W is CH—R_{w}, and Z is O;
A is selected from
(i) phenyl, which phenyl is optionally substituted with halo or haloC₁-C₃alkoxy;
(ii) a 5- or 6-membered aromatic heterocyclic ring comprising at least one heteroatom selected from N, O, and S, preferably from N and S, which aromatic heterocyclic ring is optionally substituted with hydroxy; C₁-C₃alkoxy; or oxo; and
(iii)

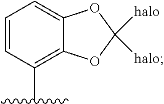

R_{w} is selected from (i) hydrogen; (ii) hydroxy; (iii) C₁-C₃alkoxy; (iv) hydroxyC₁-C₃alkyl; (v) C₁-C₃alkyl; and (vi) C₁-C₃alkoxy-C₁-C₃alkyl;
Q is selected from
   (i) —C(R⁷)₂—N(R⁸)—R₁;
   (ii) 9- or 10-membered partially saturated heteroaryl comprising at least one N heteroatom; and
   (iii) 4-, 5- or 6-membered saturated heterocyclic ring comprising at least one heteroatom or heteroatom group selected from N, O, S, —S(═O) and —S(═O)₂, with the proviso that at least one N heteroatom is present, wherein the heterocyclic ring is unsubstituted or substituted with one or more substituents independently selected from hydroxy, C₁-C₃alkyl, C₁-C₃alkoxy, halo and C₁-C₃alkylene forming a bridge between two ring atoms of the saturated heterocyclic ring, thus forming a bridged bicyclic structure;
R₁ is selected from (i) hydrogen, (ii) C₁-C₆alkyl (wherein the alkyl is in one embodiment optionally deuterated, e.g. perdeuterated), and (iii) (CH₂)₀₋₂R₁ₐ;
R₁ₐ is selected from
   (i) hydroxyC₁-C₄alkyl,
   (ii) C₁-C₃alkoxy,
   (iii) a 5- or 6-membered saturated heterocyclic ring comprising at least one heteroatom selected from N and O, which saturated heterocyclic ring is optionally substituted once or more than once independently with C₁-C₃alkyl, (CH₂)₀₋₁C(O)di(C₁-C₃alkyl)amino, SO₂C₁-C₃alkyl, C(O)C₁-C₃alkyl, or oxo,
   (iv) C₃-C₆cycloalkyl optionally substituted once or more than once independently with hydroxy, hydroxyC₁-C₄alkyl, C₁-C₆alkoxy (preferably C₁-C₄alkoxy), C(O)OC₁-C₃alkyl, CO₂H, SO₂C₁-C₃alkyl, haloC₁-C₃alkyl, NHR^{1b}, (CH₂)₀₋₁C(O)NR^{1c}R^{1d} (preferably C(O)NR^{1c}R^{1d}), C₁-C₆alkyl, haloC₁-C₃alkoxy-C₁-C₃alkyl, halo, a 5- or 6-membered aromatic heterocyclic ring comprising at least one heteroatom selected from N, O, and S, or with two R^{1e} groups,
      wherein the two R^{1e} attached at the same carbon atom form together with the carbon atom to which they are attached a 5-membered saturated heterocyclic ring comprising at least one heteroatom selected from N (which is preferred) and O, or a C₃-C₆cycloalkyl, which saturated heterocyclic ring or cycloalkyl are optionally substituted with hydroxy or oxo;
R^{1b} is selected from (i) C(O)C₁-C₃alkyl, and (ii) SO₂C₁-C₃alkyl,
R^{1c} and R^{1d} are each independently selected from (i) hydrogen, (ii) C₁-C₃alkyl, and (iii) hydroxyC₁-C₄alkyl, preferably from (i) hydrogen and (ii) C₁-C₃alkyl,
R₂ is selected from (i) hydrogen, and (ii) halo,
R₃ is selected from (i) halo, (ii) haloC₁-C₃alkyl, especially from halo and mono-, di- or preferably tri-halomethyl, and (iii) cyano,
R₄ is selected from (i) hydrogen, (ii) halo, and (iii) C₁-C₃alkyl, especially from hydrogen, halo and methyl;
R₅ is selected from
   (i) hydrogen,
   (ii) C₁-C₃alkoxy optionally substituted with C₃-C₆cycloalkyl, CO₂H, SO₂C₁-C₃alkyl, a 5- or 6-membered aromatic heterocyclic ring comprising at least one heteroatom selected from N, O, and S, preferably at least one N heteroatom, or a 5- or 6-membered saturated heterocyclic ring comprising at least one heteroatom selected from N and O, which ring is optionally substituted with C(O)C₁-C₃alkyl,
   (iii) halo,
   (iv) hydroxyC₁-C₆alkoxy (where the alkoxy part is in one embodiment optionally deuterated, e.g. perdeuterated),
   (v) haloC₁-C₃alkoxy optionally substituted with hydroxy,
   (vi) S-haloC₁-C₃alkyl optionally substituted with hydroxy,
   (vii) C₁-C₃alkoxyC₁-C₃alkoxy,
   (viii) NR^{5a}R^{5b},
   (ix) C₁-C₃alkyl,
   (x) a 5- or 6-membered aromatic heterocyclic ring comprising at least one heteroatom selected from N, O, and S, preferably at least one N heteroatom, and
   (xi) hydroxy;

R^{5a} and R^{5b} are each independently selected from (i) hydrogen, and (ii) C₁-C₃alkyl,
or
R^{5a} and R^{5b} together with the nitrogen atom to which they are attached form a 5- or 6-membered saturated heterocyclic ring, which saturated heterocyclic ring optionally in addition carries a hydroxy group;
R⁷ is each independently selected from hydrogen and C₁-C₃alkyl, and
R⁸ is hydrogen or C₁-C₃-alkyl, especially hydrogen or methyl;
and wherein [symbol] indicates the point of attachment to the linker.

Embodiment 4. The conjugate or pharmaceutically acceptable salt thereof according to Embodiment 1, wherein the YAP/TAZ-TEAD PPI inhibitor has a formula (Ic)

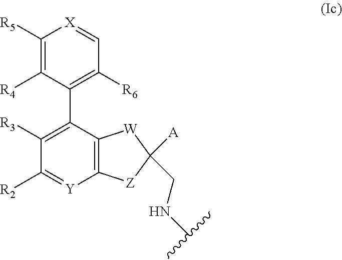

wherein:
W is selected from O; and CH—R_{w};
X is selected from CH; and N;
Y is selected from CH; and N;
Z is selected from CH₂; O; and NH;
wherein when Y is N, W is CH—R_{w}, and Z is O;
A is selected from
(i) phenyl, which phenyl is optionally substituted with halo or haloC₁-C₃alkoxy;
(ii) a 5- or 6-membered aromatic heterocyclic ring comprising at least one heteroatom selected from N, O, and S, preferably from N and S, which aromatic heterocyclic ring is optionally substituted with hydroxy; C₁-C₃alkoxy; or oxo; and
(iii)

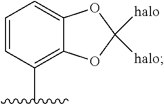

R_{w} is selected from (i) hydrogen; (ii) hydroxy; (iii) C₁-C₃alkoxy; (iv) hydroxyC₁-C₃alkyl; (v) C₁-C₃alkyl; and (vi) C₁-C₃alkoxy-C₁-C₃alkyl;
R₂ is selected from (i) hydrogen, and (ii) halo,
R₃ is selected from (i) halo, (ii) haloC₁-C₃alkyl, especially from halo and mono-, di- or preferably tri-halomethyl, and (iii) cyano,
R₄ is selected from (i) hydrogen, (ii) halo, and (iii) C₁-C₃alkyl, especially from hydrogen, halo and methyl;
R₅ is selected from
   (i) hydrogen,
   (ii) C₁-C₃alkoxy optionally substituted with C₃-C₆cycloalkyl, CO₂H, SO₂C₁-C₃alkyl, a 5- or 6-membered aromatic heterocyclic ring comprising at least one heteroatom selected from N, O, and S, preferably at least one N heteroatom, or a 5- or 6-membered saturated heterocyclic ring comprising at least one heteroatom selected from N and O, which ring is optionally substituted with C(O)C₁-C₃alkyl,
   (iii) halo,
   (iv) hydroxyC₁-C₃alkoxy (where the alkoxy part is in one embodiment optionally deuterated, e.g. perdeuterated),
   (v) haloC₁-C₃alkoxy optionally substituted with hydroxy,
   (vi) S-haloC₁-C₃alkyl optionally substituted with hydroxy,
   (vii) C₁-C₃alkoxyC₁-C₃alkoxy,
   (viii) NR^{5a}R^{5b}
   (ix) C₁-C₃alkyl,
   (x) a 5- or 6-membered aromatic heterocyclic ring comprising at least one heteroatom selected from N, O, and S, preferably at least one N heteroatom, and
   (xi) hydroxy;
R^{5a} and R^{5b} are each independently selected from (i) hydrogen, and (ii) C₁-C₃alkyl,
or
R^{5a} and R^{5b} together with the nitrogen atom to which they are attached form a 5- or 6-membered saturated heterocyclic ring, which saturated heterocyclic ring optionally in addition carries a hydroxy group;
R₆ is selected from (i) hydrogen, (ii) cyano, (iii) C(O)NHR^{6a}, (iv) NHR^{6b}, and (v) C₁-C₃alkoxy substituted with NH₂ or hydroxy,
R^{6a} is selected from (i) hydrogen, (ii) C₁-C₃alkyl, (iii) C₃-C₆cycloalkyl, (iv) a 5- or 6-membered aromatic heterocyclic ring comprising at least one heteroatom selected from N, O, and S, preferably at least one N heteroatom, which aromatic heterocyclic ring is optionally substituted with C₁-C₃alkyl;
R^{6b} is C₁-C₃alkyl substituted with NH₂ or hydroxy;
and wherein [symbol] indicates the point of attachment to the linker.

Embodiment 5. The conjugate or pharmaceutically acceptable salt thereof according to Embodiment 1 or 2, wherein the YAP/TAZ-TEAD PPI inhibitor has a formula (Ia-i)

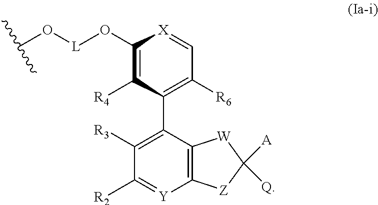

Embodiment 6. The conjugate or pharmaceutically acceptable salt thereof according to any one of Embodiments 1, 2 and 5, wherein the YAP/TAZ-TEAD PPI inhibitor has a formula (Ia-ii)

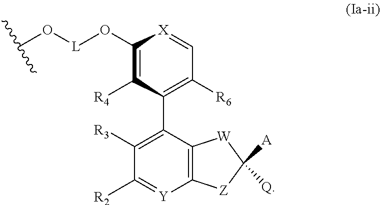

Embodiment 7. The conjugate or pharmaceutically acceptable salt thereof according to any one of Embodiments 1, 2, 5 and 6, wherein the YAP/TAZ-TEAD PPI inhibitor has a formula (Ia-ii)

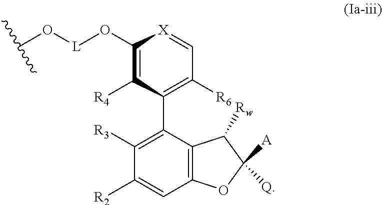

Embodiment 8. The conjugate or pharmaceutically acceptable salt thereof according to Embodiment 1 or 3, wherein the YAP/TAZ-TEAD PPI inhibitor has a formula (Ib-i)

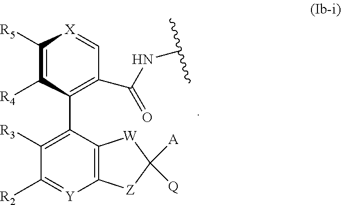

Embodiment 9. The conjugate or pharmaceutically acceptable salt thereof according to any one of Embodiments 1, 3 and 8, wherein the YAP/TAZ-TEAD PPI inhibitor has a formula (Ib-ii)

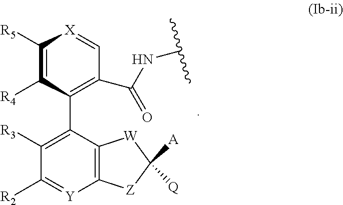

Embodiment 10. The conjugate or pharmaceutically acceptable salt thereof according to any one of Embodiments 1, 3, 8 and 9, wherein the YAP/TAZ-TEAD PPI inhibitor has a formula (Ib-iii)

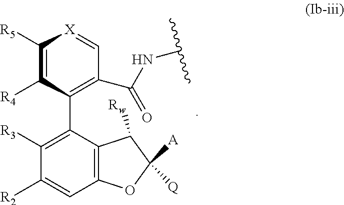

Embodiment 11. The conjugate or pharmaceutically acceptable salt thereof according to Embodiment 1 or 4, wherein the YAP/TAZ-TEAD PPI inhibitor has a formula (Ic-i)

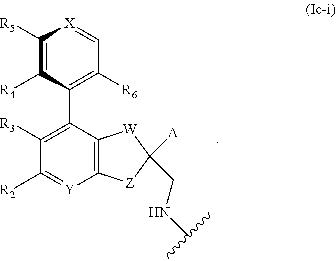

Embodiment 12. The conjugate or pharmaceutically acceptable salt thereof according to any one of Embodiments 1, 4 and 11, wherein the YAP/TAZ-TEAD PPI inhibitor has a formula (Ic-ii)

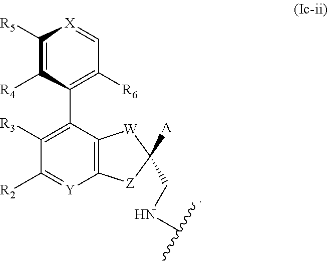

Embodiment 13. The conjugate or pharmaceutically acceptable salt thereof according to any one of Embodiments 1, 4, 11 and 12, wherein the YAP/TAZ-TEAD PPI inhibitor has a formula (Ic-iii)

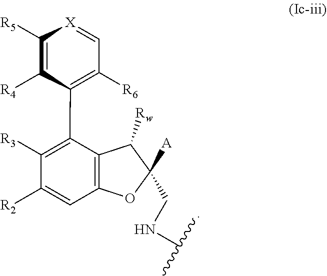

Embodiment 14. The conjugate or pharmaceutically acceptable salt thereof according to any one of the preceding embodiments, wherein:
W is CH—R_{w};
X is selected from CH; and N;
Y is CH;
Z is O;
A is selected from
(i) phenyl, which phenyl is optionally substituted with halo or haloC₁-C₃alkoxy;
(ii) a 5- or 6-membered aromatic heterocyclic ring comprising at least one heteroatom selected from N, O, and S, preferably from N and S, which aromatic heterocyclic ring is optionally substituted with hydroxy; C₁-C₃alkoxy; or oxo; for example, A is phenyl;
R_{w} is selected from (i) hydrogen; and (ii) C₁-C₃alkyl;
R₂ is halo;
R₃ is halo; and
R₄ is halo.

Embodiment 15. The conjugate of formula (Ia) according to any one of Embodiments 2, and 5 to 7, wherein
L is C₁-C₆alkylene;
W is CH—R_{w};
X is selected from CH; and N;
Y is CH;
Z is O;
A is selected from
(i) phenyl, which phenyl is optionally substituted with halo or haloC₁-C₃alkoxy;
(ii) a 5- or 6-membered aromatic heterocyclic ring comprising at least one heteroatom selected from N, O, and S, preferably from N and S, which aromatic heterocyclic ring is optionally substituted with hydroxy; C₁-C₃alkoxy; or oxo; for example, A is phenyl;
R_{w} is selected from (i) hydrogen; and (ii) C₁-C₃alkyl; Q is selected from
   (i) —C(R⁷)₂—N(R⁸)—R₁;
   (ii) 4-, 5- or 6-membered saturated heterocyclic ring comprising at least one heteroatom or heteroatom group selected from N, O, S, —S(═O) and —S(═O)₂, with the proviso that at least one N heteroatom is present, wherein the heterocyclic ring is unsubstituted or substituted with one or more substituents independently selected from hydroxy, C₁-C₃alkyl, C₁-C₃alkoxy, halo and C₁-C₃alkylene forming a bridge between two ring atoms of the saturated heterocyclic ring, thus forming a bridged bicyclic structure;
R₁ is selected from (i) hydrogen, (ii) C₁-C₆alkyl (wherein the alkyl is in one embodiment optionally deuterated, e.g. perdeuterated), and (iii) (CH₂)₀₋₂R₁ₐ;
R₁ₐ is selected from
   (i) a 5- or 6-membered saturated heterocyclic ring comprising at least one heteroatom selected from N and O, which saturated heterocyclic ring is optionally substituted once or more than once independently with C₁-C₃alkyl, (CH₂)₀₋₁C(O)di(C₁-C₃alkyl)amino, SO₂C₁-C₃alkyl, C(O)C₁-C₃alkyl, or oxo,
   (ii) C₃-C₆cycloalkyl optionally substituted once or more than once independently with hydroxy, hydroxyC₁-C₄alkyl, C₁-C₃alkoxy, C(O)OC₁-C₃alkyl, CO₂H, SO₂C₁-C₃alkyl, haloC₁-C₃alkyl, NHR^{1b}, (CH₂)₀₋₁C(O)NR^{1c}R^{1d}, C₁-C₃alkyl, haloC₁-C₃alkoxy-C₁-C₃alkyl, halo, a 5- or 6-membered aromatic heterocyclic ring comprising at least one heteroatom selected from N, O, and S, or with two R^{1e} groups,
      wherein the two R^{1e} attached at the same carbon atom form together with the carbon atom to which they are attached a 5-membered saturated heterocyclic ring comprising at least one heteroatom selected from N (which is preferred) and O, or a C₃-C₆cycloalkyl, which saturated heterocyclic ring or cycloalkyl are optionally substituted with hydroxy or oxo;
R^{1b} is selected from (i) C(O)C₁-C₃alkyl, and (ii) SO₂C₁-C₃alkyl,
R^{1c} and R^{1d} are each independently selected from (i) hydrogen, (ii) C₁-C₃alkyl, and (iii) hydroxyC₁-C₄alkyl, preferably from (i) hydrogen and (ii) C₁-C₃alkyl,
R₂ is halo;
R₃ is halo;
R₄ is halo;
R₆ is C(O)NHR^{6a};
R^{6a} is selected from (i) hydrogen, (ii) C₁-C₃alkyl, and (iii) C₃-C₆cycloalkyl;
R⁷ is each independently selected from hydrogen and C₁-C₃alkyl; and
R⁸ is hydrogen or C₁-C₃-alkyl, especially hydrogen or methyl.

Embodiment 16. The conjugate of formula (Ib) according to any one of Embodiments 3, and 8 to 10, wherein
W is CH—R_{w};
X is selected from CH; and N;
Y is CH;
Z is O;
A is selected from
(i) phenyl, which phenyl is optionally substituted with halo or haloC₁-C₃alkoxy;
(ii) a 5- or 6-membered aromatic heterocyclic ring comprising at least one heteroatom selected from N, O, and S, preferably from N and S, which aromatic heterocyclic ring is optionally substituted with hydroxy; C₁-C₃alkoxy; or oxo; for example, A is phenyl;
R_{w} is selected from (i) hydrogen; and (ii) C₁-C₃alkyl;
Q is selected from
   (i) —C(R⁷)₂—N(R⁸)—R₁;
   (ii) 4-, 5- or 6-membered saturated heterocyclic ring comprising at least one heteroatom or heteroatom group selected from N, O, S, —S(═O) and —S(═O)₂, with the proviso that at least one N heteroatom is present, wherein the heterocyclic ring is unsubstituted or substituted with one or more substituents independently selected from hydroxy, C₁-C₃alkyl, C₁-C₃alkoxy, halo and C₁-C₃alkylene forming a bridge between two ring atoms of the saturated heterocyclic ring, thus forming a bridged bicyclic structure;
R₁ is selected from (i) hydrogen, (ii) C₁-C₆alkyl (wherein the alkyl is in one embodiment optionally deuterated, e.g. perdeuterated), and (iii) (CH₂)₀₋₂R₁ₐ;
R₁ₐ is selected from
   (i) a 5- or 6-membered saturated heterocyclic ring comprising at least one heteroatom selected from N and O, which saturated heterocyclic ring is optionally substituted once or more than once independently with C₁-C₃alkyl, (CH₂)₀₋₁C(O)di(C₁-C₃alkyl)amino, SO₂C₁-C₃alkyl, C(O)C₁-C₃alkyl, or oxo,
   (ii) C₃-C₆cycloalkyl optionally substituted once or more than once independently with hydroxy, hydroxyC₁-C₄alkyl, C₁-C₃alkoxy, C(O)OC₁-C₃alkyl, CO₂H, SO₂C₁-C₃alkyl, haloC₁-C₃alkyl, NHR^{1b}, (CH₂)₀₋₁C(O)NR^{1c}R^{1d}, C₁-C₆alkyl, haloC₁-C₃alkoxy-C₁-C₃alkyl, halo, a 5- or 6-membered aromatic heterocyclic ring comprising at least one heteroatom selected from N, O, and S, or with two R^{1e} groups,
      wherein the two R^{1e} attached at the same carbon atom form together with the carbon atom to which they are attached a 5-membered saturated heterocyclic ring comprising at least one heteroatom selected from N (which is preferred) and O, or a C₃-C₆cycloalkyl, which saturated heterocyclic ring or cycloalkyl are optionally substituted with hydroxy or oxo;
R^{1b} is selected from (i) C(O)C₁-C₃alkyl, and (ii) SO₂C₁-C₃alkyl,
R^{1c} and R^{1d} are each independently selected from (i) hydrogen, (ii) C₁-C₃alkyl, and (iii) hydroxyC₁-C₄alkyl, preferably from (i) hydrogen and (ii) C₁-C₃alkyl,
R₂ is halo;
R₃ is halo;
R₄ is halo;
R₅ is selected from
   (i) C₁-C₆alkoxy optionally substituted with C₃-C₆cycloalkyl,
   (ii) halo,
   (iii) hydroxyC₁-C₆alkoxy (where the alkoxy part is in one embodiment optionally deuterated, e.g. perdeuterated),
   (iv) haloC₁-C₆alkoxy,
   (v) C₁-C₃alkoxyC₁-C₃alkoxy,
   (vi) C₁-C₃alkyl, for example, R₅ is C₁-C₆alkoxy;
R⁷ is each independently selected from hydrogen and C₁-C₃alkyl; and
R⁸ is hydrogen or C₁-C₃-alkyl, especially hydrogen or methyl.

Embodiment 17. The conjugate of formula (Ic) according to any one of Embodiments 4, and 11 to 13, wherein
W is CH—R_{w};
X is selected from CH; and N;
Y is CH;
Z is O;
A is selected from
(i) phenyl, which phenyl is optionally substituted with halo or haloC₁-C₃alkoxy;
(ii) a 5- or 6-membered aromatic heterocyclic ring comprising at least one heteroatom selected from N, O, and S, preferably from N and S, which aromatic heterocyclic ring is optionally substituted with hydroxy; C₁-C₃alkoxy; or oxo; for example, A is phenyl;
R_{w} is selected from (i) hydrogen; and (ii) C₁-C₃alkyl;
R₂ is halo;
R₃ is halo;
R₄ is halo;
R₅ is selected from
   (i) C₁-C₆alkoxy optionally substituted with C₃-C₆cycloalkyl,
   (ii) halo,
   (iii) hydroxyC₁-C₆alkoxy (where the alkoxy part is in one embodiment optionally deuterated, e.g. perdeuterated),
   (iv) haloC₁-C₆alkoxy,
   (v) C₁-C₃alkoxyC₁-C₃alkoxy,
   (vi) C₁-C₃alkyl,
for example, R₅ is C₁-C₆alkoxy;
R₆ is C(O)NHR^{6a}; and
R^{6a} is selected from (i) hydrogen, (ii) C₁-C₃alkyl, and (iii) C₃-C₆cycloalkyl.

Embodiment 18. The conjugate of formula (Ib) or (Ic) according to Embodiment 16 or 17, wherein R₅ is C₁-C₆alkoxy.

Embodiment 19. The conjugate of formula (Ia) or (Ic) according to any one of Embodiments 15, and 17 to 18, wherein R₆ is —C(O) NH₂ or —C(O) NH(C₁-C₃alkyl).

Embodiment 20. The conjugate of formula (Ia) or (Ib) according to any one of Embodiments 15, 16, and 18 to 19, wherein Q is a 5- or 6-membered saturated heterocyclic ring comprising one N heteroatom or is —CH₂—NH₂.

Embodiment 21. The conjugate according to any one of Embodiments 2 to 20, wherein A is phenyl.

Embodiment 22. The conjugate according to any one of Embodiments 2 to 6, 8, 9, 11, and 12, wherein W is CH—R_{w};
Y is CH; and
Z is O.

Embodiment 23. The conjugate according to any one of the preceding embodiments, wherein R² is F, R³ is Cl, and R⁴ is F.

Embodiment 24. The conjugate or pharmaceutically acceptable salt thereof according to any of the preceding embodiments, wherein the YAP/TAZ-TEAD PPI inhibitor has a formula YTT-1, YTT-2 or YTT-3:

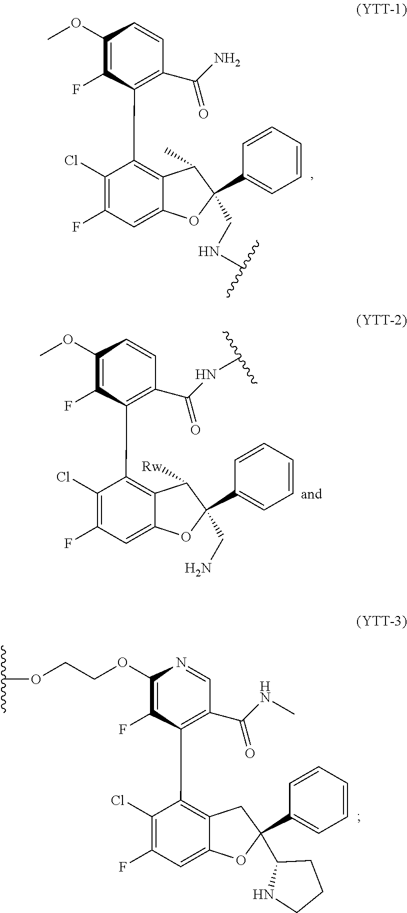

wherein R_{w} is H or CH₃ and [symbol] indicates the point of attachment to the linker.

Embodiment 25. The conjugate or pharmaceutically acceptable salt thereof according to embodiment 24, wherein the YAP/TAZ-TEAD PPI inhibitor has a formula YTT-1.

Embodiment 26. The conjugate or pharmaceutically acceptable salt thereof according to any of the preceding embodiments, wherein the linker has a formula LIN-1:
-L¹-X¹-L²-X²-L³-(LIN-1), and wherein the conjugate has a formula (II):

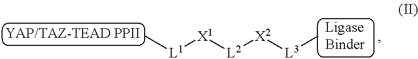

wherein:
   the YAP/TAZ-TEAD PPI inhibitor is a group that is capable of binding to TEAD;
   and
   the Ligase Binder is a group that is capable of binding to a ligase (e.g., Cereblon E3 Ubiquitin ligase);
L¹ is selected from the group consisting of a bond, —O—, —NR′—, —C(O)—, C₁-C₉alkylene, C₁-C₉heteroalkylene, *C(O)—C₁-C₃alkylene, *C(O)—C₁-C₆heteroalkylene, *C₁-C₃alkylene-C(O), *C₁-C₆ heteroalkylene-C(O), and *L^{1a}-C₄-C₇cycloalkylene, wherein * denotes the point of attachment of L¹ to X¹;
wherein L^{1a} is selected from the group consisting of C(O), *NH—C(O) and *C₁-C₃alkylene-NH—C(O) wherein * denotes the point of attachment of L^{1a} to X¹;
X¹ and X² are each independently selected from the group consisting of a bond, C₄-C₇cycloalkylene, 4 to 7 membered heterocyclylene comprising 1 to 3 heteroatoms independently selected from the group consisting of N, O and S, and 5 or 6 membered heteroarylene comprising 1 to 3 heteroatoms independently selected from the group consisting of N, O and S;
L² is selected from the group consisting of a bond, —O—, —NR′—, —C(O)—, C₁-C₃alkylene, —NR′—C₁-C₉alkylene-NR′—, *C₁-C₉alkylene-NR′—,*NR′—C₁-C₉alkylene, *C(O)NR′—C₁-C₆alkylene, polyethylene glycol, —NR′-polyethylene glycol-NR′—, polyethylene glycol, *NR′-polyethylene glycol and *polyethylene glycol-NR′— wherein * denotes the point of attachment of L² to X²; or
X¹-L²-X² form a 7 to 13 membered spiroheterocyclylene comprising 1 to 4 heteroatoms independently selected from the group consisting of N, O and S;
L³ is selected from the group consisting of a bond, C₁-C₆alkylene, C₂-C₆alkenylene, C₂-C₆alkynylene, C₁-C₆heteroalkylene, —C(O)—, —S(O)₂—, —O—, *C(O)—C₁-C₉alkylene, *C(O)—C₁-C₃alkylene-O, *C(O)—C₁-C₉heteroalkylene, *C₁-C₃alkylene-NH and *NH—C₁-C₃alkylene wherein * denotes the point of attachment of L³ to X²;
and wherein each R′ is independently hydrogen or C₁-C₃alkyl (preferably wherein each R′ is hydrogen).

Embodiment 27. The conjugate or pharmaceutically acceptable salt thereof according to Embodiment 26, wherein the YAP/TAZ-TEAD PPI inhibitor has a formula according to any one of Embodiments 2 to 25.

Embodiment 28. The conjugate or pharmaceutically acceptable salt thereof according to any one of embodiments 26 to 27 wherein L¹ is *L^{1a}-C₄-C₇cycloalkylene **, wherein L^{1a} is selected from the group consisting of a bond, C(O), *NH—C(O) and *C₁-C₃alkylene-NH—C(O), * denotes the point of attachment to X¹, and ** denotes the point of attachment to the YAP/TAZ-TEAD PPI inhibitor.

Embodiment 29. The conjugate or pharmaceutically acceptable salt thereof according to embodiment 28 wherein L¹ is *L^{1a}-C₆cycloalkylene ** wherein L^{1a} is selected from the group consisting of C(O), *NH—C(O) and *C₁-C₃alkylene-NH—C(O) (e.g. *C₂alkylene-NH—C(O)).

Embodiment 30. The conjugate or pharmaceutically acceptable salt, thereof according to embodiment 28 or 29, wherein L¹ is

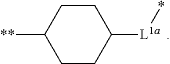

Embodiment 31. The conjugate or pharmaceutically acceptable salt thereof according to any one of embodiments 26 to 30, wherein X¹-L²-X² is selected from the group consisting of:
a)

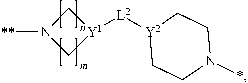

b) 7 to 11 membered spiroheterocyclylene comprising 1 to 3 heteroatoms independently selected from the group consisting of N and O (e.g. 11 membered spiroheterocyclylene comprising 2 heteroatoms which are both N); c)

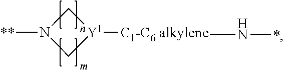

d) **NH—C₁-C₉alkylene-NH—* (e.g. **NH—C₇alkylene-NH—*), e) NH, f) C₁-C₃alkylene (e.g. C₅alkylene),g) **C₁-C₃alkylene-5 or 6 membered heteroarylene comprising 1 to 3 heteroatoms independently selected from the group consisting of N and O—* (e.g. C₅alkylene-5 membered heteroarylene comprising 3 heteroatoms which are all N—*), h)

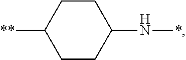

and i)

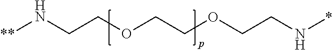

wherein: Y¹ and Y² are each independently selected from the group consisting of CH and N, n and m are independently 1 or 2, * denotes the point of attachment to L³, ** denotes the point of attachment to L¹, and p is 0 to 23, e.g. p is 0 to 5, e.g. p is 1 to 4, e.g. p is 2 or 3, e.g. p is 2.

Embodiment 32. The conjugate or pharmaceutically acceptable salt hereof according to embodiment 31, wherein X¹-L²-X² is

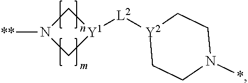

wherein: Y¹ and Y² are each independently selected from the group consisting of CH and N, n and m are independently 1 or 2, * denotes the point of attachment to L³, and ** denotes the point of attachment to L¹.

Embodiment 33. The conjugate or pharmaceutically acceptable salt thereof according to embodiment 31 or embodiment 32, wherein n and m are both 1 or both 2.

Embodiment 34. The conjugate or pharmaceutically acceptable salt thereof according to embodiment 33, wherein n and m are both 2.

Embodiment 35. The conjugate or pharmaceutically acceptable salt thereof according to any one of embodiments 31 to 34, wherein L² is selected from the group consisting of: a bond, C₁-C₃alkylene, —O— and —C(O)—.

Embodiment 36. The conjugate or pharmaceutically acceptable salt thereof according to any one of embodiments 26 to 35, wherein L³ is selected from the group consisting of: bond, —C(O)—, *C(O)—C₁-C₃alkylene-O, C₁-C₃alkylene (e.g. C₂-C₅alkylene) and *C(O)—C₁-C₃alkylene wherein * denotes the point of attachment to X².

Embodiment 37. The conjugate or pharmaceutically acceptable salt thereof according to any one of the preceding embodiments wherein the ligase binder is of the formula LIG-1:

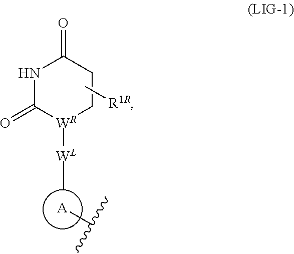

wherein [symbol] indicates the point of attachment to the linker, R^{1R} is selected from the group consisting of H, C₁-C₃alkyl, O—C₁-C₃alkyl, halo (e.g. fluoro or chloro), C₁-C₃haloalkyl, and C₁-C₃hydroxyalkyl, W^{R} is selected from the group consisting of N and CH, W^{L} is selected from the group consisting of bond and CH₂ (preferably W^{L} is a bond), and

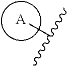

is selected from the group consisting of:

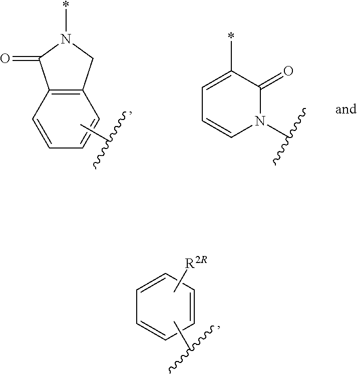

wherein * indicates the point of attachment to W^{L}, R^{2R} is selected from the group consisting of H, C₁-C₃alkyl, O—C₁-C₃alkyl and halo (e.g. fluoro or chloro), and with the caveat that when

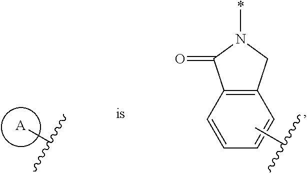

### W^{R} is CH.

Embodiment 38. The conjugate or pharmaceutically acceptable salt thereof according to Embodiment 37 wherein R^{1R} is H.

Embodiment 39. The conjugate or pharmaceutically acceptable salt thereof according to embodiment 37 or embodiment 38 wherein the ligase binder is selected from the group consisting of.

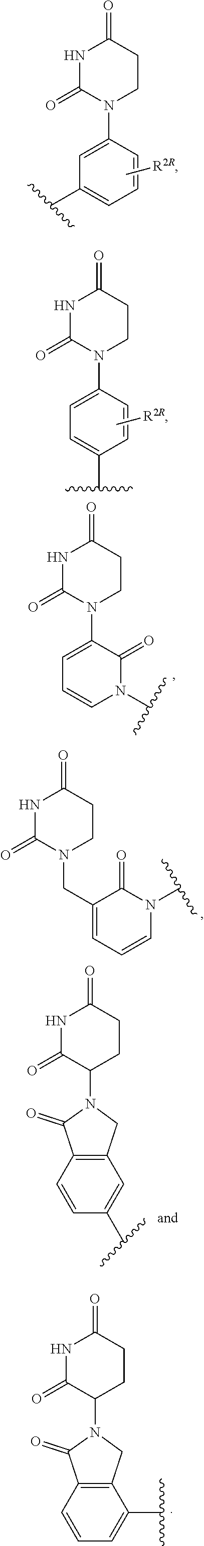

Embodiment 40. The conjugate or pharmaceutically acceptable salt thereof according to embodiment 39 wherein the ligase binder is selected from the group consisting of

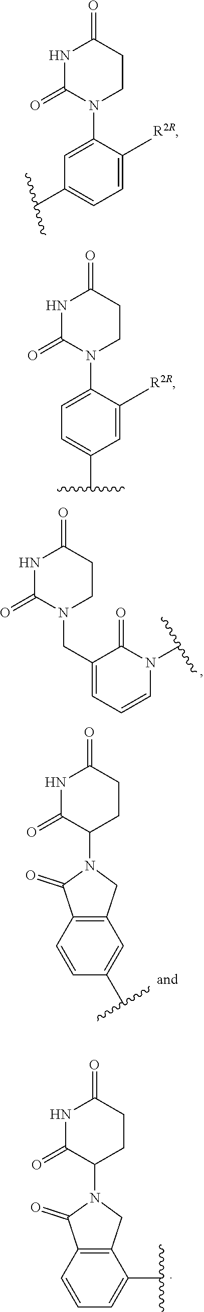

Embodiment 41. The conjugate or pharmaceutically acceptable salt thereof according to embodiment 40 wherein the ligase binder is selected from the group consisting of:

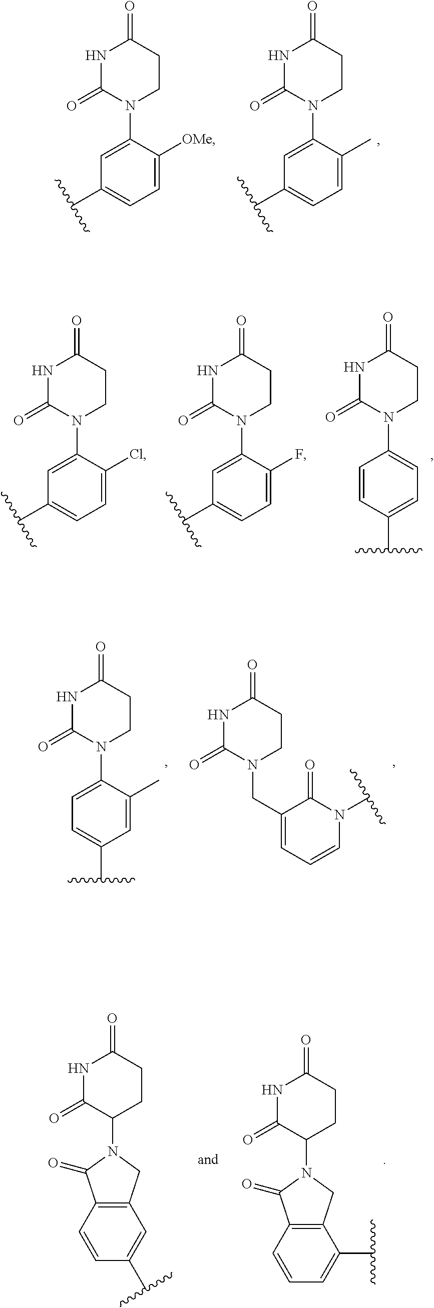

Embodiment 42. The conjugate or pharmaceutically acceptable salt hereof according to any one of embodiments 1-36 wherein the ligase binder is selected from the group consisting of:

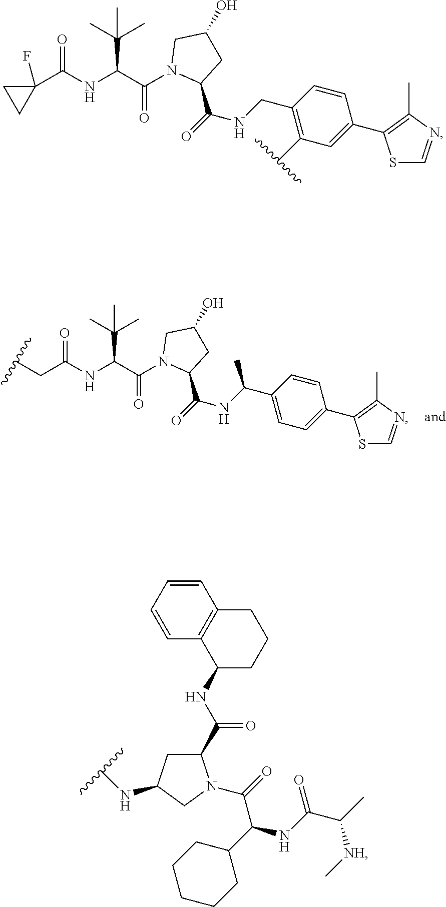 [SMILES: *c1cc(-c2scnc2C)ccc1CNC(=O)[C@@H]1C[C@@H](O)CN1C(=O)[C@@H](NC(=O)C1(F)CC1)C(C)(C)C ; *CC(=O)N[C@H](C(=O)N1C[C@H](O)C[C@H]1C(=O)N[C@@H](C)c1ccc(-c2scnc2C)cc1)C(C)(C)C ; *N[C@H]1C[C@@H](C(=O)N[C@@H]2CCCc3ccccc32)N(C(=O)[C@@H](NC(=O)[C@H](C)NC)C2CCCCC2)C1]

wherein [symbol] indicates the point of attachment to the linker.

Embodiment 43. The conjugate or pharmaceutically acceptable salt hereof according to any one of embodiments 1, and 26 to 42, wherein the YAP/TAZ-TEAD PPI inhibitor has a formula according to any one of embodiments 2 to 25.

Embodiment 44. A conjugate or pharmaceutically acceptable salt thereof selected from the group consisting of:
2-((2S,3S,4S)-5-chloro-2-((((1r,4S)-4-(4-((1-(3-(2,4-dioxotetrahydropyrimidin-1(2H)-yl)-4-methoxybenzoyl)piperidin-4-yl)methyl)piperidine-1-carbonyl)cyclohexyl)amino)methyl)-6-fluoro-3-methyl-2-phenyl-2,3-dihydrobenzofuran-4-yl)-3-fluoro-4-methoxybenzamide;
2-((2S,3S,4S)-5-chloro-2-((((1r,4S)-4-(4-((1-(3-(2,4-dioxotetrahydropyrimidin-1(2H)-yl)-4-methoxybenzoyl)piperidin-4-yl)oxy)piperidine-1-carbonyl)cyclohexyl)amino)methyl)-6-fluoro-3-methyl-2-phenyl-2,3-dihydrobenzofuran-4-yl)-3-fluoro-4-methoxybenzamide;
2-((2S,3S,4S)-5-chloro-2-((((1r,4S)-4-((2-(4-((4-(3-(2,4-dioxotetrahydropyrimidin-1(2H)-yl)-4-methoxybenzoyl) piperazin-1-yl)methyl)piperidin-1-yl)ethyl)carbamoyl)cyclohexyl)amino)methyl)-6-fluoro-3-methyl-2-phenyl-2,3-dihydrobenzofuran-4-yl)-3-fluoro-4-methoxybenzamide;
2-((2S,3S,4S)-5-chloro-2-((((1r,4S)-4-((2-(4-((1-(3-(2,4-dioxotetrahydropyrimidin-1(2H)-yl)-4-methoxybenzoyl)piperidin-4-yl)oxy)piperidin-1-yl)ethyl)carbamoyl)cyclohexyl)amino)methyl)-6-fluoro-3-methyl-2-phenyl-2,3-dihydrobenzofuran-4-yl)-3-fluoro-4-methoxybenzamide;
2-((2S,3S,4S)-5-chloro-2-((((1r,4S)-4-((2-(9-(3-(2,4-dioxotetrahydropyrimidin-1(2H)-yl)-4-methoxybenzoyl)-3,9-diazaspiro [5.5]undecan-3-yl)ethyl)carbamoyl)cyclohexyl)amino)methyl)-6-fluoro-3-methyl-2-phenyl-2,3-dihydrobenzofuran-4-yl)-3-fluoro-4-methoxybenzamide;
2-((2S,3S,4S)-5-chloro-2-((((1r,4S)-4-(4-((4-(3-(2,4-dioxotetrahydropyrimidin-1(2H)-yl)-4-methoxybenzoyl) piperazin-1-yl)methyl)piperidine-1-carbonyl)cyclohexyl)amino)methyl)-6-fluoro-3-methyl-2-phenyl-2,3-dihydrobenzofuran-4-yl)-3-fluoro-4-methoxybenzamide;
2-((2S,3S,4S)-5-chloro-2-((((1r,4S)-4-((2-(4-((1-(3-(2,4-dioxotetrahydropyrimidin-1(2H)-yl)-4-methoxybenzoyl)piperidin-4-yl)methyl)piperidin-1-yl)ethyl)carbamoyl)cyclohexyl)amino)methyl)-6-fluoro-3-methyl-2-phenyl-2,3-dihydrobenzofuran-4-yl)-3-fluoro-4-methoxybenzamide;
2-((2S,3S,4S)-5-chloro-2-((((1r,4S)-4-(3-((1-(3-(2,4-dioxotetrahydropyrimidin-1(2H)-yl)-4-methoxybenzoyl)piperidin-4-yl)oxy) azetidine-1-carbonyl)cyclohexyl)amino)methyl)-6-fluoro-3-methyl-2-phenyl-2,3-dihydrobenzofuran-4-yl)-3-fluoro-4-methoxybenzamide;
2-((2S,3S,4S)-5-chloro-2-((((1r,4S)-4-(3-((4-(3-(2,4-dioxotetrahydropyrimidin-1(2H)-yl)-4-methoxybenzoyl) piperazin-1-yl)methyl) azetidine-1-carbonyl)cyclohexyl)amino)methyl)-6-fluoro-3-methyl-2-phenyl-2,3-dihydrobenzofuran-4-yl)-3-fluoro-4-methoxybenzamide;
2-((2S,3S,4S)-5-chloro-2-((((1r,4S)-4-(1′-(3-(2,4-dioxotetrahydropyrimidin-1(2H)-yl)-4-methoxybenzoyl)-[4,4′-bipiperidine]-1-carbonyl)cyclohexyl)amino)methyl)-6-fluoro-3-methyl-2-phenyl-2,3-dihydrobenzofuran-4-yl)-3-fluoro-4-methoxybenzamide;
2-((2S,3S,4S)-5-chloro-2-((((1r,4S)-4-((2-(1′-(3-(2,4-dioxotetrahydropyrimidin-1(2H)-yl)-4-methoxybenzoyl)-[4,4′-bipiperidin]-1-yl)ethyl)carbamoyl)cyclohexyl)amino)methyl)-6-fluoro-3-methyl-2-phenyl-2,3-dihydrobenzofuran-4-yl)-3-fluoro-4-methoxybenzamide;
2-((2S,3S,4S)-5-chloro-2-((((1r,4S)-4-(4-((4-(2-(3-(2,4-dioxotetrahydropyrimidin-1(2H)-yl)-4-methylphenoxy)acetyl) piperazin-1-yl)methyl)piperidine-1-carbonyl)cyclohexyl)amino)methyl)-6-fluoro-3-methyl-2-phenyl-2,3-dihydrobenzofuran-4-yl)-3-fluoro-4-methoxybenzamide;
2-((2S,3S,4S)-5-chloro-2-((((1r,4S)-4-(4-((1-(4-chloro-3-(2,4-dioxotetrahydropyrimidin-1(2H)-yl)benzoyl)piperidin-4-yl)oxy)piperidine-1-carbonyl)cyclohexyl)amino)methyl)-6-fluoro-3-methyl-2-phenyl-2,3-dihydrobenzofuran-4-yl)-3-fluoro-4-methoxybenzamide;
2-((2S,3S,4S)-5-chloro-2-((((1r,4S)-4-(4-((1-(2-(4-(2,4-dioxotetrahydropyrimidin-1(2H)-yl)-3-methylphenoxy)acetyl)piperidin-4-yl)oxy)piperidine-1-carbonyl)cyclohexyl)amino)methyl)-6-fluoro-3-methyl-2-phenyl-2,3-dihydrobenzofuran-4-yl)-3-fluoro-4-methoxybenzamide;
2-((2S,3S,4S)-5-chloro-2-((((1r,4S)-4-(4-((1-(2-(3-((2,4-dioxotetrahydropyrimidin-1(2H)-yl)methyl)-2-oxopyridin-1(2H)-yl)ethyl)piperidin-4-yl)methyl)piperidine-1-carbonyl)cyclohexyl)amino)methyl)-6-fluoro-3-methyl-2-phenyl-2,3-dihydrobenzofuran-4-yl)-3-fluoro-4-methoxybenzamide;
2-((2S,3S,4S)-5-chloro-2-((((1r,4S)-4-(4-((1-(2-(3-((2,4-dioxotetrahydropyrimidin-1(2H)-yl)methyl)-2-oxopyridin-1(2H)-yl)acetyl)piperidin-4-yl)methyl)piperidine-1-carbonyl)cyclohexyl)amino)methyl)-6-fluoro-3-methyl-2-phenyl-2,3-dihydrobenzofuran-4-yl)-3-fluoro-4-methoxybenzamide;
2-((2S,3S,4S)-5-chloro-2-((((1r,4S)-4-(4-((1-(3-(2,4-dioxotetrahydropyrimidin-1(2H)-yl)-4-methoxybenzoyl)piperidin-4-yl)methyl)piperazine-1-carbonyl)cyclohexyl)amino)methyl)-6-fluoro-3-methyl-2-phenyl-2,3-dihydrobenzofuran-4-yl)-3-fluoro-4-methoxybenzamide;
N-(4-(1-((1S,4r)-4-((((2S,3S,4S)-4-(6-carbamoyl-2-fluoro-3-methoxyphenyl)-5-chloro-6-fluoro-3-methyl-2-phenyl-2,3-dihydrobenzofuran-2-yl)methyl)amino)cyclohexane-1-carbonyl)piperidin-4-yl)butyl)-3-(2,4-dioxotetrahydropyrimidin-1(2H)-yl)-4-methoxybenzamide;
2-((2S,3S,4S)-5-chloro-2-((((1r,4S)-4-(1′-(2-(3-(2,4-dioxotetrahydropyrimidin-1(2H)-yl)-4-methylphenoxy)acetyl)-[4,4′-bipiperidine]-1-carbonyl)cyclohexyl)amino)methyl)-6-fluoro-3-methyl-2-phenyl-2,3-dihydrobenzofuran-4-yl)-3-fluoro-4-methoxybenzamide;
2-((2S,3S,4S)-5-chloro-2-((((1r,4S)-4-(4-((1-(2-(3-(2,4-dioxotetrahydropyrimidin-1(2H)-yl)-4-methylphenoxy)acetyl)piperidin-4-yl)oxy)piperidine-1-carbonyl)cyclohexyl)amino)methyl)-6-fluoro-3-methyl-2-phenyl-2,3-dihydrobenzofuran-4-yl)-3-fluoro-4-methoxybenzamide;
2-((2S,3S,4S)-5-chloro-2-((((1r,4S)-4-(4-((1-(2-(3-(2,4-dioxotetrahydropyrimidin-1(2H)-yl)-4-methylphenoxy)acetyl)piperidin-4-yl)methyl)piperidine-1-carbonyl)cyclohexyl)amino)methyl)-6-fluoro-3-methyl-2-phenyl-2,3-dihydrobenzofuran-4-yl)-3-fluoro-4-methoxybenzamide;
2-((2S,3S,4S)-5-chloro-2-((((1r,4S)-4-(4-((1-(2-(3-(2,4-dioxotetrahydropyrimidin-1(2H)-yl)-4-methylphenoxy)acetyl)piperidin-4-yl)methyl)piperazine-1-carbonyl)cyclohexyl)amino)methyl)-6-fluoro-3-methyl-2-phenyl-2,3-dihydrobenzofuran-4-yl)-3-fluoro-4-methoxybenzamide;
2-((2S,3S,4S)-5-chloro-2-((((1r,4S)-4-((7-(2-(3-(2,4-dioxotetrahydropyrimidin-1(2H)-yl)-4-methylphenoxy) acetamido) heptyl)carbamoyl)cyclohexyl)amino)methyl)-6-fluoro-3-methyl-2-phenyl-2,3-dihydrobenzofuran-4-yl)-3-fluoro-4-methoxybenzamide;
2-((2S,3S,4S)-5-chloro-2-((((1r,4S)-4-(4-((1-(4-chloro-3-(2,4-dioxotetrahydropyrimidin-1(2H)-yl)benzoyl)piperidin-4-yl)methyl)piperidine-1-carbonyl)cyclohexyl)amino)methyl)-6-fluoro-3-methyl-2-phenyl-2,3-dihydrobenzofuran-4-yl)-3-fluoro-4-methoxybenzamide;
2-((2S,3S,4S)-5-chloro-2-((((1r,4S)-4-(4-((1-(2-(4-(2,4-dioxotetrahydropyrimidin-1(2H)-yl) phenoxy)acetyl)piperidin-4-yl)methyl)piperidine-1-carbonyl)cyclohexyl)amino)methyl)-6-fluoro-3-methyl-2-phenyl-2,3-dihydrobenzofuran-4-yl)-3-fluoro-4-methoxybenzamide;
2-((2S,3S,4S)-5-chloro-2-((((1r,4S)-4-(4-((1-(2-(4-(2,4-dioxotetrahydropyrimidin-1(2H)-yl)-3-methylphenoxy)acetyl)piperidin-4-yl)methyl)piperidine-1-carbonyl)cyclohexyl)amino)methyl)-6-fluoro-3-methyl-2-phenyl-2,3-dihydrobenzofuran-4-yl)-3-fluoro-4-methoxybenzamide;
2-((2S,3S,4S)-5-chloro-2-((((1r,4S)-4-(4-(2-(1-(3-(2,4-dioxotetrahydropyrimidin-1(2H)-yl)-4-methoxybenzoyl)piperidin-4-yl)ethyl)piperidine-1-carbonyl)cyclohexyl)amino)methyl)-6-fluoro-3-methyl-2-phenyl-2,3-dihydrobenzofuran-4-yl)-3-fluoro-4-methoxybenzamide;
2-((2S,3S,4S)-5-chloro-2-((((1r,4S)-4-(4-((1-(2-(4-chloro-3-(2,4-dioxotetrahydropyrimidin-1(2H)-yl) phenoxy)acetyl)piperidin-4-yl)methyl)piperidine-1-carbonyl)cyclohexyl)amino)methyl)-6-fluoro-3-methyl-2-phenyl-2,3-dihydrobenzofuran-4-yl)-3-fluoro-4-methoxybenzamide;
2-((2S,3S,4S)-5-chloro-2-((((1r,4S)-4-(4-((1-(2-(3-(2,4-dioxotetrahydropyrimidin-1(2H)-yl)-4-fluorophenoxy)acetyl)piperidin-4-yl)methyl)piperidine-1-carbonyl)cyclohexyl)amino)methyl)-6-fluoro-3-methyl-2-phenyl-2,3-dihydrobenzofuran-4-yl)-3-fluoro-4-methoxybenzamide;
2-((2S,3S,4S)-5-chloro-2-((((1r,4S)-4-(4-((1-(2-(3-(2,4-dioxotetrahydropyrimidin-1(2H)-yl)-4-methoxyphenoxy)acetyl)piperidin-4-yl)methyl)piperidine-1-carbonyl)cyclohexyl)amino)methyl)-6-fluoro-3-methyl-2-phenyl-2,3-dihydrobenzofuran-4-yl)-3-fluoro-4-methoxybenzamide;
2-((2S,3S,4S)-5-chloro-2-((((1r,4S)-4-((5-(2-(2,6-dioxopiperidin-3-yl)-1-oxoisoindolin-4-yl)pentyl)carbamoyl)cyclohexyl)amino)methyl)-6-fluoro-3-methyl-2-phenyl-2,3-dihydrobenzofuran-4-yl)-3-fluoro-4-methoxybenzamide;
4-((2S,4S)-5-Chloro-6-fluoro-2-phenyl-2-((S)-pyrrolidin-2-yl)-2,3-dihydrobenzofuran-4-yl)-6-(2-(2-(4-(2-(2,6-dioxopiperidin-3-yl)-1-oxoisoindolin-5-yl)-1H-1,2,3-triazol-1-yl)ethoxy)ethoxy)-5-fluoro-N-methylnicotinamide;
4-((2S,4S)-5-chloro-6-fluoro-2-phenyl-2-((S)-pyrrolidin-2-yl)-2,3-dihydrobenzofuran-4-yl)-6-(2-(2-(2-(4-(2-(2,6-dioxopiperidin-3-yl)-1-oxoisoindolin-5-yl)-1H-1,2,3-triazol-1-yl)ethoxy)ethoxy)ethoxy)-5-fluoro-N-methylnicotinamide;
4-((2S,4S)-5-chloro-6-fluoro-2-phenyl-2-((S)-pyrrolidin-2-yl)-2,3-dihydrobenzofuran-4-yl)-6-(2-(2-(2-(4-(3-(2,4-dioxotetrahydropyrimidin-1(2H)-yl)phenyl)-1H-1,2,3-triazol-1-yl)ethoxy)ethoxy)ethoxy)-5-fluoro-N-methylnicotinamide;
2-((2S,3S,4S)-5-chloro-2-(((5-(2-(2,6-dioxopiperidin-3-yl)-1-oxoisoindolin-4-yl)pentyl)amino)methyl)-6-fluoro-3-methyl-2-phenyl-2,3-dihydrobenzofuran-4-yl)-3-fluoro-4-methoxybenzamide;
2-((2S,4S)-2-(aminomethyl)-5-chloro-6-fluoro-2-phenyl-2,3-dihydrobenzofuran-4-yl)-N-(5-(2-(2,6-dioxopiperidin-3-yl)-1-oxoisoindolin-4-yl)pentyl)-3-fluoro-4-methoxybenzamide;
2-((2S,4S)-2-(aminomethyl)-5-chloro-6-fluoro-2-phenyl-2,3-dihydrobenzofuran-4-yl)-N-(5-(4-(2-(2,6-dioxopiperidin-3-yl)-1-oxoisoindolin-5-yl)-1H-1,2,3-triazol-1-yl)pentyl)-3-fluoro-4-methoxybenzamide;
(2S,4R)—N-(2-(2-(4-((1-((1S,4r)-4-((((2S,3S,4S)-4-(6-carbamoyl-2-fluoro-3-methoxyphenyl)-5-chloro-6-fluoro-3-methyl-2-phenyl-2,3-dihydrobenzofuran-2-yl)methyl)amino)cyclohexane-1-carbonyl)piperidin-4-yl)oxy)piperidin-1-yl)-2-oxoethoxy)-4-(4-methylthiazol-5-yl)benzyl)-1-((S)-2-(1-fluorocyclopropane-1-carboxamido)-3,3-dimethylbutanoyl)-4-hydroxypyrrolidine-2-carboxamide;
(2S,4R)—N-(2-(2-(((1S,4S)-4-((1S,4R)-4-((((2S,3S,4S)-4-(6-carbamoyl-2-fluoro-3-methoxyphenyl)-5-chloro-6-fluoro-3-methyl-2-phenyl-2,3-dihydrobenzofuran-2-yl)methyl)amino)cyclohexane-1-carboxamido)cyclohexyl)amino)-2-oxoethoxy)-4-(4-methylthiazol-5-yl)benzyl)-1-((S)-2-(1-fluorocyclopropane-1-carboxamido)-3,3-dimethylbutanoyl)-4-hydroxypyrrolidine-2-carboxamide;
(2S,4R)—N-(2-((1-((1S,4r)-4-((((2S,3S,4S)-4-(6-carbamoyl-2-fluoro-3-methoxyphenyl)-5-chloro-6-fluoro-3-methyl-2-phenyl-2,3-dihydrobenzofuran-2-yl)methyl)amino)cyclohexyl)-1,15-dioxo-5,8,11-trioxa-2,14-diazahexadecan-16-yl)oxy)-4-(4-methylthiazol-5-yl)benzyl)-1-((S)-2-(1-fluorocyclopropane-1-carboxamido)-3,3-dimethylbutanoyl)-4-hydroxypyrrolidine-2-carboxamide;
(2S,4R)-1-((S)-2-(4-(4-((1-((1S,4r)-4-((((2S,3S,4S)-4-(6-carbamoyl-2-fluoro-3-methoxyphenyl)-5-chloro-6-fluoro-3-methyl-2-phenyl-2,3-dihydrobenzofuran-2-yl)methyl)amino)cyclohexane-1-carbonyl)piperidin-4-yl)oxy)piperidin-1-yl)-4-oxobutanamido)-3,3-dimethylbutanoyl)-4-hydroxy-N-((S)-1-(4-(4-methylthiazol-5-yl)phenyl)ethyl) pyrrolidine-2-carboxamide;
(2S,4R)—N-(2-(2-(4-((1-((1S,4r)-4-((((2S,3S,4S)-4-(6-carbamoyl-2-fluoro-3-methoxyphenyl)-5-chloro-6-fluoro-3-methyl-2-phenyl-2,3-dihydrobenzofuran-2-yl)methyl)amino)cyclohexane-1-carbonyl)piperidin-4-yl)methyl)piperidin-1-yl)-2-oxoethoxy)-4-(4-methylthiazol-5-yl)benzyl)-1-((S)-2-(1-fluorocyclopropane-1-carboxamido)-3,3-dimethylbutanoyl)-4-hydroxypyrrolidine-2-carboxamide;
(2S,4R)—N-(2-(2-(1′-((1S,4r)-4-((((2S,3S,4S)-4-(6-carbamoyl-2-fluoro-3-methoxyphenyl)-5-chloro-6-fluoro-3-methyl-2-phenyl-2,3-dihydrobenzofuran-2-yl)methyl)amino)cyclohexane-1-carbonyl)-[4,4′-bipiperidin]-1-yl)-2-oxoethoxy)-4-(4-methylthiazol-5-yl)benzyl)-1-((S)-2-(1-fluorocyclopropane-1-carboxamido)-3,3-dimethylbutanoyl)-4-hydroxypyrrolidine-2-carboxamide; and
(2S,4S)-4-((2-(((1S,4S)-4-((1S,4S)-4-((((2S,3S,4S)-4-(6-carbamoyl-2-fluoro-3-methoxyphenyl)-5-chloro-6-fluoro-3-methyl-2-phenyl-2,3-dihydrobenzofuran-2-yl)methyl)amino)cyclohexane-1-carboxamido)cyclohexyl)amino)-2-oxoethyl)amino)-1-((S)-2-cyclohexyl-2-((S)-2-(methylamino) propanamido)acetyl)-N—((R)-1,2,3,4-tetrahydronaphthalen-1-yl) pyrrolidine-2-carboxamide, or a pharmaceutically acceptable salt thereof.

Embodiment 45. A pharmaceutical composition comprising the conjugate or pharmaceutically acceptable salt thereof according to any one of the preceding embodiments, and one or more pharmaceutically acceptable carriers.

Embodiment 46. A combination comprising the conjugate or pharmaceutically acceptable salt thereof according to any one of embodiments 1 to 44, and one or more therapeutically active agents.

Embodiment 47. The conjugate or pharmaceutically acceptable salt thereof according to any one of embodiments 1 to 44 for use as a medicament.

Embodiment 48. The conjugate or pharmaceutically acceptable salt thereof according to any one of embodiments 1 to 44 for use in the treatment of cancer, preferably wherein the cancer is selected from breast cancer, lung cancer, ovarian cancer, colorectal cancer, malignant pleural mesothelioma, pancreatic cancer, prostate cancer, gastric cancer, esophageal cancer, liver cancer and bone cancer.

Embodiment 49. The conjugate or pharmaceutically acceptable salt thereof according to any one of embodiments 1 to 44 for use in the treatment of a disease or condition mediated by YAP overexpression and/or YAP amplification and/or YAP/TAZ-TEAD interaction.

### Definitions

Terms not specifically defined herein should be given the meanings that would be given to them by one of skill in the art in light of the disclosure and the context. As used in the specification and appended claims, however, unless specified to the contrary, the following terms have the meaning indicated and the following conventions are adhered to.

In the groups, radicals, or moieties defined below, the number of carbon atoms is often specified preceding the group, for example, C₁-C₁₀alkyl means an alkyl group or radical having 1 to 10 carbon atoms.

Furthermore, the use of a term designating a monovalent radical where a divalent radical is appropriate shall be construed to designate the respective divalent radical and vice versa. Unless otherwise specified, conventional definitions of terms control and conventional stable atom valences are presumed and achieved in all formulas and groups. The articles “a” and “an” refer to one or more than one (e.g., to at least one) of the grammatical object of the article. By way of example, “an element” means one element or more than one element.

As used herein, the term “a,” “an,” “the” and similar terms used in the context of the disclosure (especially in the context of the claims) are to be construed to cover both the singular and plural unless otherwise indicated herein or clearly contradicted by the context.

The term “and/or” means either “and” or “or” unless indicated otherwise.

As used herein, the term “C₁-C₆alkyl” refers to a straight or branched hydrocarbon chain radical consisting solely of carbon and hydrogen atoms, containing no unsaturation, having from one to six carbon atoms, and which is attached to the rest of the molecule by a single bond. The terms “C₁-C₃alkyl” and “C₁-C₄alkyl” are to be construed accordingly. Examples of C₁-C₆alkyl include, but are not limited to, methyl, ethyl, n-propyl, 1-methylethyl (iso-propyl), n-butyl, n-pentyl, 1,1-dimethylethyl (t-butyl) and hexyl.

As used herein, the term “hydroxyC₁-C₄alkyl” refers to a radical of formula —Rₐ—OH, wherein Rₐ is C₁-C₄alkyl as defined above. Examples of hydroxyC₁-C₄alkyl include, but are not limited to, hydroxy-methyl, 2-hydroxy-ethyl, 2-hydroxy-propyl and 3-hydroxy-propyl.

As used herein, the term “hydroxyC₁-C₃alkyl” refers to a radical of formula —Rₐ—OH, wherein Rₐ is C₁-C₃alkyl as defined above. Examples of hydroxyC₁-C₃alkyl include, but are not limited to, hydroxy-methyl, 2-hydroxy-ethyl, 2-hydroxy-propyl and 3-hydroxy-propyl.

As used herein, the term “C₃-C₆cycloalkyl” refers to a saturated monocyclic hydrocarbon group of 3-6 carbon atoms. Examples of C₃-C₆cycloalkyl include cyclopropyl, cyclobutyl, cyclopentyl and cyclohexyl.

As used herein, the term “C₁-C₆alkoxy” refers to a radical of the formula —ORₐ where Rₐ is a C₁-C₆alkyl radical as generally defined above. The terms “C₁-C₃alkoxy” and “C₁-C₄alkoxy” are to be construed accordingly. Examples of C₁-C₆alkoxy include, but are not limited to, methoxy, ethoxy, propoxy, isopropoxy, butoxy, isobutoxy, pentoxy, and hexoxy.

As used herein, the term “C₁-C₃-alkoxy-C₁-C₃alkyl” refers to a radical of formula —Rₐ-C₁-C₃-alkoxy, wherein Rₐ is a C₁-C₃-alkyl.

“Halogen” or “halo” refers to fluoro, chloro, bromo or iodo. Preferably, halo is fluoro, chloro or bromo. More preferably, halo is fluoro or chloro.

The term “oxo” refers to the radical ═O.

The term “sulfonyl” refers to the radical —S(═O)₂—.

The term “amino” refers to the radical —NH₂.

The term “NHR^{1b}” refers to the radical —N(H)R^{1b}. Similarly, a term such as “NR^{5a}R^{5b}” refers to the radical —N(R^{5a})R^{5b}.

The term “cyano” refers to —CN.

As used herein, the term “halogenC₁-C₃alkyl” or “haloC₁-C₃alkyl” refers to a C₁-C₃alkyl radical, as defined above, substituted with one or more halo radicals, as defined above. Examples of halogenC₁-C₃alkyl include, but are not limited to, trifluoromethyl, difluoromethyl, fluoromethyl, trichloromethyl, 1,1-difluoroethyl, 2,2-difluoroethyl, 2,2,2-trifluoroethyl, 2-fluoropropyl, 3,3-difluoropropyl and 1-fluoromethyl-2-fluoroethyl.

As used herein, the term “haloC₁-C₆alkoxy” refers to C₁-C₆alkoxy as defined above, wherein at least one of the hydrogen atoms of the C₁-C₆alkoxy radical is substituted with a halo radical, as defined above. The term “haloC₁-C₃alkoxy” is to be construed accordingly. Examples of haloC₁-C₆alkoxy include, but are not limited to, trifluoromethoxy, difluoromethoxy, trifluoroethoxy, 2-fluoropropoxy, 3,3-difluoropropoxy.

As used herein, the term “hydroxyC₁-C₆alkoxy” refers to a C₁-C₆alkoxy radical as defined above, wherein at least one of the hydrogen atoms of the C₁-C₆alkoxy radical is replaced by OH. The term “hydroxyC₁-C₃alkoxy is to be construed accordingly. Examples of hydroxyC₁-C₆alkoxy include, but are not limited to, hydroxymethoxy, hydroxyethoxy, 2-hydroxypropoxy.

As used herein, the term “C₁-C₃alkoxyC₁-C₃alkoxy” refers to a C₁-C₃alkoxy radical as defined above, wherein one of the hydrogen atoms of the C₁-C₃alkoxy radical is replaced by —O—C₁-C₃alkyl. An example of C₁-C₃alkoxyC₁-C₃alkoxy includes, but is not limited to, 2-methoxyethoxy.

As used herein, the term “haloC₁-C₃alkoxy-C₁-C₃alkyl” refers to a C₁-C₃alkyl radical as defined above, wherein one of the hydrogen atoms of the C₁-C₃alkyl radical is replaced by haloC₁-C₃alkoxy as defined above. Examples of haloC₁-C₃alkoxy-C₁-C₃alkyl include, but are not limited to (difluoromethoxy)methyl (i.e. CHF₂-O-CH₂—).

As used herein, the term “C(O)NR^{1c}R^{1d}” refers to a radical of the formula —Rₐ₁-N(Rₐ₂)₂ where Rₐ₁ is a carbonyl radical and each Rₐ₂ is a R^{1c} or a R^{1d} radical, each of which may be the same or different, as defined herein.

As used herein, the term “C(O)di(C₁-C₃alkyl)amino” refers to a radical of the formula —Rₐ₁-N(Rₐ₂)₂ where Rat is a carbonyl radical and each Rₐ₂ is a C₁-C₃alkyl as defined herein, and each may be the same or different.

As used herein, the term “C(O)C₁-C₃alkyl” refers to a radical of the formula —Rₐ₁-C₁-C₃alkyl where Rat is a carbonyl radical and C₁-C₃alkyl is as defined above.

As used herein, the term “C(O)NHR^{6a}” refers to a radical of the formula —Rₐ₁-N(H)—R^{6a} where Rₐ₁ is a carbonyl radical and R^{6a} is as defined herein.

As used herein, the term “S-haloC₁-C₃alkyl” refers to a radical of the formula —S-haloC₁-C₃alkyl where haloC₁-C₃alkyl is as defined above.

As used herein, the term “C(O)OC₁-C₃alkyl” refers to a radical of the formula —Rₐ₁—O—C₁-C₃alkyl where Rai is a carbonyl radical and C₁-C₃alkyl is as defined above.

As used herein, the term “SO₂C₁-C₃alkyl” refers to a radical of the formula —S(═O)₂-Rₐ₂ where Rₐ₂ is a C₁-C₃alkyl as defined above.

The term “C₁-C₃alkylene” as used herein refers to a straight or branched hydrocarbon chain bivalent radical consisting solely of carbon and hydrogen atoms, containing no unsaturation, and having from one to six carbon atoms. The term “C₁-C₃alkylene” is to be construed accordingly. In embodiments whereby the 4-, 5- or 6-membered saturated heterocyclic ring of Q (or Q1) is substituted with a C₁-C₃alkylene forming a bridge between two ring atoms of the saturated heterocyclic ring, thus forming a bridged bicyclic structure, the C₁-C₃alkylene is preferably propylene (—CH₂—CH₂—CH₂—), ethylene (—CH₂—CH₂—) or methylene (—CH₂—). The term “(CH₂)₀₋₂R₁ₐ” refers to a radical of the formula —(CH₂)₀₋₂R₁ₐ, i.e., the radical R₁ₐ is attached to the rest of the molecule via a bond, a methylene linker or an ethylene linker. The term “(CH₂)₀₋₁C(O)di(C₁-C₃alkyl)amino” refers to a radical of the formula —(CH₂)₀₋₁-Rₐ₃ and Rₐ₃ is a C(O)di(C₁-C₃alkyl)amino radical as defined above.

The term (CH₂)₀₋₁C(O)NR^{1c}R^{1d} refers to a radical of the formula —(CH₂)₀₋₁C(O)NR^{1c}R^{1d}.

As used herein, the term “5- or 6-membered saturated heterocyclic ring comprising at least one heteroatom selected from N and O” refers to a monocyclic ring and includes, but is not limited to, piperazinyl, piperidyl, pyrrolidinyl, tetrahydrofuryl, tetrahydropyranyl, dioxanyl and morpholinyl. Preferably this term includes piperidyl, pyrrolidinyl, tetrahydrofuryl, tetrahydropyranyl and morpholinyl. The terms “5-membered saturated heterocyclic ring comprising at least one heteroatom selected from N and O” and “6-membered saturated heterocyclic ring comprising at least one heteroatom selected from N and O” are to be construed accordingly.

As used herein, the term “4-, 5- or 6-membered saturated heterocyclic ring comprising at least one heteroatom or heteroatom group selected from N, O, S, —S(═O) and —S(═O)₂” refers to a monocyclic ring and includes, but is not limited to, azetidinyl, pyrrolidinyl, piperidinyl, piperazinyl, morpholinyl, thiomorpholinyl, S-oxo-thiomorpholinyl or S,S-dioxothiomorpholinyl. For the avoidance of doubt, in certain embodiments whereby the N is present in the α-positon to the atom binding Q to the rest of the molecule, this may be represented by the following formula

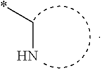 [SMILES: *CN]

As used herein, the term “5- or 6-membered aromatic heterocyclic ring comprising at least one heteroatom selected from N, O, or S, preferably from N or S” refers to a monocyclic aromatic ring. Examples of this term include but are not limited to oxazolyl, isozaolyl, pyrimidinyl, pyridazinyl, tetrazolyl, pyrazinyl, triazolyl, imidazolyl, pyrazolyl, pyridinyl and thiazolyl.

As used herein, the term “5- or 6-membered aromatic heterocyclic ring comprising at least one heteroatom selected from N, O, and S” refers to an aromatic monocyclic ring and includes, but is not limited to, pyrimidinyl, pyridazinyl, tetrazolyl, pyrazinyl, triazolyl, imidazolyl, pyrazolyl, pyridinyl, oxazolyl, and thiazolyl. The point of attachment to the imidazolyl ring is preferably to the nitrogen atom of the imidazolyl ring.

As used herein, the term “5- or 6-membered aromatic heterocyclic ring comprising at least one heteroatom selected from N and S” refers to a monocyclic aromatic ring and includes, but is not limited to, pyrimidinyl, pyridazinyl, tetrazolyl, pyrazinyl, triazolyl, imidazolyl, pyrazolyl, pyridinyl and thiazolyl.

As used herein, the term “6-membered aromatic heterocyclic ring comprising at least one N heteroatom” refers to a monocyclic aromatic ring and includes, but is not limited to, pyrimidinyl, pyridazinyl, pyrazinyl and pyridinyl.

As used herein, the term “5-membered aromatic heterocyclic ring comprising at least one N heteroatom” (where N may also be NH) refers to a monocyclic aromatic ring and includes, but is not limited to, tetrazolyl, triazolyl, imidazolyl, pyrazolyl.

As used herein, the term “5- or 6-membered aromatic heterocyclic ring comprising at least one N heteroatom” refers to a monocyclic aromatic ring and includes, but is not limited to, pyrimidinyl, pyridazinyl, tetrazolyl, pyrazinyl, triazolyl, imidazolyl, pyrazolyl and pyridinyl.

As used herein, the aromatic heterocyclic ring in the substituent defined as “5- or 6-membered aromatic heterocyclic ring comprising at least one heteroatom selected from N, O, or S, preferably from N or S” may be optionally substituted with hydroxy; C₁-C₃alkoxy; or oxo.

It will be understood that substitution of said aromatic heterocycle with oxo is meant to include 5- or 6-membered rings in which an aromatic tautomer exists, as for example in the 1H-pyridin-2-one system.

As used herein, the term “5- or 6-membered saturated heterocyclic ring” in relation to the embodiments where R^{5a} and R^{5b} together with the N atom (where N may also be NH) to which they are attached form said ring, includes as examples, but is not limited to, an azetidinyl ring, a pyrrolidine ring, or a piperidine ring.

As used herein, the term “9- or 10-membered partially saturated heteroaryl comprising at least one N heteroatom” refers to a partially saturated aromatic bicyclic heterocyclic ring system whereby a 5- or 6-membered heterocyclic ring containing one N heteroatom, is fused with a benzene ring or a heteroaromatic ring. In certain embodiments whereby the N is present in the α-positon to the atom binding Q to the rest of the molecule, this may be represented by the following formula

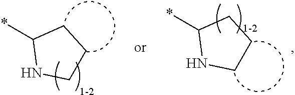

whereby the dashed ring represents the benzo or heteroaryl ring. Representative examples are indolinyl, isoindolinyl, tetrahydroquinolinyl, tetrahydroisoquinolinyl, and the like. Preferably, it is

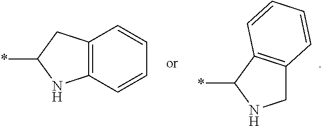 [SMILES: *C1Cc2ccccc2N1 ; *C1NCc2ccccc21]

As used herein, the term “optionally substituted” includes unsubstituted or substituted.

As used herein, the term “more than once” includes 2, 3, 4, 5, or 6 times. Preferably, it includes 2 or 3 times.

As used herein, the term “more than one” includes 2, 3, 4, 5, or 6. Preferably, it includes 2 or 3.

As used herein, the term “at least one heteroatom” includes 1, 2, 3, 4 or 5, preferably 1, 2, 3 or 4, more preferably 1 or 2 heteroatoms.

The use of any and all examples, or exemplary language (e.g. “such as” or “preferably”) provided herein is intended merely to better illuminate the invention and does not pose a limitation on the scope of the invention otherwise claimed.

As used herein, the term protecting group, e.g., a nitrogen protecting group (PG), refers to a group that should protect the functional groups concerned against unwanted secondary reactions, such as acylations, etherifications, esterifications, oxidations, solvolysis and similar reactions. It may be removed under deprotection conditions. Depending on the protecting group employed, the skilled person would know how to remove the protecting group to obtain the free amine NH₂ group by reference to known procedures. These include reference to organic chemistry textbooks and literature procedures such as J. F. W. McOmie, “Protective Groups in Organic Chemistry”, Plenum Press, London and New York 1973; T. W. Greene and P. G. M. Wuts, “Greene's Protective Groups in Organic Synthesis”, Fourth Edition, Wiley, New York 2007; in “The Peptides”; Volume 3 (editors: E. Gross and J. Meienhofer), Academic Press, London and New York 1981, and in “Methoden der organischen Chemie” (Methods of Organic Chemistry), Houben Weyl, 4th edition, Volume 15/1, Georg Thieme Verlag, Stuttgart 1974 and later editions thereof.

Preferred nitrogen protecting groups generally comprise: C₁-C₆alkyl (e.g. tert-butyl), preferably C₁-C₄alkyl, more preferably C₁-C₂alkyl, most preferably C₁alkyl which is mono-, di- or tri-substituted with trialkylsilyl-C₁-C₇alkoxy (eg. trimethylsilyethoxy), aryl, preferably phenyl, or a heterocyclic group (e.g., benzyl, cumyl, benzhydryl, pyrrolidinyl, trityl, pyrrolidinylmethyl, 1-methyl-1,1-dimethylbenzyl, (phenyl)methylbenzene) wherein the aryl ring or the heterocyclic group is unsubstituted or substituted with one or more, e.g. two or three, residues, e.g. selected from the group consisting of C₁-C₇alkyl, hydroxy, C₁-C₇alkoxy (e.g. para-methoxy benzyl (PMB)), C₂-C₈-alkanoyl-oxy, halogen, nitro, cyano, and CF₃, aryl-C₁-C₂-alkoxycarbonyl (preferably phenyl-C₁-C₂-alkoxycarbonyl (eg. benzyloxycarbonyl (Cbz), benzyloxymethyl (BOM), pivaloyloxymethyl (POM)), C₁-C₁₀-alkenyloxycarbonyl, C₁-C₆alkylcarbonyl (eg. acetyl or pivaloyl), C₆-C₁₀-arylcarbonyl; C₁-C₆-alkoxycarbonyl (eg. tertbutoxycarbonyl (Boc), methylcarbonyl, trichloroethoxycarbonyl (Troc), pivaloyl (Piv), allyloxycarbonyl), C₆-C₁₀-arylC₁-C₆-alkoxycarbonyl (e.g. 9-fluorenylmethyloxycarbonyl (Fmoc)), allyl or cinnamyl, sulfonyl or sulfenyl, succinimidyl group, silyl groups (e.g. triarylsilyl, trialkylsilyl, triethylsilyl (TES), trimethylsilylethoxymethyl (SEM), trimethylsilyl (TMS), triisopropylsilyl or tertbutyldimethylsilyl).

According to the disclosure, the preferred protecting group (PG) can be selected from the group comprising tert-butyloxycarbonyl (Boc), benzyloxycarbonyl (Cbz), para-methoxy benzyl (PMB), methyloxycarbonyl and benzyl. The protecting group (PG) is preferably tert-butyloxycarbonyl (Boc).

The term “phenyl” refers to a radical of the formula —C₆H₅.

The term “halobenzodioxole” refers to a 1,3-benzodioxole radical of the formula

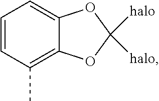

wherein halo is as defined above. Preferably, both halo groups are fluoro.

The term “stereoisomer” or “stereoisomers” refer to compounds, which have identical chemical constitution, but differ with regard to the arrangement of the atoms or groups in space.

The term “diastereoisomer” or “diastereomer” refers to stereoisomers not related as mirror images. Diastereoisomers are characterized by differences in physical properties, and by some differences in chemical behaviour. Mixtures of diastereomers may separate under analytical procedures such as chromatography or crystallisation.

The term “enantiomer” refers to one of a pair of molecular entities which are mirror images of each other and non-superimposable.

The term “enantiomeric mixture” refers to an enantiomerically enriched mixture, a composition that comprises a greater proportion or percentage of one of the enantiomers of the compounds of the invention, in relation to the other enantiomer, or a racemate.

The term “diastereomeric mixture” refers to a diastereomerically enriched mixture or a mixture of diastereoisomers of equal proportion.

The term “diastereomerically enriched” refers to a composition that comprises a greater proportion or percentage of one of the diastereomers of the compounds of the invention, in relation to the other diastereoisomer(s).

The term “atropisomer” refers to a stereoisomer resulting from restricted rotation about single bonds where the rotation barrier is high enough to permit isolation of the isomeric species. Typically, rotation about the single bond in the molecule is prevented, or greatly slowed, as a result of steric interactions with other parts of the molecule and the substituents at both ends of the single bond are asymmetrical, resulting in a stereogenic unit termed a “chiral axis”.

As used herein, the term “YAP” refers to yes-associated protein, also known as YAP1 or YAP65.

Whenever YAP is mentioned herein it can also refer to the YAP/TAZ complex.

As used herein, the term “YAP/TAZ-TEAD” refers to the complex of YAP/TAZ with TEAD transcription factor.

As used herein, the term “NF2/LATS1/LATS2” refers to “NF2”, “LATS1”, or “LATS2” or any combinations thereof.

As used herein, the term “conjugate” refers to a molecule including:
1) A YAP/TAZ-TEAD Protein-Protein Interaction Inhibitor (PPII) component;
2) A Ligase Binder component; and
3) A linker component connecting the YAP/TAZ-TEAD PPII component to the Ligase Binder component.

As used herein, the term “YAP/TAZ-TEAD PPII” or “YAP/TAZ-TEAD Protein-Protein Interaction Inhibitor” or “YAP/TAZ-TEAD PPI Inhibitor” refers to a compound which is capable of inhibiting the interaction between i) TEAD and ii) YAP and/or TAZ, for example by binding to TEAD and thus selectively disrupting TEAD's interaction with YAP and/or TAZ.

The compounds of formula (Ia-i), (Ia-ii), (Ia-iii), (Ib-i), (Ib-ii), (Ib-iii), (Ic-i), (Ic-ii), and (Ic-iii) are stereospecific atropisomers. The compounds of formula (I), (Ia), (Ib) and (Ic) include all stereoisomers, including diastereoisomers, atropisomers, enantiomers, mixtures thereof and racemic mixtures.

The presence of diastereoisomers can be identified by a person of skill in the art with tools such as NMR. Separation of diastereoisomers can be carried out by a person of skill in the art using chromatographic methods, with tools such as HPLC (High Performance Liquid Chromatography), Thin Layer Chromatography, SFC (Supercritical Fluid Chromatography), GC (Gas Chromatography), or recrystallization techniques. Separation of enantiomers can be carried out by a person of skill in the art with tools such as chiral HPLC, chiral SFC, chiral GC.

Compounds of the present invention, in particular, ortho-substituted biaryl compounds may exhibit conformational, rotational isomerism, herein referred to as atropisomerism (Eliel, E. and Wilen, S. (1994) Stereochemistry of Organic Compounds, John Wiley & Sons, Inc., pp. 1142-55). In some instances, depending upon the substituents R₄ and R₆, such biaryl compounds of the present invention exhibit atropisomerism.

Thus, the compounds including a YAP/TAZ-TEAD PPII of formula (I), and subformulae (Ia), (Ia*), (Ia-1), (Ib), (Ic), (Id) and their isomeric mixtures (including diastereomeric mixtures, enantiomeric mixtures and racemic mixtures), also form part of the invention.

The term “linker” as used herein refers to a chemical moiety which joins the YAP/TAZ-TEAD to the Fatty Acid. Preferably the linker is a long, substantially straight-chained group including from 6 to 200, for example from 10 to 100, for example from 15 to 80, for example from 20 to 60 non-hydrogen atoms (typically selected from C, N, O and S, most typically selected from C, N and O). By “substantially straight-chained” it is meant that the main chain may be substituted by one or more groups each independently containing from 1 to 6 non-hydrogen atoms, preferably 1 to 4 non-hydrogen atoms (typically selected from C, N, O and S, most typically selected from C, N, and O). Such substituents may include, purely as an example, ═O, C₁-C₄alkyl, C₁-C₃hydroxyalkyl, C₁-C₃aminoalkyl, OH, O—C₁-C₃alkyl, NH₂, N(H)C₁-C₃alkyl, N(C₁-C₂alkyl)₂, C₁-C₂alkylene-O—C₁-C₃alkyl, C₁-C₂alkylene-N(H)C₁-C₂alkyl, and CH₂N(C₁alkyl)₂.

The term “ligase binder” as used herein is a group that is capable of binding to a ligase (e.g. Cereblon E3 Ubiquitin ligase). The ligase binder is preferably a Cereblon E3 Ubiquitin Ligase binder. Alternatively, the ligase binder may be a VHL binder.

“Alkylene” refers to a straight-chain or branched divalent radical of an alkyl group, e.g., —CH₂—, —CH₂CH₂—, and —CH₂CH₂CH₂—.

“Heteroalkyl” refers to an alkyl group, which further includes at least one heteroatom (e.g., 1, 2, 3, or 4 heteroatoms) selected from oxygen, nitrogen, or sulfur within (i.e., inserted between adjacent carbon atoms of) and/or placed at one or more terminal position(s) of the parent chain.

In certain embodiments, a heteroalkyl group refers to a saturated group having from 1 to 10 carbon atoms and 1 or more heteroatoms within the parent chain (“heteroC₁-C₁₀alkyl” or “C₁-C₁₀heteroalkyl”). In some embodiments, a heteroalkyl group is a saturated group having 1 to 9 carbon atoms and 1 or more heteroatoms within the parent chain (“heteroC₁-C₉alkyl” or “C₁-C₉heteroalkyl”). In some embodiments, a heteroalkyl group is a saturated group having 1 to 8 carbon atoms and 1 or more heteroatoms within the parent chain (“heteroC₁-C₈alkyl” or “C₁-C₈heteroalkyl”). In some embodiments, a heteroalkyl group is a saturated group having 1 to 7 carbon atoms and 1 or more heteroatoms within the parent chain (“heteroC₁-C₇alkyl” or “C₁-C₇heteroalkyl”). In some embodiments, a heteroalkyl group is a saturated group having 1 to 6 carbon atoms and 1 or more heteroatoms within the parent chain (“heteroC₁-C₆alkyl” or “C₁-C₆heteroalkyl”). In some embodiments, a heteroalkyl group is a saturated group having 1 to 5 carbon atoms and 1 or 2 heteroatoms within the parent chain (“heteroC₁-C₅alkyl” or “C₁-C₅heteroalkyl”). In some embodiments, a heteroalkyl group is a saturated group having 1 to 4 carbon atoms and 1 or 2 heteroatoms within the parent chain (“heteroC₁-C₄alkyl” or “C₁-C₄heteroalkyl”). In some embodiments, a heteroalkyl group is a saturated group having 1 to 3 carbon atoms and 1 heteroatom within the parent chain (“heteroC₁-C₃alkyl” or “C₁-C₃heteroalkyl”). In some embodiments, a heteroalkyl group is a saturated group having 1 to 2 carbon atoms and 1 heteroatom within the parent chain (“heteroC₁-C₂alkyl” or “C₁-C₂heteroalkyl”). In some embodiments, a heteroalkyl group is a saturated group having 1 carbon atom and 1 heteroatom (“heteroC₁alkyl” or “C₁heteroalkyl”). In some embodiments, a heteroalkyl group is a saturated group having 2 to 6 carbon atoms and 1 or 2 heteroatoms within the parent chain (“heteroC₂-C₆alkyl” or “C₂-C₆heteroalkyl”). Unless otherwise specified, each instance of a heteroalkyl group is independently unsubstituted (an “unsubstituted heteroalkyl”) or substituted (a “substituted heteroalkyl”) with one or more substituents. In certain embodiments, the heteroalkyl group is an unsubstituted heteroC₁-C₁₀alkyl. In certain embodiments, the heteroalkyl group is a substituted heteroC₁-C₁₀alkyl. It should be understood that “heteroalkyl” therefore includes substituents such as —CH₂CH₂NH₂ and —CH₂—O—CH₃, but not —OCH₃ (because in the latter case, the heteroatom is not placed between adjacent carbon atoms and/or placed at one or more terminal position(s) of the parent chain).

“Heteroalkylene” refers to a divalent radical of a heteroalkyl group as defined herein.

As used herein, the term “aryl” refers to a stable, aromatic, mono- or bicyclic ring radical. Examples of aryl groups include, but are not limited to, phenyl, 1-naphthyl, 2-naphthyl, and the like. The related term “aryl ring” likewise refers to a stable, aromatic, mono- or bicyclic ring having the specified number of ring carbon atoms.

“Arylene” refers to a divalent radical of a aryl group as defined herein.

As used herein, the term “heteroaryl” refers to a 5- or 6-membered aromatic monocyclic ring radical which comprises 1, 2, 3 or 4 heteroatoms individually selected from nitrogen, oxygen and sulfur. The heteroaryl radical may be bonded via a carbon atom or heteroatom. Examples of heteroaryl include, but are not limited to, furyl, pyrrolyl, thienyl, pyrazolyl, imidazolyl, thiazolyl, isothiazolyl, oxazolyl, isoxazolyl, triazolyl, tetrazolyl, pyrazinyl, pyridazinyl, pyrimidyl or pyridyl.

“Heteroarylene” refers to a divalent radical of a heteroaryl group as defined herein.

As used herein, the term “carbocyclyl” or, interchangeably, “cycloalkyl” refers to a stable, saturated, non-aromatic, monocyclic ring radical. Examples of carbocyclyl groups include, but are not limited to, the cycloalkyl groups identified above, cyclobutenyl, cyclopentenyl, cyclohexenyl, In an embodiment, the carbocyclyl can be substituted with 0-4 occurrences of R_{cc}, wherein each R_{cc} is independently selected from the group consisting of C₁-C₆alkyl, C₁-C₆alkoxyl, and halogen.

“Cycloalkylene” refers to a divalent radical of a cycloalkyl group as defined herein.

As used herein, the term “heterocyclyl” or “heterocyclic” refers to a stable 4-, 5-, 6- or 7-membered non-aromatic monocyclic ring radical which comprises 1, 2, or 3, heteroatoms individually selected from nitrogen, oxygen and sulfur. The heterocyclyl radical may be bonded via a carbon atom or heteroatom. Examples of heterocyclyl include, but are not limited to, azetidinyl, oxetanyl, pyrrolinyl, pyrrolidyl, tetrahydrofuryl, tetrahydrothienyl, piperidyl, piperazinyl, tetrahydropyranyl, morpholinyl or perhydroazepinyl.

“Heterocyclylene” refers to a divalent radical of a heterocyclyl group as defined herein.

As used herein, “spirocycloalkyl” or “spirocyclyl” means carbogenic bicyclic ring systems with both rings connected through a single atom. The rings can be different in size and nature, or identical in size and nature. Examples include spiropentane, spriohexane, spiroheptane, spirooctane, spirononane, or spirodecane. One or both of the rings in a spirocycle can be fused to another ring carbocyclic, heterocyclic, aromatic, or heteroaromatic ring. For example, a C₃-C₁₂spirocycloalkyl is a spirocycle containing between 3 and 12 carbon atoms.

“Spirocyclylene” refers to a divalent radical of a spirocyclyl group as defined herein.

As used herein, “spiroheterocycloalkyl” or “spiroheterocyclyl” means a spirocyclyl as defined above, wherein one or both of the rings is heterocyclic, i.e. wherein 1 to 4 of the carbon atoms in the ring is substituted with heteroatoms independently selected from N, O and S. One or both of the rings in a spiroheterocycle can be fused to another ring carbocyclic, heterocyclic, aromatic, or heteroaromatic ring. Preferably the total number of ring atoms (including both carbon and heteroatoms selected from N, O and S) is from 3 to 12.

“Spiroheterocyclylene” refers to a divalent radical of a spiroheterocyclyl group as defined herein.

As used herein, “haloalkyl” means an alkyl group (preferably C₁-C₆alkyl) substituted with one or more halogens. Examples of haloalkyl groups include, but are not limited to, trifluoromethyl, difluoromethyl, pentafluoroethyl, and trichloromethyl.

“Haloalkylene” refers to a divalent radical of a “haloalkyl” group as defined herein.

“Alkoxy” or “alkoxyl” refers to an —O-alkyl radical. In some embodiments, the alkoxy groups are methoxy, ethoxy, n-propoxy, isopropoxy, n-butoxy, tert-butoxy, sec-butoxy, n-pentoxy, n-hexoxy, and 1,2-dimethylbutoxy. In some embodiments, alkoxy groups are lower alkoxy, i.e., with between 1 and 6 carbon atoms. In some embodiments, alkoxy groups have between 1 and 4 carbon atoms. An alternative name for “alkoxy” is “O-alkyl”.

“O-alkylene” refers to a divalent radical of an “O-alkyl” group as defined herein.

As used herein, the term “alkenyl” refers to a straight or branched hydrocarbon chain radical group consisting solely of carbon and hydrogen atoms, containing at least one double bond. An alkenyl may be a C₂-C₆alkenyl. As used herein, the term “C₂-C₆alkenyl” refers to a straight or branched hydrocarbon chain radical group consisting solely of carbon and hydrogen atoms, containing at least one double bond, having from two to six carbon atoms, which is attached to the rest of the molecule by a single bond. The term “C₂-C₄alkenyl” is to be construed accordingly. Examples of C₂-C₆alkenyl include, but are not limited to, ethenyl, prop-1-enyl, but-1-enyl, pent-1-enyl, pent-4-enyl and penta-1,4-dienyl.

“Alkenylene” refers to a divalent radical of an “alkenyl” group as defined herein.

As used herein, the term “alkoxyalkyl” refers to an alkylene, as defined herein, substituted with an alkoxy group, as defined herein, e.g.-CH₂—O—CH₂CH₃. The term “C₁-C₆alkoxyalkyl” as used herein is equivalent to “C₁-C₆alkoxyC₁-C₆alkyl”. Thus, it includes substituents of the general formula —(CH₂)₁₋₆—O—(CH₂)₁₋₅—CH₃ and branched equivalents thereof. An alternative term for “C₁-C₆alkoxyalkyl” and “C₁-C₆alkoxyC₁-C₆alkyl” is “C₁-C₆alkylene-O—C₁-C₆alkyl”.

“Alkynyl” means a straight or branched monovalent hydrocarbon chain containing at least one carbon-carbon triple bond. Representative alkynyl include —C≡CH, —C≡C—CH₃, —C≡C—CH₂—CH₃, and the like.

“Alkynylene” means a straight or branched bivalent hydrocarbon chain containing at least one carbon-carbon triple bond. Representative alkynylene include —C≡C—, —C≡C—CH₂—, —C≡C—CH(CH₃)—, and the like.

As used herein, the term “C₁-C₆hydroxyalkyl” refers to a C₁-C₆alkyl radical as defined herein, wherein one of the hydrogen atoms of the C₁-C₆alkyl radical is replaced by OH. Examples of C₁-C₆hydroxyalkyl include, but are not limited to, hydroxy-methyl, 2-hydroxy-ethyl, 2-hydroxy-propyl, 3-hydroxy-propyl and 5-hydroxy-pentyl.

“Hydroxyalkylene” refers to a divalent radical of an “hydroxyalkyl” group as defined herein.

As used herein, the term “C₁-C₆aminoalkyl” refers to a C₁-C₃alkyl radical as defined herein, wherein one of the hydrogen atoms of the C₁-C₆alkyl group is replaced by a primary amino group (i.e. NH₂). Representative examples of C₁-C₆aminoalkyl include, but are not limited to, amino-methyl, 2-aminoethyl, 2-amino-propyl, 3-amino-propyl, 3-amino-pentyl and 5-amino-pentyl.

“Aminoalkylene” refers to a divalent radical of an “aminoalkyl” group as defined herein.

The term “polyethylene glycol” as used herein refers to a group of the formula

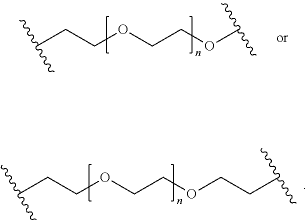

n may be, for example, from 0 to 50, for example 0 to 20, for example 0 to 10, for example 0 to 5, for example 0 to 4, for example 0 to 3, for example 0, 1 or 2.

The term “substituted”, whether preceded by the term “optionally” or not, means that the specified group or moiety bears one or more suitable substituents wherein the substituents may connect to the specified group or moiety at one or more positions. For example, an aryl substituted with a cycloalkyl may indicate that the cycloalkyl connects to one atom of the aryl with a bond or by fusing with the aryl and sharing two or more common atoms.

The term “unsubstituted” means that the specified group bears no substituents.

As used herein, the definition of each expression, e.g., R, m, n, etc., when it occurs more than once in any structure, is intended to be independent of its definition elsewhere in the same structure.

Depending on the choice of the starting materials and procedures, the conjugates can be present in the form of one of the possible stereoisomers or as mixtures thereof, for example as pure optical isomers, or as stereoisomer mixtures, such as racemates and diastereoisomer mixtures, depending on the number of asymmetric carbon atoms. The present invention is meant to include all such possible stereoisomers, including racemic mixtures, diasteriomeric mixtures and optically pure forms. Optically active (R)- and (S)-stereoisomers may be prepared using chiral synthons or chiral reagents, or resolved using conventional techniques. If the conjugate contains a double bond, the substituent may be E or Z configuration. If the conjugate contains a disubstituted cycloalkyl, the cycloalkyl substituent may have a cis- or trans-configuration. All tautomeric forms are also intended to be included.

As used herein, the terms “salt” or “salts” refers to an acid addition or base addition salt of a conjugate of the present invention. “Salts” include in particular “pharmaceutical acceptable salts”. The term “pharmaceutically acceptable salts” refers to salts that retain the biological effectiveness and properties of the conjugate of this invention and, which typically are not biologically or otherwise undesirable. In many cases, the conjugates of the present invention are capable of forming acid and/or base salts by virtue of the presence of amino and/or carboxyl groups or groups similar thereto. When both a basic group and an acid group are present in the same molecule, the conjugates of the present invention may also form internal salts, e.g., zwitterionic molecules.

Pharmaceutically acceptable acid addition salts can be formed with inorganic acids and organic acids.

Inorganic acids from which salts can be derived include, for example, hydrochloric acid, hydrobromic acid, sulfuric acid, nitric acid, phosphoric acid, and the like.

Organic acids from which salts can be derived include, for example, acetic acid, propionic acid, glycolic acid, oxalic acid, maleic acid, malonic acid, succinic acid, fumaric acid, tartaric acid, citric acid, benzoic acid, mandelic acid, methanesulfonic acid, ethanesulfonic acid, toluenesulfonic acid, sulfosalicylic acid, and the like.

Pharmaceutically acceptable base addition salts can be formed with inorganic and organic bases. Inorganic bases from which salts can be derived include, for example, ammonium salts and metals from columns I to XII of the periodic table. In certain embodiments, the salts are derived from sodium, potassium, ammonium, calcium, magnesium, iron, silver, zinc, and copper; particularly suitable salts include ammonium, potassium, sodium, calcium and magnesium salts. Organic bases from which salts can be derived include, for example, primary, secondary, and tertiary amines, substituted amines including naturally occurring substituted amines, cyclic amines, basic ion exchange resins, and the like. Certain organic amines include isopropylamine, benzathine, cholinate, diethanolamine, diethylamine, lysine, meglumine, piperazine and tromethamine.

In another aspect, the present invention provides conjugates of the present invention in acetate, ascorbate, adipate, aspartate, benzoate, besylate, bromide/hydrobromide, bicarbonate/carbonate, bisulfate/sulfate, camphorsulfonate, caprate, chloride/hydrochloride, chlortheophyllonate, citrate, ethandisulfonate, fumarate, gluceptate, gluconate, glucuronate, glutamate, glutarate, glycolate, hippurate, hydroiodide/iodide, isethionate, lactate, lactobionate, laurylsulfate, malate, maleate, malonate, mandelate, mesylate, methylsulphate, mucate, naphthoate, napsylate, nicotinate, nitrate, octadecanoate, oleate, oxalate, palmitate, pamoate, phosphate/hydrogen phosphate/dihydrogen phosphate, polygalacturonate, propionate, sebacate, stearate, succinate, sulfosalicylate, sulfate, tartrate, tosylate trifenatate, trifluoroacetate or xinafoate salt form.

Any formula given herein is also intended to represent unlabeled forms as well as isotopically labeled forms of the conjugates. Isotopically labeled conjugates have structures depicted by the formulae given herein except that one or more atoms are replaced by an atom having a selected atomic mass or mass number. Isotopes that can be incorporated into conjugates of the invention include, for example, isotopes of hydrogen.

Further, incorporation of certain isotopes, particularly deuterium (i.e., 2H or D) may afford certain therapeutic advantages resulting from greater metabolic stability, for example increased in vivo half-life or reduced dosage requirements or an improvement in therapeutic index or tolerability. It is understood that deuterium in this context is regarded as a substituent of a conjugate of the present invention. The concentration of deuterium, may be defined by the isotopic enrichment factor. The term “isotopic enrichment factor” as used herein means the ratio between the isotopic abundance and the natural abundance of a specified isotope. If a substituent in a conjugate of this invention is denoted as being deuterium, such compound has an isotopic enrichment factor for each designated deuterium atom of at least 3500 (52.5% deuterium incorporation at each designated deuterium atom), at least 4000 (60% deuterium incorporation), at least 4500 (67.5% deuterium incorporation), at least 5000 (75% deuterium incorporation), at least 5500 (82.5% deuterium incorporation), at least 6000 (90% deuterium incorporation), at least 6333.3 (95% deuterium incorporation), at least 6466.7 (97% deuterium incorporation), at least 6600 (99% deuterium incorporation), or at least 6633.3 (99.5% deuterium incorporation). It should be understood that the term “isotopic enrichment factor” can be applied to any isotope in the same manner as described for deuterium.

Other examples of isotopes that can be incorporated into conjugates of the invention include isotopes of hydrogen, carbon, nitrogen, oxygen, phosphorous, fluorine, and chlorine, such as 3H, 11C, 13C, 14C, 15N, 18F 31P, 32P, 35S, 36Cl, 123I, 124I, 125I respectively. Accordingly it should be understood that the invention includes conjugates that incorporate one or more of any of the aforementioned isotopes, including for example, radioactive isotopes, such as 3H and 14C, or those into which non-radioactive isotopes, such as 2H and 13C are present. Such isotopically labelled conjugates are useful in metabolic studies (with 14C), reaction kinetic studies (with, for example 2H or 3H), detection or imaging techniques, such as positron emission tomography (PET) or single-photon emission computed tomography (SPECT) including drug or substrate tissue distribution assays, or in radioactive treatment of patients. In particular, an 18F or labeled conjugate may be particularly desirable for PET or SPECT studies. Isotopically-labeled conjugates of the present invention can generally be prepared by conventional techniques known to those skilled in the art or by processes analogous to those described in the accompanying Examples using an appropriate isotopically-labeled reagents in place of the non-labeled reagent previously employed.

As used herein, the term “pharmaceutical composition” refers to a conjugate of the invention, or a pharmaceutically acceptable salt thereof, together with at least one pharmaceutically acceptable carrier, in a form suitable for oral or parenteral administration.

As used herein, the term “pharmaceutically acceptable carrier” refers to a substance useful in the preparation or use of a pharmaceutical composition and includes, for example, suitable diluents, solvents, dispersion media, surfactants, antioxidants, preservatives, isotonic agents, buffering agents, emulsifiers, absorption delaying agents, salts, drug stabilizers, binders, excipients, disintegration agents, lubricants, wetting agents, sweetening agents, flavoring agents, dyes, and combinations thereof, as would be known to those skilled in the art (see, for example, Remington The Science and Practice of Pharmacy, 22nd Ed. Pharmaceutical Press, 2013, pp. 1049-1070). The term “a therapeutically effective amount” of a conjugate of the present disclosure refers to an amount of the conjugate of the present disclosure that will elicit the biological or medical response of a subject, for example, reduction, inhibition or degradation of an enzyme or a protein activity, or ameliorate symptoms, alleviate conditions, slow or delay disease progression, or prevent a disease, etc.

As used herein, the term “subject” refers to primates (e.g., humans, male or female), dogs, rabbits, guinea pigs, pigs, rats and mice. In certain embodiments, the subject is a primate. In yet other embodiments, the subject is a human.

As used herein, the term “treat”, “treating” or “treatment” of any disease or disorder refers to alleviating or ameliorating the disease or disorder (i.e., slowing or arresting the development of the disease or at least one of the clinical symptoms thereof); or alleviating or ameliorating at least one physical parameter or biomarker associated with the disease or disorder, including those which may not be discernible to the patient.

As used herein, the term “prevent”, “preventing” or “prevention” of any disease or disorder refers to the prophylactic treatment of the disease or disorder; or delaying the onset or progression of the disease or disorder

As used herein, a subject is “in need of” a treatment if such subject would benefit biologically, medically or in quality of life from such treatment.

As used herein, the term “a,” “an,” “the” and similar terms used in the context of the present invention (especially in the context of the claims) are to be construed to cover both the singular and plural unless otherwise indicated herein or clearly contradicted by the context.

All methods described herein can be performed in any suitable order unless otherwise indicated herein or otherwise clearly contradicted by context. The use of any and all examples, or exemplary language (e.g. “such as”) provided herein is intended merely to better illuminate the invention and does not pose a limitation on the scope of the invention otherwise claimed.

Any asymmetric atom (e.g., carbon or the like) of the conjugate(s) of the present invention can be present in racemic or enantiomerically enriched, for example the (R)-, (S)- or (R,S)-configuration.

In certain embodiments, each asymmetric atom has at least 50% enantiomeric excess, at least 60% enantiomeric excess, at least 70% enantiomeric excess, at least 80% enantiomeric excess, at least 90% enantiomeric excess, at least 95% enantiomeric excess, or at least 99% enantiomeric excess in the (R)- or(S)-configuration. Substituents at atoms with unsaturated double bonds may, if possible, be present in cis-(Z)- or trans-(E)-form.

Accordingly, as used herein a conjugate of the present invention can be in the form of one of the possible stereoisomers, rotamers, atropisomers, tautomers or mixtures thereof, for example, as substantially pure geometric (cis or trans) stereoisomers, diastereomers, optical isomers (antipodes), racemates or mixtures thereof.

Any resulting mixtures of stereoisomers can be separated on the basis of the physicochemical differences of the constituents, into the pure or substantially pure geometric or optical isomers, diastereomers, racemates, for example, by chromatography and/or fractional crystallization.

Any resulting racemates of conjugates of the present invention or of intermediates can be resolved into the optical antipodes by known methods, e.g., by separation of the diastereomeric salts thereof, obtained with an optically active acid or base, and liberating the optically active acidic or basic conjugate. In particular, a basic moiety may thus be employed to resolve the conjugates of the present invention into their optical antipodes, e.g., by fractional crystallization of a salt formed with an optically active acid, e.g., tartaric acid, dibenzoyl tartaric acid, diacetyl tartaric acid, di-O,O′-p-toluoyl tartaric acid, mandelic acid, malic acid or camphor-10-sulfonic acid. Racemic conjugates of the present invention or racemic intermediates can also be resolved by chiral chromatography, e.g., high pressure liquid chromatography (HPLC) using a chiral adsorbent.

In another aspect, the present invention provides a pharmaceutical composition comprising a conjugate of the present invention, or a pharmaceutically acceptable salt thereof, and a pharmaceutically acceptable carrier. In a further embodiment, the composition comprises at least two pharmaceutically acceptable carriers, such as those described herein. The pharmaceutical composition can be formulated for particular routes of administration such as oral administration, parenteral administration (e.g. by injection, infusion, transdermal or topical administration), and rectal administration. Topical administration may also pertain to inhalation or intranasal application. The pharmaceutical compositions of the present invention can be made up in a solid form (including, without limitation, capsules, tablets, pills, granules, powders or suppositories), or in a liquid form (including, without limitation, solutions, suspensions or emulsions). Tablets may be either film coated or enteric coated according to methods known in the art. Typically, the pharmaceutical compositions are tablets or gelatin capsules comprising the active ingredient together with one or more of:
a) diluents, e.g., lactose, dextrose, sucrose, mannitol, sorbitol, cellulose and/or glycine;
b) lubricants, e.g., silica, talcum, stearic acid, its magnesium or calcium salt and/or polyethyleneglycol; for tablets also
c) binders, e.g., magnesium aluminum silicate, starch paste, gelatin, tragacanth, methylcellulose, sodium carboxymethylcellulose and/or polyvinylpyrrolidone; if desired
d) disintegrants, e.g., starches, agar, alginic acid or its sodium salt, or effervescent mixtures; and
e) absorbents, colorants, flavors and sweeteners.

The conjugates of the present invention in free form or in pharmaceutically acceptable salt form, exhibit valuable pharmacological properties, e.g. TEAD degradation properties, e.g. as indicated in vitro and in vivo tests as provided in the next sections, and are therefore indicated for therapy or for use as research chemicals, e.g. as tool compounds.

Conjugates of the present invention may be useful in the treatment of cancer, preferably wherein the cancer is selected from breast cancer, lung cancer, ovarian cancer, colorectal cancer, malignant pleural mesothelioma, pancreatic cancer, prostate cancer, gastric cancer, esophageal cancer, liver cancer and bone cancer.

Thus, as a further aspect, the present invention provides the use of a conjugate of the present invention in therapy. In a further embodiment, the therapy is selected from a disease which may be treated by modulation or degradation of TEAD. In an embodiment, the disease is cancer, for example wherein the cancer is selected from breast cancer, lung cancer, ovarian cancer, colorectal cancer, malignant pleural mesothelioma, pancreatic cancer, prostate cancer, gastric cancer, esophageal cancer, liver cancer and bone cancer.

In another aspect, the invention provides a method of treating a disease which is treated by degradation of TEAD, comprising administration of a therapeutically acceptable amount of a conjugate of the present invention. In an embodiment, the disease is cancer, for example wherein the cancer is selected from breast cancer, lung cancer, ovarian cancer, colorectal cancer, malignant pleural mesothelioma, pancreatic cancer, prostate cancer, gastric cancer, esophageal cancer, liver cancer and bone cancer.

Thus, as a further aspect, the present invention provides the use of a conjugate of the present invention for the manufacture of a medicament. In a further embodiment, the medicament is for treatment of a disease which may be treated by modulation or degradation of TEAD. In an embodiment, the disease is cancer, for example wherein the cancer is selected from breast cancer, lung cancer, ovarian cancer, colorectal cancer, malignant pleural mesothelioma, pancreatic cancer, prostate cancer, gastric cancer, esophageal cancer, liver cancer and bone cancer.

The pharmaceutical composition or combination of the present invention may, for example, be in unit dosage of about 1-1000 mg of active ingredient(s) for a subject of about 50-70 kg.

The conjugate of the present invention may be administered either simultaneously with, or before or after, one or more other therapeutic agent. The conjugate of the present invention may be administered separately, by the same or different route of administration, or together in the same pharmaceutical composition as the other agents. A therapeutic agent is, for example, a chemical compound, peptide, antibody, antibody fragment or nucleic acid, which is therapeutically active or enhances the therapeutic activity when administered to a patient in combination with a conjugate of the present invention.

In one embodiment, the invention provides a product comprising a conjugate of the present invention and at least one other therapeutic agent as a combined preparation for simultaneous, separate or sequential use in therapy. In one embodiment, the therapy is the treatment of a disease or condition mediated by TEAD. Products provided as a combined preparation include a composition comprising the conjugate of the present invention and the other therapeutic agent(s) together in the same pharmaceutical composition, or the conjugate of the present invention and the other therapeutic agent(s) in separate form, e.g. in the form of a kit.

In one embodiment, the invention provides a pharmaceutical composition comprising a conjugate of the present invention and another therapeutic agent(s). Optionally, the pharmaceutical composition may comprise a pharmaceutically acceptable carrier, as described above. In one embodiment, the invention provides a kit comprising two or more separate pharmaceutical compositions, at least one of which contains a conjugate of the present invention. In one embodiment, the kit comprises means for separately retaining said compositions, such as a container, divided bottle, or divided foil packet. An example of such a kit is a blister pack, as typically used for the packaging of tablets, capsules and the like.

The kit of the invention may be used for administering different dosage forms, for example, oral and parenteral, for administering the separate compositions at different dosage intervals, or for titrating the separate compositions against one another. To assist compliance, the kit of the invention typically comprises directions for administration.

In the combination therapies of the invention, the conjugate of the present invention and the other therapeutic agent may be manufactured and/or formulated by the same or different manufacturers. Moreover, the conjugate of the present invention and the other therapeutic may be brought together into a combination therapy: (i) prior to release of the combination product to physicians (e.g. in the case of a kit comprising the conjugate of the present invention and the other therapeutic agent); (ii) by the physician themselves (or under the guidance of the physician) shortly before administration; (iii) in the patient themselves, e.g. during sequential administration of the conjugate of the present invention and the other therapeutic agent.

Accordingly, the invention provides the use of a conjugate of the present invention for treating a disease or condition mediated by TEAD, wherein the medicament is prepared for administration with another therapeutic agent. The invention also provides the use of another therapeutic agent for treating a disease or condition mediated by TEAD, wherein the medicament is administered with a conjugate of the present invention.

The invention also provides a conjugate of the present invention for use in a method of treating a disease or condition mediated by TEAD, wherein the conjugate of the present invention is prepared for administration with another therapeutic agent. The invention also provides another therapeutic agent for use in a method of treating a disease or condition mediated by TEAD, wherein the other therapeutic agent is prepared for administration with a conjugate of the present invention. The invention also provides a conjugate of the present invention for use in a method of treating a disease or condition mediated by TEAD, wherein the conjugate of the present invention is administered with another therapeutic agent. The invention also provides another therapeutic agent for use in a method of treating a disease or condition mediated by TEAD, wherein the other therapeutic agent is administered with a conjugate of the present invention.

The invention also provides the use of a conjugate of the present invention for treating a disease or condition mediated by TEAD, wherein the patient has previously (e.g. within 24 hours) been treated with another therapeutic agent. The invention also provides the use of another therapeutic agent for treating a disease or condition mediated by TEAD, wherein the patient has previously (e.g. within 24 hours) been treated with a conjugate of the present invention.

### EXAMPLES

### Biological Assays and Data

The activity of a conjugate according to the present invention can be assessed by the following in vitro methods:

### In-Vitro Biochemical TR-FRET Assay

Avi-humanTEAD4²¹⁷⁻⁴³⁴ (1 nM, produced as described in Hau et al. ChemBioChem 14, 1218, 2013) and LANCE Eu-W1024 Streptavidin (0.5 nM, PerkinElmer) were first pre-incubated for 1 h at room temperature in HEPES (pH 7.4, 50 mM), KCl (100 mM), Tween-20 (0.05%), TCEP (0.25 mM), EDTA (1 mM), and BSA (0.05%)]. N-terminus Cy5 labeled humanYAP⁶⁰⁻¹⁰⁰ (20 nM) was then added to this preparation. Compounds were dissolved at 10 mM in 100% DMSO and serial dilutions were made in 100% DMSO. The diluted compound solutions were incubated in white 384-well plates (Greiner Bio-One) for 1 h at room temperature with the above described mix. The final DMSO concentration present in the assay was 1%. The fluorescence was measured (50 us delay between excitation and fluorescence, 75 us integration time) with a Genios Pro reader (Tecan) and use of an excitation wavelength of 340 nm and emission wavelengths of 620 nm and 665 nm. Data analyses were carried out by using the TR-FRET ratio emission 655 nm/620 nm. The IC₅₀ values were estimated by fitting the data by nonlinear fit regression (GraphPad Prism).

| Example | IC₅₀ [μM] |
|---|---|
| C-I-k | 0.0019 |
| C-I | 0.0022 |
| C-II | 0.0065 |
| 1 | 0.0019 |
| 2 | 0.0014 |
| 3 | <0.001 |
| 4 | 0.0011 |
| 5 | <0.001 |
| 6 | 0.0015 |
| 7 | <0.001 |
| 8 | 0.0016 |
| 9 | 0.0012 |
| 10 | 0.0026 |
| 11 | <0.001 |
| 12 | 0.0016 |
| 13 | 0.0018 |
| 14 | 0.0014 |
| 15 | <0.001 |
| 16 | 0.0013 |
| 17 | 0.001 |
| 18 | 0.0039 |
| 19 | 0.0021 |
| 20 | 0.0014 |
| 21 | 0.0016 |
| 22 | 0.001 |
| 23 | <0.001 |
| 24 | 0.0034 |
| 25 | 0.0024 |
| 26 | 0.004 |
| 27 | 0.004 |
| 28 | 0.0027 |
| 29 | 0.0033 |
| 30 | 0.0026 |
| 31 | 0.0018 |
| 32 | 0.023 |
| 33 | 0.094 |
| 34 | 0.0069 |
| 35 | 0.0072 |
| 36 | 0.092 |
| 37 | 0.12 |
| 38 | 0.007 |
| 39 | 0.003 |
| 40 | 0.0047 |
| 41 | 0.0023 |
| 42 | 0.019 |
| 43 | 0.0087 |
| 44 | 0.0023 |

### In-Vitro Cellular Assays

### Cell Lines

NCI-H2052 mesothelioma cells (RRID: CVCL_1518) bearing Hippo-pathway inactivating mutations upstream of YAP (homozygous deletion of LATS2 and mutation of NF2) were obtained from ATCC (American Type Culture Collection), while SF268 glioma cells (RRID: CVCL_1689) overexpressing YAP due to genomic amplification were obtained from the NCI (National Cancer Institute). MKN-45 gastric adenocarcinoma cells (RRID: CVCL_0434) genomically deleted for YAP were obtained from the JCRB (Japanese Collection of Research Bioresources) Cell Bank.

To enable monitoring of YAP-dependent pathway activity using a reporter gene assay approach, two YAP-driven cancer cell lines were engineered to express firefly-luciferase in a MCAT-promoter-dependent fashion. The MCAT-Luc reporter consists of 10 tandem copies of a seven nucleotide sequence (ATTCCTC, based on the muscle-specific cytidine-adenosine-thymidine (MCAT) promoter (5′-CATTCCT-3′)-element) present in the YAP/TEAD-responsive promoter, that was cloned 5′ to the minimal promoter element driving firefly luciferase in the pGL4.27 vector plasmid (Promega), followed by subcloning into pLENTI6_TR (Invitrogen). This approach was first applied to the SF268 cancer cell model. In brief, SF268 cells were first transduced with a lentivirus carrying a dox-inducible short-hairpin directed against the 3′-UTR of the YAP1 mRNA (shYAP_2371: 5′-catgagacaatttccatataattcaagagattatatggaaattgtctcatg-3′, Levy et al, Cell Deat Differ 2007 14 743) cloned into the Tet-pLKO-H1 vector (Wiederschain et al. Cell Cycle 2009 8 498), followed by transduction of the resulting neomycin-resistant cell pool with the pLENTI6MCAT_TR based MCAT_Luc reporter described above. Among the single cell clones of the resulting blasticidin-resistant cell pool, Clone 13 showed the highest extent of doxycyclin induced reduction in MCAT-driven luciferase-expression (approximately 70%). The resulting cell line, SF268-MCAT_Luc_dox-ishYAPLevy (Clone 13), was maintained in growth medium consisting of RPMI 1640 (Amimed Cat #1-41F01-I), 2 mM L-Glutamine (Amimed Cat #5-10K50-H), 1% MEM Non-essential amino acids (Amimed Cat #5-13K00-H), 10% tetracyline-free fetal calf serum (Gibco Cat #A31608-01 Lot 42F0863K), 1 mM sodium pyruvate (Amimed Cat #5-60F00-H), 1× Penicillin/Streptomycin (Amimed Cat #4-01F00-H)) containing 1 mg/ml G418 (Invitrogen #10131-027) and 4 μg/ml blasticidin (Gibco Cat #A11139-03). In addition, a similar approach was applied to the NCI-H2052 cancer cell model. Thus, this cell line was first transduced with a lentiviral Tet-pLKO-puro-based construct (pLKO Tet-On shYAP_2371) mediating tetracycline-inducible expression of a short-hairpin directed against the 3′-UTR of the YAP1 mRNA as described above. The resulting NCI-H2052 pLKO Tet-On shYAP_2371 cells were subsequently infected with pLenti6-TR MCAT_4.27 lentivirus. To this end, lentiviruses were generated by co-transfecting HEK293 FT cells with 3 μg of shRNA-encoding plasmid, 3 μg of gag/pol (pLP1), rev (pLP2) and 900 ng of VSV-G env (pLP/VSV-G) plasmids using Lipofectamine 2000 (Invitrogen Cat #11668019). Growth media was exchanged the following day and lentivirus-containing supernatant was harvested 48 hrs later. For lentiviral transduction, NCI-H2052 cells were seeded in a 12-well plate and centrifuged at 1′500 rpm for 90 min in media containing polybrene (8 μg/ml, Sigma Cat #H9268) and lentiviruses. After 24 hrs of recovery in normal media, transduced cells were selected in media containing 2 μg/ml puromycin (for pLKO Tet-ON constructs) or 2 μg/ml blasticidin (for pLenti6 MCAT_Luc). The resulting cell line, NCI-H2052 Tet-On shYAP_2371 pLenti6 MCAT_Luc, was maintained in growth medium (RPMI 1640 (Amimed Cat #1-41F01-I), 2 mM L-Glutamine (Amimed Cat #5-10K50-H), 1% MEM Non-essential amino acids (Amimed Cat #5-13K00-H), 10% tetracyline-free fetal calf serum (Gibco Cat #A31608-01 Lot 42F0863K), 1 mM sodium pyruvate (Amimed Cat #5-60F00-H), 1× Penicillin/Streptomycin (Amimed Cat #4-01F00-H)) containing 0.5 μg/ml puromycin (Gibco Cat #A11138-03) and 1 μg/ml blasticidin (Gibco Cat #A11139-03).

As a specificity control, MKN-45 cells were stably transduced with a Ubc-Luc reporter construct, in which constitutive expression of firefly luciferase is driven by the promoter of the ubiquitin C housekeeping gene. The resulting cell line, MKN-45/Ubc-luc, was selected and maintained in growth medium (RPMI 1640 (Amimed Cat #1-41F01-I), 2 mM L-Glutamine (Amimed Cat #5-10K50-H), 10% fetal calf serum (Amimed Cat #2-01F30-I Lot K08815P), 1% MEM Non-essential amino acids (Amimed Cat #5-13K00-H), 1× Penicillin/Streptomycin (Amimed Cat #4-01F00-H)) containing 1 μg/ml blasticidin (Gibco Cat #A11139-03).

All cells were cultured at 37° C. in a humidified 5% CO₂ incubator.

### Compound Solutions

Stock solutions of compounds were prepared at a concentration of 10 mM in DMSO and stored at 4° C. Where necessary to afford a full dose-response curve, the stock solutions were pre-diluted in DMSO to 1′000-fold the desired reduced start concentration. On the day after cell seeding, eleven 2-fold serial dilutions of each compound were dispensed directly into the cell assay plates using a HP 300D non-contact Digital Dispenser (TECAN). The final concentration of DMSO was normalized to 0.1% in all wells.

### YAP Reporter Gene Activity Assay

The ability of compounds to inhibit YAP-dependent transcription was assessed in NCI-H2052 Tet-On shYAP_2371 pLenti6 MCAT_Luc and SF268-MCAT_Luc, dox-i-shYAPLevy (Clone 13) cells, while specificity (lack of unspecific inhibition of luciferase expression or activity) was assessed in MKN-45/Ubc-luc cells. The individual cell lines were seeded at 2′500 cells/20 μl/well into white-wall, clear-bottom 384-well plates (Greiner, Cat #781098) and incubated over night at 37° C. prior to addition of serial compound dilutions as described above. Following incubation for 24 hrs at 37° C., compound-mediated modulation of reporter-gene activity was quantified 5 min after addition 20 μL Steady Glo BrightGlo (Promega Cat #E2620), by measuring luminescence intensity on a multi-mode plate-reader (TECAN) with an integration time of 100 ms.

### Proliferation Assay

The functional effect of compounds on cell proliferation was assessed using NCI-H2052 Tet-On shYAP_2371 pLenti6 MCAT_Luc, SF268-MCAT_Luc_dox-i-shYAPLevy (Clone 13) and MKN-45/Ubc-luc cells by quantifying cellular reducing capacity using the resazurin sodium salt dye reduction assay commercially known as AlamarBlue assay (O'Brien et al, Eur J Biochem 2000 267 5421). Briefly, individual cell lines were seeded at 750 cells/20 μl/well (NCI-H2052, SF268) or 500 cells/20 L/well (MKN-45) into black-wall, clear-bottom 384-well plates (Corning Cat #3712) and incubated over night at 37° C. prior to addition of serial compound dilutions as described above. After incubation for 72 hours at 37° C., compound-mediated modulation of cell viability was quantified as follows. After addition of 5 μL 5× resazurin stock solution (resazurin sodium salt (SIGMA Cat #R7017) dissolved at 3.25 μg/ml in PBS/0), cell plates were incubated for an additional 4 hours at 37° C. and 5% CO₂. Following equilibration of the plates at room temperature for 15 min, the levels of resorufin (reduced form of resazurin) were quantified using a TECAN M200 multi-purpose plate reader, with fluorescence excitation and emission wavelengths set to 544 and 590 nm, respectively. To enable differentiation of cytotoxic from cytostatic compound effects, the number of viable cells on the day of compound addition (day 0) was assessed in a separate cell plate and used to assess the extent of cell viability suppression as described in the following section.

### Data Analysis

For data analysis, the assay background value determined in wells containing medium, but no cells, was subtracted from all data points. Dose-dependent compound effects for individual treatment conditions in the RGA assay were expressed relative to the signal obtained in vehicle-treated control wells (set to 100%, while assay background was set to 0%), and IC50s were calculated following curve fitting with a four-parametric fit (model 203) using XLfit software (IDBS), or in HELIOS, an in-house software applying a multi-step decision tree to arrive at optimal concentration response curve fits (Gubler et al, SLAS Discovery 2018 23 474).

The extent of growth inhibition and potential cell kill was assessed by comparing the resorufin levels in compound-treated cells with those present at the time of compound addition. To this end, the following conditional concept was programmatically applied in HELIOS to calculate % growth (% G) for each compound-treated well: % G=(T−V0)/V0))*100 when T<V0, and % G=(T−V0)/(V−V0)))*100 when T≥V0, where V0 is the viability level at time of compound addition, while V and T represent vehicle-control and compound-treated viability levels, respectively, at the end of the compound incubation. 100%, 0% and −100% signify absence of growth inhibition, growth stasis, and complete cell kill, respectively. Compound concentrations leading to half-maximal growth inhibition (GI50) and residual cell viability at the highest tested compound concentration (Data(cmax), expressed in percent) were routinely calculated.

| | Reporter gene assay | | | Proliferation assay | | |
|---|---|---|---|---|---|---|
| | NCI-H2052 | SF268 | MKN-45 | NCI-H2052 | SF268 | MKN-45 |
| Example | IC₅₀ [μM] | IC₅₀ [μM] | IC₅₀ [μM] | GI₅₀ [μM] | GI₅₀ [μM] | GI₅₀ [μM] |
| C-I-k | 0.04 | 0.033 | 14.3 | 0.027 | 0.39 | 4.11 |
| C-I | 0.13 | 0.18 | >2 | 0.17 | >2 | >2 |
| C-II | 0.051 | 0.091 | >20 | 0.04 | 1.11 | >20 |
| 1 | 0.0036 | 0.0003 | >20 | 0.015 | 0.014 | 11 |
| 2 | 0.013 | 0.0009 | >20 | 0.048 | 0.041 | >20 |
| 3 | 0.098 | 0.0016 | >20 | 0.32 | 0.054 | >20 |
| 4 | 0.049 | 0.0017 | >20 | 0.14 | 0.033 | >20 |
| 5 | 0.16 | 0.0025 | >20 | 0.36 | 0.089 | >20 |
| 6 | 0.016 | 0.0005 | >20 | 0.061 | 0.039 | >20 |
| 7 | 0.029 | 0.0004 | >20 | 0.077 | 0.026 | >20 |
| 8 | 0.12 | 0.0031 | >20 | 0.31 | 0.12 | >20 |
| 9 | 0.35 | 0.0035 | >2 | 1.19 | 0.12 | >2 |
| 10 | 0.01 | 0.00043 | >20 | 0.038 | 0.03 | 12.5 |
| 11 | 0.091 | 0.0014 | >20 | 0.28 | 0.051 | >20 |
| 12 | 0.004 | 0.00038 | >2 | 0.015 | 0.02 | >2 |
| 13 | 0.0045 | 0.00049 | >2 | 0.013 | 0.023 | >2 |
| 14 | 0.0031 | 0.00063 | >2 | 0.011 | 0.035 | >2 |
| 15 | 0.017 | 0.0026 | >2 | 0.031 | 0.085 | 1.55 |
| 16 | 0.014 | 0.0012 | >2 | 0.052 | 0.064 | >2 |
| 17 | 0.015 | 0.00085 | >2 | 0.062 | 0.049 | >2 |
| 18 | 0.0099 | 0.0013 | >2 | 0.027 | 0.06 | >2 |
| 19 | 0.0046 | 0.00041 | >2 | 0.012 | 0.029 | >2 |
| 20 | 0.0039 | 0.00022 | >20 | 0.019 | 0.018 | >20 |
| 21 | 0.0014 | 0.0003 | >2 | 0.0043 | 0.0094 | >2 |
| 22 | 0.002 | 0.00018 | >2 | 0.0095 | 0.0095 | >2 |
| 23 | 0.013 | 0.0014 | >2 | 0.035 | 0.074 | >2 |
| 24 | 0.001 | 0.0002 | >2 | 0.0035 | 0.0062 | >2 |
| 25 | 0.0032 | 0.00054 | >2 | 0.0085 | 0.022 | >2 |
| 26 | 0.0013 | 0.00026 | >2 | 0.0025 | 0.012 | >2 |
| 27 | 0.00066 | 0.00017 | >2 | 0.0038 | 0.005 | >2 |
| 28 | 0.0044 | 0.00092 | >2 | 0.016 | 0.031 | >2 |
| 29 | 0.0038 | 0.00064 | >2 | 0.013 | 0.017 | >0.2 |
| 30 | 0.0069 | 0.0011 | >2 | 0.027 | 0.035 | >0.2 |
| 31 | 0.0019 | 0.00028 | >20 | 0.0066 | 0.019 | >20 |
| 32 | 5.90 | 2.75 | >20 | 6.29 | >20 | >20 |
| 33 | 3.14 | 1.34 | >20 | 4.40 | >20 | >20 |
| 34 | 2.80 | 3.53 | >20 | 2.27 | 19.3 | >20 |
| 35 | 0.032 | 0.023 | 0.044 | 0.035 | <0.02 | 0.033 |
| 36 | 0.024 | 0.0093 | 8.76 | 0.16 | 8.42 | 8.76 |
| 37 | 3.35 | 0.42 | 15.86 | 6.05 | 14.91 | 12.45 |
| 38 | 0.004 | 0.00042 | >2 | 0.022 | 0.024 | >2 |
| 39 | 0.056 | 0.0018 | >2 | 0.17 | 0.063 | >2 |
| 40 | 0.16 | 0.047 | >20 | 0.41 | 1.86 | >20 |
| 41 | 0.079 | 0.011 | >2 | 0.27 | 0.31 | >2 |
| 42 | 0.0042 | 0.00045 | >2 | 0.031 | 0.031 | >0.2 |
| 43 | 0.017 | 0.0023 | >2 | 0.073 | 0.068 | >0.2 |
| 44 | 0.63 | 0.0019 | >2 | 0.67 | 1.30 | >2 |

### TEAD1 Protein Abundance Flow Cytometry Assay in HEK293A

Degradation of TEAD1 was measured in HEK293A cells (Invitrogen R70507) expressing TEAD1-GFP and mCherry from a stably integrated bicistronic TEAD1-GFP-CHYSEL-mCherry construct. Reduction of the GFP signal measured by flow cytometry served as readout for TEAD1 degradation after degrader treatment.

Cloning of the pLenti6-TEAD1-GFP-CHYSEL-mCherry Sensor Vector

pLenti6-TEAD1-GFP-CHYSEL-mCherry was generated by gateway LR cloning (according to the manufacture protocol) between pENTR221-TEAD1 no stop codon and pLenti6_POI-GFP-CHYSEL-mCherry vector (cloning described in PAT058639).

pLenti6-TEAD1-GFP-CHYSEL-mCherry was sequenced for verification.

### Engineering of Stably Expressing 293A TEAD1-GFP-CHYSEL-mCherry Construct Sensor Cells

293A TEAD1-GFP-CHYSEL-mCherry sensor cells were generated by lentiviral vector transduction using the pLenti6-TEAD1-GFP-CHYSEL-mCherry sensor construct described before. Lentiviral particles were produced in HEK293 FT cells (Invitrogen R70007) by co-transfection of 500 ng pLenti6-TEAD1-GFP-CHYSEL-mCherry, 500 ng delta8.71 and 200 ng pVSVG diluted in 100 μL OptiMEM serum free medium (Invitrogen #11058-021) that was mixed after 5 min preincubation with 3 μL of Lipofectamine2000 (Invitrogen #11668-019) in 97 μL OptiMEM serum free medium. The mix was incubated for another 20 min at RT and then added on 1 mL of a freshly prepared suspension of HEK293 FT cells in a well of a 6-well plate (concentration 1.2×106 cells/mL). 1 day after transfection, the medium was replaced with 1.5 mL of complete growth medium (DMEM high Glucose+10% FCS+1% L-Glutamine+1% NEAA+1% NaPyr.). 48 h post transfection supernatant containing viral transducing particles was collected and frozen at −80° C.

2 days before transduction with viral particles, 1×105 HEK293A cells (Invitrogen R70507) were seeded in 2 mL growth medium in a well of a 6-well plate. Infection was performed with 150 μL of collected supernatant containing viral transducing particles in 1 mL medium including 8 μg/mL polybrene. 24 h post infection, stably transfected cells were selected with blasticidin at a concentration of 8 μg/mL referred to as stable HEK293A sensor cells.

### Quantitative TEAD1-GFP Abundance Measurements in Stable HEK293A Sensor Cells

Stable HEK293A sensor cells were maintained in complete growth medium (DMEM high Glucose+10% FCS+1% L-Glutamine+1% NEAA+1% NaPyr.+4 μg/mL blasticidin) with passaging performed twice per week. On Day 0, stable HEK293A sensor cells were seeded at 5,000 cells/well in a 96-well microtiter plate in 100 μL complete medium. On Day 1, cells were treated in duplicate with 10-point 1:3 dilution series of compound using the HP D300 Digital Dispenser (Tecan). DMSO concentrations were normalized across the plate to 0.1%. On Day 2, after 24 h of incubation at 37° C., treatment media was discarded, cells rinsed with 100 μL/well PBS and then detached using 40 μL trypsin/well for 5 min. Trypsin was neutralized with 100 μL/well PBS+20% FCS).

Flow cytometry was performed on the samples using the CytoFlex S (Beckman Coulter). Cell identification was then performed using forward (FSC) vs. side scatter (SSC) plots. Single cell discrimination was performed using SSC-Width (SSC-W) vs. SSC-Height (SSCH) plots. Median GFP values for 2,500 single cells were used to determine TEAD1 levels. Median GFP values from HEK293A were used as a background signal and thus defining 0% TEAD1 signal. Median GFP values from DMSO treated HEK293A TEAD1-GFP CHYSEL-mCherry were used to define 100%

TEAD1 signal for subsequent DC₅₀ curves (concentration at 50% TAED1 degradation). GFP and RFP were read in the channels called FITC and PE, respectively. Concentration response curves plotting relative reduction of the GFP signal (measured by flow cytometry) versus 10 compound concentrations (starting concentration 10 μM, 3 fold dilution steps) of the compounds allowed generation of DC₅₀ values.

| Example | TEAD1 DC₅₀ [μM] |
|---|---|
| 1 | 0.0006 |
| 2 | 0.002 |
| 3 | 0.006 |
| 5 | 0.035 |
| 6 | 0.002 |
| 7 | 0.003 |
| 9 | 0.112 |
| 10 | 0.001 |
| 11 | 0.011 |
| 12 | 0.001 |
| 13 | 0.0005 |
| 14 | 0.003 |
| 15 | 0.003 |
| 16 | 0.006 |
| 17 | 0.004 |
| 18 | 0.002 |
| 19 | 0.0005 |
| 20 | 0.0004 |
| 21 | 0.0003 |
| 22 | 0.0004 |
| 23 | 0.003 |
| 24 | <0.0003 |
| 25 | 0.0005 |
| 26 | 0.0003 |
| 31 | 0.0006 |
| 38 | 0.001 |
| 39 | 0.009 |
| 40 | 0.02 |
| 41 | 0.027 |
| 44 | 2.8 |

### Preparation of Compounds

Conjugates of the present invention can be prepared as described in the following Examples. The following examples are intended to illustrate the invention and are not to be construed as being limitations thereof. Temperatures are given in degrees Celsius. If not mentioned otherwise, all evaporations are performed under reduced pressure, typically between about 15 mm Hg and 100 mm Hg (=20-133 mbar). The structure of final products, intermediates and starting materials is confirmed by standard analytical methods, e.g., spectroscopic characteristics, e.g., MS, IR, NMR.

All starting materials, building blocks, reagents, acids, bases, dehydrating agents, solvents, and catalysts utilized to synthesize the compounds of the present invention are either commercially available or can be produced by organic synthesis methods known to one of ordinary skill in the art. Further, the compounds of the present invention can be produced by organic synthesis methods known to one of ordinary skill in the art as shown in the following examples.

### Abbreviations

| Abbreviation | Description |
|---|---|
| ACN | acetonitrile |
| aq. | aqueous |
| AcOH | Acetic acid |
| Ar | Argon |
| brine | Saturated aqueous sodium chloride |
| calcd | calculated |
| conc | concentrated |
| dba | Dibenzylideneacetone |
| DBU | 1,8-Diazabicxclo[5.4.0]undec-7-ene |
| DCE | 1,2-Dichloroethane |
| DCM | Dichloromethane |
| DEA | Diethylamine |
| DEAD | Diethyl azodicarboxylate |
| DIAD | Diisopropyl azodicarboxylate |
| DIPEA | N,N-Diisopropylethylamine, |
| | N-Ethyl-N-isopropylpropan-2-amine |
| DMA | N,N-Dimethylacetamide |
| DMAP | 4-Dimethylaminopyridine |
| DMF | N,N-Dimethylformamide |
| DMSO | Dimethylsulfoxide |
| DMSO-d₆ or DMSO-d6 | Hexadeuterodimethyl sulfoxide |
| dppf | 1,1′-bis(diphenylphosphino)ferrocene |
| DSC | Differential scanning calorimetry |
| ee | Enantiomeric excess |
| ESI-MS | Electrospray ionization mass spectroscopy |
| EtOAc | Ethyl acetate |
| EtOH | Ethanol |
| Et₂O | Diethylether |
| h | hour |
| HPLC | High-performance liquid chromatography |
| HV | High vacuum |
| IPA | 2-Propanol |
| L/mL | litre/millilitre |
| LDA | Lithium diisopropylamide |
| LC-MS | liquid chromatography and mass spectroscopy |
| LHMDS | Lithium hexamethyldisilazide |
| M | molar |
| mCPBA | meta-chloroperoxybenzoic acid |
| MeOH | methanol |
| min | minutes |
| mp | melting point |
| MW, mw | microwave |
| m/z | mass to charge ratio |
| NaOtBu | Sodium tert-butoxide |
| NBS | N-Bromosuccinimide |
| n-Buli | n-Butyllithium |
| n.d. | not determined |
| NMP | N-methylpyrrolidinone |
| NMR | Nuclear magnetic resonance |
| 1H-NMR, ¹H-NMR | ¹H-Nuclear Magnetic Resonance |
| org. | organic |
| PE | Petrol ether |
| p-TsOH | para-toulene sulfonic acid |
| RM or rm | Reaction mixture |
| RP | reversed phase |
| RT | Room temperature |
| sat | saturated |
| TBAF | Tetrabutylammonium fluoride |
| TBME | 2-Methoxy-2-methylpropane |
| TEA | Triethylamine |
| TFA | Trifluoroacetic acid |
| THF | Tetrahydrofuran |
| t_{R} | Retention time (if not indicated, in minutes) |
| UPLC | Ultra-performance liquid chromatography |

### General Conditions:

Mass spectra were acquired on LC-MS systems using electrospray ionization methods with a range of instruments of the following configurations: Waters Acquity UPLC with Waters SQ detector, [M+H]⁺ refers to the protonated molecular ion of the chemical species.

NMR spectra were run with Bruker Ultrashield™400 (400 MHZ) spectrometers, both with and without trimethylsilane as an internal standard. Chemical shifts (d-values) are reported in ppm downfield from tetramethylsilane, spectra splitting pattern are designated as singlet(s), doublet (d), triplet (t), quartet (q), multiplet, unresolved or more overlapping signals (m), broad signal (br). Solvents are given in parentheses.

### Instrumentation

UPLC-MS Methods: Using Waters Acquity UPLC with Waters SQ detector.

Method UPLC-MS 1: UPLC-MS instrument: Waters Acquity UPLC with Waters SQ detector; column: Acquity UPLC HSS T3, 1.8 μm, 2.1×50 mm, column temperature: 60° C.; eluent: A: water+0.05% formic acid+3.75 mM ammonium acetate (pH 3.8), B: acetonitrile+0.04% formic acid; flow rate: 1.0 mL/min; gradient: 5 to 98% B in 1.40 min, 98% B for 0.40 min.

Method UPLC-MS 2: UPLC-MS instrument: Waters Acquity UPLC with Waters SQ detector; column: Acquity UPLC HSS T3, 1.8 μm, 2.1×100 mm, column temperature: 60° C.; eluent: A: water+0.05% formic acid+3.75 mM ammonium acetate (pH 3.8), B: acetonitrile+0.04% formic acid; flow rate: 0.8 mL/min; gradient: 5 to 98% B in 9.40 min, 98% B for 0.40 min.

Method UPLC-MS 3: UPLC-MS instrument: Waters Acquity UPLC with Waters SQ detector; column: XBridge BEH C18, 2.5 μm, 2.1×50 mm, column temperature: 80° C.; eluent: A: water+5 mM NH₄OH, B: acetonitrile+5 mM NH₄OH; flow rate: 1.0 mL/min; gradient: 2 to 98% B in 1.40 min.

Method UPLC-MS 4: UPLC-MS instrument: Waters Acquity UPLC with Waters SQ detector; column: CORTECS C18+, 2.7 μm, 2.1×50 mm, column temperature: 80° C.; eluent: A: water+4.76% isopropanol+0.05% formic acid+3.75 mM ammonium acetate, B: isopropanol+0.05% formic acid; flow rate: 1.0 mL/min; gradient: 1 to 50% B in 1.40 min, 50 to 98% B in 0.30 min.

Method UPLC-MS 5: UPLC-MS instrument: Waters Acquity UPLC with Waters SQ detector; column: CORTECS C18+, 2.7 μm, 2.1×50 mm, column temperature: 80° C.; eluent: A: water+0.05% formic acid+3.75 mM ammonium acetate, B: isopropanol+0.05% formic acid; flow rate: 1.0 mL/min; gradient: 1 to 98% B in 1.40 min.

### Section A: Preparation of Intermediates

The intermediate compounds described herein are useful in the preparation of conjugates of formula (I). Thus, in an aspect, the disclosure provides an intermediate compound as provided in any one of Sections A and B below, or a salt thereof. In another aspect, the disclosure provides the use of an intermediate compound as provided in any one of Sections A and B below, or a salt thereof, in the manufacture of a compound of formula (I) (e.g., formula (Ia), (Ia-i), (Ia-ii), (Ia-iii), (Ib), (Ib-i), (Ib-ii), (Ib-iii), (Ic), (Ic-i), (Ic-ii) or (Ic-iii).

### Synthesis of 1-(2-Methoxy-5-(4-(piperidin-4-ylmethyl)piperidine-1-carbonyl)phenyl)dihydropyrimidine-2,4(1H,3H)-dione (L-I)

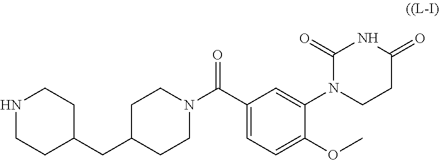 [SMILES: COc1ccc(C(=O)N2CCC(CC3CCNCC3)CC2)cc1N1CCC(=O)NC1=O]

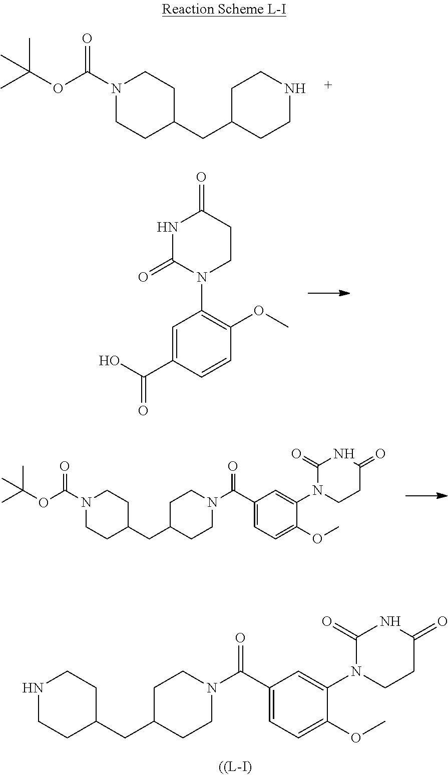

### Step 1: Tert-butyl 4-((1-(3-(2,4-dioxotetrahydropyrimidin-1(2H)-yl)-4-methoxybenzoyl)piperidin-4-yl)methyl)piperidine-1-carboxylate

To a stirred solution of 3-(2,4-dioxotetrahydropyrimidin-1(2H)-yl)-4-methoxybenzoic acid (PCT/IB2019/052346) (257 mg, 0.974 mmol) in DMF (9 mL) was added DIPEA (464 μl, 2.66 mmol) followed by HATU (404 mg, 1.062 mmol). After 30 min, 4-piperidin-4-ylmethyl-piperidine-1-carboxylic acid tert-butyl ester (250 mg, 0.885 mmol) was added and the yellow reaction mixture was stirred overnight at RT. The reaction mixture was quenched with water and extracted twice with EtOAc. The organic layers were combined and washed with a sat solution of brine, dried (phase separation cartouche) and concentrated. The residue was purified by reverse phase chromatography (C18 silica, (water+0.1% NH₄OH)/ACN; gradient: 0% to 100% ACN) to give the title compound (480 mg) as a yellow oil. UPLC-MS 4: m/z 529.3 [M+H]⁺, t_{R}=0.96 min.

### Step 2: 1-(2-Methoxy-5-(4-(piperidin-4-ylmethyl)piperidine-1-carbonyl)phenyl)dihydropyrimidine-2,4(1H, 3H)-dione (L-I)

To a stirred yellow solution of tert-butyl 4-((1-(3-(2,4-dioxotetrahydropyrimidin-1(2H)-yl)-4-methoxybenzoyl)piperidin-4-yl)methyl)piperidine-1-carboxylate (480 mg, 0.91 mmol) in dioxane (6 mL) was added HCl (2.27 mL, 9.08 mmol, 4 M in dioxane), the resulting mixture was stirred at RT for 18 h. The reaction mixture was concentrated to give the title compound (544 mg) as a light yellow residue as HCl salt. UPLC-MS 4: m/z 429.4 [M+H]⁺, t_{R}=0.18 min

### Synthesis of 1-(2-Methoxy-5-(4-(piperidin-4-yloxy)piperidine-1-carbonyl)phenyl)dihydropyrimidine-2,4(1H,3H)-dione (L-II) HCL Salt

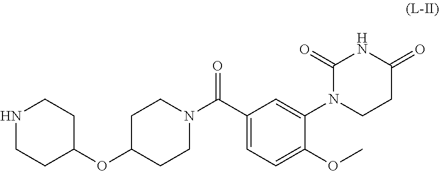 [SMILES: COc1ccc(C(=O)N2CCC(OC3CCNCC3)CC2)cc1N1CCC(=O)NC1=O]

### Step 1: Tert-butyl 4-((1-(3-(2,4-dioxotetrahydropyrimidin-1(2H)-yl)-4-methoxybenzoyl)piperidin-4-yl)oxy)piperidine-1-carboxylate

DIPEA (0.198 mL, 1.135 mmol) followed by HATU (173 mg, 0.454 mmol) was added to a solution of 3-(2,4-dioxotetrahydropyrimidin-1(2H)-yl)-4-methoxybenzoic acid (100 mg, 0.378 mmol) in DMF (2.5 mL). After 30 min, tert-butyl 4-(piperidin-4-yloxy)piperidine-1-carboxylate (108 mg, 0.378 mmol) was added and stirring was continued overnight at RT. The reaction mixture was quenced by the addition of a sat solution of NaHCO₃ and extracted twice with EtOAc. The combined organic phases were washed with water and brine, dried (phase separation catrouche) and concentrated. The crude product was purified by reverse phase chromatography (C18 silica, (water+0.1% NH₄OH)/ACN; gradient: 0% to 100% ACN) to give the title compound (177 mg) as a colorless powder. UPLC-MS 4: m/z 431.6 [M+H-Boc]⁺, t_{R}=0.81 min.

### Step 2: 1-(2-Methoxy-5-(4-(piperidin-4-yloxy)piperidine-1-carbonyl)phenyl)dihydropyrimidine-2,4(1H,3H)-dione hydrochloride (L-II)

Tert-butyl 4-((1-(3-(2,4-dioxotetrahydropyrimidin-1(2H)-yl)-4-methoxybenzoyl)piperidin-4-yl)oxy)piperidine-1-carboxylate (227 mg, 0.428 mmol) was dissolved in HCl (5.35 mL, 21.4 mmol, 4 M in dioxane) and the resulting solution was stirred at RT for 2 h. The reaction mixture was concentrated and dried under HV to afford the title product as its hydrochloride salt (233 mg) which was used without further purification in the next step. UPLC-MS 3: m/z 431.4 [M+H]⁺, t_{R}=0.62 min.

The following compounds were synthesized accordingly:

| | | UPLC MS m/z |
|---|---|---|
| Intermdiate | Structure/Chemical Name | t_{R} [min] (method) |
| L-III | 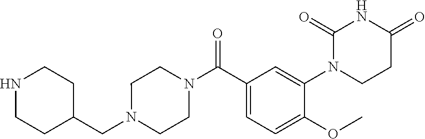 [SMILES: COc1ccc(C(=O)N2CCN(CC3CCNCC3)CC2)cc1N1CCC(=O)NC1=O] | 430.5 [M + H]⁺ 0.51 (3) |
| | 1-(2-methoxy-5-(4-(piperidin-4-ylmethyl)piperazine-1- | |
| | carbonyl)phenyl)dihydropyrimidine-2,4(1H,3H)-dione | |
| L-IV | 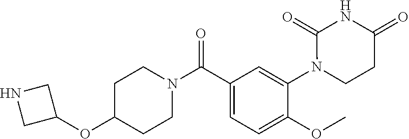 [SMILES: COc1ccc(C(=O)N2CCC(OC3CNC3)CC2)cc1N1CCC(=O)NC1=O] | 403.4 [M + H]⁺ 0.48 (3) |
| | 1-(5-(4-(azetidin-3-yloxy)piperidine-1-carbonyl)-2- | |
| | methoxyphenyl)dihydropyrimidine-2,4(1H,3H)-dione | |
| L-V | 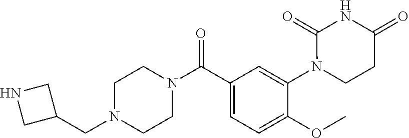 [SMILES: COc1ccc(C(=O)N2CCN(CC3CNC3)CC2)cc1N1CCC(=O)NC1=O] | 402.5 [M + H]⁺ 0.54 (3) |
| | 1-(5-(4-(azetidin-3-ylmethyl)piperazine-1-carbonyl)-2- | |
| | methoxyphenyl)dihydropyrimidine-2,4(1H,3H)-dione | |
| L-VI | 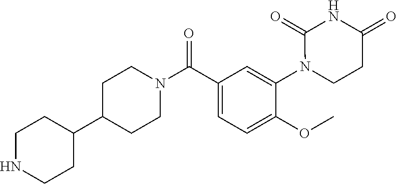 [SMILES: COc1ccc(C(=O)N2CCC(C3CCNCC3)CC2)cc1N1CCC(=O)NC1=O] | 415.5 [M + H]⁺ 0.54 (5) |
| | 1-(5-([4,4′-bipiperidine]-1-carbonyl)-2- | |
| | methoxyphenyl)dihydropyrimidine-2,4(1H,3H)-dione | |
| L-VII | 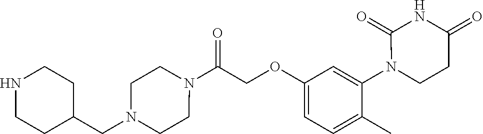 [SMILES: Cc1ccc(OCC(=O)N2CCN(CC3CCNCC3)CC2)cc1N1CCC(=O)NC1=O] | 444.5 [M + H]⁺ 0.62 (3) |
| | 1-(2-methyl-5-(2-oxo-2-(4-(piperidin-4-ylmethyl)piperazin-1-yl)ethoxy)phenyl) | |
| | dihydropyrimidine-2,4(1H,3H)-dione | |
| | 2-(3-(2,4-dioxotetrahydropyrimidin-1(2H)-yl)-4- | |
| | methylphenoxy)acetic acid synthesized according to WO2021053495 | |
| L-VIII | 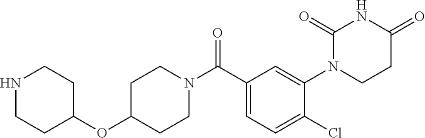 [SMILES: O=C1CCN(c2cc(C(=O)N3CCC(OC4CCNCC4)CC3)ccc2Cl)C(=O)N1] | 435.3 [M + H]⁺ 0.19 (4) |
| | 1-(2-chloro-5-(4-(piperidin-4-yloxy)piperidine-1- | |
| | carbonyl)phenyl)dihydropyrimidine-2,4(1H,3H)-dione | |
| | 4-chloro-3-(2,4-dioxotetrahydropyrimidin-1(2H)- | |
| | yl)benzoic acid synthesized according to PCT/IB2019/052346 | |
| L-IX | 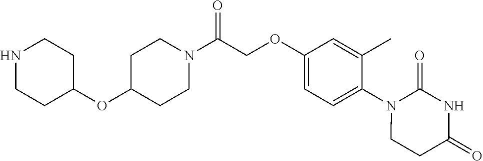 [SMILES: Cc1cc(OCC(=O)N2CCC(OC3CCNCC3)CC2)ccc1N1CCC(=O)NC1=O] | 445.3 [M + H]⁺ 0.17 (4) |
| | 1-(2-methyl-4-(2-oxo-2-(4-(piperidin-4-yloxy)piperidin-1- | |
| | yl)ethoxy)phenyl)dihydropyrimidine-2,4(1H,3H)-dione | |
| | 2-(4-(2,4-dioxotetrahydropyrimidin-1(2H)-yl)-3- | |
| | methylphenoxy)acetic acid synthesized according to WO2021053495 | |

### Synthesis of 1-(5-(4-((1-(2-aminoethyl)piperidin-4-yl)methyl)piperazine-1-carbonyl)-2-methoxyphenyl)dihydropyrimidine-2,4(1H,3H)-dione (L-X)

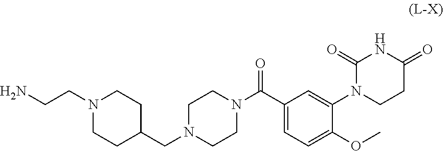 [SMILES: COc1ccc(C(=O)N2CCN(CC3CCN(CCN)CC3)CC2)cc1N1CCC(=O)NC1=O]

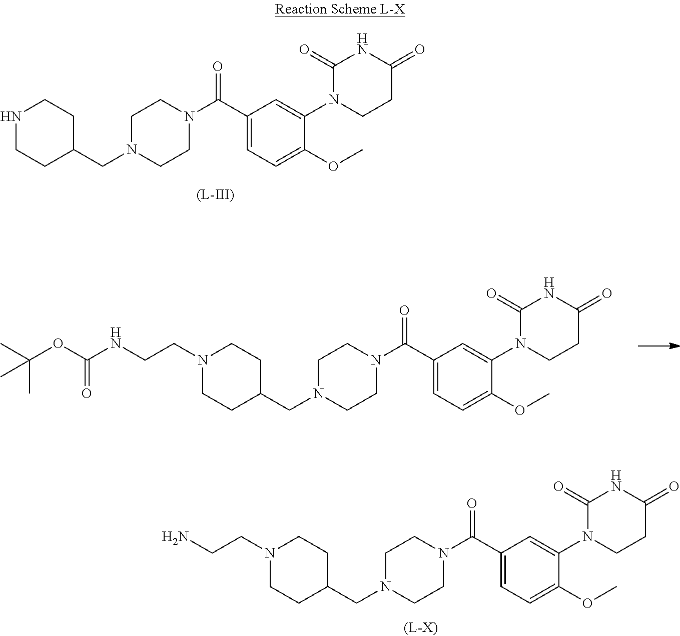 [SMILES: COc1ccc(C(=O)N2CCN(CC3CCNCC3)CC2)cc1N1CCC(=O)NC1=O ; COc1ccc(C(=O)N2CCN(CC3CCN(CCNC(=O)OC(C)(C)C)CC3)CC2)cc1N1CCC(=O)NC1=O ; COc1ccc(C(=O)N2CCN(CC3CCN(CCN)CC3)CC2)cc1N1CCC(=O)NC1=O]

### Step 1: Tert-butyl (2-(4-((4-(3-(2,4-dioxotetrahydropyrimidin-1(2H)-yl)-4-methoxybenzoyl) piperazin-1-yl)methyl)piperidin-1-yl)ethyl)carbamate

Acetic acid (0.056 mL, 0.97 mmol), tert-butyl (2-oxoethyl)carbamate (309 mg, 1.94 mmol) amd NaCNBH₃ (122 mg, 1.94 mmol) were added to a solution of 1-(2-methoxy-5-(4-(piperidin-4-ylmethyl)piperazine-1-carbonyl)phenyl)dihydropyrimidine-2,4(1H,3H)-dione hydrochloride (L-III) (180 mg, 0.328 mmol) in MeOH (4 mL) and DMF (2.67 mL) and the reaction mixture was stirred overnight at RT. A sat solution of NaHCO₃ was added and the mixture was extracted twice with EtOAc. The combined organic phases were washed with water and brine, dried (phase separation cartridge) and concentrated. The crude product was purified by flash chromatography (silica, DCM/MeOH, gradient: 0 to 20% MeOH) to give the title product (169 mg) as a colorless powder. UPLC-MS 5: m/z 573.5 [M+H]⁺, t_{R}=0.57 min.

### Step 2: 1-(5-(4-((1-(2-Aminoethyl)piperidin-4-yl)methyl)piperazine-1-carbonyl)-2-methoxyphenyl)dihydropyrimidine-2,4(1H,3H)-dione (L-X)

A suspension of tert-butyl (2-(4-((4-(3-(2,4-dioxotetrahydropyrimidin-1(2H)-yl)-4-methoxybenzoyl) piperazin-1-yl)methyl)piperidin-1-yl)ethyl)carbamate (169 mg, 0.295 mmol) in HCl (7.08 mL, 8.85 mmol, 1.25 N in EtOH) was stirred at RT overnight. The reaction mixture was concentrated and the residue was purified by prep HPLC to afford the title product as its TFA salt (79 mg). UPLC-MS 3: m/z 473.5 [M+H]⁺, t_{R}=0.60 min.

The following compounds were synthesized accordingly:

| | | UPLC MS m/z |
|---|---|---|
| Intermdiate | Structure/Chemical Name | t_{R} [min] (method) |
| L-XI | 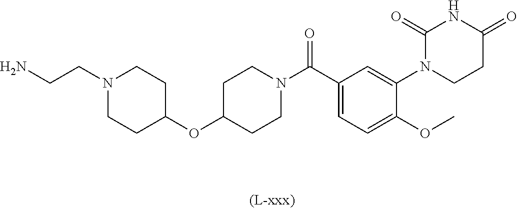 [SMILES: COc1ccc(C(=O)N2CCC(OC3CCN(CCN)CC3)CC2)cc1N1CCC(=O)NC1=O ; "(L-xxx)"] | 474.4 [M + H]⁺ 0.12 (4) |
| L-XII | 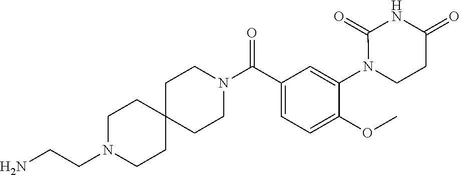 [SMILES: COc1ccc(C(=O)N2CCC3(CCN(CCN)CC3)CC2)cc1N1CCC(=O)NC1=O] | 444.3 [M + H]⁺ 0.26 (5) |
| | 1-(5-(9-(2-aminoethyl)-3,9-diazaspiro[5.5]undecane-3- | |
| | carbonyl)-2-methoxyphenyl)dihydropyrimidine-2,4(1H,3H)-dione | |
| L-XIII | 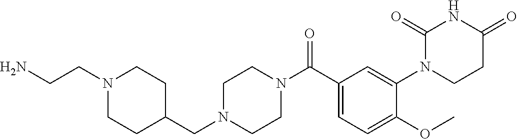 [SMILES: COc1ccc(C(=O)N2CCN(CC3CCN(CCN)CC3)CC2)cc1N1CCC(=O)NC1=O] | 473.5 [M + H]⁺ 0.60 (3) |
| | 1-(5-(4-((1-(2-aminoethyl)piperidin-4-yl)methyl)piperazine-1-carbonyl)-2- | |
| | methoxyphenyl)dihydropyrimidine-2,4(1H,3H)-dione | |
| L-XIV | 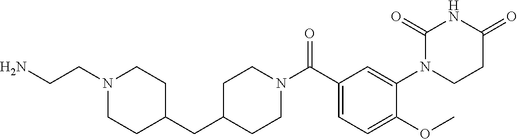 [SMILES: COc1ccc(C(=O)N2CCC(CC3CCN(CCN)CC3)CC2)cc1N1CCC(=O)NC1=O] | 472.5 [M + H]⁺ 0.62 (3) |
| | 1-(5-(4-((1-(2-aminoethyl)piperidin-4-yl)methyl)piperidine-1-carbonyl)-2- | |
| | methoxyphenyl)dihydropyrimidine-2,4(1H,3H)-dione | |
| L-XV | 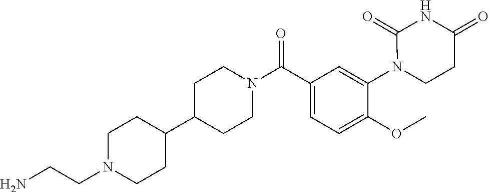 [SMILES: COc1ccc(C(=O)N2CCC(C3CCN(CCN)CC3)CC2)cc1N1CCC(=O)NC1=O] | 458.5 [M + H]⁺ 0.72 (3) |
| | 1-(5-(1′-(2-aminoethyl)-[4,4′-bipiperidine]-1-carbonyl)-2- | |
| | methoxyphenyl)dihydropyrimidine-2,4(1H,3H)-dione | |

### Synthesis of 1-((2-oxo-1-(2-(4-(piperidin-4-ylmethyl)piperidin-1-yl)ethyl)-1,2-dihydropyridin-3-yl)methyl)dihydropyrimidine-2,4(1H,3H)-dione (L-XVI)

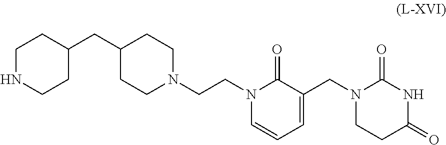 [SMILES: O=C1CCN(Cc2cccn(CCN3CCC(CC4CCNCC4)CC3)c2=O)C(=O)N1]

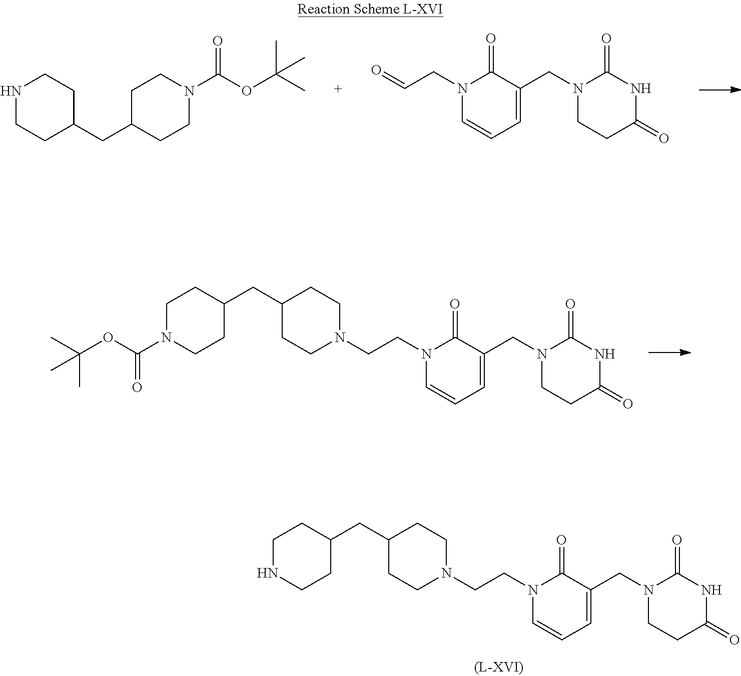 [SMILES: CC(C)(C)OC(=O)N1CCC(CC2CCNCC2)CC1 ; O=CCn1cccc(CN2CCC(=O)NC2=O)c1=O ; CC(C)(C)OC(=O)N1CCC(CC2CCN(CCn3cccc(CN4CCC(=O)NC4=O)c3=O)CC2)CC1 ; O=C1CCN(Cc2cccn(CCN3CCC(CC4CCNCC4)CC3)c2=O)C(=O)N1]

### Step 1: Tert-butyl 4-((1-(2-(3-((2,4-dioxotetrahydropyrimidin-1(2H)-yl)methyl)-2-oxopyridin-1(2H)-yl)ethyl)piperidin-4-yl)methyl)piperidine-1-carboxylate

2-(3-((2,4-Dioxotetrahydropyrimidin-1(2H)-yl)methyl)-2-oxopyridin-1(2H)-yl) acetaldehyde (synthesized according to WO2021053495) (43.6 mg, 0.166 mmol) followed by NaBH(Oac)3 (63.8 mg, 0.30 mmol) was added to a suspension of tert-butyl 4-(piperidin-4-ylmethyl)piperidine-1-carboxylate (50 mg, 0.15 mmol) in DCE (1.5 mL) and the reaction mixture was stirred at RT for 18 h. The reaction mixture was concentrated and purified by prep HPLC to afford the title product (30 mg) as a colorless solid. UPLC-MS 4: m/z 530.5 [M+H]⁺, t_{R}=0.59 min.

### Step 2: 1-((2-Oxo-1-(2-(4-(piperidin-4-ylmethyl)piperidin-1-yl)ethyl)-1,2-dihydropyridin-3-yl)methyl)dihydropyrimidine-2,4(1H,3H)-dione (L-XVI)

HCl (212 □L, 0.85 mmol, 4 M in dioxane) was added to a solution of tert-butyl 4-((1-(2-(3-((2,4-dioxotetrahydropyrimidin-1(2H)-yl)methyl)-2-oxopyridin-1(2H)-yl)ethyl)piperidin-4-yl)methyl)piperidine-1-carboxylate (30 mg, 0.057 mmol) in dioxane (0.38 mL) and the reaction mixture was stirred at RT for 18 h. After concentration of the reaction mixture the residue was taken up in ACN/water and lyophilized to give the title product (26 mg) as its hydochloride salt. UPLC-MS 4: m/z 430.4 [M+H]⁺, t_{R}=0.11 min.

### Synthesis of 2-(3-(2,4-dioxotetrahydropyrimidin-1(2H)-yl)-4-fluorophenoxy) acetic acid (L-XVII)

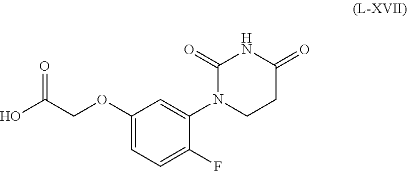 [SMILES: O=C(O)COc1ccc(F)c(N2CCC(=O)NC2=O)c1]

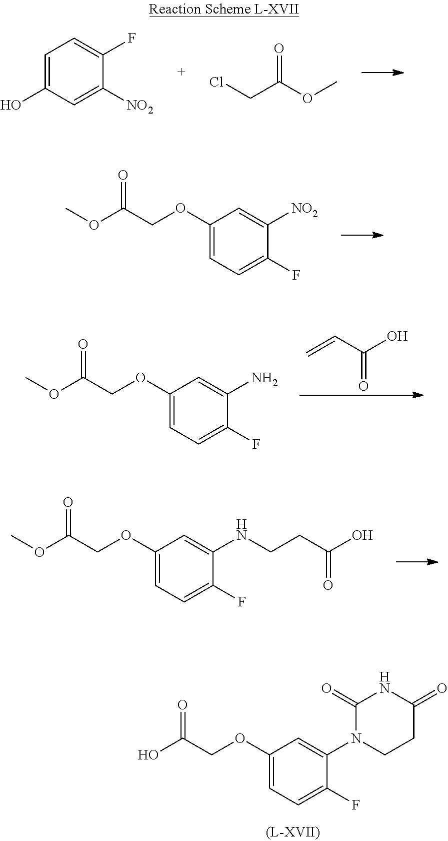

### Step 1: Methyl 2-(4-fluoro-3-nitrophenoxy)acetate

K₂CO₃ (1.935 g, 14.00 mmol) was added to a solution of 4-fluoro-3-nitrophenol (2 g, 12.73 mmol) in acetone (42.4 ml). Then methyl chloroacetate (1.236 ml, 14.00 mmol) was added and the resulting brown suspension was heated at 55° C. for 18 h. The reaction mixture was allowed to cool to RT and filtered. The filtrate was diluted with water and extracted with EtOAc, the organic layer was dried by passing through a phase separation cartridge and concentrated under reduced pressure to afford the crude product. Purification by flash chromatography (silica, cyclohexyne/EtOAc, gradient: 0 to 30% EtOAc) afforded the title product (1.6 g) as an oil. ¹H NMR (400 MHZ, DMSO-d₆) δ (ppm) 7.68 (dd, J=6.0, 3.2 Hz, 1H), 7.54 (dd, J=10.8, 9.2 Hz, 1H), 7.43 (dt, J=9.2, 3.5 Hz, 1H), 4.95 (s, 2H), 3.71 (s, 3H).

### Step 2: Methyl 2-(3-amino-4-fluorophenoxy)acetate (L-XVII)

A solution of methyl 2-(4-fluoro-3-nitrophenoxy)acetate (1.6 g, 6.98 mmol) in MeOH (70 mL) was purged three times with Ar. Then Pd/C (0.743 g, 0.698 mmol) was added and the reaction mixture was vigorously stirred for 22 h under a H₂ atmosphere. The black suspension was filtered through Celite and concentrated to afford the title compound (1.23 g) as a red oil which was used in the next step without further purification. UPLC-MS 4: m/z 199.8 [M+H]⁺, t_{R}=0.38 min.

### Step 3 and 4: 2-(3-(2,4-Dioxotetrahydropyrimidin-1(2H)-yl)-4-fluorophenoxy) acetic acid

Methyl 2-(3-amino-4-fluorophenoxy)acetate (1.23 g, 5.22 mmol) was suspended in acrylic acid (1.43 mL, 20.88 mmol) and the reaction mixture was stirred at 100° C. for 2 h. After cooling down the reaction mixture acetic acid (5.85 mL) and urea (1.87 g, 31.2 mmol) were added and the resulting reaction mixture was heated at 120° C. for 18 h. The resulting brown solution was poured into a cold solution of water (26 mL) and conc HCl (1-8 mL) and extracted twice with EtOAc and with DCM/MeOH 9:1. The combined organic layers were dried by passing through a phase separation cartridge and concentrated. The resulting residue was washed with DCM, isolated by filtration, washed with MeOH and dried under HV to afford the title compound (450 mg) as a beige solid. UPLC-MS 4: m/z 283.1 [M+H]⁺, t_{R}=0.18 min

### Synthesis of 2-(3-(2,4-dioxotetrahydropyrimidin-1(2H)-yl)-4-methoxyphenoxy) acetic acid (L-XVIII)

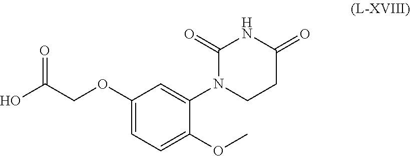 [SMILES: COc1ccc(OCC(=O)O)cc1N1CCC(=O)NC1=O]

The title compound was prepared in analogy to L-XVII starting from methyl (3-amino-4-methoxyphenoxy)acetate HCl salt (CAS 1170855-79-4). UPLC-MS 4: m/z 295.2 [M+H]⁺, t_{R}=0.17 min

### Synthesis of 2-(2-(((2S,4R)-1-((S)-2-(1-fluorocyclopropane-1-carboxamido)-3,3-dimethylbutanoyl)-4-hydroxypyrrolidine-2-carboxamido)methyl)-5-(4-methylthiazol-5-yl) phenoxy) acetic acid (L-XIX)

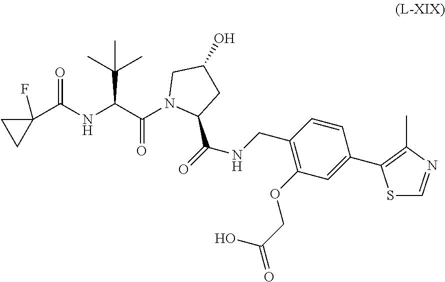 [SMILES: Cc1ncsc1-c1ccc(CNC(=O)[C@@H]2C[C@@H](O)CN2C(=O)[C@@H](NC(=O)C2(F)CC2)C(C)(C)C)c(OCC(=O)O)c1]

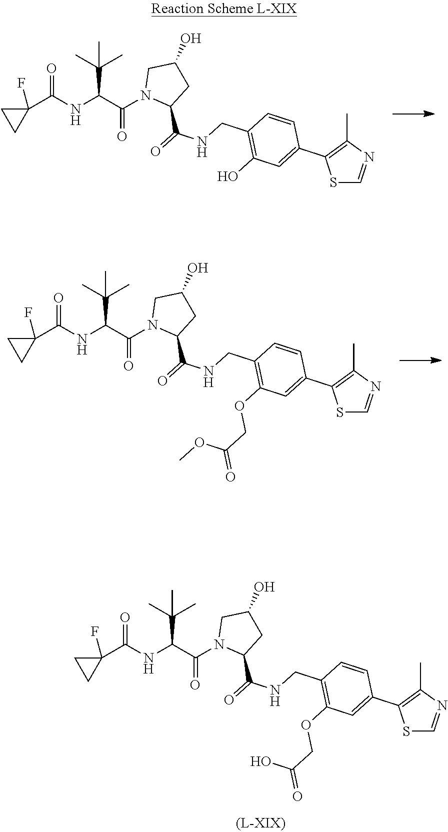 [SMILES: Cc1ncsc1-c1ccc(CNC(=O)[C@@H]2C[C@@H](O)CN2C(=O)[C@@H](NC(=O)C2(F)CC2)C(C)(C)C)c(O)c1 ; COC(=O)COc1cc(-c2scnc2C)ccc1CNC(=O)[C@@H]1C[C@@H](O)CN1C(=O)[C@@H](NC(=O)C1(F)CC1)C(C)(C)C ; Cc1ncsc1-c1ccc(CNC(=O)[C@@H]2C[C@@H](O)CN2C(=O)[C@@H](NC(=O)C2(F)CC2)C(C)(C)C)c(OCC(=O)O)c1]

### Step 1: 2-(2-(((2S,4R)-1-((S)-2-(1-fluorocyclopropane-1-carboxamido)-3,3-Methyl dimethylbutanoyl)-4-hydroxypyrrolidine-2-carboxamido)methyl)-5-(4-methylthiazol-5-yl) phenoxy)acetate

To a solution of (2S,4R)-1-((S)-2-(1-fluorocyclopropane-1-carboxamido)-3,3-dimethylbutanoyl)-4-hydroxy-N-(2-hydroxy-4-(4-methylthiazol-5-yl)benzyl) pyrrolidine-2-carboxamide (J. Med. Chem. 2019, 62, 699-726) (1.12 g, 2.06 mmol) in DMF (15 mL) was added K₂CO₃ (0.684 g, 4.95 mmol) followed by methyl 2-bromoacetate (0.312 mL, 3.30 mmol). The resulting suspension was stirred at RT for 5 h. More methyl 2-bromoacetate (0.312 mL, 3.30 mmol) and DMF (1 mL) was added and stirring was continued overnight and at 50° C. for 2 h. The reaction mixture was poured into water and extracted twice with EtOAc. The combined organic layers were washed with brine, dried (MgSO₄) and concentrated. Purification by flash chromatography (silica, DCM/MeOH, gradient: 0 to 25% MeOH) afforded the title product (914 mg) as a colorless foam. UPLC-MS 4: m/z 605.5 [M+H]⁺, t_{R}=0.88 min.

### Step 2: 2-(2-(((2S,4R)-1-((S)-2-(1-fluorocyclopropane-1-carboxamido)-3,3-dimethylbutanoyl)-4-hydroxypyrrolidine-2-carboxamido)methyl)-5-(4-methylthiazol-5-yl) phenoxy) acetic acid (L-XIX)

To a solution of methyl 2-(2-(((2S,4R)-1-((S)-2-(1-fluorocyclopropane-1-carboxamido)-3,3-dimethylbutanoyl)-4-hydroxypyrrolidine-2-carboxamido)methyl)-5-(4-methylthiazol-5-yl) phenoxy)acetate (790 mg, 1.306 mmol) in THF (5 mL) and MeOH (5 mL) was added NaOH (1.306 mL, 1.306 mmol, 1 N in water) and the reaction mixture was stirred at RT for 18 h. The mixture was concentrated, the residue was dissolved in water, filtered through a syringe filter and lyophilized to afford the title product (750 mg) as its sodium salt. UPLC-MS 4: m/z 591.4 [M+H]⁺, t_{R}=0.77 min.

### Synthesis of ((3S,5S)-1-((S)-2-((S)-2-((tert-butoxycarbonyl)(methyl)amino) propanamido)-2-cyclohexylacetyl)-5-(((R)-1,2,3,4-tetrahydronaphthalen-1-yl)carbamoyl) pyrrolidin-3-yl)glycine (L-XX)

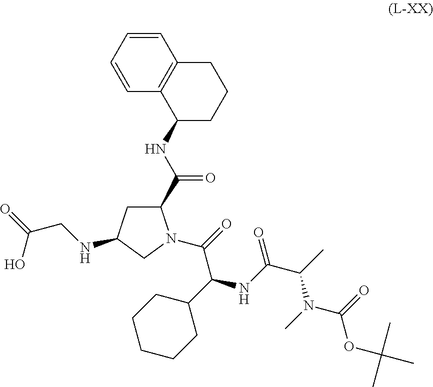 [SMILES: C[C@@H](C(=O)N[C@H](C(=O)N1C[C@@H](NCC(=O)O)C[C@H]1C(=O)N[C@@H]1CCCc2ccccc21)C1CCCCC1)N(C)C(=O)OC(C)(C)C]

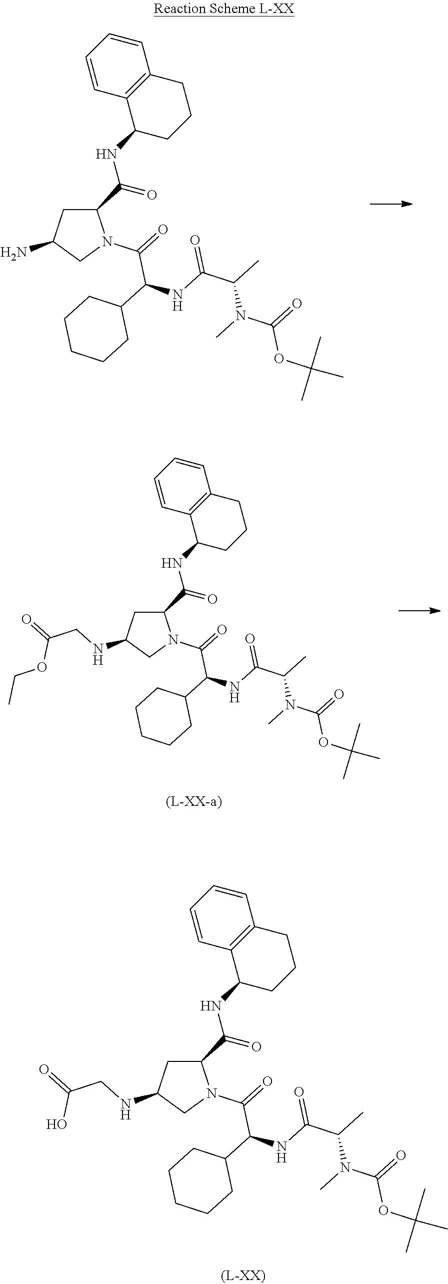

### Step 1: Ethyl ((3S,5S)-1-((S)-2-((S)-2-((tert-butoxycarbonyl)(methyl)amino) propanamido)-2-cyclohexylacetyl)-5-(((R)-1,2,3,4-tetrahydronaphthalen-1-yl)carbamoyl) pyrrolidin-3-yl)glycinate

Tert-butyl ((S)-1-(((S)-2-((2S,4S)-4-amino-2-(((R)-1,2,3,4-tetrahydronaphthalen-1-yl)carbamoyl) pyrrolidin-1-yl)-1-cyclohexyl-2-oxoethyl)amino)-1-oxopropan-2-yl)(methyl)carbamate (WO2014/090709) (1.37 g, 2.347 mmol) was dissolved in MeCN (9.39 ml) and TEA (0.654 ml, 4.69 mmol) was added with stirring. The reaction mixture was cooled to 0° C. and ethyl 2-bromoacetate (0.292 ml, 2.58 mmol) diluted with MeCN (2.347 ml) was added dropwise. The reaction mixture was stirred for 2 h at RT. After concentration the residue was dissolved in EtOAc (150 mL). The organic layer was washed with water (2×30 mL), a sat solution of NaHCO₃ (2×20 mL) and brine (2×10 mL), dried (Na₂SO₄) and concentrated to give the crude product. Purification by flash chromatography (DCM/Meoh, gradient: 0 to 10% MeOH) afforded the title compound (1.365 g).

### Step 2: ((3S,5S)-1-((S)-2-((S)-2-((tert-butoxycarbonyl)(methyl)amino) propanamido)-2-cyclohexylacetyl)-5-(((R)-1,2,3,4-tetrahydronaphthalen-1-yl)carbamoyl) pyrrolidin-3-yl)glycine

To a solution of ethyl ((3S,5S)-1-((S)-2-((S)-2-((tert-butoxycarbonyl)(methyl)amino) propanamido)-2-cyclohexylacetyl)-5-(((R)-1,2,3,4-tetrahydronaphthalen-1-yl)carbamoyl) pyrrolidin-3-yl)glycinate (1.3 g, 1.941 mmol) in MeCN (9.70 ml) and water (9.70 ml) was added LiOH (0.093 g, 3.88 mmol). After stirring for 16 h, the pH of the mixture was adjusted to 3, the mixture was extracted with EtOAc and concentrated to afford the title compound (970 mg) which was used without further purification. UPLC-MS 4: m/z 642.7 [M+H]⁺, t_{R}=1.12 min.

### Synthesis of 3-fluoro-4-methoxy-2-(4,4,5,5-tetramethyl-1,3,2-dioxaborolan-2-yl)benzonitrile (N-I)

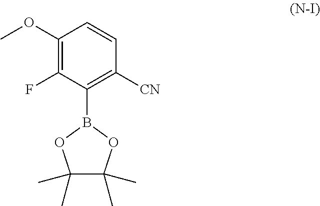 [SMILES: COc1ccc(C#N)c(B2OC(C)(C)C(C)(C)O2)c1F]

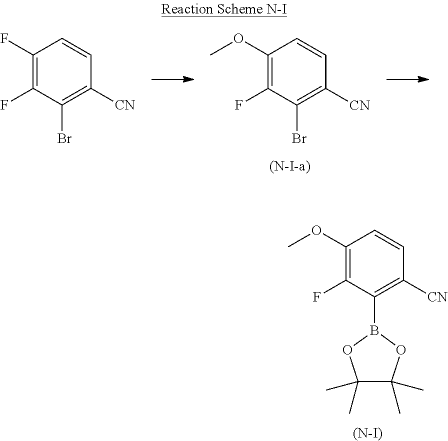

### Step 1: 2-Bromo-3-fluoro-4-methoxybenzonitrile (N-I-a)

To a stirred solution of 2-bromo-3,4-difluorobenzonitrile (4 g, 18.35 mmol) in MeOH (50 mL) under Ar was added sodium methoxide (6.29 mL, 27.5 mmol, 25% in MeOH). Then, the reaction mixture was stirred for 16 h at RT. The reaction mixture was quenched with a sat solution of NaHCO₃ and extracted with EtOAc. The organic layers were combined and washed with a sat solution of NaHCO₃, dried over Na₂SO₄ and concentrated. The residue was purified by flash chromatography (silica, hexane/EtOAc; gradient: 0% to 80% EtOAc) to give the title compound (4.17 g). ¹H NMR (400 MHZ, DMSO-d₆) δ (ppm) 7.87-7.67 (m, 1H), 7.44-7.20 (m, 1H), 3.95 (s, 3H).

### Step 2:3-Fluoro-4-methoxy-2-(4,4,5,5-tetramethyl-1,3,2-dioxaborolan-2-yl)benzonitrile (N-I)

A solution of 2-bromo-3-fluoro-4-methoxybenzonitrile (N-I-a) (25 g, 109 mmol) was cooled to −78° C. under Ar. A solution of n-BuLi (102 mL, 163 mmol, 1.6 M in hexanes) was slowly added over min. After another 10 min at −78° C. 2-isopropoxy-4,4,5,5-tetramethyl-1,3,2-dioxaborolane (33.0 mL, 163 mmol) was added and stirring at −78° C. was continued for another 60 min. The reaction mixture was allowed to warm to RT and quenched by the addition of aqueous 1 N HCl and extracted with EtOAc. The combined organic phases were washed with brine, dried (phase separation cartridge) and concentrated. The crude product was purified by flash chromatography (silica, cyclohexane/EtOAc; gradient: 0% to 25% EtOAc) to afford the title product (18.51 g) as a yellow oil. UPLC-MS 4: not ionizable, t_{R}=0.23 min. ¹H NMR (400 MHZ, DMSO-d₆) δ (ppm) 7.70 (dd, J=8.5, 1.0 Hz, 1H), 7.41 (t, J=8.6 Hz, 1H), 3.92 (s, 3H), 1.33 (s, 12H).

### Synthesis of methyl 4-chloro-5-fluoro-6-(2-((tetrahydro-2H-pyran-2-yl)oxy)ethoxy)nicotinate (N-II)

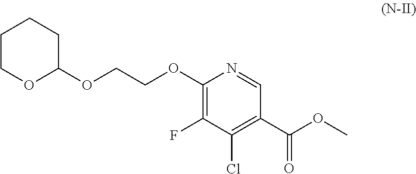 [SMILES: COC(=O)c1cnc(OCCOC2CCCCO2)c(F)c1Cl]

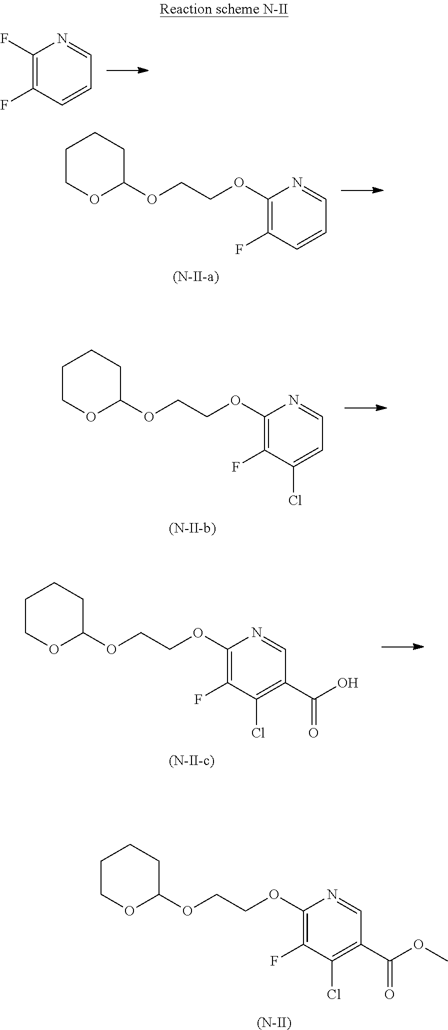

### Step 1: 3-Fluoro-2-(2-((tetrahydro-2H-pyran-2-yl)oxy)ethoxy)pyridine (N-II-a)

A reactor was charged with t-BuOK (7.80 kg, 69.51 mol) and THF (20 L), the mixture was cooled to −5-0° C. 2-((Tetrahydro-2H-pyran-2-yl)oxy)ethan-1-ol (6.60 kg, 45.15 mol) was added dropwise over 1 h. and the resulting mixture was stirred for 1 h at −5-0° C. 2,3-Difluoropyridine (4.0 kg, 34.76 mol) was added dropwise. The mixture was stirred for another 30 min, quenched with water (20 L) and extracted with EtOAc (2×20 L). The organic phase was washed with brine (20 L), dried over anhydrous Na₂SO₄ and concentrated. The crude product was purified by flash chromatography (silica, heptane/EtOAc 10:1) to give the title compound (7.46 kg) as a yellow oil. ¹H NMR (400 MHZ, CDCl₃) δ 7.89 (dd, J=5.0, 1.6 Hz, 1H), 7.32 (ddd, J=10.3, 7.8, 1.6 Hz, 1H), 6.84 (ddd, J=8.0, 5.0, 3.2 Hz, 1H), 4.72 (t, J=3.6 Hz, 1H), 4.64-4.53 (m, 2H), 4.16-4.04 (m, 1H), 3.96-3.81 (m, 2H), 3.58-3.46 (m, 1H), 1.91-1.78 (m, 1H), 1.77-1.67 (m, 1H), 1.67-1.47 (m, 4H). UPLC-MS 5: HRMS m/z calcd for C₁₂H₁₇FNO₃ [M+H]⁺ 242.1187, found 242.1182.

### Step 2: 4-Chloro-3-fluoro-2-(2-((tetrahydro-2H-pyran-2-yl)oxy)ethoxy)pyridine (N-II-b)

A reactor was charged with 3-fluoro-2-(2-((tetrahydro-2H-pyran-2-yl)oxy)ethoxy)pyridine (N-II-a) (3.60 kg, 14.92 mol) and THF (18 L). The solution was cooled to −78° C. n-BuLi (7.79 L, 19.40 mol, 2.5 M in hexanes) was added dropwise and the reaction was stirred for 4 h at −78° C. C₂Cl₆ (4.59 kg, 19.40 mol) was added at −78° C. The mixture was stirred for 30 min, quenched by addition of water (18 L), stirred for another 2 h and extracted with EtOAc (2×18 L). The organic phase was washed with brine (18 L), dried over anhydrous Na₂SO₄ and concentrated. The crude product was purified by flash chromatography (silica, heptane/EtOAc 10:1) to give the title compound (3.74 kg) as a light-yellow oil. ¹H NMR (400 MHZ, CDCl₃) δ 7.79 (dd, J=5.5, 1.0 Hz, 1H), 6.91 (dd, J=5.4, 4.4 Hz, 1H), 4.72 (t, J=3.6 Hz, 1H), 4.58 (dt, J=6.0, 3.7 Hz, 2H), 4.08 (ddd, J=11.5, 5.6, 3.9 Hz, 1H), 3.94-3.80 (m, 2H), 3.52 (ddd, J=10.6, 6.0, 4.2 Hz, 1H), 1.90-1.77 (m, 1H), 1.77-1.67 (m, 1H), 1.66-1.47 (m, 4H). UPLC-MS 5: HRMS m/z calcd for C₁₂H₁₅ClFNNaO₃ [M+Na]⁺ 298.0617, found 298.0657.

### Step 3: 4-Chloro-5-fluoro-6-(2-((tetrahydro-2H-pyran-2-yl)oxy)ethoxy) nicotinic acid (N-II-c)

A reactor was charged with diisopropylamine (1.91 kg, 18.88 mol) and THF (20 L). n-BuLi (7.56 L, 18.88 mol, 2.5 M in hexanes) was added dropwise at −78° C. The mixture was stirred for 30 min and 4-chloro-3-fluoro-2-(2-((tetrahydro-2H-pyran-2-yl)oxy)ethoxy)pyridine (N-II-b) (4.00 kg, 14.51 mol) was added dropwise. The reaction was stirred for 3 h and poured into dry ice (12.8 kg). The mixture was stirred for 2 h. Water (16 L) was added and the mixture was extracted with MTBE (20 L). The organic phase was washed with brine (4 L), then the combined aqueous phases were added to a mixture of EtOAc (40 L) and citric acid solution (3.77 kg citric acid dissolved in 22 L of water). The mixture was stirred for 30 min and the organic phase was separated. The aqueous phase was extracted with EtOAc (40 L). The combined organic phases were washed with brine (16 L), dried over anhydrous Na₂SO₄ and concentrated to give the title compound (3.68 kg) as an off-white solid. ¹H NMR (400 MHZ, CDCl₃) δ 8.64 (d, J=1.3 Hz, 1H), 8.02 (brs, 1H), 4.77 (t, J=3.4 Hz, 1H), 4.68 (ddd, J=5.9, 4.0, 1.8 Hz, 2H), 4.11 (ddd, J=11.6, 5.4, 4.0 Hz, 1H), 3.97-3.84 (m, 2H), 3.62-3.53 (m, 1H), 1.90-1.70 (m, 2H), 1.68-1.49 (m, 4H). UPLC-MS 5: HRMS m/z calcd for C₁₃H₁₄ClFNO₅ [M−H]⁻ 318.0550, found 318.0482.

### Step 4: Methyl 4-chloro-5-fluoro-6-(2-((tetrahydro-2H-pyran-2-yl)oxy)ethoxy)nicotinate (N-II)

To a solution of 4-chloro-5-fluoro-6-(2-((tetrahydro-2H-pyran-2-yl)oxy)ethoxy) nicotinic acid (N-II-c) (4.00 kg, 12.5 mol) in DMF (20 L) were added K₂CO₃ (4.32 kg, 31.3 mol) and Mel (2.67 kg, 18.8 mol). The mixture was stirred overnight at RT. MTBE (40 L) and water (40 L) were added. The mixture was stirred for 10 min and the phases were separated. The organic phase was washed with water (40 L) twice, dried over anhydrous Na₂SO₄ and concentrated. The crude product was purified by slurring with n-heptane (4 L) to give the title compound (3.37 kg) as a colorless solid. ¹H NMR (400 MHZ, DMSO-d₆) δ 8.51 (d, J=1.2 Hz, 1H), 4.64 (t, J=2.9, 1H), 4.63-4.52 (m, 2H), 3.99-3.92 (m, 1H), 3.86 (s, 3H), 3.81-3.71 (m, 2H), 3.46-3.38 (m, 1H), 1.75-1.56 (m, 2H), 1.52-1.37 (m, 4H). UPLC-MS 5: HRMS m/z calcd for C₁₄H₁₈ClFNO₅ [M+H]⁺ 334.0852, found 334.0823.

### Synthesis of methyl 2-bromo-3-fluoro-4-methoxybenzoate (N-III)

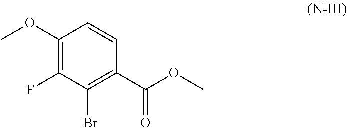

At RT DBU (0.50 mL, 3.3 mmol) was added to a solution of methyl 2-bromo-3,4-difluorobenzoate (550 mg, 2.2 mmol) in MeOH (10 mL) and the reaction mixture was stirred at 50° C. for 22 h. A sat solution of NaHCO₃ was added and the mixture was extracted with EtOAc. The combined organic layers were dried over anhydrous MgSO₄ and concentrated. The crude product was purified by flash chromatography (silica, cyclohexane/EtOAc; gradient: 0% to 20% EtOAc) to afford the title compound (390 mg) as a colorless powder. UPLC-MS 1: m/z 263.1 [M+H]⁺, t_{R}=1.00 min.

### Synthesis of (1S,4r)-4-((((2S,3S,4S)-4-(6-carbamoyl-2-fluoro-3-methoxyphenyl)-5-chloro-6-fluoro-3-methyl-2-phenyl-2,3-dihydrobenzofuran-2-yl)methyl)amino)cyclohexane-1-carboxylic acid (C-I)

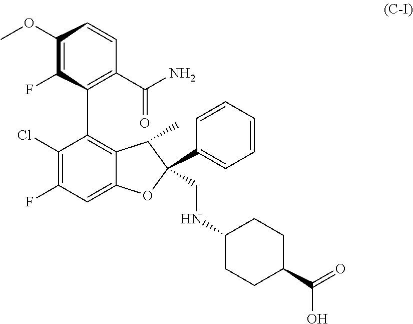

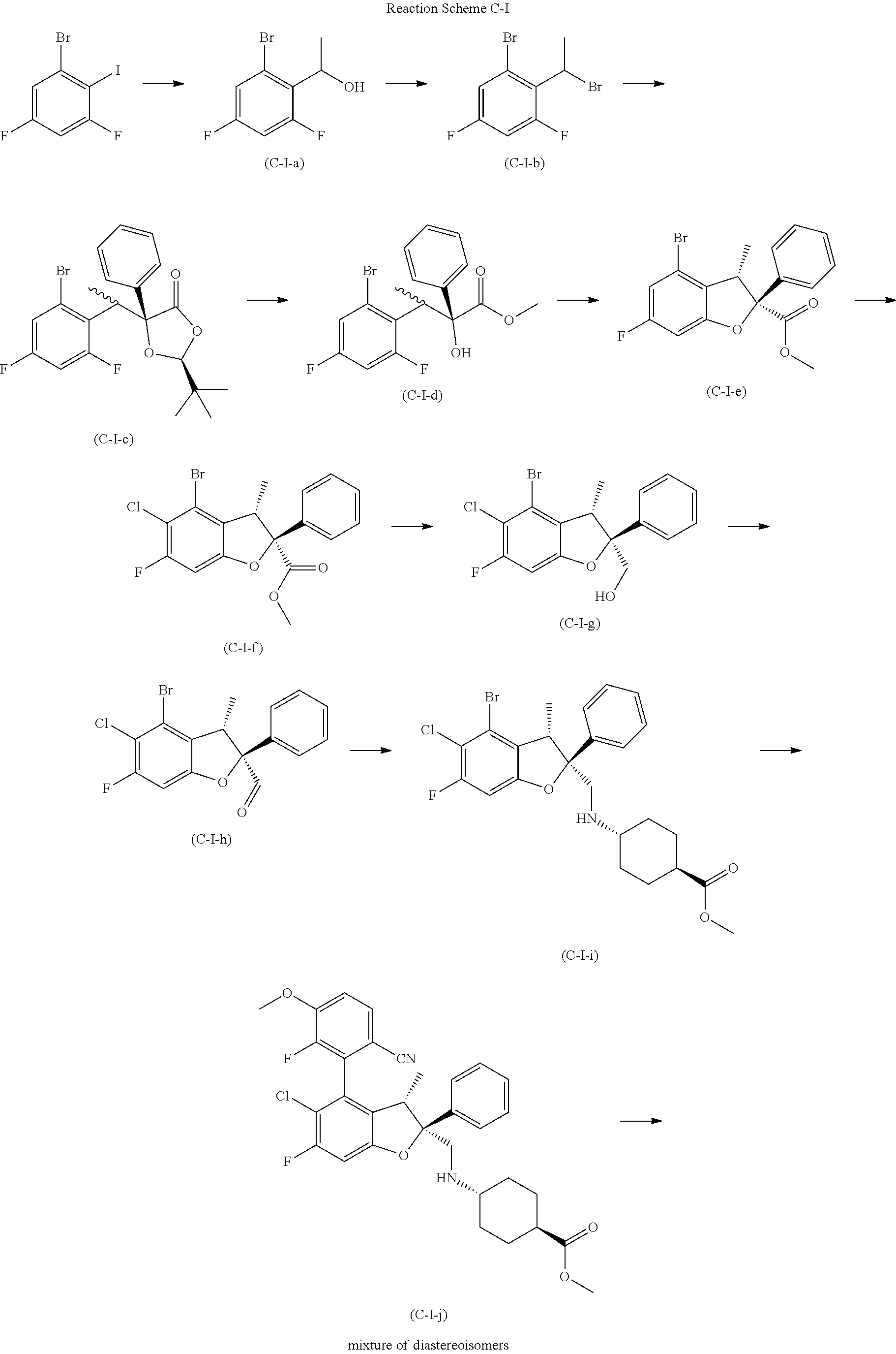

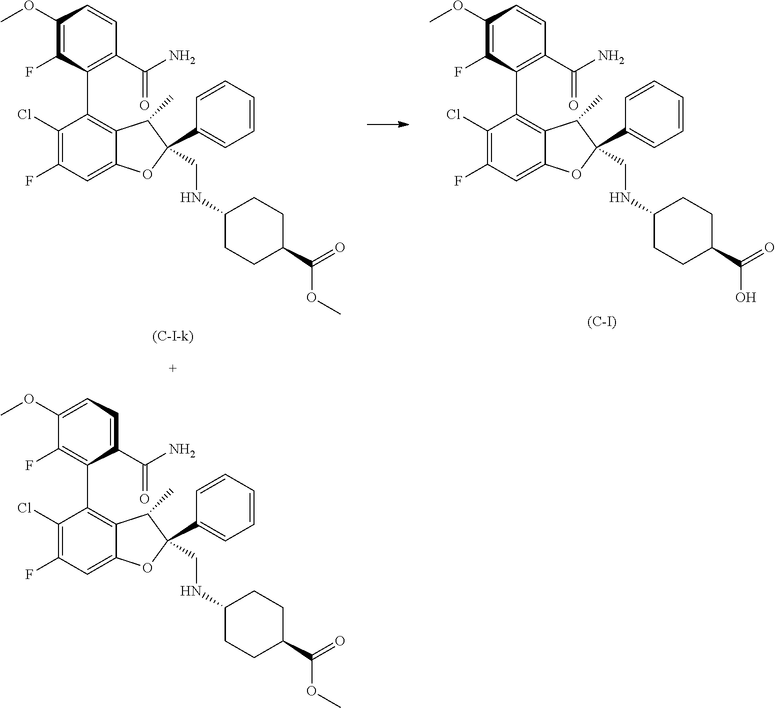

### Step 1: 1-(2-Bromo-4,6-difluorophenyl)ethan-1-ol (C-I-a)

Two reactions were carried out in parallel as follows. To a stirred solution of 1-bromo-3,5-difluoro-2-iodobenzene (500 g, 1.57 mol) in 2-methyltetrahydrofuran (5000 mL) was added lithium.chloro(isopropyl) magnesiumchloride (1.3 M, 1.89 mol, 1.45 L) dropwise at −65° C. under N₂. The reaction mixture was stirred for 30 min at −65° C. before acetaldehyde (138.15 g, 3.14 mol) was added dropwise. Stirring of the reaction mixture at −65° C. was continued for 30 min. For workup a sat solution of NH₄Cl was added followed by extraction with MTBE. The combined organic layers were washed with brine, dried over anhydrous Na₂SO₄, filtered and concentrated under reduced pressure to furnish the racemic title compound (800 g) as a colorless oil which was used in the next step without further purification. ¹HNMR (400 MHZ, DMSO-d₆) 7.43 (td, J=2.0, 8.0 Hz, 1H), 7.30 (ddd, J=2.4, 9.2, 11.6 Hz, 1H), 5.42 (d, J=4.4 Hz, 1H), 5.21-5.10 (m, 1H), 1.41 (d, J=6.4 Hz, 3H).

### Step 2: 1-Bromo-2-(1-bromoethyl)-3,5-difluorobenzene (C-I-b)

Five reaction were carried out in parallel as follows. To a stirred solution of 1-(2-bromo-4,6-difluorophenyl)ethan-1-ol (C-I-a) (220.0 g, 928.11 mmol) in DCM (4000 mL) were added CBr₄ (461.68 g, 1.39 mol) and PPh₃ (365.15 g, 1.39 mol) under N₂ at 0° C. and stirring was continued for 30 min. The reaction mixture was quenched by the addition of a sat solution of NaHCO₃ and extracted with DCM. The combined organic layers were washed with brine, dried over anhydrous Na₂SO₄ and concentrated under reduced pressure. The five batches of crude product were combined and purified by flash chromatography (silica, PE) followed by distillation at 100° C. to give the title product (1.10 kg) as a racemate. ¹H NMR (400 MHZ, DMSO-d₆) 7.52 (td, J=2.0, 8.0 Hz, 1H), 7.41 (ddd, J=2.4, 9.2, 11.6 Hz, 1H), 5.71-5.52 (m, 1H), 2.03 (dd, J=2.0, 7.2 Hz, 3H).

### Step 3: (2S,5S)-5-(1-(2-Bromo-4,6-difluorophenyl)ethyl)-2-(tert-butyl)-5-phenyl-1,3-dioxolan-4-one (C-I-c)

Six reactions were carried out in parallel as follows. To a stirred suspension of 1-bromo-2-(1-bromoethyl)-3,5-difluorobenzene (C-I-b) (185 g, 616.8 mmol) and (2S,5S)-2-(tert-butyl)-5-phenyl-1,3-dioxolan-4-one (CAS 81036-97-7) (203.79 g, 925.2 mmol) in DMF (2500 mL) was added NaH (37.0 g, 925.2 mmol, 60% in mineral oil) under N₂ at 0° C. The reaction mixture was stirred for 30 min at 0° C. For workup the mixture was quenched by the addition of a sat solution of NaHCO₃ and extracted with MTBE. The combined organic layers were washed with brine, dried over anhydrous Na₂SO₄, filtered and concentrated under reduced pressure. The crude material was purified by flash chromatography (silica, PE/EtOAc, gradient: 0% to 5% EtOAc) to give the title compound (810 g) as a mixture of diastereoisomers as a light yellow oil.

### Step 4: Methyl (2S)-3-(2-bromo-4,6-difluorophenyl)-2-hydroxy-2-phenylbutanoate (C-I-d)

Two reactions were carried out in parallel as follows. To a stirred solution of (2S,5S)-5-(1-(2-bromo-4,6-difluorophenyl)ethyl)-2-(tert-butyl)-5-phenyl-1,3-dioxolan-4-one (C-I-c) (425 g, 967 mmol) in MeOH (4500 mL) was added sodium methanolate (261 g, 1.45 mol, 30% in MeOH) at RT and stirring at 60° C. was continued for 3 h. The reaction mixture was concentrated and the residue was poured into a solution of NH₄Cl (200 g) and citric acid (40 g) in water 2.5 L) The organic layer was separated and washed with brine (1 L), dried and filtered. The filtrate was concentrated under reduced pressure to afford the title product (800 g) as a mixture of diastereoisomers and used in the next step without any additional purification.

### Step 5: Methyl (2S,3S)-4-bromo-6-fluoro-3-methyl-2-phenyl-2,3-dihydrobenzofuran-2-carboxylate (C-I-e)

Three reactions were carried out in parallel as follows. At 15° C. NaH (40.2 g, 1.00 mol, 60% in mineral oil) was added to a stirred solution of methyl (2S)-3-(2-bromo-4,6-difluorophenyl)-2-hydroxy-2-phenylbutanoate (C-I-d) (430 g, 1.12 mol) in NMP (2.6 L) under N₂. The resulting reaction mixture was stirred at 15° C. for 15 min. The reaction mixture was poured into a sat solution of NH₄Cl (5 L) and extracted three times with TBME (2 L). before it was quenched by the addition of a sat solution of NaHCO₃ and extracted tree times with MTBE (2 L). The combined organic layers were washed with brine (3 L), dried, filtered and concentrated. The crude product was purified by flash chromatography (silica, PE/EtOAc, gradient: 2% to 20% EtOAc) to afford a yellow solid (360 g) The solid was recrystallized from PE/EtOAc (1.8 L, heated to 70° C. with stirring to dissolve all solid and allowed to cool to 10° C., the solid was collected by filtration). The title product (260 g) was isolated as a colorless solid with an enantiomeric excess of >99%. Chiral SFC: ChiralPak AD-3, 150×4.6 mm I.D., 3 μm, CO₂/ethanol (0.05% DEA), gradient: from 95/5 to 60/40 in 5.5 min and hold at 60/40 for 3 min, then 95/5 for 1.5 min; column temperature 40° C., flow rate: 2.5 mL/min, t_{R}=1.72 min. ¹H NMR (400 MHZ, DMSO-d₆) 7.62 (d, J=7.2 Hz, 2H), 7.43-7.30 (m, 3H), 7.07 (br d, J=9.2 Hz, 1H), 7.03 (br d, J=9.2 Hz, 1H), 3.92 (q, J=6.8 Hz, 1H), 3.74 (s, 3H), 1.31 (d, J=6.8 Hz, 3H). In addition, a second batch of the title compound (100 g) with a purity of 70% was isolated.

### Step 6: Methyl (2S,3S)-4-bromo-5-chloro-6-fluoro-3-methyl-2-phenyl-2,3-dihydrobenzofuran-2-carboxylate (C-I-f)

Two reactions were carried out in parallel as follows To a stirred solution of methyl (2S,3S)-4-bromo-6-fluoro-3-methyl-2-phenyl-2,3-dihydrobenzofuran-2-carboxylate (C-I-e) (110 g, 301.2 mmol) in ACN (1870 mL) were added N-chlorosuccinimide (46.8 g, 350.5 mmol) and 4-methylbenzenesulfonic acid hydrate (85.9 g, 451.8 mmol) under N₂. The resulting reaction mixture was stirred at 45° C. for 12 h before it was allowed to cool to 15° C. Both batches were combined and concentrated. The residue was dissolved in EtOAc (700 mL) and washed with a sat solution of NaHCO₃ (2*400 mL), dried over anhydrous Na₂SO₄, filtered and concentrated under reduced pressure. The crude product thus obtained was recrystallized from n-hexane (800 mL) to afford the title product (200 g) as a colorless powder with an enantiomeric excess of >99%. Chiral SFC: Chiralpak AD-3, 150×4.6 mm I.D., 3 μm, CO₂/IPA (0.05% DEA), gradient: from 95/5 to 60/40 in 5.5 min and hold at 60/40 for 3 min, then 95/5 for 1.5 min; column temperature 40° C., flow rate: 2.5 mL/min, t_{R}=2.57 min. ¹H NMR (400 MHZ, DMSO-d₆) 7.75-7.55 (m, 2H), 7.45-7.26 (m, 4H), 4.01-3.91 (m, 1H), 3.75 (s, 3H), 1.32 (d, J=6.8 Hz, 3H).

### Step 7: ((2S,3S)-4-Bromo-5-chloro-6-fluoro-3-methyl-2-phenyl-2,3-dihydrobenzofuran-2-yl)methanol (C-I-g)

Two reactions were carried out in parallel as follows. MeOH (40.1 mL, 990.9 mmol) was added to a stirred solution of methyl (2S,3S)-4-bromo-6-fluoro-3-methyl-2-phenyl-2,3-dihydrobenzofuran-2-carboxylate (C-I-f) (99.0 g, 247.7 mmol) in THF (1180 mL) under N₂ followed by portion wise addition of LiBH₄ (10.79 g, 495.5 mmol). The resulting suspension was stirred for 30 min at 15° C. The reaction mixture was quenched by the addition of a sat solution of NaHCO₃ (2500 mL) and extracted with EtOAc (2*1500 mL). The combined organic layers were washed with a sat solution of NaHCO₃ (1000 mL), dried over anhydrous Na₂SO₄, filtered and concentrated under reduced pressure to afford the title product (160.2 g) as a light yellow solid with an enantiomeric excess of >99% (Chiral SFC: Lux Cellulose-2, 150 mm×4.6 mm I.D., 3 μm, CO₂/EtOH (0.05% DEA), gradient: from 95/5 to 60/40 in 5.5 min and hold at 60/40 for 3 min, then 95/5 for 1.5 min; column temperature 40° C.; flow rate: 2.5 mL/min, t_{R}=4.22 min). ¹H NMR (400 MHZ, DMSO-d₆) 7.50-7.41 (m, 2H), 7.32 (t, J=7.2 Hz, 2H), 7.28-7.18 (m, 2H), 5.08 (t, J=5.2 Hz, 1H), 3.95 (dq, J=5.8, 13.6 Hz, 2H), 3.56 (q, J=6.8 Hz, 1H), 1.44 (d, J=6.8 Hz, 3H).

### Step 8: (2S,3S)-4-Bromo-5-chloro-6-fluoro-3-methyl-2-phenyl-2,3-dihydrobenzofuran-2-carbaldehyde (C-I-h)

To a stirred solution of oxalyl chloride (3.01 mL, 34.4 mmol) in DCM (60 mL) was added DMSO (4.9 mL, 68.9 mmol) in DCM (10 mL) at −78° C. and the reaction mixture was stirred at −78° C. for 15 min. A solution of ((2S,3S)-4-bromo-5-chloro-6-fluoro-3-methyl-2-phenyl-2,3-dihydrobenzofuran-2-yl)methanol (C-I-g) (8 g, 21.5 mmol) in DCM (30 mL) was then added and stirring at −78° C. was continued for 15 min. TEA (15.0 mL, 108 mmol) was added and the reaction mixture was allowed to warm to 0° C. over 30 min. The mixture was diluted in DCM and water, extracted twice with DCM and the combined organic extracts were washed with water and brine, dried over anhydrous Na₂SO₄ and concentrated to afford the title compound (8.6 g) as a yellow foam, which was used without purification. UPLC-MS 1: m/z 367.0/368.8 [M−H]⁻, t_{R}=1.39 min.

### Step 9: Methyl (1S,4r)-4-((((2S,3S)-4-bromo-5-chloro-6-fluoro-3-methyl-2-phenyl-2,3-dihydrobenzofuran-2-yl)methyl)amino)cyclohexane-1-carboxylate (C-I-i)

A solution of methyl trans-4-amino cyclohexane carboxylate (20 g, 127 mmol) in DCE (100 ml) was added to a stirred solution of (2S,3S)-4-bromo-5-chloro-6-fluoro-3-methyl-2-phenyl-2,3-dihydrobenzofuran-2-carbaldehyde (C-I-h) (49 g, 117 mmol) in DCE (300 ml), AcOH (6.68 ml, 117 mmol) and molecular sieves. The reaction mixture was stirred at RT for 16 h. After completion of the imine formation, NaBH(OAc)₃ (49.5 g, 233 mmol) was added in portions and the reaction mixture was stirred at RT for an additional 16 h. For workup, a sat solution of Na₂CO₃ was added followed by extraction with EtOAc. The combined organic layers were washed with water and brine, dried over anhydrous Na₂SO₄, filtered and concentrated under reduced pressure. The crude material was purified by flash chromatography (silica, cyclohexane/EtOAc, gradient: 0% to 25% EtOAc) to give the title compound (55.4 g) as white powder. UPLC-MS 4: m/z 510.2/512.2 [M+H]⁺, t_{R}=0.89 min.

Step 10: Methyl (1S,4r)-4-((((2S,3S,4S)-5-chloro-4-(6-cyano-2-fluoro-3-methoxyphenyl)-6-fluoro-3-methyl-2-phenyl-2,3-dihydrobenzofuran-2-yl)methyl)amino)cyclohexane-1-carboxylate and methyl (1S,4r)-4-((((2S,3S,4R)-5-chloro-4-(6-cyano-2-fluoro-3-methoxyphenyl)-6-fluoro-3-methyl-2-phenyl-2,3-dihydrobenzofuran-2-yl)methyl)amino)cyclohexane-1-carboxylate (C-I-j) Thirty two reactions were carried out in parallel as follows. To a stirred solution of methyl (1S,4r)-4-((((2S,3S)-4-bromo-5-chloro-6-fluoro-3-methyl-2-phenyl-2,3-dihydrobenzofuran-2-yl)methyl)amino)cyclohexane-1-carboxylate (C-I-i) (1.25 g, 2.447 mmol) and 3-fluoro-4-methoxy-2-(4,4,5,5-tetramethyl-1,3,2-dioxaborolan-2-yl)benzonitrile (N-I) (1.356 g, 4.89 mmol) in cyclopentyl methyl ether (8.1 ml) was added K₃PO₄ (1.558 g, 7.34 mmol). The reaction mixture was purged three times with argon before SPhos Pd G3 [1448085-82-4] (0.191 g, 0.245 mmol) was added. The reaction mixture was stirred for 5 days at 100° C. All batches were combined and concentrated to be purified by flash chromatography (silica, heptane/EtOAc; gradient: 0% to 60% EtOAc) followed by a second purification (silica, heptane/EtOAc; gradient: 0% to 50% EtOAc) to afford a mixture of the title compounds (26.19 g) as a yellow oil. UPLC-MS 4: m/z 581.2 [M+H]⁺, t_{R}=0.75/0.79 min.

### Step 11: Methyl (1S,4r)-4-((((2S,3S,4S)-4-(6-carbamoyl-2-fluoro-3-methoxyphenyl)-5-chloro-6-fluoro-3-methyl-2-phenyl-2,3-dihydrobenzofuran-2-yl)methyl)amino)cyclohexane-1-carboxylate (C-I-k)

To a stirred solution of a mixture of methyl (1S,4r)-4-((((2S,3S,4S)-5-chloro-4-(6-cyano-2-fluoro-3-methoxyphenyl)-6-fluoro-3-methyl-2-phenyl-2,3-dihydrobenzofuran-2-yl)methyl)amino)cyclohexane-1-carboxylate and methyl (1S,4r)-4-((((2S,3S,4R)-5-chloro-4-(6-cyano-2-fluoro-3-methoxyphenyl)-6-fluoro-3-methyl-2-phenyl-2,3-dihydrobenzofuran-2-yl)methyl)amino)cyclohexane-1-carboxylate (C-I-j) (26.19 g, 41.9 mmol) in ethanol (116 ml) and water (23.2 ml) was added hydrido(dimethylphosphinous acid-kP)[hydrogen bis(dimethylphosphinito-kP)]platinum (II) [CAS 173416 May 2] (0.900 g, 2.096 mmol) and the yellow suspension was stirred overnight at 80° C. The reaction mixture was quenched with a sat solution of NaHCO₃ and extracted twice with EtOAc. The organic layers were combined and washed with water and brine, dried (phase separation cartridge) and concentrated. The crude product was purified and both diastereoisomers were separated by flash chromatography (silica, heptane/EtOAc; gradient: 0% to 100% EtOAc).

Methyl (1S,4r)-4-((((2S,3S,4S)-4-(6-carbamoyl-2-fluoro-3-methoxyphenyl)-5-chloro-6-fluoro-3-methyl-2-phenyl-2,3-dihydrobenzofuran-2-yl)methyl)amino)cyclohexane-1-carboxylate (C-I-k) (10.29 g). UPLC-MS 4: m/z 599.3 [M+H]⁺, t_{R}=0.66 min.

Other diasteroisomer methyl (1S,4r)-4-((((2S,3S,4R)-4-(6-carbamoyl-2-fluoro-3-methoxyphenyl)-5-chloro-6-fluoro-3-methyl-2-phenyl-2,3-dihydrobenzofuran-2-yl)methyl)amino)cyclohexane-1-carboxylate (14.7 g): UPLC-MS 4: m/z 599.6 [M+H]⁺, t_{R}=0.62 min.

### Step 12: (1S,4r)-4-((((2S,3S,4S)-4-(6-carbamoyl-2-fluoro-3-methoxyphenyl)-5-chloro-6-fluoro-3-methyl-2-phenyl-2,3-dihydrobenzofuran-2-yl)methyl)amino)cyclohexane-1-carboxylic acid (C-I)

To a stirred solution of methyl (1S,4r)-4-((((2S,3S,4S)-4-(6-carbamoyl-2-fluoro-3-methoxyphenyl)-5-chloro-6-fluoro-3-methyl-2-phenyl-2,3-dihydrobenzofuran-2-yl)methyl)amino)cyclohexane-1-carboxylate (C-I-k) (10.24 g, 16.58 mmol) in dioxane (138 ml) and water (27.6 ml) was added LiOH·H₂O (1.044 g, 24.87 mmol) and the reaction mixture was stirred at RT for 48 h. More LiOH·H₂O (1.044 g, 9.53 mmol) was added and stirring was continued for 16 h. The reaction mixture was concentrated under reduced pressure. The crude product was purified by reverse phase chromatography (C18 silica, (water+0.1% TFA)/ACN; gradient: 0% to 40% ACN) to afford the title compound (7.02 g) as a white powder as a TFA salt. UPLC-MS 4: m/z 585.5 [M+H]⁺, t_{R}=0.68 min. ¹H NMR (400 MHZ, DMSO-d₆) δ 12.17 (br s, 1H), 8.59-8.41 (m, 1H), 7.98-7.84 (m, 1H), 7.79 (br s, 1H), 7.60 (dd, J=8.7, 1.4 Hz, 1H), 7.55-7.49 (m, 2H), 7.47-7.41 (m, 2H), 7.41-7.35 (m, 1H), 7.28 (t, J=8.6 Hz, 1H), 7.15 (d, J=9.3 Hz, 1H), 7.12 (br s, 1H), 3.91-3.84 (m, 4H), 3.58-3.51 (m, 1H), 3.19 (q, J=7.1 Hz, 1H), 3.02-2.91 (m, 1H), 2.15-1.90 (m, 5H), 1.45-1.19 (m, 4H), 0.94 (d, J=7.2 Hz, 3H).

### Synthesis of 4-((2S,4S)-5-Chloro-6-fluoro-2-phenyl-2-((S)-pyrrolidin-2-yl)-2,3-dihydrobenzofuran-4-yl)-5-fluoro-6-(2-hydroxyethoxy)-N-methylnicotinamide (C-II)

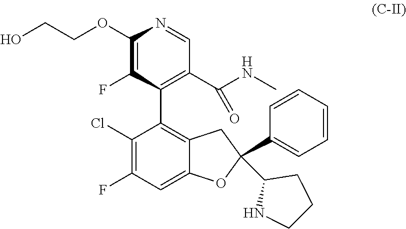

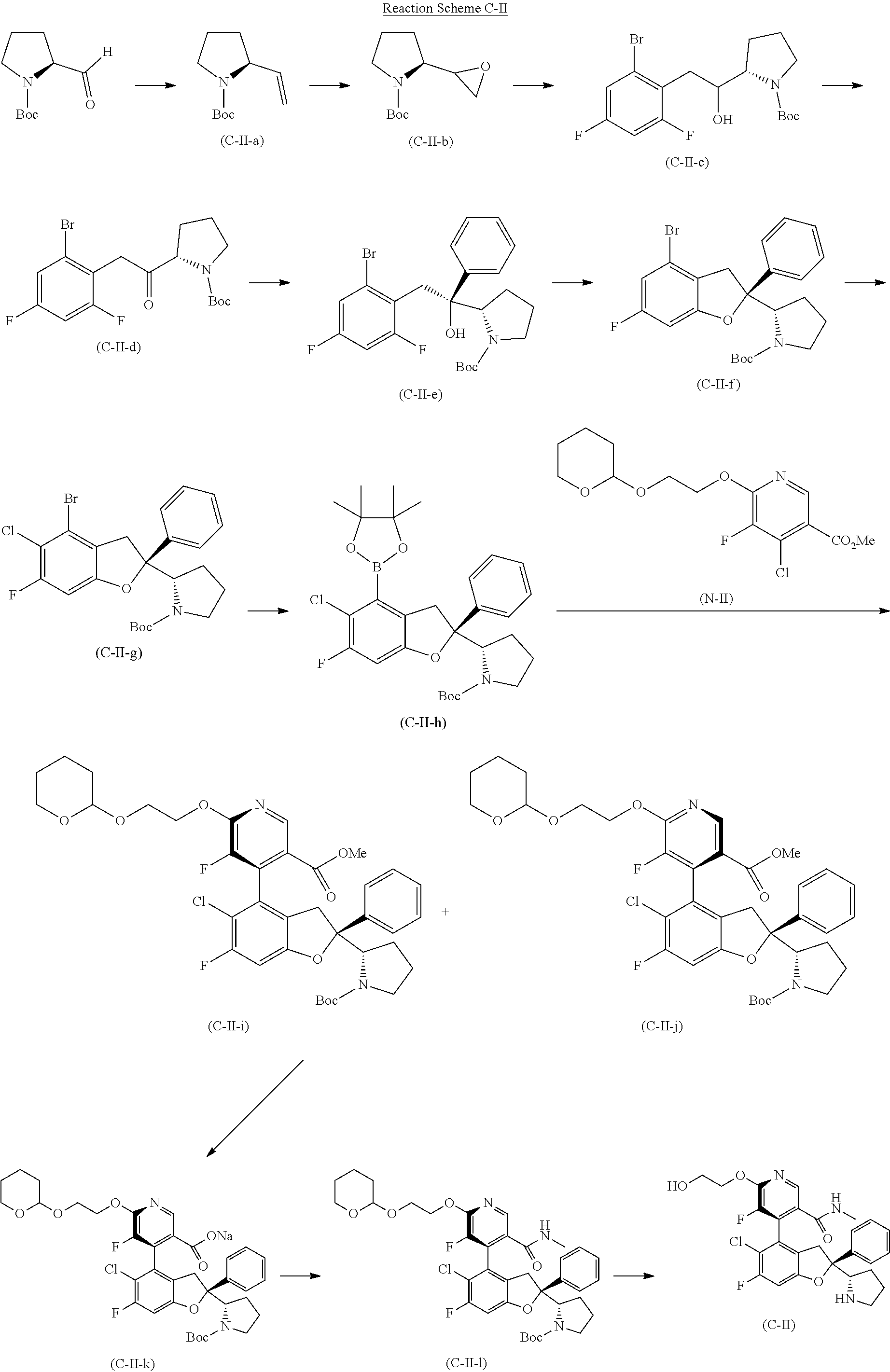

### Step 1: Tert-butyl (S)-2-vinylpyrrolidine-1-carboxylate (C-II-a)

At RT, under a nitrogen atmosphere, a suspension of Ph₃PCH₃Br (161.4 g, 451.7 mmol) and KO Bu (50.7 g, 451.7 mmol) in THF (500 mL) was stirred for 4 h, then cooled to −70° C. A solution of(S)-tert-butyl 2-formylpyrrolidine-1-carboxylate (75.0 g, 376 mmol) in THF (150 mL) was added dropwise over 30 min while maintaining the internal temperature below −20° C. The reaction mixture was then stirred for 16 h at RT. Upon completion of the reaction, 20 wt % NH₄Cl (200 mL) was added and the organic layer was separated. The water layer was extracted with EtOAC (100 mL). The combined organic layers were washed with brine, dried over anhydrous Na₂SO₄, filtered and concentrated to give a pale yellow oil. Heptane (400 mL) was added and the resulting suspension was stirred vigorously at 0˜5° C. for 1 h and filtered. The filtrate was dried under HV to give the title product (76 g) as a yellow oil which was directly used in the next step without further purification.

### Step 2: Tert-butyl (2S)-2-(oxiran-2-yl) pyrrolidine-1-carboxylate (C-II-b)

At 0° C., under a nitrogen atmosphere, mCPBA (155.4 g, 693.5 mmol, 77% w/w) was added portionwise to tert-butyl (S)-2-vinylpyrrolidine-1-carboxylate (C-II-a) (76 g, 424 mmol) dissolved in DCM (700 mL), while maintaining the internal temperature below 10° C. The reaction mixture was then stirred for 2 h at RT. Upon completion of the reaction, a solution of Na₂S₂O₃ (48.6 g, 385.3 mmol) in water (300 mL) was added slowly to quench the excess of mCPBA. Saturated aq. Na₂CO₃ (200 mL) was added to adjust the pH to 7-8. The organic layer was separated and the water layer was extracted with DCM (100 mL). The combined organic layers were washed with 5% NaHCO₃ (100 mL), then with brine (100 mL), dried over Na₂SO₄, filtered and concentrated to a volume of ca. 100 mL. Heptane (600 mL) was added and the resulting suspension was filtered. The filtrate was concentrated and the obtained residue was purified by flash chromatography on silica gel with n-heptane/EtOAc to give the title product (62 g) as a yellow oil.

### Step 3: Tert-butyl (2S)-2-(2-(2-bromo-4,6-difluorophenyl)-1-hydroxyethyl) pyrrolidine-1-carboxylate (C-II-c)

At −70° C., under a nitrogen atmosphere, iPrMgCl (190 mL, 2.0 M in THF, 102.8 mmol) was added dropwise over 30 min to a solution of 1-bromo-3,5-difluoro-2-iodobenzene (120.5 g, 377.9 mmol) in THF (800 mL) while maintaining an internal temperature at −40° C. to −35° C. The reaction mixture was stirred at—this temperature for 1 h. Upon completion of the reaction CuI (11.1 g, 58.1 mmol) was added quickly in one portion. A solution of tert-butyl (2S)-2-(oxiran-2-yl) pyrrolidine-1-carboxylate (C-II-b) (62 g, 290.7 mmol) in THF (100 mL) was added dropwise over 10 min while maintaining the internal temperature at −40° C. to −30° C. The reaction mixture was then gradually warmed to RT and stirred overnight. 20 wt % aq. NH₄Cl (700 mL) was added carefully to quench the reaction followed by MTBE (400 mL). The organic layer was separated and the water layer was extracted with MTBE (200 mL). The combined organic layers were washed with 20 wt % brine (300 mL), dried over anhydrous Na₂SO₄, filtered and concentrated to give the title product (118 g) as a pale yellow oil which was used directly in the next step. ¹H NMR (400 MHZ, CDCl₃) δ 7.13 (dt, J=8.1, 2.2 Hz, 1H), 6.80 (td, J=9.1, 2.6 Hz, 1H), 4.63 (s, 1H), 4.04-3.94 (m, 1H), 3.86-3.72 (m, 1H), 3.58-3.44 (m, 1H), 3.41-3.28 (m, 1H), 3.01-2.78 (m, 2H), 2.14-1.81 (m, 4H), 1.47 (s, 9H). UPLC-MS 5: HRMS m/z calcd for C₁₂H₁₅BrF₂NO [M−Boc]⁺ 306.0300, found 306.0292.

### Step 4: Tert-butyl (S)-2-(2-(2-bromo-4,6-difluorophenyl)acetyl) pyrrolidine-1-carboxylate (C-II-d)

At RT, under a nitrogen atmosphere, a solution of tert-butyl (2S)-2-(2-(2-bromo-4,6-difluorophenyl)-1-hydroxyethyl) pyrrolidine-1-carboxylate (C-II-c) (118 g, 290 mmol) in DCM (700 mL) was added dropwise over 30 min at RT to a solution of Dess-Martin periodinane (135.5 g, 319.5 mmol) in DCM (700 mL). The reaction mixture was stirred at RT for 45 min. Upon completion of the reaction a solution of Na₂SO₃ (58.6 g) in water (300 mL) was added carefully to quench the reaction while maintaining the internal temperature at 0 to 5° C. 15 wt % Na₂CO₃ (350 mL) was added to adjust the pH to 7-8 while maintaining the internal temperature below 10° C. The organic layer was separated and the water layer was extracted with DCM (500 mL). The combined organic layers were washed with 5 wt % NaHCO₃ (300 mL) then with 20 wt % brine (300 mL), dried over anhydrous Na₂SO₄, filtered and concentrated. The residue was purified by flash chromatography (silica; heptane/MTBE; gradient: 3% to 20% MTBE) to give the title product (75 g). ¹H NMR (400 MHZ, CDCl₃) δ 7.21-7.12 (m, 1H), 6.88-6.78 (m, 1H), 4.49 (dd, J=8.5, 4.5 Hz, 0.3H), 4.37 (dd, J=8.8, 5.2 Hz, 0.7H), 4.08-3.86 (m, 2H), 3.67-3.39 (m, 2H), 2.35-2.05 (m, 2H), 2.02-1.86 (m, 2H), 1.48 (s, 2.7H), 1.47 (s, 6.3H). UPLC-MS 5: HRMS m/z calcd for C₁₂H₁₃BrF₂NO [M−Boc]⁺ 304.0143, found 303.9271.

### Step 5: Tert-butyl (S)-2-((S)-2-(2-bromo-4,6-difluorophenyl)-1-hydroxy-1-phenylethyl) pyrrolidine-1-carboxylate (C-II-e)

At 0° C., under a nitrogen atmosphere PhMgBr (40 mL, 128.6 mmol, 2.5 M in Et₂O) was added dropwise over 30 min to tert-butyl (S)-2-(2-(2-bromo-4,6-difluorophenyl)acetyl) pyrrolidine-1-carboxylate (C-II-d) (26 g, 64.3 mmol) in a mixture of DCM (260 mL) and heptane (260 mL), while maintaining the internal temperature at −5-0° C. The reaction mixture was stirred at 0° C. for 10 min before it was quenched by adding slowly sat. aq. NH₄Cl (150 mL). MTBE (200 mL) was added and the organic layer was separated. The water layer was extracted with MTBE (200 mL). The combined organic layers were washed with 20 wt % brine (300 mL), dried over anhydrous Na₂SO₄, filtered and concentrated. The residue was purified by flash chromatography (silica; heptane/MTBE; gradient: 2% to 3% MTBE) to afford the title product (14 g), as a light yellow solid . . . ¹H NMR (400 MHZ, DMSO-d₆) δ 7.48 (s, 2H), 7.31 (dt, J=8.4, 2.1 Hz, 1H), 7.26-7.16 (m, 3H), 7.08-7.00 (m, 1H), 4.42-4.19 (m, 1H), 3.62-3.45 (m, 2H), 3.45-3.34 (m, 2H), 1.93-1.82 (m, 1H), 1.75-1.60 (m, 1H), 1.43 (s, 9H), 1.41-1.21 (m, 2H). UPLC-MS 5: HRMS m/z calcd for C₂₃H₂₇BrF₂NO₃ [M+H]⁺ 482.1137, found 481.9654.

### Step 6: Tert-butyl (S)-2-((S)-4-bromo-6-fluoro-2-phenyl-2,3-dihydrobenzofuran-2-yl) pyrrolidine-1-carboxylate (C-II-f)

At 0° C., under a nitrogen atmosphere, KOtBu (37.3 mL, 37.3 mmol, 1 M in THF) was added dropwise over 15 min to a solution of tert-butyl (S)-2-((S)-2-(2-bromo-4,6-difluorophenyl)-1-hydroxy-1-phenylethyl) pyrrolidine-1-carboxylate (C-II-e) (15 g, 31.1 mmol) in THF (150 mL), while maintaining the internal temperature at −5-0° C. The reaction mixture was stirred at −5-0° C. for 30 min. Upon completion, the mixture was diluted with MTBE (200 mL) and was quenched by adding 5 wt % aq. NaHCO₃ (150 mL) while maintaining the internal temperature at −5-0° C. The mixture was stirred for 15 min at −5-0° C. The organic layer was separated and the aqueous layer was extracted with MTBE (200 mL). The combined organic phases were washed with brine, dried over anhydrous Na₂SO₄, filtered and concentrated to afford the title product (14.3 g) which was used directly in the next step. ¹H NMR (400 MHZ, DMSO-d₆) δ 7.45-7.28 (m, 5H), 6.98 (dd, J=9.1, 2.2 Hz, 1H), 6.90 (dd, J=9.5, 2.2 Hz, 1H), 4.41 (dd, J=6.9, 3.5 Hz, 1H), 4.19-3.78 (m, 1H), 3.44-3.19 (m, 2H), 2.91-2.72 (m, 1H), 1.90-1.83 (m, 2H), 1.57-1.47 (m, 1H), 1.35 (s, 2H), 1.34 (s, 7H), 1.28-1.21 (m, 1H). UPLC-MS 5: HRMS m/z calcd for C₁₈H₁₈BrFNO [M−Boc]⁺ 362.0550, found 361.9654.

### Step 7: Tert-butyl (S)-2-((S)-4-bromo-5-chloro-6-fluoro-2-phenyl-2,3-dihydrobenzofuran-2-yl) pyrrolidine-1-carboxylate (C-II-g)

At 0° C., under a nitrogen atmosphere, p-TsOH (6.9 g, 36.3 mmol) and N-chlorosuccinimide (4.85 g, 36.3 mmol) were added to a solution of tert-butyl (S)-2-((S)-4-bromo-6-fluoro-2-phenyl-2,3-dihydrobenzofuran-2-yl) pyrrolidine-1-carboxylate (C-II-f) (14 g, 30.3 mmol) in ACN (280 mL) and THF (140 mL), while maintaining the internal temperature at −8-0° C. The reaction mixture was stirred at −8-0° C. for 3 h. The mixture was diluted with MTBE (200 mL) and quenched by adding 6 wt % aq. Na₂CO₃ (150 mL). The organic layer was separated and the aqueous layer was extracted with MTBE (200 mL). The combined organic phases were washed with 6 wt % aq. Na₂CO₃, brine, dried over anhydrous Na₂SO₄, filtered and concentrated. The residue was purified by flash chromatography (silica; heptane/MTBE; 5% MTBE) to afford the title product (14 g), as a light yellow foam.

### Step 8: Tert-butyl (S)-2-((S)-5-chloro-6-fluoro-2-phenyl-4-(4,4,5,5-tetramethyl-1,3,2-dioxaborolan-2-yl)-2,3-dihydrobenzofuran-2-yl) pyrrolidine-1-carboxylate (C-II-h)

A 250 mL three-necked round bottomed flask was charged with tert-butyl (S)-2-((S)-4-bromo-5-chloro-6-fluoro-2-phenyl-2,3-dihydrobenzofuran-2-yl) pyrrolidine-1-carboxylate (C-II-g) (20.0 g, 40.26 mmol), bis(pinacolato)diboron (13.3 g, 52.34 mmol), KOAc (11.9 g, 120.77 mmol) and toluene (140 mL). The mixture was degassed with nitrogen for 20 min. PdCl₂(dppf) (2.4 g, 3.22 mmol) was added under a nitrogen atmosphere in one portion. The mixture was heated to 100° C. and stirred for 16 h. The mixture was then cooled to RT, filtered through Celite and concentrated to dryness. The residue was purified by flash chromatography (silica, heptane/MTBE 5:1) to give the title compound (14.5) as a foam. ¹H NMR (400 MHZ, CDCl₃) δ 7.46-7.41 (m, 2H), 7.34-7.27 (m, 3H), 6.70 (d, J=9.3 Hz, 1H), 4.54-4.38 (m, 1H), 4.34-3.98 (m, 1H), 3.45 (d, J=16.7 Hz, 2H), 3.06-2.83 (m, 1H), 2.09-1.78 (m, 2H), 1.58-1.46 (m, 2H), 1.42 (s, 9H), 1.36 (s, 12H). UPLC-MS 5: HRMS m/z calcd for C₂₉H₃₇BClFNO₅ [M+H]⁺ 544.2432, found 544.2897.

### Step 9: Methyl 4-((2S,4S)-2-((S)-1-(tert-butoxycarbonyl) pyrrolidin-2-yl)-5-chloro-6-fluoro-2-phenyl-2,3-dihydrobenzofuran-4-yl)-5-fluoro-6-(2-((tetrahydro-2H-pyran-2-yl)oxy)ethoxy)nicotinate (C-II-i) and methyl 4-((2S,4R)-2-((S)-1-(tert-butoxycarbonyl) pyrrolidin-2-yl)-5-chloro-6-fluoro-2-phenyl-2,3-dihydrobenzofuran-4-yl)-5-fluoro-6-(2-((tetrahydro-2H-pyran-2-yl)oxy)ethoxy)nicotinate (C-II-j)

A 1 L three-necked round bottomed flask was charged with(S)-2-((S)-5-chloro-6-fluoro-2-phenyl-4-(4,4,5,5-tetramethyl-1,3,2-dioxaborolan-2-yl)-2,3-dihydrobenzofuran-2-yl) pyrrolidine-1-carboxylate (C-II-h) (22.1 g, 40.65 mmol), methyl 4-chloro-5-fluoro-6-(2-((tetrahydro-2H-pyran-2-yl)oxy)ethoxy)nicotinate (N-II) (14.9 g, 44.72 mmol), K₃PO₄ (25.9 g, 121.96 mmol), water (100 mL) and toluene (300 mL). The mixture was degassed with nitrogen for 20 min. Pd₂(dba)₃ (1.86 g, 2.03 mmol) and N-Xantphos (2.24 g, 4.06 mmol) were added under nitrogen in one portion. The mixture was heated at 100° C. for 20 h. The mixture was cooled to RT and the organic phase was separated. The aqueous phase was extracted with toluene (100 mL). The combined organic phases were filtered through Celite and concentrated to dryness. The residue was purified by slurrying in a mixture of toluene, MTBE and n-heptane. The resulting suspension was filtered. The filter cake was recrystallized from toluene and n-heptane to give methyl 4-((2S,4S)-2-((S)-1-(tert-butoxycarbonyl) pyrrolidin-2-yl)-5-chloro-6-fluoro-2-phenyl-2,3-dihydrobenzofuran-4-yl)-5-fluoro-6-(2-((tetrahydro-2H-pyran-2-yl)oxy)ethoxy)nicotinate (7.5 g) as an off-white solid. ¹H NMR (400 MHz, CDCl₃) δ 8.72 (s, 1H), 7.39-7.34 (m, 2H), 7.34-7.27 (m, 3H), 6.75 (d, J=9.1 Hz, 1H), 4.76-4.61 (m, 3H), 4.41-4.28 (m, 1H), 4.19-4.08 (m, 1H), 3.92-3.81 (m, 2H), 3.76 (s, 3H), 3.72-2.84 (m, 5H), 2.01-1.44 (m, 10H), 1.27 (s, 9H). UPLC-MS 5: HRMS m/z calcd for C₃₇H₄₂ClF₂N₂O₈ [M+H]⁺ 715.2592, found 715.2544.

### Step 10: Sodium 4-((2S,4S)-2-((S)-1-(tert-butoxycarbonyl) pyrrolidin-2-yl)-5-chloro-6-fluoro-2-phenyl-2,3-dihydrobenzofuran-4-yl)-5-fluoro-6-(2-((tetrahydro-2H-pyran-2-yl)oxy)ethoxy)nicotinate (C-II-k)

A 1 L three-necked round bottomed flask was charged with methyl 4-((2S,4S)-2-((S)-1-(tert-butoxycarbonyl) pyrrolidin-2-yl)-5-chloro-6-fluoro-2-phenyl-2,3-dihydrobenzofuran-4-yl)-5-fluoro-6-(2-((tetrahydro-2H-pyran-2-yl)oxy)ethoxy)nicotinate (C-II-i) (50.0 g, 69.91 mmol) and 1,4-dioxane (300 mL). A solution of NaOH (5.6 g, 139.83 mmol) in water (150 mL) was added dropwise at RT. The mixture was heated to 40° C. and stirred for 16 h. The mixture was concentrated to remove most of the 1,4-dioxane and extracted with MTBE (500 mL). The organic layer was washed with brine (500 mL), separated and concentrated to dryness to afford the title compound (53.0 g) as an off-white solid. ¹H NMR (400 MHZ, CDCl₃) δ 8.18 (s, 1H), 7.47 (d, J=7.5 Hz, 2H), 7.32 (dt, J=12.8, 7.1 Hz, 3H), 6.70 (d, J=8.9 Hz, 1H), 4.67 (s, 1H), 4.63-4.43 (m, 2H), 4.37-4.22 (m, 2H), 4.11-3.94 (m, 1H), 3.91-3.75 (m, 2H), 3.56-3.34 (m, 2H), 3.33-3.23 (m, 1H), 2.72-2.54 (m, 1H), 1.98-1.35 (m, 10H), 1.24 (s, 9H). UPLC-MS 5: HRMS m/z calcd for C₃₆H₃₈ClF₂N₂O₈ [M−Na]-699.2290, found 699.2238.

### Step 11: Tert-butyl (2S)-2-((2S,4S)-5-chloro-6-fluoro-4-(3-fluoro-5-(methylcarbamoyl)-2-(2-((tetrahydro-2H-pyran-2-yl)oxy)ethoxy)pyridin-4-yl)-2-phenyl-2,3-dihydrobenzofuran-2-yl) pyrrolidine-1-carboxylate (C-II-l)

A 1 L three-necked round bottomed flask was charged with sodium 4-((2S,4S)-2-((S)-1-(tert-butoxycarbonyl) pyrrolidin-2-yl)-5-chloro-6-fluoro-2-phenyl-2,3-dihydrobenzofuran-4-yl)-5-fluoro-6-(2-((tetrahydro-2H-pyran-2-yl)oxy)ethoxy)nicotinate (C-II-k) (20.0 g, 27.66 mmol) and DMA (100 mL). The solution was cooled to 5-10° C. DIPEA (16.1 g, 124.46 mmol) and MeNH₂ HCl (3.7 g, 55.32 mmol) were then added in one portion. The mixture was stirred for 20 min. HATU (15.8 g, 41.49 mmol) was added portionwise at 5-10° C. The reaction mixture was then stirred at RT for 2 h before it was diluted with MTBE (300 mL). A solution of NaOH (4.4 g, 110.6 mmol) in water (300 mL) was added dropwise at RT. The mixture was stirred for 10 min and the phases were separated. The aqueous phase was extracted with MTBE (200 mL). The combined organic phases were washed with water (200 mL), 15 wt % aq. citric acid (200 mL), brine (200 mL), and then concentrated to dryness to give the title compound (18.2 g) as a foam. ¹H NMR (400 MHZ, CDCl₃) δ 8.37 (s, 1H), 7.45-7.32 (m, 5H), 7.07 (s, 1H), 6.81 (d, J=9.0 Hz, 1H), 4.77-4.53 (m, 3H), 4.20 (d, J=8.3 Hz, 1H), 4.15-4.05 (m, 1H), 3.94-3.78 (m, 2H), 3.74-3.61 (m, 1H), 3.55-3.45 (m, 1H), 3.30-3.09 (m, 2H), 2.90 (s, 1H), 2.72 (s, 3H), 2.05-1.45 (m, 10H), 1.28 (s, 9H). UPLC-MS 5: HRMS m/z calcd for C₃₇H₄₃ClF₂N₃O₇ [M+H]⁺ 714.2752, found 714.2723.

### Step 12: 4-((2S,4S)-5-Chloro-6-fluoro-2-phenyl-2-((S)-pyrrolidin-2-yl)-2,3-dihydrobenzofuran-4-yl)-5-fluoro-6-(2-hydroxyethoxy)-N-methylnicotinamide (C-II)

A 250 mL three-necked round bottomed flask was charged with tert-butyl (2S)-2-((2S,4S)-5-chloro-6-fluoro-4-(3-fluoro-5-(methylcarbamoyl)-2-(2-((tetrahydro-2H-pyran-2-yl)oxy)ethoxy)pyridin-4-yl)-2-phenyl-2,3-dihydrobenzofuran-2-yl) pyrrolidine-1-carboxylate (C-II-l) (20.0 g, 28.00 mmol), IPA (100 mL) and ethane-1,2-diol (20 mL). HCl (28 mL, 5-6 N in IPA) was then added in one portion. The reaction mixture was stirred at RT for 16 h. The reaction was cooled to 10° C. IPAc (200 mL) and a solution of NaOH (7.8 g, 195.0 mmol) in water (200 mL) were added. The mixture was stirred at RT for 10 min and the phases were separated. The aqueous phase was extracted with IPAc (200 mL). The combined organic phases were washed with brine (200 mL) and concentrated to dryness. The residue was dissolved in THF (100 mL) to give a clear yellow solution. A solution of succinic acid (3.64 g, 30.8 mmol) in THF (75 mL) was added dropwise. The resulting white suspension was stirred for 13 h and filtered. The filter cake was dissolved in water (340 mL) and cooled to 10° C. A solution of NaOH (2.3 g, 57.5 mmol) in water (85 mL) was added dropwise. The resulting suspension was stirred for 1 h and filtered. The filter cake was washed with water (85 mL×2) and dried under vacuum to give the title compound (10.3 g) as an off-white solid. ¹H NMR (600 MHZ, DMSO-d₆) δ (ppm) 8.42 (q, J=4.6 Hz, 1H), 8.27 (s, 1H), 7.46-7.42 (m, 2H), 7.33 (t, J=7.6 Hz, 2H), 7.26 (dd, J=8.3, 6.2 Hz, 1H), 7.07 (d, J=9.6 Hz, 1H), 4.95 (t, J=5.5 Hz, 1H), 4.44 (ddt, J=21.1, 10.9, 5.5 Hz, 2H), 3.76 (q, J=5.1 Hz, 2H), 3.51 (t, J=6.8 Hz, 1H), 3.43 (d, J=16.0 Hz, 1H), 2.96 (d, J=16.0 Hz, 1H), 2.74 (dt, J=12.6, 6.4 Hz, 1H), 2.66 (d, J=4.6 Hz, 3H), 2.65-2.60 (m, 1H), 2.29 (s br, 1H), 1.55-1.43 (m, 2H), 1.42-1.33 (m, 2H). UPLC-MS 1: m/z 530.5/532.5 [M+H]⁺, t_{R}=0.69 min. UPLC-MS 2: m/z 530.5/532.5 [M+H]⁺, t_{R}=3.02 min.

### Synthesis of ((2S,3S)-5-chloro-6-fluoro-3-methyl-2-phenyl-4-(4,4,5,5-tetramethyl-1,3,2-dioxaborolan-2-yl)-2,3-dihydrobenzofuran-2-yl)methanol (C-III)

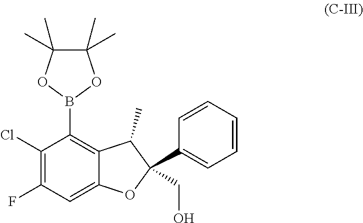 [SMILES: C[C@H]1c2c(cc(F)c(Cl)c2B2OC(C)(C)C(C)(C)O2)O[C@]1(CO)c1ccccc1]

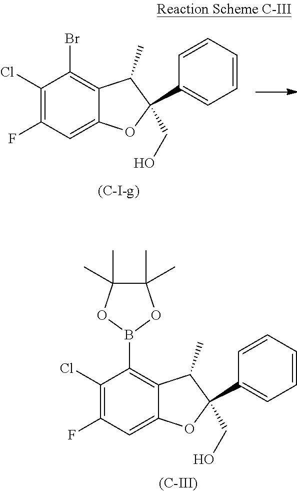

At 60° C. PdCl₂(dppf)·CH₂Cl₂ adduct (4.39 g, 5.38 mmol) was added to a stirred suspension of ((2S,3S)-4-bromo-5-chloro-6-fluoro-3-methyl-2-phenyl-2,3-dihydrobenzofuran-2-yl)methanol (C-I-g) (20 g, 53.8 mmol), bis(pinacolato)diboron (20.50 g, 81 mmol) and potassium hydroxide (6.04 g, 108 mmol) in toluene (200 mL). The reaction mixture was stirred at 100° C. for 3 h before it was filtered through Celite and concentrated under reduced pressure. The obtained crude material was purified twice by flash chromatography (silica, heptane/EtOAc, gradient: 0% to 30% EtOAc) to give the title product (16.7 g) as a colorless foam. ¹H NMR (400 MHZ, DMSO-d₆) δ 7.45-7.40 (m, 2H), 7.33 (t, J=7.5 Hz, 2H), 7.28-7.22 (m, 1H), 7.12 (d, J=9.5 Hz, 1H), 4.98 (t, J=5.1 Hz, 1H), 3.95 (dd, J=11.7, 5.6 Hz, 1H), 3.87 (dd, J=11.7, 5.4 Hz, 1H), 3.55 (q, J=7.0 Hz, 1H), 1.37 (d, J=7.1 Hz, 3H), 1.32 (s, 6H), 1.30 (s, 6H). UPLC-MS 1: m/z 463.3 [M+formate]⁻, t_{R}=1.30 min.

### Synthesis of tert-butyl (S)-((5-chloro-6-fluoro-2-phenyl-4-(4,4,5,5-tetramethyl-1,3,2-dioxaborolan-2-yl)-2,3-dihydrobenzofuran-2-yl)methyl)carbamate (C-IV)

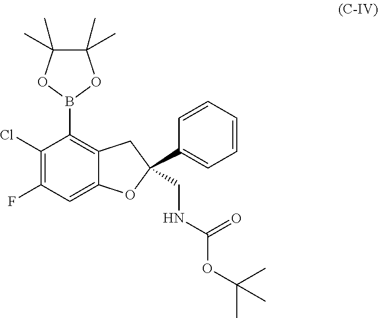 [SMILES: CC(C)(C)OC(=O)NC[C@@]1(c2ccccc2)Cc2c(cc(F)c(Cl)c2B2OC(C)(C)C(C)(C)O2)O1]

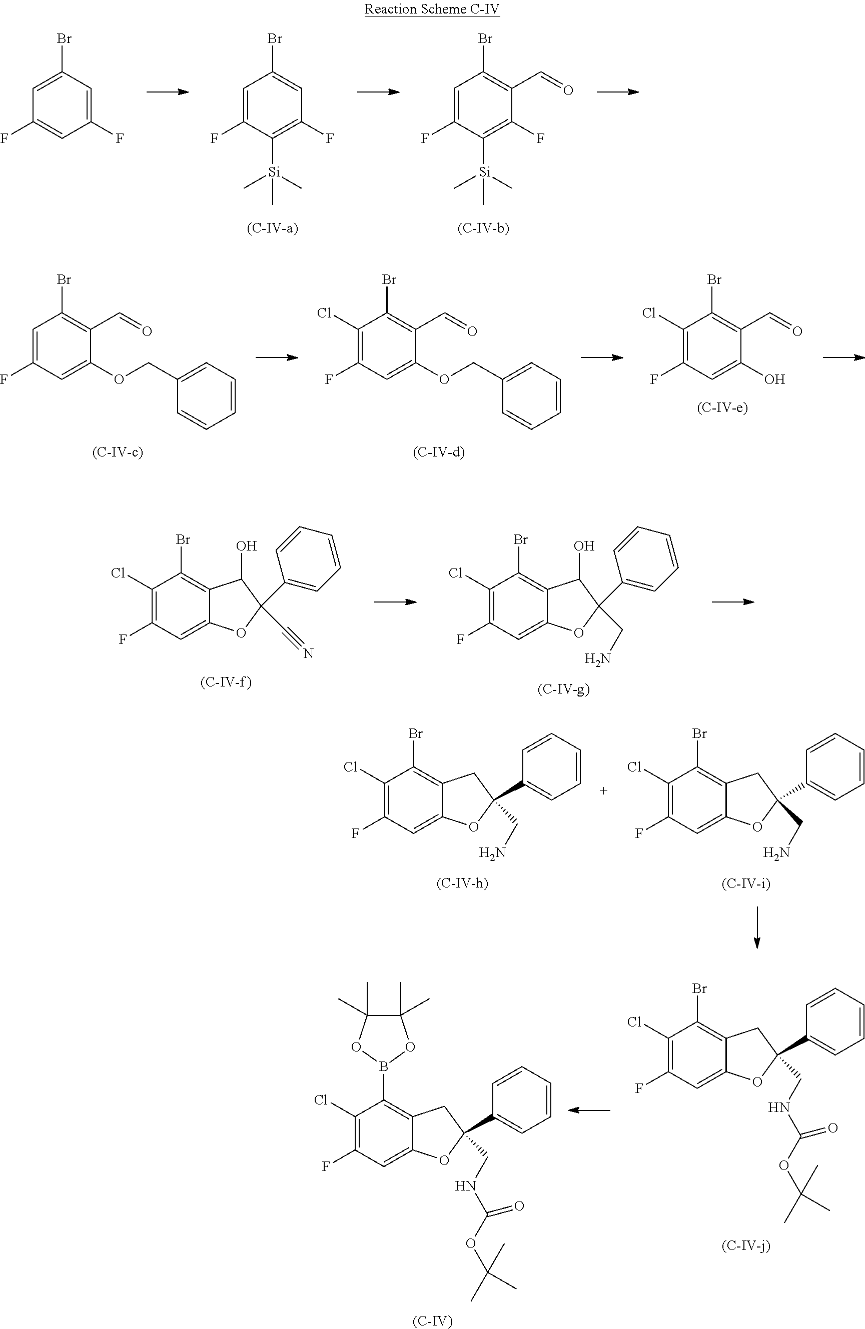

### Step 1: (4-Bromo-2,6-difluorophenyl)trimethylsilane (C-IV-a)

At −78° C. diisopropylamine (555 mL, 3.9 mol) was added dropwise to a solution of n-butyllithium (1560 mL, 3.9 mol, 2.5 M in hexane) in THF (3000 mL) within 10 min. After 10 min, 1-bromo-3,5-difluorobenzene (500 g, 2.6 mol) was added dropwise to the freshly prepared LDA solution. The reaction mixture was stirred at −78° C. for 2 h before chlorotrimethylsilane (488 mL, 3.9 mol) was added dropwise at −78° C. within 10 min. The resulting solution was stirred at −78° C. for 1 h. After evaporation of the solvents, the resulting crude material was distilled to afford the title compound (470 g) as a colorless oil. UPLC-MS 1: m/z 282.4 [M+NH₄]⁺, t_{R}=1.53 min. ¹HNMR (300 MHZ, CDCl₃): δ 7.01 (s, 1H), 6.99 (s, 1H), 0.37 (s, 9H).

### Step 2: 6-Bromo-2,4-difluoro-3-(trimethylsilyl)benzaldehyde (C-IV-b)

At −78° C. redistilled diisopropylamine (186 mL, 1.13 mol) was added dropwise to a solution of n-butyllithium in hexane (453 mL, 1.13 mol) in THF (1000 mL) cooled at −78° C. within 10 min and stirring was continued for 1 h. Then, the freshly prepared LDA solution was added dropwise to a solution of (4-bromo-2,6-difluorophenyl)trimethylsilane (C-IV-a) (200 g, 0.754 mol) in THF (1000 mL) cooled to −78° C. The yellow solution was stirred for 1 h at −78° C. before DMF (104 mL, 1.36 mol) was added dropwise within 5 min. The resulting yellow reaction mixture was stirred at −78° C. for 1 h. Then, an acetic acid solution (188 mL) and water (800 mL) were added. The yellow suspension was stirred at RT for an additional 1.5 h. The two phases were separated and the aqueous phase was extracted with EtOAc. The combined organic phases were washed with brine, dried over anhydrous Na₂SO₄, filtered and concentrated under reduced pressure affording the title product (176 g). UPLC-MS 1: m/z 293.0/295.0 [M+H]⁺, t_{R}=1.25/1.38 min. 1H-NMR (600 MHz, DMSO-d₆) δ ppm 10.13 (s, 1H), 7.61 (d, J=8.3 Hz, 1H), 0.35 (s, 9H).

### Step 3: 2-(Benzyloxy)-6-bromo-4-fluorobenzaldehyde (C-IV-c)

At RT a solution of sodium benzyloxide (1020 mL, 1.02 mol freshly prepared: 23.46 g Na in 1020 mL benzyl alcohol) was added dropwise to a solution of 6-bromo-2,4-difluoro-3-(trimethylsilyl)benzaldehyde (C-IV-b) (300 g, 1.02 mol) in benzyl alcohol (500 mL) within 30 min. The light yellow reaction mixture was stirred at 40° C. for 15 min. The resulting suspension was diluted with EtOAc, then extracted successively with a sat solution of NH₄Cl and brine. The separated aqueous phase was back-extracted with EtOAc. The combined organic phases were dried over anhydrous Na₂SO₄, filtered and concentrated under reduced pressure to give the crude product which was purified by flash chromatography (silica, PE) to give the title compound (90 g) as a light beige solid. UPLC-MS 1: m/z 309.0/311.0 [M+H]⁺, t_{R}=1.24 min. 1H-NMR (600 MHZ, DMSO-d₆) δ ppm 10.24 (s, 1H), 7.48 (d, J=7.5 Hz, 2H), 7.41 (t, J=7.5 Hz, 2H), 7.37-7.28 (m, 3H), 5.27 (s, 2H).

### Step 4: 6-(Benzyloxy)-2-bromo-3-chloro-4-fluorobenzaldehyde (C-IV-d)

To a solution of 2-(benzyloxy)-6-bromo-4-fluorobenzaldehyde (C-IV-c) (240 g, 777 mmo) in ACN (3000 mL) were added N-chlorosuccinimide (134 g, 1010 mmol) and p-TsOH monohydrate (221 g, 1165 mmol) at RT. The light yellow solution was stirred for 24 h at RT. The reaction mixture was diluted with EtOAc and extracted with a sat solution of NaHCO₃ and brine. The combined aqueous phases were back-extracted with EtOAc. The combined organic phases were dried over anhydrous Na₂SO₄, filtered and concentrated under reduced pressure to give the crude product which was suspended in a mixture of MTBE and PE (ratio 1:8). The crystallized material was filtered, washed with PE and dried under HV at 50° C. overnight to give the title product (186 g) as a colorless powder. UPLC-MS 1: m/z 342.8/344.8 [M+H]⁺, t_{R}=1.31 min. ¹HNMR (300 MHZ, CDCl₃): δ 10.37 (s, 1H), 7.43-7.37 (m, 5H), 6.91 (d, J=10.2 Hz, 1H), 5.18 (s, 2H).

### Step 5: 2-Bromo-3-chloro-4-fluoro-6-hydroxybenzaldehyde (C-IV-e)

To a suspension of 6-(benzyloxy)-2-bromo-3-chloro-4-fluorobenzaldehyde (C-IV-d) (300 g, 873 mmol) in DCM (3 L), placed under nitrogen and cooled to −78° C., was added a solution of boron tribromide (960 mL, 960 mmol, 1 M in DCM) within 5 min. The resulting brown solution was stirred at −78° C. for 1.5 h. The reaction mixture was slowly quenched with MeOH and the solvents were removed under reduced pressure. The crude material was redissolved in MeOH and concentrated under reduced pressure again. The crude product was purified by flash chromatography (silica, PE/EtOAc, gradient 0% to 10% EtOAc) to give the title compound (180 g) as a beige powder. UPLC-MS 1: m/z 251.0/252.9 [M−1]⁻, t_{R}=1.12 min. 1H-NMR (600 MHZ, DMSO-d₆) δ ppm 12.05 (s, 1H), 10.19 (s, 1H), 7.18 (d, J=10.6 Hz, 1H).

### Step 6: 4-Bromo-5-chloro-6-fluoro-3-hydroxy-2-phenyl-2,3-dihydrobenzofuran-2-carbonitrile (C-IV-f)

At RT 2-bromo-2-phenylacetonitrile (196 g, 1 mol) followed by DIPEA (238 mL, 1.4 mol) were added to a solution of 2-bromo-3-chloro-4-fluoro-6-hydroxybenzaldehyde (C-IV-e) (230 g, 910 mmol) in DCM (4.5 L) and stirring was continued for 5 h at RT. For workup the reaction mixture was diluted with DCM and washed with water. The aqueous layer was back-extracted with DCM. The combined organic phases were dried over anhydrous Na₂SO₄, filtered and concentrated under reduced pressure. The crude material was purified by flash chromatography (silica, heptane/EtOAc then DCM) to give the title compound as a diastereomeric mixture (230 g). UPLC-MS 1: m/z 366.0/368.0 [M−H]⁻, t_{R}=1.20 min (both diastereoisomers coelute).

### Step 7: 2-(Aminomethyl)-4-bromo-5-chloro-6-fluoro-2-phenyl-2,3-dihydrobenzofuran-3-ol (C-IV-g)

At RT a solution of borane-methyl sulfide complex (1.6 L, 3.25 mol, 2 M in THF) was added to a solution of 4-bromo-5-chloro-6-fluoro-3-hydroxy-3-methyl-2-phenyl-2,3-dihydrobenzofuran-2-carbonitrile (C-IV-f) (240 g, 0.65 mol) in THF (2.4 L). The reaction mixture was stirred at 65° C. for 2 h. The solution was cooled to RT and MeOH (5 L) was carefully added dropwise within 3 min. After 30 min at RT, 1 N HCl was added and stirring at RT was continued for 18 h. The reaction solution was quenched by the addition of a sat solution of NaHCO₃ and extracted with DCM. The combined organic phases were washed with water, dried over anhydrous Na₂SO₄, filtered and concentrated under reduced pressure to afford the title compound as a diastereomeric mixture (175 g) as a yellow foam. UPLC-MS 1: m/z 372.1/374.1 [M+1]⁺, t_{R}=0.65 min and 0.78 min.

### Step 8: (S)-(4-Bromo-5-chloro-6-fluoro-2-phenyl-2,3-dihydrobenzofuran-2-yl) methanamine (C-IV-h) and (R)-(4-bromo-5-chloro-6-fluoro-2-phenyl-2,3-dihydrobenzofuran-2-yl) methanamine (C-IV-i)

At RT triethylsilane (930 mL, 5.8 mol) followed by boron trifluoride diethyletherate (244 mL, 2 mol) were added to a solution of 2-(aminomethyl)-4-bromo-5-chloro-6-fluoro-2-phenyl-2,3-dihydrobenzofuran-3-ol (C-IV-g) (250 g, 671 mmol) in DCM (3 L) The reaction solution was stirred at RT overnight before it was quenched by the addition of a sat solution of NaHCO₃ (sat.) and extracted with DCM. The combined organic phases were washed with brine, dried over anhydrous Na₂SO₄, filtered and concentrated under reduced pressure. The crude product was purified by flash chromatography (silica, DCM/MeOH/NH₃ 100:1:0.5) to give racemic (4-bromo-5-chloro-6-fluoro-2-phenyl-2,3-dihydrobenzofuran-2-yl) methanamine (115 g). UPLC-MS 1: m/z 356.0/358.0 [M+H]⁺, t_{R}=0.89 min.

The racemate (4-bromo-5-chloro-6-fluoro-2-phenyl-2,3-dihydrobenzofuran-2-yl) methanamine was subjected to chiral SFC (ChiralPak IC, 300×50 mm I.D., 10 μm. CO₂/IPA (0.1% ammonia) 7:3, 40° C., flow rate: 200 mL/min, 7 mL/injection, cycle time 7 min) to afford the two enantiomers (S)-(4-bromo-5-chloro-6-fluoro-2-phenyl-2,3-dihydrobenzofuran-2-yl) methanamine (C-IV-h) and (R)-(4-bromo-5-chloro-6-fluoro-2-phenyl-2,3-dihydrobenzofuran-2-yl) methanamine (C-IV-i) with an enantiomeric excess of >98%, respectively.

(S)-(4-bromo-5-chloro-6-fluoro-2-phenyl-2,3-dihydrobenzofuran-2-yl) methanamine (C-IV-h): Chiral SFC: (Chiralpak IC 150×4.6 mm I.D., 3 μm, CO₂/IPA (0.05% DEA) 8:2, flow rate: 2.4 mL/min) t_{R}=5.64 min; UPLC-MS 1: m/z 356.0/358.0 [M+H]⁺, t_{R}=0.89 min. ¹H NMR (600 MHZ, DMSO-d₆) δ 7.46 (d, J=7.6 Hz, 2H), 7.40 (t, J=7.6 Hz, 2H), 7.32 (t, J=7.3 Hz, 1H), 7.16 (d, J=9.6 Hz, 1H), 3.81 (dd, J=16.2, 1.8 Hz, 1H), 3.19 (d, J=16.2 Hz, 1H), 3.01 (s, 2H).

An X-ray crystal structure of(S)-(4-bromo-5-chloro-6-fluoro-2-phenyl-2,3-dihydrobenzofuran-2-yl) methanamine (C-IV-h) as a besyalte salt confirmed the absolute configuration(S): (R)-(4-bromo-5-chloro-6-fluoro-2-phenyl-2,3-dihydrobenzofuran-2-yl) methanamine C-IV-i): Chiral SFC: (Chiralpak IC 150×4.6 mm I.D., 3 μm, CO₂/IPA (0.05% DEA) 8:2, flow rate: 2.4 mL/min) t_{R}=6.55 min; UPLC-MS 1: m/z 356.0/358.0 [M+H]⁺, t_{R}=0.89 min.

### Step 9: Tert-butyl (S)-((4-bromo-5-chloro-6-fluoro-2-phenyl-2,3-dihydrobenzofuran-2-yl)methyl)carbamate (C-IV-j)

Boc₂O (46.27 g, 212.00 mmol, 48.71 mL) was added in portions to a solution of(S)-(4-bromo-5-chloro-6-fluoro-2-phenyl-2,3-dihydrobenzofuran-2-yl) methanamine (C-IV-h) (72.00 g, 201.9 mmol) in DCM (1500 mL). The reaction mixture was stirred at RT for 16 h. After removal of the solvents under reduced pressure the crude product was purified by flash chromatography (silica, PE/EtOAc, gradient 0% to 15% EtOAc) to afford the title compound (102 g) as a colorless oil. UPLC-MS 1: m/z 458.1/460.0 [M+H]⁺, t_{R}=1.48 min. ¹H NMR (600 MHz, DMSO-d₆) δ 7.46 (d, J=7.6 Hz, 2H), 7.41 (t, J=7.7 Hz, 2H), 7.33 (t, J=7.3 Hz, 1H), 7.21 (t, J=6.2 Hz, 1H), 7.12 (d, J=9.5 Hz, 1H), 3.75 (d, J=16.4 Hz, 1H), 3.54 (dd, J=14.5, 6.5 Hz, 1H), 3.38 (dd, J=14.6, 6.1 Hz, 1H), 3.25 (d, J=16.6 Hz, 1H), 1.30 (s, 9H).

### Step 10: Tert-butyl (S)-((5-chloro-6-fluoro-2-phenyl-4-(4,4,5,5-tetramethyl-1,3,2-dioxaborolan-2-yl)-2,3-dihydrobenzofuran-2-yl)methyl)carbamate (C-IV)

At 100° C. PdCl₂(dppf)·CH₂Cl₂ adduct (3.58 g, 4.38 mmol) was added to a stirred solution of tert-butyl (S)-((4-bromo-5-chloro-6-fluoro-2-phenyl-2,3-dihydrobenzofuran-2-yl)methyl)carbamate (C-IV-j) (20 g, 43.8 mmol), bis(pinacolato)diboron (16.68 g, 65.7 mmol) and KOAc (12.89 g, 131 mmol) in dioxane (100 mL) under Ar and stirring at 100° C. was continued for 16 h. The reaction mixture was filtered over Celite and concentrated. The crude product was purified by flash chromatography (silica, hexane/EtOAc; gradient: 0% to 25% EtOAc) to afford the title compound (15.1 g) as a colorless powder. ¹H NMR (600 MHZ, DMSO-d₆) δ 7.44-7.38 (m, 4H), 7.34-7.31 (m, 1H), 7.17 (d, J=6.2 Hz, 1H), 7.08 (d, J=9.9 Hz, 1H), 3.70 (d, J=16.5 Hz, 1H), 3.56 (dd, J=14.7, 6.8 Hz, 1H), 3.37 (dd, J=14.5, 5.9 Hz, 1H), 3.22 (d, J=16.9 Hz, 1H), 1.32 (s, 9H), 1.31 (s, 6H), 1.30 (s, 6H). UPLC-MS 1: m/z 521.3/523.3 [M+17]⁺, t_{R}=1.52 min.

Section B: Final Compound Examples and their Syntheses

### General Procedure 1 (GP1)

### Example 1: 2-((2S,3S,4S)-5-chloro-2-((((1r,4S)-4-(4-((1-(3-(2,4-dioxotetrahydropyrimidin-1(2H)-yl)-4-methoxybenzoyl)piperidin-4-yl)methyl)piperidine-1-carbonyl)cyclohexyl)amino)methyl)-6-fluoro-3-methyl-2-phenyl-2,3-dihydrobenzofuran-4-yl)-3-fluoro-4-methoxybenzamide

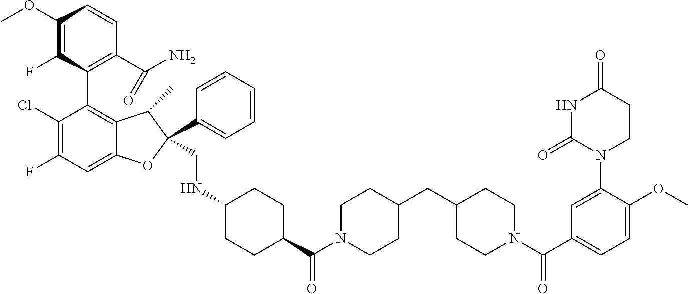

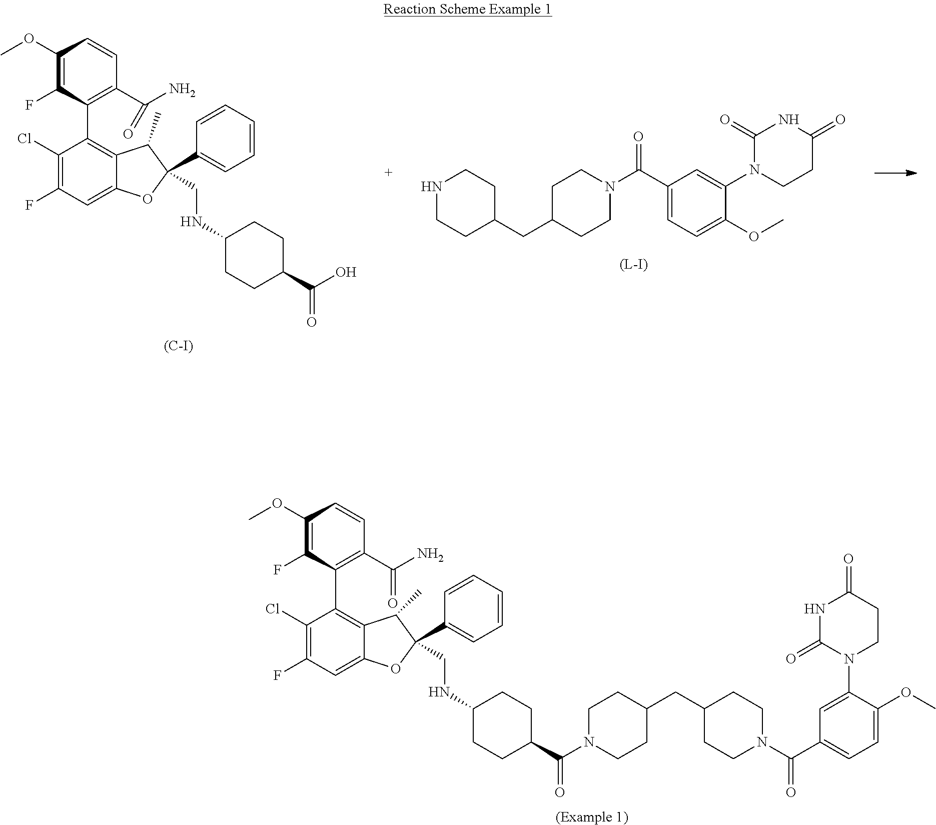

To a stirred solution of (1S,4r)-4-((((2S,3S,4S)-4-(6-carbamoyl-2-fluoro-3-methoxyphenyl)-5-chloro-6-fluoro-3-methyl-2-phenyl-2,3-dihydrobenzofuran-2-yl)methyl)amino)cyclohexane-1-carboxylic acid (C-I) (70 mg, 0.100 mmol) in DMF (1.4 mL) was added TEA (97 μl, 0.701 mmol) followed by HATU (57.1 mg, 0.150 mmol). After 5 min, 1-(2-methoxy-5-(4-(piperidin-4-ylmethyl)piperidine-1-carbonyl)phenyl)dihydropyrimidine-2,4(1H,3H)-dione (L-I) (69.8 mg, 0.120 mmol) was added and stirring at RT was continued for 18 h. After concentration the residue was purified by prep HPLC followed by SFC to give the title compound (36 mg) as a colorless solid. UPLC-MS 4: m/z 995.8 [M+H]⁺, t_{R}=0.78 min. ¹H NMR (400 MHZ, DMSO-d₆) δ (ppm) 10.33 (s, 1H), 7.63-7.57 (m, 2H), 7.43-7.38 (m, 2H), 7.38-7.23 (m, 6H), 7.14 (d, J=8.6 Hz, 1H), 7.11 (s br, 1H), 7.07 (d, J=9.6 Hz, 1H), 4.33 (d, J=12.3 Hz, 1H), 3.91-3.80 (m, 2H), 3.88 (s, 3H), 3.84 (s, 3H), 3.59 (t, J=6.8 Hz, 2H), 3.28-3.18 (m, 1H), 3.18-3.07 (m, 2H), 3.01-2.87 (m, 1H), 2.71-2.68 (m, 2H), 2.47-2.38 (m, 2H), 2.26-2.08 (m, 1H), 1.81-1.50 (m, 8H), 1.32-1.21 (m, 2H), 1.15-1.10 (m, 2H), 1.08-1.02 (m, 2H), 0.92 (d, J=7.1 Hz, 3H), 0.90-0.79 (m, 4H); not all signals visible in aliphatic region due to overlap with solvent peaks.

The following Examples were prepared according to General Procedure 1:

| | | UPLC MS | |
|---|---|---|---|
| | | m/z | |
| | | t_{R} [min] | |
| Ex. | Structure/Chemical Name | (method) | ¹H NMR |
| 2 | 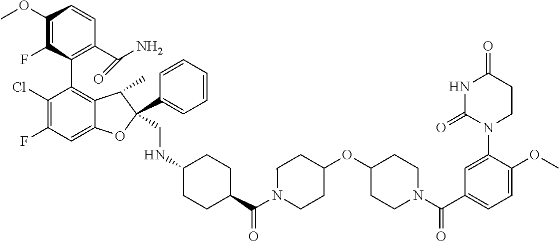 | 997.8 [M + H]⁺ 0.71 (4) | (400 MHz, DMSO-d₆) δ (ppm) 10.31 (s, 1H), 7.60 (dd, J = 8.7, 1.4 Hz, 2H), 7.44-7.23 (m, 8H), 7.15 (d, J = 8.5 Hz, 1H), 7.10 (s br, 1H), 7.07 (d, J = 9.5 Hz, 1H), 3.88 (s, 3H), 3.84 (s, 3H), 3.87-3.80 (m, 1H), 3.75-3.62 (m, 3H), 3.59 (t, J = 6.7 Hz, 2H), 3.27- 3.09 (m, 6H), 3.05-2.97 (m, 1H), 2.68 (t, J = 6.6 Hz, 2H), 2.48-2.40 (m, 1H), 2.24-2.14 (m, 1H), 1.85- 1.68 (m, 6H), 1.60-1.50 (m, 2H), 1.48-1.20 (m, 8H), |
| | 2-((2S,3S,4S)-5-chloro-2-((((1r,4S)-4-(4- | | 0.93 (d, J = 7.2 Hz, 3H), 0.93- |
| | ((1-(3-(2,4-dioxotetrahydropyrimidin- | | 0.83 (m, 3H) |
| | 1(2H)-yl)-4-methoxybenzoyl)piperidin-4- | | |
| | yl)oxy)piperidine-1- | | |
| | carbonyl)cyclohexyl)amino)methyl)-6- | | |
| | fluoro-3-methyl-2-phenyl-2,3- | | |
| | dihydrobenzofuran-4-yl)-3-fluoro-4-methoxybenzamide Synthesized from C-I and L-II | | |
| 3 | 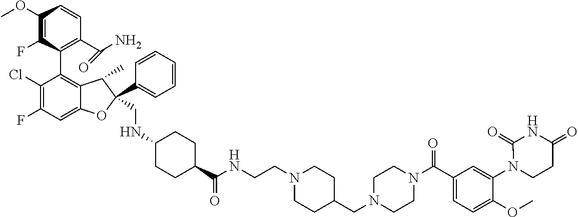 | 1039.4 [M + H]⁺ 0.50 (4) | (400 MHz, DMSO-d₆) δ (ppm) 10.32 (s, 1H), 7.59 (d, J = 8.6 Hz, 2H), 7.55-7.50 (m, 1H), 7.43-7.24 (m, 7H), 7.15 (d, J = 8.5 Hz, 1H), 7.10-7.07 (m, 1H), 7.06 (d, J = 10.2 Hz, 1H), 3.88 (s, 3H), 3.84 (s, 3H), 3.59 (t, J = 7.2 Hz, 2H), 3.54-3.42 (m, 4H), 3.28- 3.20 (m, 1H), 3.17-3.04 (m, 4H), 2.83-2.74 (m, 2H), 2.30-2.22 (m, 2H), 2.20- 2.08 (m, 2H), 2.04-1.80 (m, |
| | 2-((2S,3S,4S)-5-chloro-2-((((1r,4S)-4-((2- | | 3H), 1.80-1.68 (m, 2H), |
| | (4-((4-(3-(2,4-dioxotetrahydropyrimidin- | | 1.14-1.00 (m, 3H), 1.67- |
| | 1(2H)-yl)-4-methoxybenzoyl)piperazin-1- | | 1.55 (m, 4H), 1.53-1.39 (m, |
| | yl)methyl)piperidin-1-yl)ethyl)carbamoyl)cyclohexyl)amino)methyl)- | | 2H), 1.32-1.17 (m, 6H), 0.92 |
| | 6-fluoro-3-methyl-2-phenyl-2,3-dihydrobenzofuran-4-yl)-3-fluoro-4- | | (d, J = 7.2 Hz, 3H), 0.88- |
| | methoxybenzamide Synthesized from C-I and L-X | | 0.71 (m, 4H) |
| 4 | 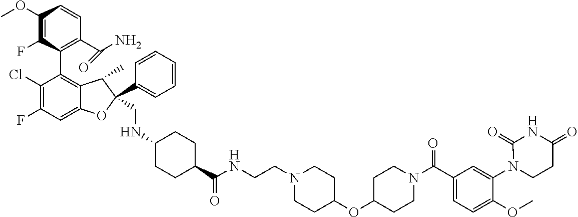 | 520.9 [M + 2H]^{2+/2} 0.56 (4) | (400 MHz, DMSO-d₆) δ (ppm) 10.32 (s, 1H), 7.59 (dd, J = 8.8, 1.4 Hz, 2H), 7.55-7.51 (m, 1H), 7.44-7.23 (m, 7H), 7.14 (d, J = 8.6 Hz, 1H), 7.0 (s br, 1H), 7.06 (d, J = 9.5 Hz, 1H), 3.88 (s, 3H), 3.84 (s, 3H), 3.69-3.63 (m, 1H), 3.59 (t, J = 6.6 Hz, 2H), 3.43- 3.37 (m, 1H), 3.27-3.03 (m, 7H), 2.70-2.61 (m, 4H), 2.29-2.23 (m, 2H), 2.19-2.10 (m, 2H), 2.08-1.98 (m, 2H), |
| | 2-((2S,3S,4S)-5-chloro-2-((((1r,4S)-4-((2- | | 1.98-1.88 (m, 2H), 1.82-1.69 |
| | (4-((1-(3-(2,4-dioxotetrahydropyrimidin- | | (m, 6H), 1.64-1.56 (m, 2H), |
| | 1(2H)-yl)-4-methoxybenzoyl)piperidin-4-yl)oxy)piperidin-1- | | 1.44-1.33 (m, 4H), 1.30-1.18 |
| | yl)ethyl)carbamoyl)cyclohexyl)amino)methyl)- | | (m, 3H), 0.92 (d, J = 7.1 Hz, |
| | 6-fluoro-3-methyl-2-phenyl-2,3- | | 3H), 0.90-0-73 (m, 3H). |
| | dihydrobenzofuran-4-yl)-3-fluoro-4- | | |
| | methoxybenzamide Synthesized from C-I and L-XI | | |
| 5 | 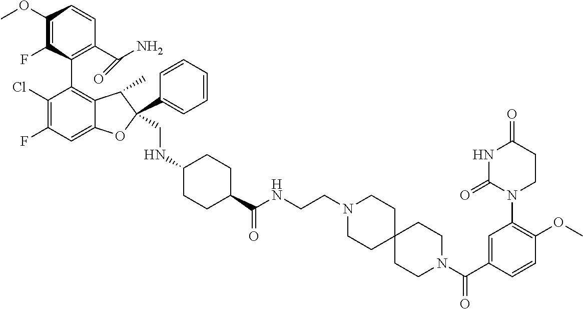 | 1010.4 [M + H]⁺ 0.61 (4) | (400 MHZ, DMSO-d₆) δ (ppm) 10.32 (s, 1H), 7.65-7.58 (m, 2H), 7.56-7.52 (m, 1H), 7.43-7.38 (m, 2H), 7.38-7.23 (m, 5H), 7.14 (d, J = 8.6 Hz, 1H), 7.09 (s br, 1H), 7.06 (d, J = 9.6 Hz, 1H), 3.88 (s, 3H), 3.84 (s, 3H), 3.59 (t, J = 6.7 Hz, 2H), 3.52-3.38 (m, 1H), 3.28-3.2.0 (m, 1H), 3.16-3.03 (m, 4H), 2.72-2.61 (m, 2H), 2.38-2.22 (m, 8H), 2.20-2.10 (m. 1H), 1.99-1.90 (m, 1H), 1.80-1.71 (m, 2H), 1.64-1.55 (m, 2H), 1.49-1.32 (m, 8H), 1.30-1.18 (m, 2H), 0.92 (d, J = 7.2 Hz, 3H), 0.90-0.73 (m, 3H) |
| | 2-((2S,3S,4S)-5-chloro-2-((((1r,4S)-4-((2- | | |
| | (9-(3-(2,4-dioxotetrahydropyrimidin- | | |
| | 1(2H)-yl)-4-methoxybenzoyl)-3,9- | | |
| | diazaspiro[5.5]undecan-3- | | |
| | yl)ethyl)carbamoyl)cyclohexyl)amino) | | |
| | methyl)-6-fluoro-3-methyl-2-phenyl-2,3- | | |
| | dihydrobenzofuran-4-yl)-3-fluoro-4-methoxybenzamide | | |
| | Synthesized from C-II and L-XII | | |
| 6 | 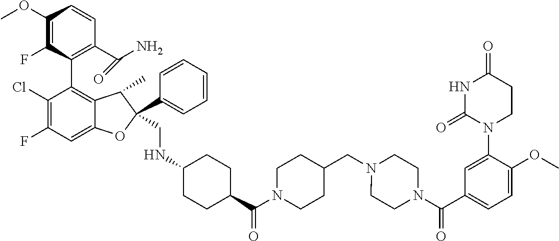 | 996.7 [M + H]⁺ 0.98 (3) | (400 MHZ, DMSO-d₆) δ (ppm) 10.34 (s, 1H), 7.63-7.57 (m, 2H), 7,43-7.28 (m, 6H), 7.287.24 (m, 2H), 7.15 (d, J = 8.6 Hz, 1H), 7.11 (s br, 1H), 7.07 (d, J = 9.6 Hz, 1H), 4.33 (d, J = 12.7 Hz, 1H), 3.88 (s, 3H), 3.84 (s, 3H), 3.59 (t, J = 6.7 Hz, 2H), 3.55- 3.37 (m, 4H), 3.27-3.19 (m, 1H), 3.19-3.08 (m, 2H), 2.99-2.89 (m, 1H), 2.72-2.65 (m, 4H), 2.47-2.41 (m, 1H), 2.38-2.29 (m, 4H), 2.22-2.10 (m, 3H), 1.81-1.64 (m, 5H), |
| | 2-((2S,3S,4S)-5-chloro-2-((((1r,4S)-4-(4-((4-(3-(2,4-dioxotetrahydropyrimidin-1(2H)-yl)-4- | | 1.60-1.50 (m, 2H), 1.34-1.20 |
| | methoxybenzoyl)piperazin-1-yl)methyl)piperidine-1-carbonyl)cyclohexyl)amino)methyl)- | | (m, 2H),0.92 (d, J = 7.1 Hz, |
| | 6-fluoro-3-methyl-2-phenyl-2,3-dihydrobenzofuran-4-yl)-3-fluoro-4-methoxybenzamide | | 3H), 1.00-0.77 (m, 4H) |
| | Synthesized from C-I and L-III | | |
| 7 | 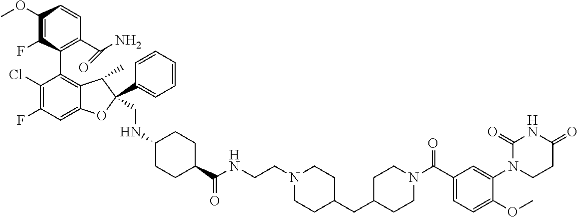 | 1038.8 [M + H]⁺ 0.60 (4) | (400 MHZ, DMSO-d₆) δ (ppm) 10.33 (s, 1H), 7.65-7.52 (m, 3H), 7.43-7.39 (m, 2H), 7.37-7.23 (m, 6H), 7.14 (d, J = 8.6 Hz, 1H), 7.10 (s br, 1H), 7.07 (d, J = 9.6 Hz, 1H), 3.88 (s, 3H), 3.84 (s, 3H), 3.59 (t, J = 6.7 Hz, 2H), 3.28- 3.19 (m, 2H), 3.17-3.03 (m, 4H), 2.82-2.73 (m, 2H), 2.70-2.65 (m, 2H), 2.30-2.20 (m, 2H), 2.20-2.11 (m, 1H), 1.99-1.70 (m, 6H), 1.69-1.53 (m, 7H), 1.33-1.18 (m, 4H), |
| | 2-((2S,3S,4S)-5-chloro-2-((((1r,4S)-4-((2-(4-((1-(3-(2,4-dioxotetrahydropyrimidin- | | 1.15-0.99 (m, 6H), 0.92 |
| | 1(2H)-yl)-4-methoxybenzoyl)piperidin-4-yl)methyl)piperidin-1- | | (d, J = 7.1 Hz, 3H), |
| | yl)ethyl)carbamoyl)cyclohexyl)amino)methyl)-6-fluoro-3-methyl-2-phenyl-2,3- | | 0.90-0.72 (m, 3H) |
| | dihydrobenzofuran-4-yl)-3-fluoro-4-methoxybenzamide | | |
| | Synthesized from C-I and L-XIV | | |
| 8 | 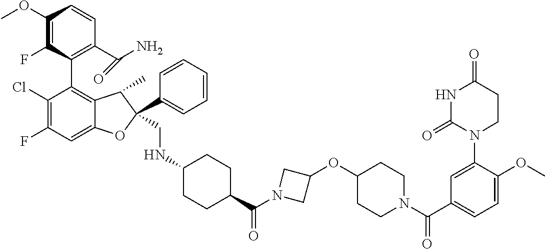 | 485.5 [M + 2H]^{2+/2} 0.70 (4) | (400 MHZ, DMSO-d₆) δ (ppm) 10.33 (s, 1H), 7.62 (s br, 1H), 7.61-7.56 (m, 1H), 7.42- 7.35 (m, 3H), 7.35-7.22 (m, 5H), 7.15 (d, J = 8.6 Hz, 1H), 7.11 (s br, 1H), 7.07 (d, J = 9.5 Hz, 1H), 4.46-4.36 (m, 1H), 4.29 (t, J = 7.9 Hz, 1H), 3.99 (dd, J = 10.3, 6.8 Hz, 1H), 3.91-3.85 (m, 1H), 3.88 (s, 3H), 3.84 (s, 3H), 3.61-3.54 (m, 4H), 3.26-3.08 (m, 6H), 2.71-2.63 (m, 2H), 2.20-2.11 (m, 1H), 2.09-1.96 (m, 1H), 1.86-1.70 (m, 4H), 1.60-1.51 (m, 2H), 1.47-1.38 |
| | 2-((2S,3S,4S)-5-chloro-2-((((1r,4S)-4-(3-((1-(3-(2,4-dioxotetrahydropyrimidin- | | (m, 2H), 1.27-1.13 (m, 2H), |
| | 1(2H)-yl)-4-methoxybenzoyl)piperidin-4-yl)oxy)azetidine-1- | | 0.97-0.74 (m, 6H) |
| | carbonyl)cyclohexyl)amino)methyl)-6-fluoro-3-methyl-2-phenyl-2,3- | | |
| | dihydrobenzofuran-4-yl)-3-fluoro-4-methoxybenzamide | | |
| | Synthesized from C-I and L-IV | | |
| 9 | 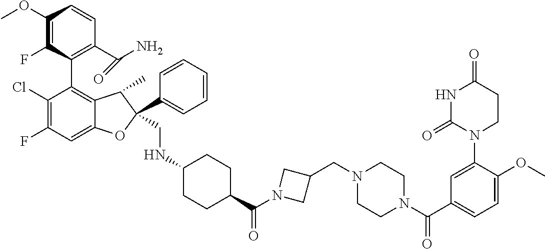 | 484.9 [M + 2H]²⁺/2 0.60 (4) | (400 MHZ, DMSO-d₆) δ (ppm) 10.34 (s, 1H), 7.65-7.55 (m, 2H), 7.44-7.21 (m, 8H), 7.15 (d, J = 8.5 Hz, 1H), 7.11 (s br, 1H), 7.07 (d, J = 9.6 Hz, 1H), 4.19-4.10 (m, 1H), 3.88 (s, 3H), 3.84 (s, 3H), 3.85-3.80 (m, 1H), 3.71 (t, J = 7.2 Hz, 1H), 3.59 (t, J = 6.6 Hz, 2H), 3.54-3.37 (m, 4H), 3.27-3.19 (m, 1H), 3.16-3.08 (m, 2H), 2.80-2.71 (m, 1H), 2.68 (t, J = 6.7 Hz, 2H), 2.57-2.51 (m, 1H), 2.43-2.31 (m, 4H), 2.21- 2.10 (m, 1H), 2.06-1.95 (m, 1H), 1.79-1.68 (m, 2H), 1.59- |
| | 2-((2S,3S,4S)-5-chloro-2-((((1r,4S)-4-(3-((4-(3-(2,4-dioxotetrahydropyrimidin- | | 1.51 (m, 2H), 1.28-1.14 (m, |
| | 1(2H)-yl)-4-methoxybenzoyl)piperazin-1-yl)methyl)azetidine-1- | | 3H), 0.92 (d, J = 7.2 Hz, 3H), |
| | carbonyl)cyclohexyl)amino)methyl)-6-fluoro-3-methyl-2-phenyl-2,3- | | 0.90-0.72 (m, 3H) |
| | dihydrobenzofuran-4-yl)-3-fluoro-4-methoxybenzamide | | |
| | Synthesized from C-I and L-V | | |
| 10 | 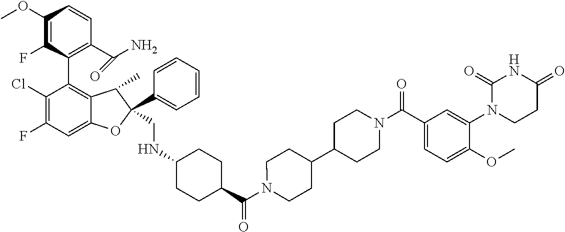 | 981.7 [M + H]⁺ 1.00 (3) | (400 MHZ, DMSO-d₆) δ (ppm) 10.33 (s, 1H), 7.63-7.57 (m, 2H), 7.43-7.38 (m, 2H), 7.38-7.22 (m, 6H), 7.14 (d, J = 8.6 Hz, 1H), 7.11 (s br, 1H), 7.07 (d, J = 9.5 Hz, 1H), 4.39 (d, J = 14.4 Hz, 1H), 3.93-3.85 (m, 2H), 3.88 (s, 3H), 3.84 (s, 3H), 3.59 (t, J = 6.6 Hz, 2H), 3.25-3.20 (m, 1H), 3.18-3.15 (m, 1H), 3.14- 3.11 (m, 2H), 2.95-2.80 (m, 1H), 2.70-2.64 (m, 2H),2.23-2.12 (m, 1H), 1.81-1.60 (m, 6H), |
| | 2-((2S,3S,4S)-5-chloro-2-((((1r,4S)-4-(1′-(3-(2,4-dioxotetrahydropyrimidin-1(2H)- | | 1.59-1.48 (m, 2H), 1.39-1.17 |
| | yl)-4-methoxybenzoyl)-[4,4′-bipiperidine]-1-carbonyl)cyclohexyl)amino)methyl)-6- | | (m, 5H), 1.16-0.81 (m, 9H), |
| | fluoro-3-methyl-2-phenyl-2,3-dihydrobenzofuran-4-yl)-3-fluoro-4-methoxybenzamide | | 0.92 (d, J = 7.2 Hz, 3H) |
| | Synthesized from C-II and L-VI | | |
| 11 | 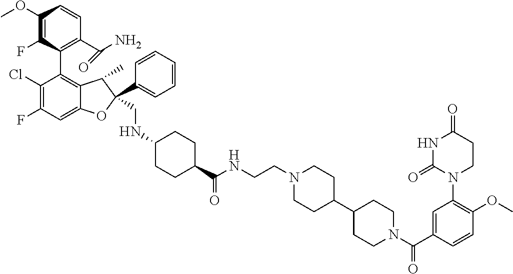 | 1024.7 [M + H ]⁺ 1.06 (3) | ¹H NMR (400 MHZ, DMSO-d₆) δ 10.31 (s, 1H), 7.63-7.56 (m, 2H), 7.56-7.50 (m, 1H), 7.43-7.38 (m, 2H), 7.38-7.28 (m, 4H), 7.28-7.22 (m, 2H), 7.14 (d, J = 8.5 Hz, 1H), 7.11-7.07 (s br, 1H), 7.06 (d, J = 10.3 Hz, 1H), 3.88 (s, 3H), 3.84 (s, 3H), 3.59 (t, J = 6.7 Hz, 2H), 3.27-3.19 (m, 1H), 3.18-3.03 (m, 4H), 2.83 (d, J = 10.5 Hz, 2H), 2.71-2.64 (m, 2H), 2.29-2.20 (m, 2H), 2.19-2.10 (m, 1H), 1.93 (t, J = 12.1 Hz, 1H), 1.86-1.54 (m, 11H), 1.37-1.19 (m, 3H), 1.19-0.97 |
| | 2-((2S,3S,4S)-5-chloro-2-((((1r,4S)-4-((2-(1′-(3-(2,4-dioxotetrahydropyrimidin-1(2H)-yl)- | | (m, 6H), 0.92 (d, J = 7.1 Hz, |
| | 4-methoxybenzoyl)-[4,4′-bipiperidin]-1-yl)ethyl)carbamoyl)cyclohexyl)amino)methyl)- | | 3H), 0.90-0.69 (m, 4H) |
| | 6-fluoro-3-methyl-2-phenyl-2,3-dihydrobenzofuran-4-yl)-3-fluoro-4-methoxybenzamide | | |
| | Synthesized from C-I and L-XII | | |
| 12 | 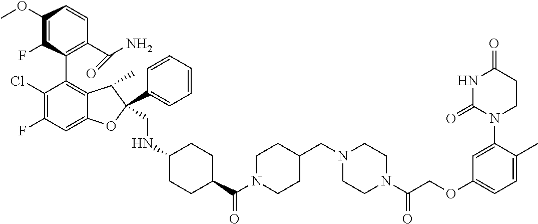 | 1011.1 [M + H]⁺ 1.00 (3) | ¹H NMR (400 MHZ, DMSO- d₆) δ 10.33 (s, 1H), 7.64-7.56 (m, 2H), 7.44-7.36 (m,2H), 7.36-7.22 (m, 4H), 7.16 (d, J = 8.6 Hz, 1H), 7.11 (s, 1H), 7.07 (d, J = 9.5 Hz, 1H), 6.88 (s, 1H), 6.80 (d, J = 8.5 Hz, 1H), 4.75 (s, 2H), 4.33 (d, J = 12.8 Hz, 1H), 3.88 (s, 3H), 3.87- 3.80 (m, 1H), 3.79-3.70 (m, 1H), 3.51-3.37 (m, 4H), 3.25- 3.19 (m, 1H), 3.16-3.08 (m, 2H), 2.99-2.83(m, 2H), 2.81-2.59 (m, 3H), 2.48-2.39 (m, 2H), 2.38- |
| | 2-((2S,3S,4S)-5-chloro-2-((((1r,4S)-4-(4-((4-(2-(3-(2,4-dioxotetrahydropyrimidin-1(2H)-yl)- | | 2.25 (m, 4H), 2.14-2.09 (m, 2H), |
| | 4-methylphenoxy)acetyl)piperazin-1-yl)methyl)piperidine-1-carbonyl)cyclohexyl)amino)methyl)- | | 2.09 (s, 3H), 1.82-1.62 (m, 5H), |
| | 6-fluoro-3-methyl-2-phenyl-2,3-dihydrobenzofuran-4-yl)-3-fluoro-4-methoxybenzamide | | 1.58-1.49 (m, 2H), 1.32-1.20 |
| | Synthesized from C-I and L-VII | | (m, 2H), 0.98-0.77 (m, 5H), |
| | | | 0.92 (d, J = 7.1 Hz, 3H) |
| 13 | 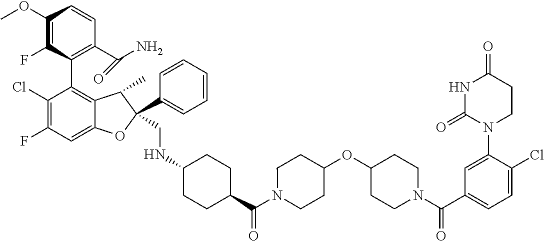 | 501.3 [M + 2H]²⁺/2 0.77 (4) | ¹H NMR (400 MHZ, DMSO- d₆) δ 10.51 (s, 1H), 7.66-7.55 (m, 4H), 7.43-7.37 (m, 3H), 7.36-7.23 (m, 4H), 7.11 (s br, 1H), 7.07 (d, J = 9.6 Hz, 1H), 3.86-3.78 (m, 1H), 3.76-3.57 (m, 5H), 3.54-3.42 (m, 1H), 3.28-3.19 (m, 2H), 3.17-3.08 (m, 4H), 3.05-2.95 (m, 1H), 2.77-2.70 (m, 2H), 2.45-2.39 (m, 1H), 2.22-2.12 (m, 1H), 1.88-1.68 (m, 6H), 1.59-1.49 (m, 2H), 1.37- 1.19 (m, 8H), 0.93-0.81 (m, 3H), 0.92 (d, J = 7.2 Hz, 3H) |
| | 2-((2S,3S,4S)-5-chloro-2-((((1r,4S)-4-(4-((1-(4-chloro-3-(2,4-dioxotetrahydropyrimidin- | | |
| | 1(2H)-yl)benzoyl)piperidin-4-yl)oxy)piperidine-1-carbonyl)cyclohexyl)amino)methyl)-6- | | |
| | fluoro-3-methyl-2-phenyl-2,3-dihydrobenzofuran-4-yl)-3-fluoro-4-methoxybenzamide | | |
| | Synthesized from C-I and L-VIII | | |
| 14 | 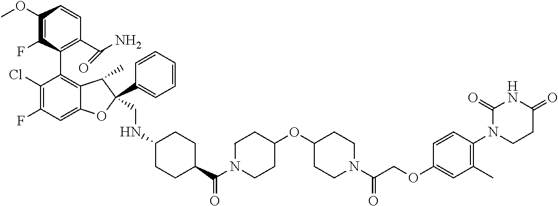 | 1011.8 [M + H]⁺ 0.74 (4) | ¹H NMR (400 MHZ, DMSO- d₆) δ 10.28 (s, 1H), 7.63-7.57 (m, 2H), 7.43-7.38 (m, 2H), 7.36-7.23 (m, 4H), 7.14 (d, J = 8.6 Hz, 1H), 7.13-7.10 (m, 1H), 7.07 (d, J = 9.5 Hz, 1H), 6.83 (d, J = 2.9 Hz, 1H), 6.76 (dd, J = 8.6, 2.9 Hz, 1H), 4.78 (s, 2H), 3.88 (s, 3H), 3.86-3.78 (m, 2H), 3.73-3.59 (m, 4H), 3.52-3.42 (m, 1H), 3.27-3.06 (m, 6H), 3.05-2.93 (m, 1H), 2.81-2.60 (m, 3H), 2.46-2.38 |
| | 2-((2S,3S,4S)-5-chloro-2-((((1r,4S)-4-(4-((1-(2-(4-(2,4-dioxotetrahydropyrimidin- | | (m, 1H), 2.23-2.15 (m,1H), 2.13 |
| | 1(2H)-yl)-3-methylphenoxy)acetyl)piperidin-4-yl)oxy)piperidine-1- | | (s, 3H), 1.88-1.70 (m, 6H), 1.60- |
| | carbonyl)cyclohexyl)amino)methyl)-6-fluoro-3-methyl-2-phenyl-2,3- | | 1.39 (m, 3H), 1.39-1.18 (m, 6H), |
| | dihydrobenzofuran-4-yl)-3-fluoro-4-methoxybenzamide | | 0.99-0.81 (m, 3H), 0.92 |
| | Synthesized from C-land L-IX | | (d, J = 7.2 Hz, 3H) |
| 15 | 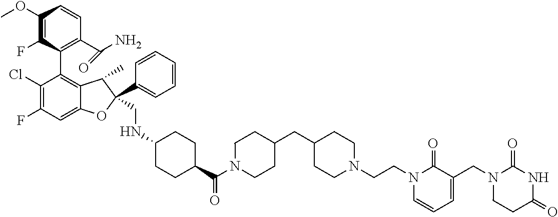 | 996.5 [M+H]⁺ 0.57 (4) | ¹H NMR (400 MHZ, DMSO- d₆) δ 10.15 (s, 1H), 7.65-7.53 (m, 3H), 7.41 (d, J = 7.6 Hz, 2H), 7.37-7.21 (m, 5H), 7.11 (s br, 1H), 7.07 (d, J = 9.5 Hz, 1H), 6.19 (t, J = 6.8 Hz, 1H), 4.32 (d, J = 12.9 Hz ,1H), 4.25 (s, 2H), 3.98 (t, J = 6.5 Hz, 2H), 3.88 (s, 3H), 3.83 (d, J = 14.5 Hz, 1H), 3.41 (t, J = 6.8 Hz, 2H), 3.26-3.18 (m, 1H), 3.17-3.07 (m, 2H), 2.97-2.86 (m, 1H), 2.84 (d, J = 11.0 Hz, |
| | 2-((2S,3S,4S)-5-chloro-2-((((1r,4S)-4-(4-((1-(2-(3-((2,4-dioxotetrahydropyrimidin- | | 2H), 2.56 (t, J = 6.8 Hz, 2H), |
| | 1(2H)-yl)methyl)-2-oxopyridin-1(2H)-yl)ethyl)piperidin-4-yl)methyl)piperidine- | | 2.46-2.37 (m, 2H), 2.22-2.13 |
| | 1-carbonyl)cyclohexyl)amino)methyl)-6-fluoro-3-methyl-2-phenyl-2,3- | | (m, 1H), 1.92 (t, J = 11.3 Hz, |
| | dihydrobenzofuran-4-yl)-3-fluoro-4-methoxybenzamide | | 2H), 1.80-1.69 (m, 2H), 1.66- |
| | Synthesized from C-I and L-XVI | | 1.48 (m, 7H), 1.35- 1.19 |
| | | | (m, 4H), 1.08 - 0.97 (m, |
| | | | 4H), 0.96-0.76 (m, 5H), |
| | | | 0.92 (d, J = 7.2 Hz, 3H) |

### General Procedure 2:

### Example 16: 2-((2S,3S,4S)-5-chloro-2-((((1r,4S)-4-(4-((1-(2-(3-((2,4-dioxotetrahydropyrimidin-1(2H)-yl)methyl)-2-oxopyridin-1(2H)-yl)acetyl)piperidin-4-yl)methyl)piperidine-1-carbonyl)cyclohexyl)amino)methyl)-6-fluoro-3-methyl-2-phenyl-2,3-dihydrobenzofuran-4-yl)-3-fluoro-4-methoxybenzamide

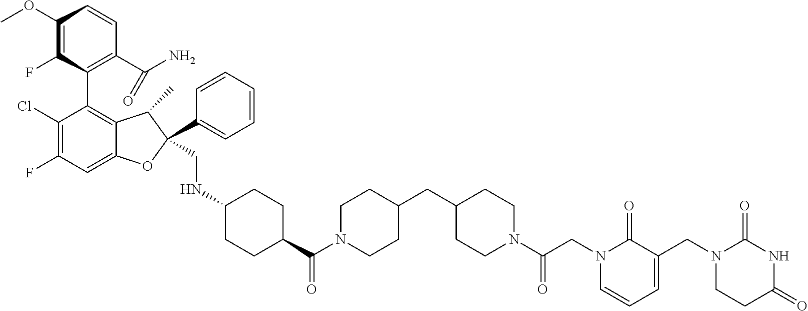

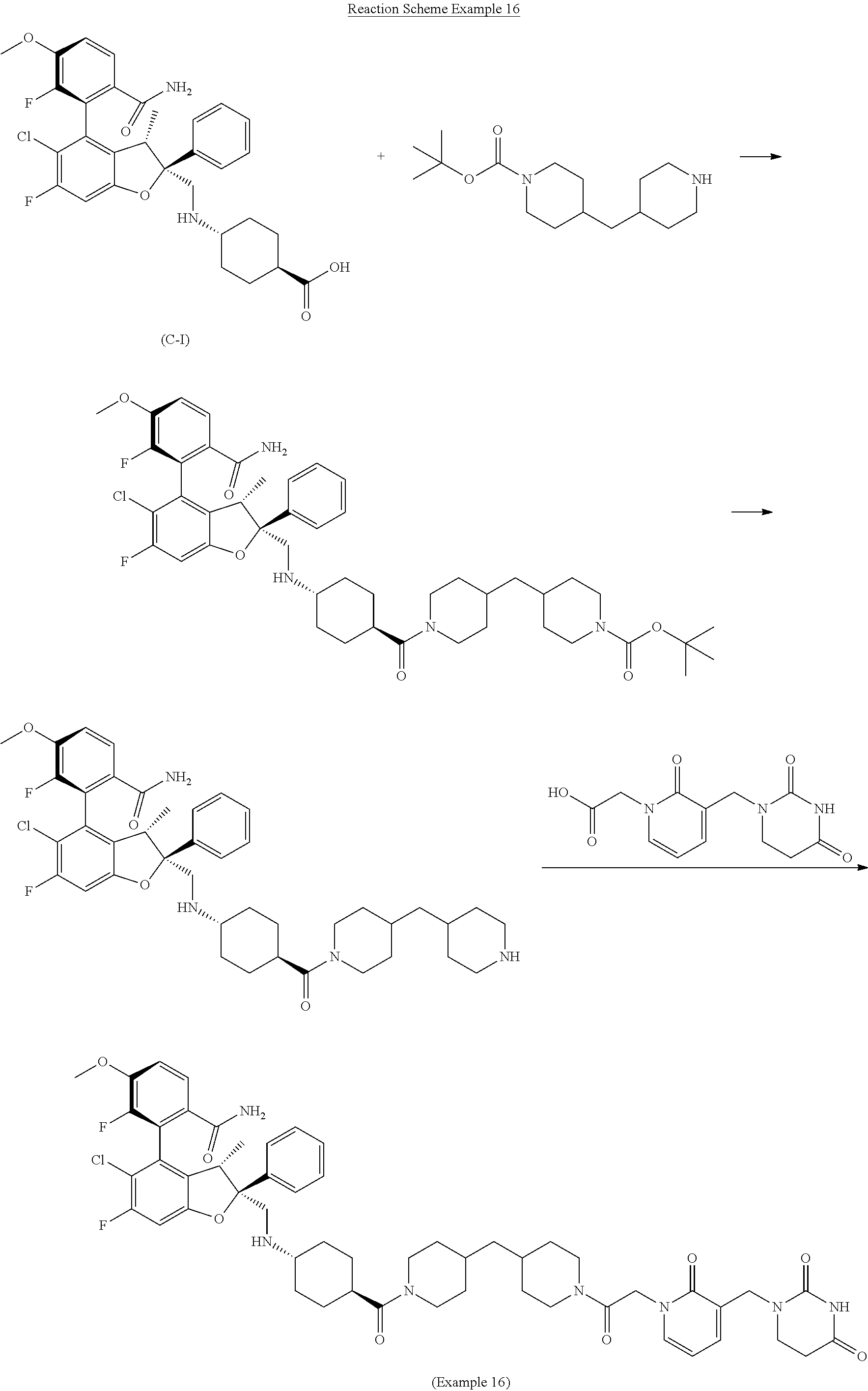

### Step 1: tert-butyl 4-((1-((1S,4r)-4-((((2S,3S,4S)-4-(6-carbamoyl-2-fluoro-3-methoxyphenyl)-5-chloro-6-fluoro-3-methyl-2-phenyl-2,3-dihydrobenzofuran-2-yl)methyl)amino)cyclohexane-1-carbonyl)piperidin-4-yl)methyl)piperidine-1-carboxylate

To a stirred solution of (1S,4r)-4-((((2S,3S,4S)-4-(6-carbamoyl-2-fluoro-3-methoxyphenyl)-5-chloro-6-fluoro-3-methyl-2-phenyl-2,3-dihydrobenzofuran-2-yl)methyl)amino)cyclohexane-1-carboxylic acid (C-I) (150 mg, 0.215 mmol, TFA salt) in DMF (2.1 mL), was added DIPEA (187 μl, 1.07 mmol) followed by HATU (98 mg, 0.257 mmol). After 5 min, tert-butyl 4-(piperidin-4-ylmethyl)piperidine-1-carboxylate (73 mg, 0.257 mmol) was added and the reaction mixture was stirred at RT for 18 h. The reaction mixture was quenched with water and extracted twice with EtOAc. The organic layers were combined and dried (phase separation cartridge) and concentrated to give the crude title compound. UPLC-MS 4: m/z 849.4 [M+H]⁺, t_{R}=1.07 min.

### Step 2: 2-((2S,3S,4S)-5-chloro-6-fluoro-3-methyl-2-phenyl-2-((((1r,4S)-4-(4-(piperidin-4-ylmethyl)piperidine-1-carbonyl)cyclohexyl)amino)methyl)-2,3-dihydrobenzofuran-4-yl)-3-fluoro-4-methoxybenzamide

To a stirred solution of tert-butyl 4-((1-((1S,4r)-4-((((2S,3S,4S)-4-(6-carbamoyl-2-fluoro-3-methoxyphenyl)-5-chloro-6-fluoro-3-methyl-2-phenyl-2,3-dihydrobenzofuran-2-yl)methyl)amino)cyclohexane-1-carbonyl)piperidin-4-yl)methyl)piperidine-1-carboxylate (crude, 0.215 mmol) in DCE/MeOH 1/1 (2 mL) was added HCl (0.8 mL, 3.22 mmol, 4 M in dioxane), the resulting mixture was stirred at RT for 2 d. The reaction mixture was concentrated to give the title compound (110 mg). UPLC-MS 4: m/z 749.4 [M+H]⁺, t_{R}=0.57 min

### Step 3: 2-((2S,3S,4S)-5-chloro-2-((((1r,4S)-4-(4-((1-(2-(3-((2,4-dioxotetrahydropyrimidin-1(2H)-yl)methyl)-2-oxopyridin-1(2H)-yl)acetyl)piperidin-4-yl)methyl)piperidine-1-carbonyl)cyclohexyl)amino)methyl)-6-fluoro-3-methyl-2-phenyl-2,3-dihydrobenzofuran-4-yl)-3-fluoro-4-methoxybenzamide (Example 16)

To a stirred solution of 2-(3-((2,4-dioxotetrahydropyrimidin-1(2H)-yl)methyl)-2-oxopyridin-1(2H)-yl) acetic acid (synthesized according to WO2021053495) (8.5 mg, 0.030 mmol) in DMF (0.27 mL) was added DIPEA (48 μl, 0.276 mmol) followed by HATU (12.6 mg, 0.033 mmol) and by 2-((2S,3S,4S)-5-chloro-6-fluoro-3-methyl-2-phenyl-2-((((1r,4S)-4-(4-(piperidin-4-ylmethyl)piperidine-1-carbonyl)cyclohexyl)amino)methyl)-2,3-dihydrobenzofuran-4-yl)-3-fluoro-4-methoxybenzamide (27 mg, 0.028 mmol) and the resulting mixture was stirred at RT for 1 h. After concentration the crude product was purified by prep HPLC to give the title compound (17.5 mg) as a colorless solid. UPLC-MS 4: m/z 505.9 [M+2H]²⁺/2, t_{R}=0.74 min. ¹H NMR (400 MHZ, DMSO-d₆) δ (ppm) 10.16 (s, 1H), 7.63-7.57 (m, 2H), 7.49 (d, J=6.3 Hz, 1H), 7.43-7.39 (m, 2H), 7.36-7.22 (m, 5H), 7.11 (s br, 1H), 7.07 (d, J=9.6 Hz, 1H), 6.21 (t, J=6.8 Hz, 1H), 4.81 (d, J=2.9 Hz, 2H), 4.35 (d, J=12.3 Hz, 1H), 4.27 (d, J=12.3 Hz, 1H), 4.24 (s, 2H), 3.94-3.80 (m, 2H), 3.88 (s, 3H), 3.40 (t, J=6.8 Hz, 2H), 3.28-3.20 (m, 2H), 3.17-3.12 (m, 2H), 3.05 (t, J=12.7 Hz, 1H), 2.93 (t, J=12.3 Hz, 1H), 2.56 (t, J=6.8 Hz, 2H), 2.48-2.38 (m, 2H), 2.25-2.15 (m, 1H), 1.82-1.45 (m, 10H), 1.37-1.20 (m, 2H), 1.18-1.05 (m, 3H), 0.93 (d, J=7.1 Hz, 3H), 1.00-0.78 (m, 5H).

The following Examples were prepared according to General Procedure 2:

| | | UPLC MS | |
|---|---|---|---|
| | | m/z | |
| | | t_{R} [min] | |
| Ex. | Structure/Chemical Name | (method) | ¹H NMR |
| 17 | 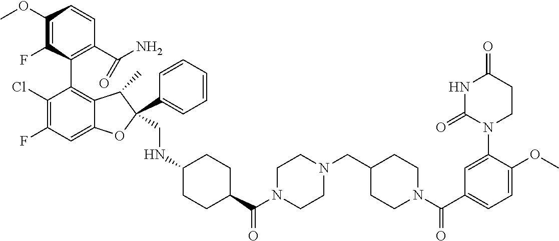 | 996.8 [M + H]⁺ 0.99 (3) | (400 MHz, DMSO-d₆) δ (ppm) 10.34 (s, 1H), 7.63-7-53 (m, 2H), 7.41 (d, J = 7.3 Hz, 2H), 7.37-7.22 (m, 6H), 7.15 (d, J = 8.6 Hz, 1H), 7.11 (s br, 1H), 7.07 (d, J = 9.6 Hz, 1H), 4.34 (s br, 1H), 3.88 (s, 3H), 3.84 (s, 3H), 3.59 (t, J = 6.6 Hz, 2H), 3.44-3.35 (m, 4H), 3.31-3.20 (m, 2H), 3.16- 3.09 (m, 2H), 3.01-2.92 (m, 1H), 2.68 (t, J = 6.6 Hz, 2H), 2.46-2.37 (m, 2H), 2.33- 2.19 (m, 4H), 2.19-2.08 (m, 2H), 1.83-1.63 (m, 4H), 1.60-1.51 (m, 2H), 1.33- |
| | 2-((2S,3S,4S)-5-chloro-2-((((1r,4S)-4-(4- | | 1.19 (m, 2H), 1.11-1.00 (m, |
| | ((1-(3-(2,4-dioxotetrahydropyrimidin- | | 2H), 0.97-0.81 (m, 8H) |
| | 1(2H)-yl)-4-methoxybenzoyl)piperidin-4- | | |
| | yl)methyl)piperazine-1- | | |
| | carbonyl)cyclohexyl)amino)methyl)-6- | | |
| | fluoro-3-methyl-2-phenyl-2,3- | | |
| | dihydrobenzofuran-4-yl)-3-fluoro-4- | | |
| | methoxybenzamide | | |
| | Synthesized from C-I, | | |
| | tert-butyl 4-(piperazin-1- | | |
| | ylmethyl)piperidine-1-carboxylate | | |
| | and | | |
| | 3-(2,4-dioxotetrahydropyrimidin-1(2H)- | | |
| | yl)-4-methoxybenzoic acid | | |
| 18 | 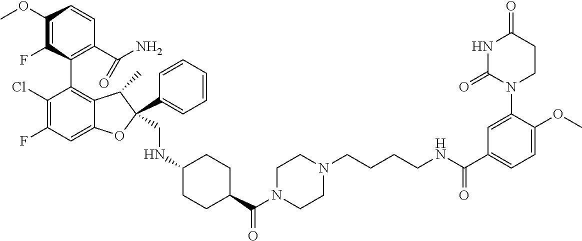 | 969.7 [M + H]⁺ 0.79 (4) | n.d. |
| | N-(4-(1-((1S,4r)-4-((((2S,3S,4S)-4-(6- | | |
| | carbamoyl-2-fluoro-3-methoxyphenyl)-5- | | |
| | chloro-6-fluoro-3-methyl-2-phenyl-2,3- | | |
| | dihydrobenzofuran-2- | | |
| | yl)methyl)amino)cyclohexane-1- | | |
| | carbonyl)piperidin-4-yl)butyl)-3-(2,4- | | |
| | dioxotetrahydropyrimidin-1(2H)-yl)-4- | | |
| | methoxybenzamide | | |
| | Synthesized from C-I, tert-butyl (4- | | |
| | (piperidin-4-yl)butyl)carbamate and 3- | | |
| | (2,4-dioxotetrahydropyrimidin-1(2H)-yl)- | | |
| | 4-methoxybenzoic acid | | |
| 19 | 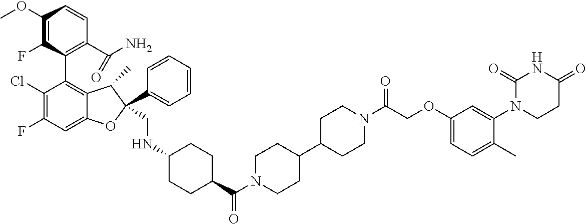 | 498.5 [M + 2H]²⁺/2 0.76 (4) | (400 MHz, DMSO-d₆) δ (ppm) 10.33 (s, 1H), 7.64-7.56 (m, 2H), 7.37-7.22 (m, 4H), 7.15 (d, J = 8.6 Hz, 1H), 7.11 (s, 1H), 7.07 (d, J = 9.6 Hz, 1H), 6.87 (d, J = 2.8 Hz, 1H), 6.79 (d, J = 8.6 Hz, 1H), 4.72 (t, J = 11.2 Hz, 2H), 4.37 (t, J = 14.9 Hz, 2H), 3.93-3.82 (m, 1H), 3.87 (s, 3H), 3.79- 3.70 (m, 1H), 3.55-3.42 (m, 1H), 3.23 (d, J = 7.3 Hz, 1H), 3.18-3.10 (m, 2H), 3.01- 2.82 (m, 2H), 2.81-2.62 (m, 2H), 2.47-2.32 (m, 2H), |
| | 2-((2S,3S,4S)-5-chloro-2-((((1r,4S)-4-(1′- | | 2.24-2.14 (m, 1H), 2.09 (s, |
| | (2-(3-(2,4-dioxotetrahydropyrimidin- | | 3H), 1.81-1.49 (m, 9H), |
| | 1(2H)-yl)-4-methylphenoxy)acetyl)-[4,4′- | | 1.36-1.10 (m, 5H), 1.06- |
| | bipiperidine]-1- | | 0.81 (m, 6H), 0.92 (d, J = 7.3 |
| | carbonyl)cyclohexyl)amino)methyl)-6- | | Hz, 3H) |
| | fluoro-3-methyl-2-phenyl-2,3- | | |
| | dihydrobenzofuran-4-yl)-3-fluoro-4- | | |
| | methoxybenzamide | | |
| | Synthesized from C-I, | | |
| | tert-butyl [4,4′-bipiperidine]-1-carboxylate | | |
| | and 2-(3-(2,4-dioxotetrahydropyrimidin- | | |
| | 1(2H)-yl)-4-methylphenoxy)acetic acid | | |
| 20 | 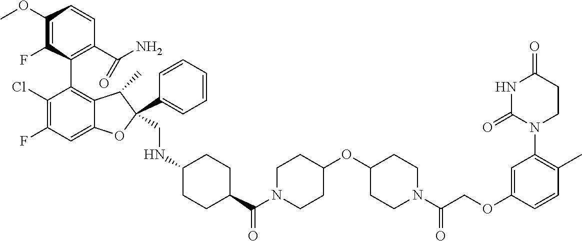 | 1011.8 [M + H]⁺ 0.76 (4) | (400 MHz, DMSO-d₆) δ (ppm) 10.33 (s, 1H), 7.62-7.57 (m, 2H), 7.42-7.38 (m, 2H), 7.36-7.20 (m, 4H), 7.15 (d, J = 8.6 Hz, 1H), 7.11 (s br, 1H), 7.07 (d, J = 9.8 Hz, 1H), 6.88 (s, 1H), 6.80 (d, J = 8.7 Hz, 1H), 4.75 (s, 2H),3.88 (s, 3H), 3.86-3.70 (m, 2H), 3.70-3.56 (m, 2H), 3.52- 3.43 (m, 1H), 3.24-3.17 (m, 2H), 3.18-3.05 (m, 4H), 3.04-2.91 (m, 1H), 2.79- 2.63 (m, 2H), 2.46-2.39 (m, 1H), 2.24-2.13 (m, 1H), 2.09 (s, 3H), 1.88-1.68 (m, |
| | 2-((2S,3S,4S)-5-chloro-2-((((1r,4S)-4-(4- | | 6H), 1.60-1.51 (m, 2H), |
| | ((1-(2-(3-(2,4-dioxotetrahydropyrimidin- | | 1.49-1.40 (m, 1H), 1.37- |
| | 1(2H)-yl)-4- | | 1.20 (m, 7H), 0.92 (d, J = 7.3 |
| | methylphenoxy)acetyl)piperidin-4- | | Hz, 3H), 0.90-0.80 (m, 3H) |
| | yl)oxy)piperidine-1- | | |
| | carbonyl)cyclohexyl)amino)methyl)-6- | | |
| | fluoro-3-methyl-2-phenyl-2,3- | | |
| | dihydrobenzofuran-4-yl)-3-fluoro-4- | | |
| | methoxybenzamide | | |
| | Synthesized from C-I, | | |
| | 4-(piperidin-4-yloxy)-piperidine-1- | | |
| | carboxylic acid tert-butyl ester and 2-(3- | | |
| | (2,4-dioxotetrahydropyrimidin-1(2H)-yl)- | | |
| | 4-methylphenoxy)acetic acid | | |
| 21 | 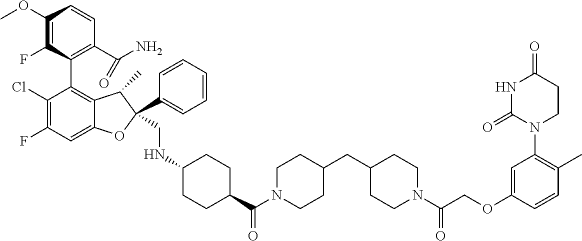 | 1009.7 [M + H]⁺ 0.80 (4) | (400 MHz, DMSO-d₆) δ (ppm) 10.33 (s, 1H), 7.63-7.57 (m, 2H), 7.44-7.38 (m, 2H), 7.36-7.22 (m, 4H), 7.15 (d, J = 8.5 Hz, 1H), 7.11 (s br, 1H), 7.07 (d, J = 9.5 Hz, 1H), 6.87 (d, J = 2.7 Hz, 1H), 6.79 (dd, J = 8.4, 2.7 Hz, 1H), 4.80- 4.66 (m, 2H), 4.32 (t, J = 14.1 Hz, 2H), 3.88 (s, 3H), 3.85-3.68 (m, 3H), 3.54- 3.43 (m, 1H), 3.28-3.19 (m, 1H), 3.17-3.10 (m, 2H), 3.05-2.88 (m, 2H), 2.79- 2.63 (m, 2H), 2.24-2.15 (m, 1H), 2.09 (s, 3H), 1.82-1.49 |
| | 2-((2S,3S,4S)-5-chloro-2-((((1r,4S)-4-(4- | | (m, 10 H), 1.36-1.21 (m, |
| | ((1-(2-(3-(2,4-dioxotetrahydropyrimidin- | | 2H), 1.18-1.05 (m, 3H), |
| | 1(2H)-yl)-4- | | 1.00-0.78 (m, 4H), 0.92 (d, |
| | methylphenoxy)acetyl)piperidin-4- | | J = 7.2 Hz, 3H) |
| | yl)methyl)piperidine-1- | | not all signals visible in |
| | carbonyl)cyclohexyl)amino)methyl)-6- | | aliphatic region due to overlap |
| | fluoro-3-methyl-2-phenyl-2,3- | | with solvent peaks. |
| | dihydrobenzofuran-4-yl)-3-fluoro-4- | | |
| | methoxybenzamide | | |
| | Synthesized from C-I, | | |
| | tert-butyl 4-(piperidin-4- | | |
| | ylmethyl)piperidine-1-carboxylate and 2- | | |
| | (3-(2,4-dioxotetrahydropyrimidin-1 (2H)- | | |
| | yl)-4-methylphenoxy)acetic acid | | |
| 22 | 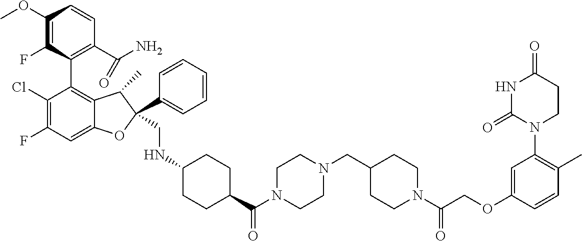 | 1010.8 [M + H]⁺ 1.01 (3) | (400 MHz, DMSO-d₆) δ (ppm) 10.33 (s, 1H), 7.62-7.56 (m, 2H), 7.43-7.38 (m, 2H), 7.37-7.22 (m, 4H), 7.15 (d, J = 8.5 Hz, 1H), 7.10 (s br, 1H), 7.06 (d, J = 9.5 Hz, 1H), 6.87 (d, J = 2.7 Hz, 1H), 6.79 (dd, J = 8.5, 2.7 Hz, 1H), 4.80- 4.67 (m, 2H), 4.29 (d, J = 12.6 Hz, 1H), 3.88 (s, 3H), 3.85-3.68 (m, 2H), 3.53- 3.45 (m, 1H), 3.44-3-36 (m, 4H), 3.23 (q, J = 7.1 Hz, 1H), 3.18-3.09 (m, 2H), 3.00 (t, J = 12.8 Hz, 1H), 2.83-2.53 (m, 4H), 2.47-2.37 (m, 1H), |
| | 2-((2S,3S,4S)-5-chloro-2-((((1r,4S)-4-(4- | | 2.35-2.13 (m, 6H), 2.10 (s, |
| | ((1-(2-(3-(2,4-dioxotetrahydropyrimidin- | | 3H), 1.83-1.67 (m, 6H), |
| | 1(2H)-yl)-4- | | 1.60-1.50 (m, 2H), 1.32- |
| | methylphenoxy)acetyl)piperidin-4- | | 1.18 (m, 1H), 0.99-0.81 (m, |
| | yl)methyl)piperazine-1- | | 4H), 0.92 (d, J = 7.2 Hz, 5H). |
| | carbonyl)cyclohexyl)amino)methyl)-6- | | |
| | fluoro-3-methyl-2-phenyl-2,3- | | |
| | dihydrobenzofuran-4-yl)-3-fluoro-4- | | |
| | methoxybenzamide | | |
| | Synthesized from C-I, | | |
| | tert-butyl 4-(piperazin-1- | | |
| | ylmethyl)piperidine-1-carboxylate and 2- | | |
| | (3-(2,4-dioxotetrahydropyrimidin-1(2H)- | | |
| | yl)-4-methylphenoxy)acetic acid | | |
| 23 | 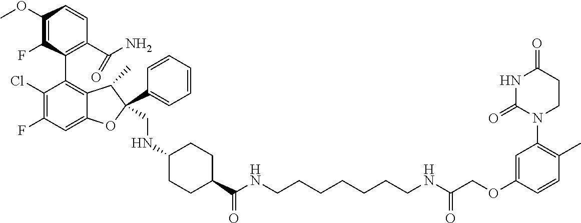 | 957.7 [M + H]⁺ 0.76 (4) | n.d. |
| | 2-((2S,3S,4S)-5-chloro-2-((((1r,4S)-4-((7- | | |
| | (2-(3-(2,4-dioxotetrahydropyrimidin- | | |
| | 1(2H)-yl)-4- | | |
| | methylphenoxy)acetamido)heptyl)carbamoyl) | | |
| | cyclohexyl)amino)methyl)-6-fluoro-3- | | |
| | methyl-2-phenyl-2,3-dihydrobenzofuran- | | |
| | 4-yl)-3-fluoro-4-methoxybenzamide | | |
| | Synthesized from C-I, | | |
| | tert-butyl(7-aminoheptyl)carbamate and | | |
| | 2-(3-(2,4-dioxotetrahydropyrimidin-1(2H)- | | |
| | yl)-4-methylphenoxy)acetic acid | | |
| 24 | 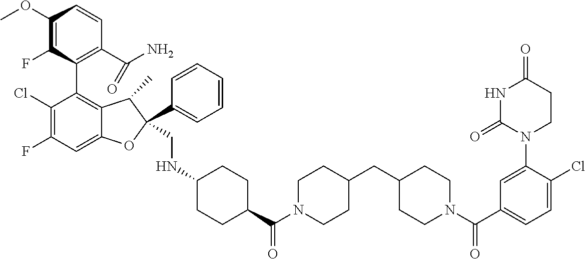 | 999.7 [M + H]⁺ 0.80 (4) | (400 MHz, DMSO-d₆) δ (ppm) 10.51 (s, 1H), 7.65-7.56 (m, 3H), 7.54 (d, J = 2.0 Hz, 1H), 7.44-7.22 (m, 7H), 7.11 (s br, 1H), 7.07 (d, J = 9.6 Hz, 1H), 4.49-4.38 (m, 1H), 4.34 (d, J = 13.0 Hz, 1H), 3.88 (s, 3H), 3.85-3.81 (m, 1H), 3.80-3.72 (m, 1H), 3.68-3.50 (m, 2H), 3.23 (q, J = 7.2 Hz, 1H), 3.18-3.08 (m, 2H), 3.07-2.98 (m, 1H), 2.98-2.88 (m, 1H), 2.83- 2.68 (m, 2H), 2.56-2.37 (m, 2H), 2.25-2.13 (m, 1H), 1.81-1.47 (m, 11H), 1.33- 1.16 (m, 2H), 1.16-1.02 (m, |
| | 2-((2S,3S,4S)-5-chloro-2-((((1r,4S)-4-(4- | | 4H), 0.94-0.76 (m, 4H), |
| | ((1-(4-chloro-3-(2,4- | | 0.92 (d, J = 7.2 Hz, 3H) |
| | dioxotetrahydropyrimidin-1(2H)- | | |
| | yl)benzoyl)piperidin-4- | | |
| | yl)methyl)piperidine-1- | | |
| | carbonyl)cyclohexyl)amino)methyl)-6- | | |
| | fluoro-3-methyl-2-phenyl-2,3- | | |
| | dihydrobenzofuran-4-yl)-3-fluoro-4- | | |
| | methoxybenzamide | | |
| | Synthesized from C-I, tert-butyl 4- | | |
| | (piperidin-4-ylmethyl)piperidine-1- | | |
| | carboxylate and 4-chloro-3-(2,4- | | |
| | dioxotetrahydropyrimidin-1(2H)- | | |
| | yl)benzoic acid | | |
| 25 | 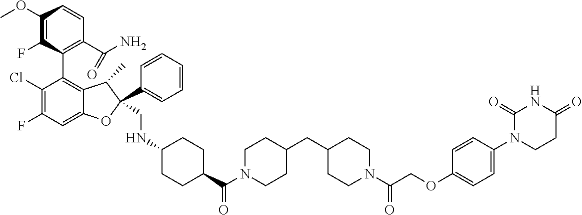 | 995.4 [M +H]⁺ 0.74 (4) | (400 MHz, DMSO-d₆) δ (ppm) 10.30 (s, 1H), 7.62-7.57 (m, 2H), 7.43-7.39 (m, 2H), 7.36-7.16 (m, 6H), 7.11 (s br, 1H), 7.07 (d, J = 9.5 Hz, 1H), 6.96-6.84 (m, 2H), 4.78 (d, J = 5.0 Hz, 2H), 4.32 (t, J = 14.7 Hz, 2H), 3.88 (s, 3H), 3.84-3.72 (m, 2H), 3.71 (t, J = 6.7 Hz, 2H), 3.26- 3.18 (m, 1H), 3.15-3.07 (m, 2H), 3.06-2.88 (m, 2H), 2.69 (t, J = 6.7 Hz, 2H), 2.63- 2.53 (m, 1H), 2.47-2.36 |
| | 2-((2S,3S,4S)-5-chloro-2-((((1r,4S)-4-(4- | | (m, 2H), 2.22-2.12 (m, 1H), |
| | ((1-(2-(4-(2,4-dioxotetrahydropyrimidin- | | 1.80-1.51 (m, 10H), 1.32- |
| | 1(2H)-yl)phenoxy)acetyl)piperidin-4- | | 1.17 (m, 2H), 1.16-1.04 (m, |
| | yl)methyl)piperidine-1- | | 3H), 1.00-0.77 (m, 5H), |
| | carbonyl)cyclohexyl)amino)methyl)-6- | | 0.92 (d, J = 7.1 Hz, 3H) |
| | fluoro-3-methyl-2-phenyl-2,3- | | |
| | dihydrobenzofuran-4-yl)-3-fluoro-4- | | |
| | methoxybenzamide | | |
| | Synthesized from C-I, tert-butyl 4- | | |
| | (piperidin-4-ylmethyl)piperidine-1- | | |
| | carboxylate and 2-(4-(2,4- | | |
| | dioxotetrahydropyrimidin-1 (2H)- | | |
| | yl)phenoxy)acetic acid (synthesized | | |
| | according to WO2021053495) | | |
| 26 | 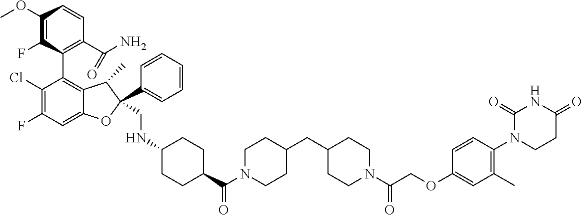 | 1009.6 [M + H]⁺ 0.77 (4) | (400 MHz, DMSO-d₆) δ (ppm) 10.27 (s, 1H), 7.63-7.57 (m, 2H), 7.43-7.39 (m, 2H), 7.36-7.23 (m, 4H), 7.14 (d, J = 8.6 Hz, 1H), 7.10 (s br, 1H), 7.07 (d, J = 9.5 Hz, 1H), 6.82 (d, J = 2.9 Hz, 1H), 6.75 (dd, J = 8.6, 2.9 Hz, 1H), 4.76 (d, J = 4.2 Hz, 2H), 4.32 (t, J = 14.2 Hz, 2H), 3.88 (s, 3H), 3.86-3.77 (m, 1H), 3.77- 3.66 (m, 1H), 3.53-3.43 (m, 1H), 3.23 (q, J = 7.2 Hz, 1H), 3.18-3.08 (m, 2H), 3.06- |
| | 2-((2S,3S,4S)-5-chloro-2-((((1r,4S)-4-(4- | | 2.89 (m, 2H), 2.78-2.64 (m, |
| | ((1-(2-(4-(2,4-dioxotetrahydropyrimidin- | | 2H), 2.48-2.37 (m, 2H), |
| | 1(2H)-yl)-3- | | 2.24-2.17 (m, 1H), 2.13 (s, |
| | methylphenoxy)acetyl)piperidin-4- | | 3H), 1.80-1.49 (m, 10 H), |
| | yl)methyl)piperidine-1- | | 1.32-1.18 (m, 3H), 1.15- |
| | carbonyl)cyclohexyl)amino)methyl)-6- | | 1.03 (m, 3H), 0.99-0.78 (m, |
| | fluoro-3-methyl-2-phenyl-2,3- | | 5H), 0.92 (d, J = 7.1 Hz, 3H) |
| | dihydrobenzofuran-4-yl)-3-fluoro-4- | | |
| | methoxybenzamide | | |
| | Synthesized from C-I, tert-butyl 4- | | |
| | (piperidin-4-ylmethyl)piperidine-1- | | |
| | carboxylate and 2-(4-(2,4- | | |
| | dioxotetrahydropyrimidin-1(2H)-yl)-3- | | |
| | methylphenoxy)acetic acid (synthesized | | |
| | according to WO2021053495) | | |
| 27 | 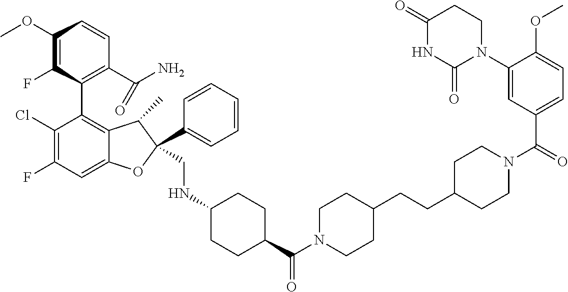 | 1009.4 [M + H]⁺ 0.81 (4) | (400 MHz, DMSO-d₆) δ (ppm) 10.34 (s, 1H), 7.65-7.57 (m, 2H), 7.43-7.40 (m, 2H), 7.38-7.22 (m, 6H), 7.15 (d, J = 8.6 Hz, 1H), 7.12 (s br, 1H), 7.08 (d, J = 9.6 Hz, 1H), 4.34 (d, J = 12.9 Hz, 1H), 3.91-3.80 (m, 2H), 3.88 (s, 3H), 3.84 (s, 3H), 3.60 (t, J = 6.6 Hz, 2H), 3.24 (q, J = 7.1 Hz, 1H), 3.18-3.10 (m, 2H), 2.91 (t, J = 12.7 Hz, 2H), 2.68 (t, J = 6.6 Hz, 2H), 2.42 (t, J = 12.1 Hz, 2H), 2.24-2.14 (m, 1H), 1.81-1.60 (m, 6H), 1.57-1.50 (m, 2H), 1.51- 1.34 (m, 2H), 1.32-1.17 (m, 7H), 1.11-0.97 (m, 2H), 0.97-0.79 (m, 5H), 0.93 (d, |
| | 2-((2S,3S,4S)-5-chloro-2-((((1r,4S)-4-(4- | | J = 7.2 Hz, 3H) |
| | (2-(1-(3-(2,4-dioxotetrahydropyrimidin- | | |
| | 1(2H)-yl)-4-methoxybenzoyl)piperidin-4- | | |
| | yl)ethyl)piperidine-1- | | |
| | carbonyl)cyclohexyl)amino)methyl)-6- | | |
| | fluoro-3-methyl-2-phenyl-2,3- | | |
| | dihydrobenzofuran-4-yl)-3-fluoro-4- | | |
| | methoxybenzamide | | |
| | Synthesized from C-I, tert-butyl 4-(2- | | |
| | (piperidin-4-yl)ethyl)piperidine-1- | | |
| | carboxylate and 3-(2,4- | | |
| | dioxotetrahydropyrimidin-1(2H)-yl)-4- | | |
| | methoxybenzoic acid | | |
| 28 | 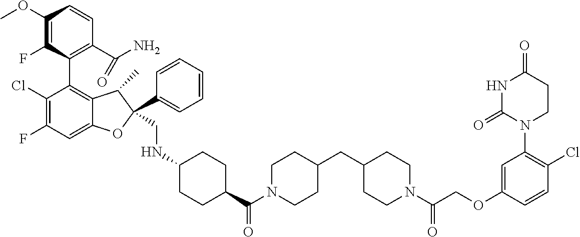 | 1029.4 [M + H]⁺ 0.82 (4) | (400 MHz, DMSO-d₆) δ (ppm) 10.45 (s, 1H), 7.64-7.55 (m, 2H), 7.46-7.38 (m, 3H), 7.36-7.23 (m, 4H), 7.10 (d, J = 3.0 Hz, 2H), 7.07 (d, J = 9.0 Hz, 1H), 6.92 (dd, J = 9.0, 3.0 Hz, 1H), 4.90-4.74 (m, 2H), 4.31 (t, J = 16.0 Hz, 2H), 3.88 (s, 3H), 3.86-3.64 (m, 3H), 3.65-3.51 (m, 1H), 3.27-3.20 (m, 1H), 3.17- 3.08 (m, 2H), 3.06-2.90 (m, 2H), 2.77-2.69 (m, 2H), 2.48-2.36 (m, 2H), 2.24- 2.15 (m, 1H), 1.80-1.51 (m, 10H), 1.35-1.19 (m, 3H), |
| | 2-((2S,3S,4S)-5-chloro-2-((((1r,4S)-4-(4- | | 1.15-1.04 (m, 3H), 1.00- |
| | ((1-(2-(4-chloro-3-(2,4- | | 0.78 (m, 7H), 0.93 (d, J = 7.2 |
| | dioxotetrahydropyrimidin-1(2H)- | | Hz, 3H) |
| | yl)phenoxy)acetyl)piperidin-4- | | |
| | yl)methyl)piperidine-1- | | |
| | carbonyl)cyclohexyl)amino)methyl)-6- | | |
| | fluoro-3-methyl-2-phenyl-2,3- | | |
| | dihydrobenzofuran-4-yl)-3-fluoro-4- | | |
| | methoxybenzamide | | |
| | Synthesized from C-I, tert-butyl 4- | | |
| | (piperidin-4-ylmethyl)piperidine-1- | | |
| | carboxylate and 2-(4-chloro-3-(2,4- | | |
| | dioxotetrahydropyrimidin-1(2H)- | | |
| | yl)phenoxy)acetic acid (synthesized | | |
| | according to WO2021053495) | | |
| 29 | 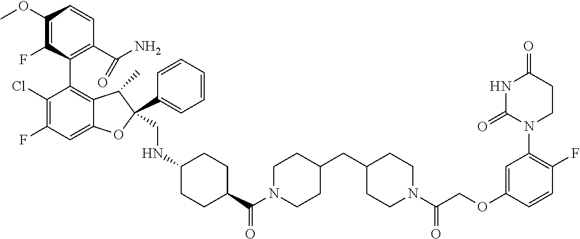 | 1013.7 [M + H]⁺ 0.79 (4) | (400 MHz, DMSO-d₆) δ (ppm) 10.42 (s, 1H), 7.58-7.50 (m, 2H), 7.37-7.32 (m, 2H), 7.29-7.10 (m, 5H), 7.05 (s br, 1H), 7.00 (d, J = 9.5 Hz, 1H), 6.95 (dd, J = 6.3, 3.1 Hz, 1H), 6.80 (dt, J = 9.2, 3.5 Hz, 1H), 4.78-4.64 (m, 2H), 4.25 (t, J = 14.9 Hz, 2H), 3.81 (s, 3H), 3.78-3.69 (m, 2H), 3.63 (t, J = 6.7 Hz, 2H), 3.16 (q, J = 7.1 Hz, 1H), 3-12- 3.02 (m, 2H), .2.64 (t, J = 6.7 Hz, 2H), 2.40-2.31 (m, 2H), 2.18-2.07 (m, 1H), 1.75- 1.43 (m, 10H), 1.27-1.13 |
| | 2-((2S,3S,4S)-5-chloro-2-((((1r,4S)-4-(4- | | (m, 3H), 1.08-0.97 (m, 3H), |
| | ((1-(2-(3-(2,4-dioxotetrahydropyrimidin- | | 0.93-0.71 (m, 7H), 0.86 (d, |
| | 1(2H)-yl)-4- | | J = 7.2 Hz, 3H) |
| | fluorophenoxy)acetyl)piperidin-4- | | |
| | yl)methyl)piperidine-1- | | |
| | carbonyl)cyclohexyl)amino)methyl)-6- | | |
| | fluoro-3-methyl-2-phenyl-2,3- | | |
| | dihydrobenzofuran-4-yl)-3-fluoro-4- | | |
| | methoxybenzamide | | |
| | Synthesized from C-I, tert-butyl 4- | | |
| | (piperidin-4-ylmethyl)piperidine-1- | | |
| | carboxylate and L-XVII | | |
| 30 | 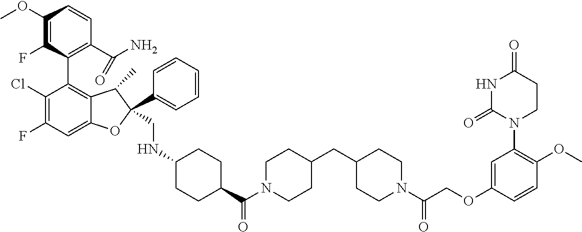 | 1025.7 [M + H]⁺ 0.79 (4) | (400 MHz, DMSO-d₆) δ (ppm) 10.31 (s, 1H), 7.64-7.58 (m, 2H), 7.43-7.40 (m, 2H), 7.37-7.23 (m, 4H), 7.12 (s br, 1H), 7.08 (d, J = 9.6 Hz, 1H), 7.02 (d, J = 9.0 Hz, 1H), 6.92-6.84 (m, 2H), 4.77- 4.65 (m, 2H), 4.33 (t, J = 14.1 Hz, 2H), 3.88 (s, 3H), 3.87- 3.79 (m, 2H), 3.74 (s, 3H), 3.56 (t, J = 6.7 Hz, 2H), 3.24 (q, J = 7.1 Hz, 1H), 3.20- 3.11 (m, 2H), 3.06-2.89 (m, 2H), 2.67 (t, J = 6.6 Hz, 2H), 2.49-2.39 (m, 2H), 2.25- |
| | 2-((2S,3S,4S)-5-chloro-2-((((1r,4S)-4-(4- | | 2.16 (m, 1H), 1.80-1.51 (m, |
| | ((1-(2-(3-(2,4-dioxotetrahydropyrimidin- | | 10H), 1.33-1.19 (m, 3H), |
| | 1(2H)-yl)-4- | | 1.15-1.05 (m, 3H), 1.00- |
| | methoxyphenoxy)acetyl)piperidin-4- | | 0.78 (m, 7H), 0.93 (d, J = 7.2 |
| | yl)methyl)piperidine-1- | | Hz, 3H) |
| | carbonyl)cyclohexyl)amino)methyl)-6- | | |
| | fluoro-3-methyl-2-phenyl-2,3- | | |
| | dihydrobenzofuran-4-yl)-3-fluoro-4- | | |
| | methoxybenzamide | | |
| | Synthesized from C-I, tert-butyl 4- | | |
| | (piperidin-4-ylmethyl)piperidine-1- | | |
| | carboxylate and L-XVIII | | |

### Example 31: 2-((2S,3S,4S)-5-chloro-2-((((1r,4S)-4-((5-(2-(2,6-dioxopiperidin-3-yl)-1-oxoisoindolin-4-yl)pentyl)carbamoyl)cyclohexyl)amino)methyl)-6-fluoro-3-methyl-2-phenyl-2,3-dihydrobenzofuran-4-yl)-3-fluoro-4-methoxybenzamide

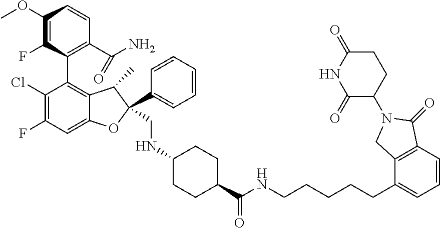

HATU (90 mg, 0.236 mmol) was added to a solution of (1S,4r)-4-((((2S,3S,4S)-4-(6-carbamoyl-2-fluoro-3-methoxyphenyl)-5-chloro-6-fluoro-3-methyl-2-phenyl-2,3-dihydrobenzofuran-2-yl)methyl)amino)cyclohexane-1-carboxylic acid (C-I) and TEA (0.109 mL, 0.787 mmol) in DMF (1 mL) at RT. After 5 min, 3-(4-(5-aminopentyl)-1-oxoisoindolin-2-yl)piperidine-2,6-dione hydrochloride (J. Med Chem. 2019, 62, 448-466) (60.4 mg, 0.165 mmol) was added and stirring at RT was continued for 1 h. The residue was purified by reverse phase chromatography (C18 silica, (water+0.1% TFA)/ACN; gradient: 5% to 60% ACN) followed by prep HPLC to afford the title product (37 mg) as a colorless powder. UPLC-MS 4: m/z 896.6 [M+H]⁺, t_{R}=0.79 min. ¹H NMR (400 MHz, DMSO-d₆) δ (ppm) 10.97 (s, 1H), 7.62-7.57 (m, 3H), 7.46-7.38 (m, 4H), 7.33 (t, J=7.6 Hz, 2H), 7.30-7.23 (m, 2H), 7.10-7.04 (m, 2H), 5.20-4.99 (m, 1H), 4.45 (d, J=17.4 Hz, 1H), 4.29 (d, J=, 17.2 Hz, 1H), 3.88 (s, 3H), 3.19-3.08 (m, 3H), 3.04-2.84 (m, 3H), 2.63-2.55 (m, 3H), 2.47-2.37 (m, 1H), 2.11-1.96 (m, 2H), 1.88 (d, J=13.2 Hz, 1H), 1.74 (t, J=13.5 Hz, 2H), 1.63-1.51 (m, 4H), 1.44-1.35 (m, 2H), 1.31-1.15 (m, 5H), 0.93 (d, J=6.9 Hz, 3H), 0.90-0-71 (m, 3H).

### Example 32: 4-((2S,4S)-5-Chloro-6-fluoro-2-phenyl-2-((S)-pyrrolidin-2-yl)-2,3-dihydrobenzofuran-4-yl)-6-(2-(2-(4-(2-(2,6-dioxopiperidin-3-yl)-1-oxoisoindolin-5-yl)-1H-1,2,3-triazol-1-yl)ethoxy)ethoxy)-5-fluoro-N-methylnicotinamide IVD129

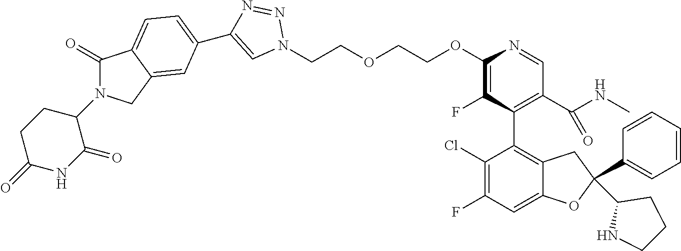

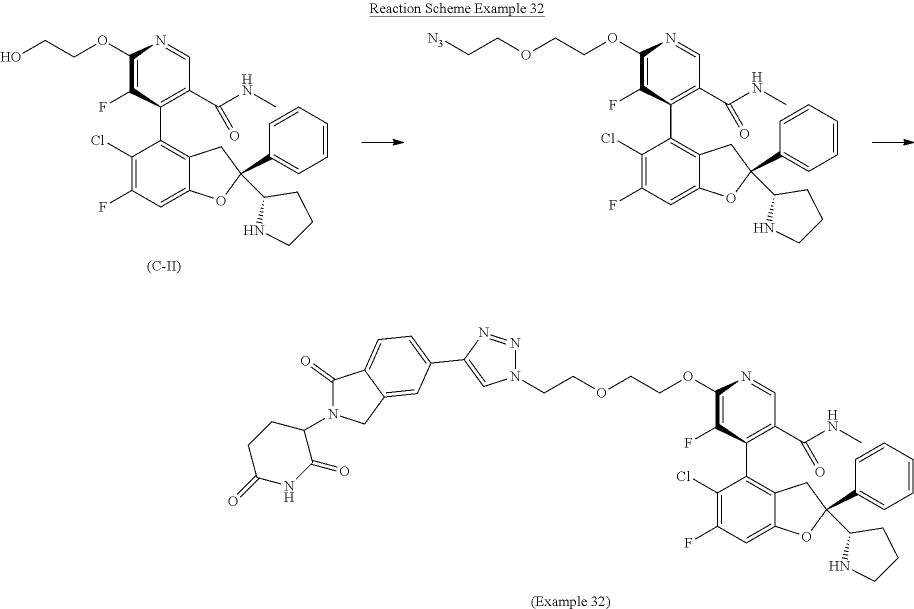

### Step 1: 6-(2-(2-Azidoethoxy)ethoxy)-4-((2S,4S)-5-chloro-6-fluoro-2-phenyl-2-((S)-pyrrolidin-2-yl)-2,3-dihydrobenzofuran-4-yl)-5-fluoro-N-methylnicotinamide

A microwave vial was charged with 4-((2S,4S)-5-chloro-6-fluoro-2-phenyl-2-((S)-pyrrolidin-2-yl)-2,3-dihydrobenzofuran-4-yl)-5-fluoro-6-(2-hydroxyethoxy)-N-methylnicotinamide (C-II) (150 mg, 0.283 mmol) in dioxane (5 mL) and 2-(2-azidoethoxy)ethan-1-ol (20% in toluene) (2784 mg, 4.25 mmol) was added. NaH (7.15 mg, 0.283 mmol, 95%) was added, the vial was sealed and the reaction mixture was stirred at 65° C. for 3 d. After cooling to RT NaHSO₄ (34 mg, 0.283 mmol) was added and the reaction mixture was concentrated. The residue was purified by flash chromatography (silica, DCM/MeOH+5% (7M NH₃ in MeOH); gradient: 10% to 25% (MeOH+5% 7M NH₃ in MeOH)) to afford the title compound (122 mg). UPLC-MS 4: m/z 599.3 [M+H]⁺, t_{R}=0.78 min.

### Step 2: 4-((2S,4S)-5-Chloro-6-fluoro-2-phenyl-2-((S)-pyrrolidin-2-yl)-2,3-dihydrobenzofuran-4-yl)-6-(2-(2-(4-(2-(2,6-dioxopiperidin-3-yl)-1-oxoisoindolin-5-yl)-1H-1,2,3-triazol-1-yl)ethoxy)ethoxy)-5-fluoro-N-methylnicotinamide (Example 32)

6-(2-(2-Azidoethoxy)ethoxy)-4-((2S,4S)-5-chloro-6-fluoro-2-phenyl-2-((S)-pyrrolidin-2-yl)-2,3-dihydrobenzofuran-4-yl)-5-fluoro-N-methylnicotinamide (40 mg, 0.067 mmol) and 3-(5-ethynyl-1-oxoisoindolin-2-yl)piperidine-2,6-dione (PubChem CID 146410789) (19.7 mg, 0.073 mmol) were dissolved in DMSO (4 mL). A solution of L-ascorbic acid (23.5 mg, 0.134 mmol) in water (0.5 mL) was added followed by a solution of CuSO₄·5H₂O (4.2 mg, 0.017 mmol) in water (0.5 mL). The reaction mixture was stirred at 50° C. for 45 min. The reaction mixture was concentrated and the residue was purified by flash chromatography (silica, DCM/MeOH+5% (7M NH₃ in MeOH); gradient: 10% to 25% (MeOH+5% 7M NH₃ in MeOH)) to give the title compound (36 mg) as a colorless powder. UPLC-MS 4: m/z 867.3 [M+H]⁺, t_{R}=0.58 and 0.59 min. ¹H NMR (400 MHZ, DMSO-d₆) δ (ppm) 11.00 (s, 1H), 8.65 (s, 1H), 8.51-8.45 (m, 1H), 8.28 (d, J=2.7 Hz, 1H), 8.04 (d, J=3.6 Hz, 1H), 7.94-7.88 (m, 1H), 7.72 (t, J=7.6 Hz, 1H), 7.51-7.46 (m, 2H), 7.43-7.35 (m, 2H), 7.34-7.29 (m, 1H), 7.14 (dd, J=9.5, 2.5 Hz, 1H), 5.13 (dd, J=13.3, 5.1 Hz, 1H), 4.61 (t, J=5.1 Hz, 2H), 4.56-4.49 (m, 3H), 4.36 (dd, J=17.4, 4.5 Hz, 1H), 3.95 (t, J=5.1 Hz, 2H), 3.84 (t, J=4.5 Hz, 2H), 3.52-3.43 (m, 2H), 3.06-2.87 (m, 5H), 2.67 (d, J=4.5 Hz, 3H), 2.64-2.58 (m, 1H), 2.45-2.37 (m, 1H), 2-07-1.98 (m, 1H), 1.95-1.85 (m, 1H), 1.79-1.70 (m, 2H), 1.67-1.55 (m, 1H).

The following Examples were prepared according to Example 32:

| | | UPLC MS | |
|---|---|---|---|
| | | m/z | |
| | | t_{R} [min] | |
| Ex. | Structure/Chemical Name | (method) | ¹H NMR |
| 33 | 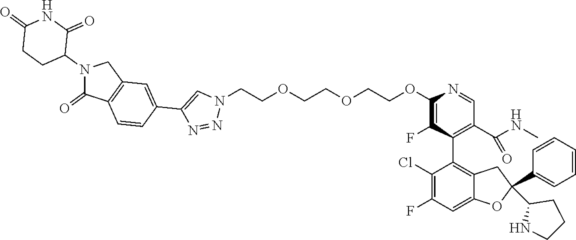 | 911.7 [M + H]⁺ 0.61 (4) | (400 MHz, DMSO-d₆) δ (ppm) 11.00 (s, 1H), 8.65 (s, 1H), 8.51 (d, J = 4.8 Hz, 1H), 8.29 (s, 1H), 8.08 (s, 1H), 7.95 (dd, J = 7.9, 1.4 Hz, 1H), 7.75 (dd, J = 7.9, 1.6 Hz, 1H), 7.52- 7.46 (m, 2H), 7.42 (t, J = 7.5 Hz, 2H), 7.37-7.32 (m, 1H), 7.17 (d, J = 9.4 Hz, 1H), .5.18- 5.08 (m, 1H), 4.56 (t, J = 5.2 Hz, 2H), 4.53-4.35 (m, 4H), 3.86 (t, J = 5.1 Hz, 2H), 3.77- 3.71 (m, 2H), 3.58-3.55 (m, 4H), 3.51-3.44 (m, 2H), 3.15- 2.85 (m, 5H), 2.67 (d, J = 4.5 Hz, 3H), 2.65-2.57 (m, 1H), |
| | 4-((2S,4S)-5-chloro-6-fluoro-2-phenyl-2- | | 2.41 (dd, J = 13.1, 4.4 Hz, |
| | ((S)-pyrrolidin-2-yl)-2,3- | | 1H), 2-05-1.96 (m, 2H), 1.94- |
| | dihydrobenzofuran-4-yl)-6-(2-(2-(2-(4-(2- | | 1.75 (m, 2H), 1.73-1.63 (m, |
| | (2,6-dioxopiperidin-3-yl)-1-oxoisoindolin- | | 1H) |
| | 5-yl)-1H-1,2,3-triazol-1- | | |
| | yl)ethoxy)ethoxy)ethoxy)-5-fluoro-N- | | |
| | methylnicotinamide APO423 | | |
| | Synthesized from C-II, | | |
| | 2-(2-(2-azidoethoxy)ethoxy)ethan-1-ol | | |
| | and 3-(5-ethynyl-1-oxoisoindolin-2- | | |
| | yl)piperidine-2,6-dione | | |
| 34 | 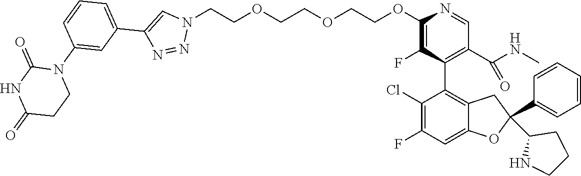 | 857.5 [M + H]⁺ 0.62 (4) | (400 MHz, DMSO-d₆) δ (ppm) 10.40 (s, 1H), 8.54-8.49 (m, 2H), 8.30 (s, 1H), 7.81 (t, J = 1.9 Hz, 1H), 7.66 (dt, J = 7.7, 1.4 Hz, 1H), 7.51-7.45 (m, 2H), 7.44-7.39 (m, 3H), 7.37- 7.32 (m, 1H), 7.29-7.26 (m, 1H), 7.16 (d, J = 9.4 Hz, 1H), 4.56-4.43 (m, 4H), 3.88- 3.80 (m, 4H), 3.77-3.73 (m, 2H), 3.58-3.54 (m, 4H), 3.50- 3.44 (m, 2H), 3.09-2.97 (m, |
| | 4-((2S,4S)-5-chloro-6-fluoro-2-phenyl-2- | | 3H), 2.72 (t, J = 6.6 Hz, 2H), |
| | ((S)-pyrrolidin-2-yl)-2,3- | | 2.69-2.65 (m, 4H), 2,34- |
| | dihydrobenzofuran-4-yl)-6-(2-(2-(2-(4-(3- | | 2.31 (m, 1H), 2.06-1.93 (m, |
| | (2,4-dioxotetrahydropyrimidin-1(2H)- | | 1H), 1.88-1.74 (m, 2H), 1.71- |
| | yl)phenyl)-1H-1,2,3-triazol-1- | | 1.59 (m, 1H) |
| | yl)ethoxy)ethoxy)ethoxy)-5-fluoro-N- | | |
| | methylnicotinamide FSK018 | | |
| | Synthesized from C-II, | | |
| | 2-(2-(2-azidoethoxy)ethoxy)ethan-1-ol | | |
| | and 1-(3- | | |
| | ethynylphenyl)dihydropyrimidine- | | |
| | 2,4(1H,3H)-dione (synthesized according | | |
| | to WO2021053495) | | |

### Example 35: 2-((2S,3S,4S)-5-chloro-2-(((5-(2-(2,6-dioxopiperidin-3-yl)-1-oxoisoindolin-4-yl)pentyl)amino)methyl)-6-fluoro-3-methyl-2-phenyl-2,3-dihydrobenzofuran-4-yl)-3-fluoro-4-methoxybenzamide

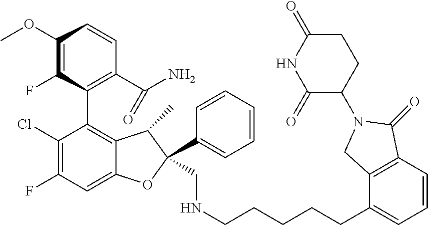

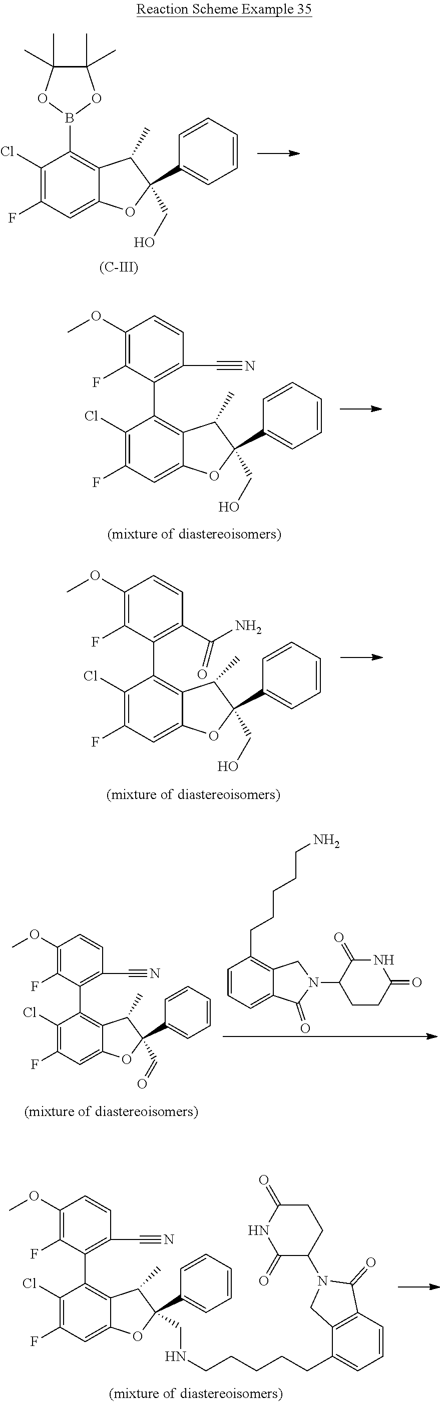

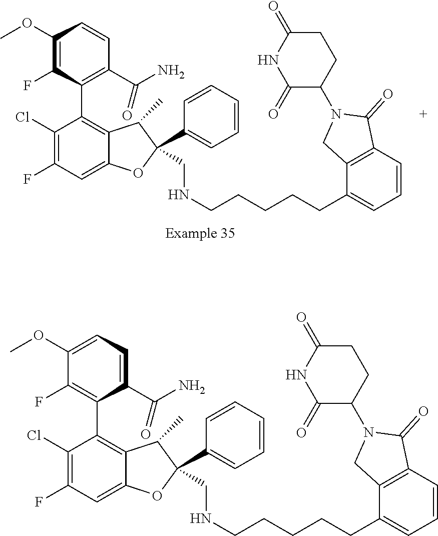

### Step 1: 2-((2S,3S,4S)-5-Chloro-6-fluoro-2-(hydroxymethyl)-3-methyl-2-phenyl-2,3-dihydrobenzofuran-4-yl)-3-fluoro-4-methoxybenzonitrile and 2-((2S,3S,4R)-5-chloro-6-fluoro-2-(hydroxymethyl)-3-methyl-2-phenyl-2,3-dihydrobenzofuran-4-yl)-3-fluoro-4-methoxybenzonitrile

A microwave vial was charged with ((2S,3S)-5-chloro-6-fluoro-3-methyl-2-phenyl-4-(4,4,5,5-tetramethyl-1,3,2-dioxaborolan-2-yl)-2,3-dihydrobenzofuran-2-yl)methanol (C-III) (1.65 g, 3.94 mmol), 2-bromo-3-fluoro-4-methoxybenzonitrile (N-I-a) (1.088 g, 4.73 mmol), toluene (10 mL) and water (2 mL). K₃PO₄ (2.5089 g, 11.82 mmol) was added and the mixture was degassed with Ar. N-XantPhos (217 mg, 0.394 mmol) was added, followed by Pd₂(dba)₃ (180 mg, 0.197 mmol), the vial was sealed and the reaction mixture was stirred at 110° C. overnight. After cooling to RT the mixture was diluted with EtOAc and washed with a 10% citric acid solution, a sat solution of NaHCO₃ and brine. Drying (Na₂SO₄) and concentration of the organic phase afforded the cude product which was purified by flash chromatography (silica, cyclohexane/EtOAc; gradient: 15% to 100% EtOAc) to give a mixture of the title compounds (900 mg). UPLC-MS 4: m/z 459.1 [M+H₂O]⁺, t_{R}=1.06 and 1.10 min.

### Step 2: 2-((2S,3S,4S)-5-chloro-6-fluoro-2-(hydroxymethyl)-3-methyl-2-phenyl-2,3-dihydrobenzofuran-4-yl)-3-fluoro-4-methoxybenzamide and 2-((2S,3S,4R)-5-chloro-6-fluoro-2-(hydroxymethyl)-3-methyl-2-phenyl-2,3-dihydrobenzofuran-4-yl)-3-fluoro-4-methoxybenzamide

Hydrido (dimethylphosphinous acid-kP) [hydrogen bis(dimethylphosphinito-kP)]platinum (II) (48.6 mg, 0.113 mmol) was added to a solution of a mixture of 2-((2S,3S,4S)-5-chloro-6-fluoro-2-(hydroxymethyl)-3-methyl-2-phenyl-2,3-dihydrobenzofuran-4-yl)-3-fluoro-4-methoxybenzonitrile and 2-((2S,3S,4R)-5-chloro-6-fluoro-2-(hydroxymethyl)-3-methyl-2-phenyl-2,3-dihydrobenzofuran-4-yl)-3-fluoro-4-methoxybenzonitrile in EtOH (10 mL) and water (5 mL) and the reaction mixture was stirred at 85° C. for 90 min. After cooling to RT the reaction mixture was diluted with EtOAc and washed with brine. The organic phase was dried (Na₂SO₄), filtered over Celite and concentrated to give a mixture of the title compounds (530 mg). UPLC-MS 4: m/z 460.2 [M+H]⁺, t_{R}=0.88 and 0.99 min.

### Step 3: 2-((2S,3S,4S)-5-chloro-6-fluoro-2-formyl-3-methyl-2-phenyl-2,3-dihydrobenzofuran-4-yl)-3-fluoro-4-methoxybenzonitrile and 2-((2S,3S,4R)-5-chloro-6-fluoro-2-formyl-3-methyl-2-phenyl-2,3-dihydrobenzofuran-4-yl)-3-fluoro-4-methoxybenzonitrile

To a stirred solution of oxalyl chloride (0.153 mL, 1.752 mmol) in DCM (6 mL) was added DMSO (0.249 mL, 3.50 mmol) in DCM (1 mL) at −78° C. over 5 min and the reaction mixture was stirred at −78° C. for 15 min. A solution of a mixture of 2-((2S,3S,4S)-5-chloro-6-fluoro-2-(hydroxymethyl)-3-methyl-2-phenyl-2,3-dihydrobenzofuran-4-yl)-3-fluoro-4-methoxybenzamide and 2-((2S,3S,4R)-5-chloro-6-fluoro-2-(hydroxymethyl)-3-methyl-2-phenyl-2,3-dihydrobenzofuran-4-yl)-3-fluoro-4-methoxybenzamide (530 mg, 1.095 mmol) in DCM (3 mL) was then added over 5 min and stirring at −78° C. was continued for 15 min. TEA (0.759 mL, 5.47 mmol) in DCM (1 mL) was added over 5 min and the reaction mixture was allowed to warm to 0° C. over 30 min. The reaction mixture was diluted with DCM and water, extracted twice with DCM and the combined organic extracts were washed with water and brine, dried over anhydrous Na₂SO₄ and concentrated to afford a mixture of the title compounds (475 mg) as a yellow foam, which was used without purification (under the conditions of the reaction the amide group was transformed back to the nitrile). UPLC-MS 4: m/z 457.2 [M+H₂O]⁺, t_{R}=1.22 min

### Steps 4 and 5: 2-((2S,3S,4S)-5-chloro-2-(((5-(2-(2,6-dioxopiperidin-3-yl)-1-oxoisoindolin-4-yl)pentyl)amino)methyl)-6-fluoro-3-methyl-2-phenyl-2,3-dihydrobenzofuran-4-yl)-3-fluoro-4-methoxybenzamide (Example 35) and 2-((2S,3S,4R)-5-chloro-2-(((5-(2-(2,6-dioxopiperidin-3-yl)-1-oxoisoindolin-4-yl)pentyl)amino)methyl)-6-fluoro-3-methyl-2-phenyl-2,3-dihydrobenzofuran-4-yl)-3-fluoro-4-methoxybenzamide

A flask was charged with a mixture of 2-((2S,3S,4S)-5-chloro-6-fluoro-2-formyl-3-methyl-2-phenyl-2,3-dihydrobenzofuran-4-yl)-3-fluoro-4-methoxybenzonitrile and 2-((2S,3S,4R)-5-chloro-6-fluoro-2-formyl-3-methyl-2-phenyl-2,3-dihydrobenzofuran-4-yl)-3-fluoro-4-methoxybenzonitrile (100 mg, 0.218 mmol), DCE (2 mL) and 3-(4-(5-aminopentyl)-1-oxoisoindolin-2-yl)piperidine-2,6-dione hydrochloride (J. Med Chem. 2019, 62, 448-466) (80 mg, 0.218 mmol). NaOAc (17.92 mg, 0.218 mmol) was added and the reaction mixture was stirred at 80° C. for 2 h. After cooling to 60° C. NaBH(OAc)₃ (93 mg, 0.437 mmol) was added in portions and stirring at 60° C. was continued for 45 min. After cooling to RT the reaction mixture was diluted with DCM and quenched with a sat solution of NaHCO₃, the phases were separated and the aqueous phase was extracted with DCM. The combined organic phases were dried (Na₂SO₄) and concentrated to afford a mixture of the intermediates 2-((2S,3S,4S)-5-chloro-2-(((5-(2-(2,6-dioxopiperidin-3-yl)-1-oxoisoindolin-4-yl)pentyl)amino)methyl)-6-fluoro-3-methyl-2-phenyl-2,3-dihydrobenzofuran-4-yl)-3-fluoro-4-methoxybenzonitrile and 2-((2S,3S,4R)-5-chloro-2-(((5-(2-(2,6-dioxopiperidin-3-yl)-1-oxoisoindolin-4-yl)pentyl)amino)methyl)-6-fluoro-3-methyl-2-phenyl-2,3-dihydrobenzofuran-4-yl)-3-fluoro-4-methoxybenzonitrile. UPLC-MS 4: m/z 753.3 [M+H]⁺, t_{R}=0.84 and 0.87 min.

The mixture of nitriles was converted to the corresponding amides using similar reaction conditions as in step 2. The crude product was subjected to flash chromatography (silica, DCM/MeOH+5% (7M NH₃ in MeOH); gradient: 5% to 20% (MeOH+5% 7M NH₃ in MeOH)) to afford the title compounds.

2-((2S,3S,4S)-5-chloro-2-(((5-(2-(2,6-dioxopiperidin-3-yl)-1-oxoisoindolin-4-yl)pentyl)amino)methyl)-6-fluoro-3-methyl-2-phenyl-2,3-dihydrobenzofuran-4-yl)-3-fluoro-4-methoxybenzamide (Example 35) (15 mg): ¹H NMR (400 MHZ, DMSO-d₆) δ (ppm) 10.98 (s, 1H), 7.63 (s br, 1H), 7.60 (dd, J=8.8, 1.4 Hz, 1H), 7.56 (dd, J=7.0, 1.6 Hz, 1H), 7.47-7.39 (m, 4H), 7.36-7.29 (m, 2H), 7.29-7.23 (m, 2H), 7.09 (s br, 1H), 7.05 (d, J=9.5 Hz, 1H), 5.13 (dd, J=13.3, 5.1 Hz, 1H), 4.43 (d, J=17.1 Hz, 1H), 4.28 (d, J=17.1 Hz, 1H), 3.89 (s, 3H), 3.29-3.22 (m, 1H), 3.16-3.07 (m, 2H), 2.98-2.87 (m, 1H), 2.63-2.54 (m, 2H), 2.47-2.36 (m, 2H), 2.04-1.97 (m, 1H), 1.68-1.61 (m, 1H), 1.56-1.46 (m, 2H), 1.35-1.28 (m, 2H), 1.26-1.15 (m, 3H), 0.93 (d, J=7.2 Hz, 3H). UPLC-MS 4: m/z 771.3 [M+H]⁺, t_{R}=0.80 min.

Other diasteroisomer 2-((2S,3S,4R)-5-chloro-2-(((5-(2-(2,6-dioxopiperidin-3-yl)-1-oxoisoindolin-4-yl)pentyl)amino)methyl)-6-fluoro-3-methyl-2-phenyl-2,3-dihydrobenzofuran-4-yl)-3-fluoro-4-methoxybenzamide (22 mg): UPLC-MS 4: m/z 771.3 [M+H]⁺, t_{R}=0.71 min.

### Example 36: 2-((2S,4S)-2-(aminomethyl)-5-chloro-6-fluoro-2-phenyl-2,3-dihydrobenzofuran-4-yl)-N-(5-(2-(2,6-dioxopiperidin-3-yl)-1-oxoisoindolin-4-yl)pentyl)-3-fluoro-4-methoxybenzamide

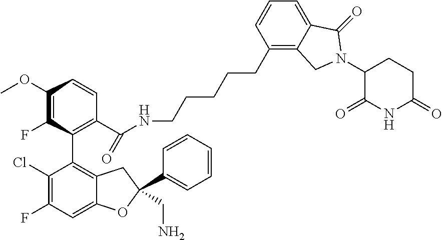

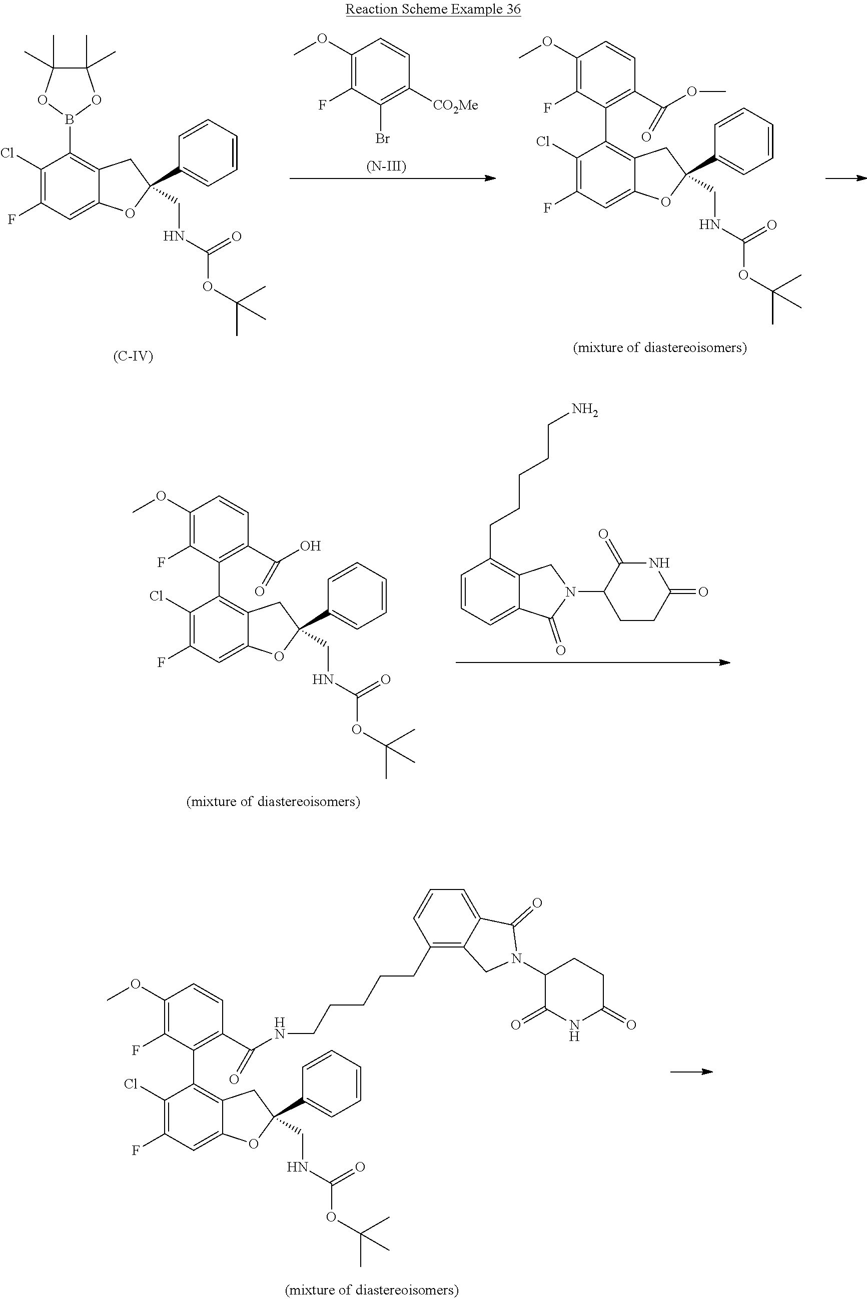

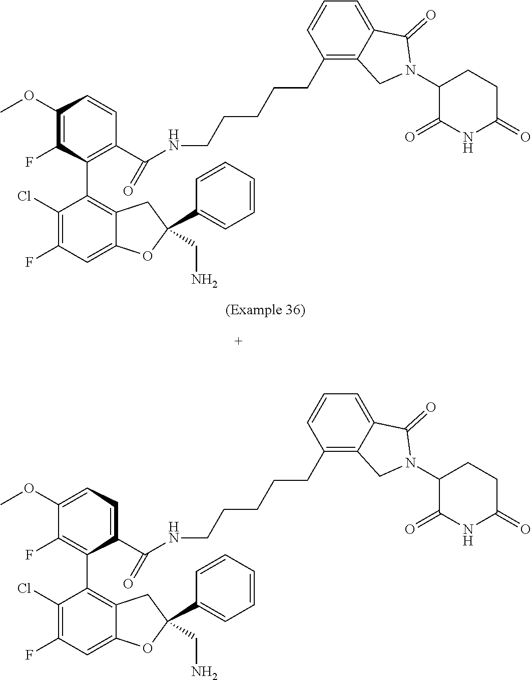

### Step 1: Methyl 2-((2S,4S)-2-(((tert-butoxycarbonyl)amino)methyl)-5-chloro-6-fluoro-2-phenyl-2,3-dihydrobenzofuran-4-yl)-3-fluoro-4-methoxybenzoate and methyl 2-((2S,4R)-2-(((tert-butoxycarbonyl)amino)methyl)-5-chloro-6-fluoro-2-phenyl-2,3-dihydrobenzofuran-4-yl)-3-fluoro-4-methoxybenzoate

A mixture of the title compounds (550 mg) was obtained from tert-butyl (S)-((5-chloro-6-fluoro-2-phenyl-4-(4,4,5,5-tetramethyl-1,3,2-dioxaborolan-2-yl)-2,3-dihydrobenzofuran-2-yl)methyl)carbamate (C-IV) (900 mg, 1.786 mmol) and methyl 2-bromo-3-fluoro-4-methoxybenzoate (N-III) (517 mg, 1.965 mmol) using similar reaction conditions as described in step 1 of Example 35. UPLC-MS 4: m/z 460.1 [M−Boc+H]⁺, t_{R}=1.43 and 1.45 min.

### Step 2: 2-((2S,4S)-2-(((tert-butoxycarbonyl)amino)methyl)-5-chloro-6-fluoro-2-phenyl-2,3-dihydrobenzofuran-4-yl)-3-fluoro-4-methoxybenzoic acid and 2-((2S,4R)-2-(((tert-butoxycarbonyl)amino)methyl)-5-chloro-6-fluoro-2-phenyl-2,3-dihydrobenzofuran-4-yl)-3-fluoro-4-methoxybenzoic acid

NaOH (1.96 mL, 1.96 mmol, 1 M in water) was added dropwise to a solution of a mixture of methyl 2-((2S,4S)-2-(((tert-butoxycarbonyl)amino)methyl)-5-chloro-6-fluoro-2-phenyl-2,3-dihydrobenzofuran-4-yl)-3-fluoro-4-methoxybenzoate and methyl 2-((2S,4R)-2-(((tert-butoxycarbonyl)amino)methyl)-5-chloro-6-fluoro-2-phenyl-2,3-dihydrobenzofuran-4-yl)-3-fluoro-4-methoxybenzoate (550 mg, 0.982 mmol) in dioxane (10 mL). After 3 d of stirring at 40° C. more NaOH (1.96 mL, 1.96 mmol, 1 M in water) and MeOH (5 mL) were added and stirring at 80° C. was continued overnight. The reaction mixture was concentrated, acidified with an aqueous citric acid solution and extracted with DCM. The combined organic extracts were washed with an aqueous citric acid solution, water and brine, dried (Na₂SO₄) and concentrated to afford a mixture of the title compounds (480 mg). UPLC-MS 4: m/z 544.3 [M−H]⁻, t_{R}=1.33 and 1.38 min.

### Step 3: tert-butyl (((2S,4S)-5-chloro-4-(6-((5-(2-(2,6-dioxopiperidin-3-yl)-1-oxoisoindolin-4-yl)pentyl)carbamoyl)-2-fluoro-3-methoxyphenyl)-6-fluoro-2-phenyl-2,3-dihydrobenzofuran-2-yl)methyl)carbamate and tert-butyl (((2S,4R)-5-chloro-4-(6-((5-(2-(2,6-dioxopiperidin-3-yl)-1-oxoisoindolin-4-yl)pentyl)carbamoyl)-2-fluoro-3-methoxyphenyl)-6-fluoro-2-phenyl-2,3-dihydrobenzofuran-2-yl)methyl)carbamate

A flask was charged with a mixture of 2-((2S,4S)-2-(((tert-butoxycarbonyl)amino)methyl)-5-chloro-6-fluoro-2-phenyl-2,3-dihydrobenzofuran-4-yl)-3-fluoro-4-methoxybenzoic acid and 2-((2S,4R)-2-(((tert-butoxycarbonyl)amino)methyl)-5-chloro-6-fluoro-2-phenyl-2,3-dihydrobenzofuran-4-yl)-3-fluoro-4-methoxybenzoic acid (140 mg, 0.244 mmol), DMF (Volume: 5 mL) and TEA (0.169 mL, 1.218 mmol). 3-(4-(5-Aminopentyl)-1-oxoisoindolin-2-yl)piperidine-2,6-dione hydrochloride (J. Med Chem. 2019, 62, 448-466) (98 mg, 0.268 mmol) was added, followed by HATU (139 mg, 0.365 mmol) and stirring at RT was continued overnight. The reaction mixture was diluted with EtOAc, washed with a 10% aqueous citric acid solution, water, a sat solution of NaHCO₃ and brine. The organic layer was dried (Na₂SO₄) and concentrated. The crude product was purified by flash chromatography (silica, DCM/MeOH; gradient: 1% to 6% MeOH) to give a mixture of the title compounds (160 mg). UPLC-MS 4: m/z 857.4 [M+H]⁺, t_{R}=1.22 and 1.28 min.

### Step 4: 2-((2S,4S)-2-(aminomethyl)-5-chloro-6-fluoro-2-phenyl-2,3-dihydrobenzofuran-4-yl)-N-(5-(2-(2,6-dioxopiperidin-3-yl)-1-oxoisoindolin-4-yl)pentyl)-3-fluoro-4-methoxybenzamide (Example 36) and 2-((2S,4R)-2-(aminomethyl)-5-chloro-6-fluoro-2-phenyl-2,3-dihydrobenzofuran-4-yl)-N-(5-(2-(2,6-dioxopiperidin-3-yl)-1-oxoisoindolin-4-yl)pentyl)-3-fluoro-4-methoxybenzamide

TFA (1 mL, 12.98 mmol) was slowly added to a solution of a mixture of tert-butyl (((2S,4S)-5-chloro-4-(6-((5-(2-(2,6-dioxopiperidin-3-yl)-1-oxoisoindolin-4-yl)pentyl)carbamoyl)-2-fluoro-3-methoxyphenyl)-6-fluoro-2-phenyl-2,3-dihydrobenzofuran-2-yl)methyl)carbamate and tert-butyl (((2S,4R)-5-chloro-4-(6-((5-(2-(2,6-dioxopiperidin-3-yl)-1-oxoisoindolin-4-yl)pentyl)carbamoyl)-2-fluoro-3-methoxyphenyl)-6-fluoro-2-phenyl-2,3-dihydrobenzofuran-2-yl)methyl)carbamate (160 mg, 0.177 mmol) in DCM (4 mL). After stirring at RT overnight the reaction mixture was concentrated and purified by flash chromatography (silica, DCM/MeOH+5% (7M NH₃ in MeOH); gradient: 5% to 15% (MeOH+5% 7M NH₃ in MeOH)) to afford the title compounds:

2-((2S,4S)-2-(aminomethyl)-5-chloro-6-fluoro-2-phenyl-2,3-dihydrobenzofuran-4-yl)-N-(5-(2-(2,6-dioxopiperidin-3-yl)-1-oxoisoindolin-4-yl)pentyl)-3-fluoro-4-methoxybenzamide (53 mg) (Example 36): ¹H NMR (400 MHZ, DMSO-d₆) δ (ppm) 8.08 (t, J=5.7 Hz, 1H), 7.58 (dd, J=6.0, 2.6 Hz, 1H), 7.49-7.26 (m, 10H), 7.05 (d, J=9.6 Hz, 1H), 5.14 (dd, J=13.3, 5.1 Hz, 1H), 4.47 (d, J=17.1 Hz, 1H), 4.32 (d, J=17.1 Hz, 1H), 3.90 (s, 3H), 3.42 (d, J=15.9 Hz, 1H), 3.20-2.99 (m, 2H), 2.99-2.89 (m, 2H), 2.88-2.81 (m, 1H), 2.71-2.59 (m, 3H), 2.46-2.40 (m, 2H), 2.06-1.99 (m, 1H), 1.63-1.54 (m, 2H), 1.45-1.34 (m, 2H), 1.31-1.23 (m, 2H). UPLC-MS 4: m/z 757.2 [M+H]⁺, t_{R}=0.85.

Other diastereoisomer 2-((2S,4R)-2-(aminomethyl)-5-chloro-6-fluoro-2-phenyl-2,3-dihydrobenzofuran-4-yl)-N-(5-(2-(2,6-dioxopiperidin-3-yl)-1-oxoisoindolin-4-yl)pentyl)-3-fluoro-4-methoxybenzamide (50 mg): UPLC-MS 4: m/z 757.2 [M+H]⁺, t_{R}=0.75.

### Example 37: 2-((2S,4S)-2-(aminomethyl)-5-chloro-6-fluoro-2-phenyl-2,3-dihydrobenzofuran-4-yl)-N-(5-(4-(2-(2,6-dioxopiperidin-3-yl)-1-oxoisoindolin-5-yl)-1H-1,2,3-triazol-1-yl)pentyl)-3-fluoro-4-methoxybenzamide

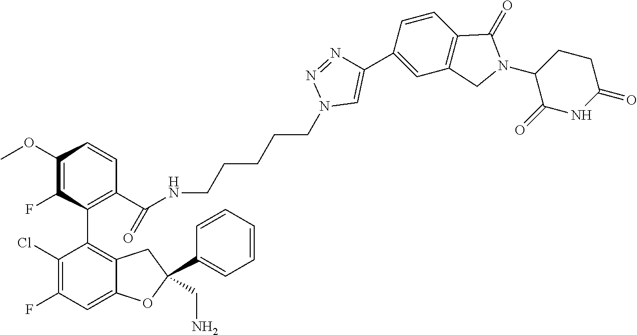

The title compound (isolated as TFA salt) was prepared in analogy to Example 36 by first coupling the acid intermediate with 5-azidopentan-1-amine (see step 3 in Example 36) followed by cycloaddition with 3-(5-ethynyl-1-oxoisoindolin-2-yl)piperidine-2,6-dione (PubChem CID 146410789) (see step 2 in Example 32). NMR (400 MHZ, DMSO-d₆) δ (ppm) 11.00 (s, 1H), 8.73 (s, 1H), 8.36 (t, J=5.8 Hz, 1H), 8.10 (s, 1H), 8.00 (dd, J=7.9, 1.4 Hz, 1H), 7.81 (d, J=8.0 Hz, 1H), 7.52-7.35 (m, 6H), 7.20 (td, J=8.5, 3.2 Hz, 1H), 7.12 (d, J=9.3 Hz, 1H), 5.14 (dd, J=13.2, 5.1 Hz, 1H), 4.53 (d, J=16.7 Hz, 1H), 4.44-4.34 (m, 3H), 3.86 (s, 3H), 3.44 (d, J=16.4 Hz, 1H), 3.14-3.01 (m, 3H), 2.98-2.88 (m, 2H), 2.68-2.58 (m, 1H), 2.50-2.33 (m, 2H), 2.07-1.98 (m, 1H), 1.93-1.82 (m, 2H), 1.50-1.41 (m, 2H), 1.31-1.20 (m, 2H). UPLC-MS 4: m/z 824.3 [M+H]⁺, t_{R}=0.75.

The following Examples were prepared according to General Procedures 1 or 2:

| | | UPLC MS | |
|---|---|---|---|
| | | m/z | |
| | | t_{R} [min] | |
| Ex. | Structure/Chemical Name | (method) | ¹H NMR |
| 38 | 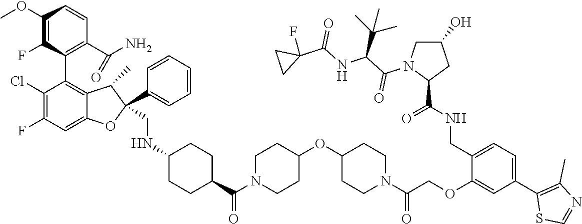 | 662.7 [M + 2H]²⁺/2 1.09 (3) | (400 MHz, DMSO-d₆) δ (ppm) 8.97 (s, 1H), 8.56 (s br, 1H), 7.63-7.57 (m, 2H), 7.43- 7.38 (m, 3H), 7.36-7.23 (m, 5H), 7.10 (s br, 1H), 7.07 (d, J = 9.6 Hz, 1H), 7.00-6.92 (m, 2H), 5.15 (d, J = 3.6 Hz, 1H), 4.96 (s, 2H), 4.59 (d, J = 9.2 Hz, 1H), 4.50 (t, J = 8.2 Hz, 1H), 4.39-4.23 (m, 3H), 3.88 (s, 3H), 3.87-3.80 (m, 2H), 3.72-3.56 (m, 6H), 3.27- 3.19 (m, 2H), 3.18-3.07 (m, 4H), 3.00 (t, J = 10.4 Hz, 1H), |
| | (2S,4R)-N-(2-(2-(4-((1-((1S,4r)-4- | | 2.46-2.39 (m, 1H), 2.43 (s, |
| | ((((2S,3S,4S)-4-(6-carbamoyl-2-fluoro-3- | | 3H), 2.24-2.13 (m, 1H), 2.13- |
| | methoxyphenyl)-5-chloro-6-fluoro-3- | | 2.05 (m, 1H), 1.96-1.86 (m, |
| | methyl-2-phenyl-2,3-dihydrobenzofuran- | | 1H), 1.85-1.68 (m, 6H), 1.59- |
| | 2-yl)methyl)amino)cyclohexane-1- | | 1.51 (m, 2H), 1.48-1.18 (m, |
| | carbonyl)piperidin-4-yl)oxy)piperidin-1- | | 10H), 0.99-0.81 (m, 15H) |
| | yl)-2-oxoethoxy)-4-(4-methylthiazol-5- | | |
| | yl)benzyl)-1-((S)-2-(1- | | |
| | fluorocyclopropane-1-carboxamido)-3,3- | | |
| | dimethylbutanoyl)-4-hydroxypyrrolidine- | | |
| | 2-carboxamide | | |
| | Synthesized from C-I, tert-butyl 4- | | |
| | (piperidin-4-yloxy)piperidine-1- | | |
| | carboxylate and L-XIX according to GP 2 | | |
| 39 | 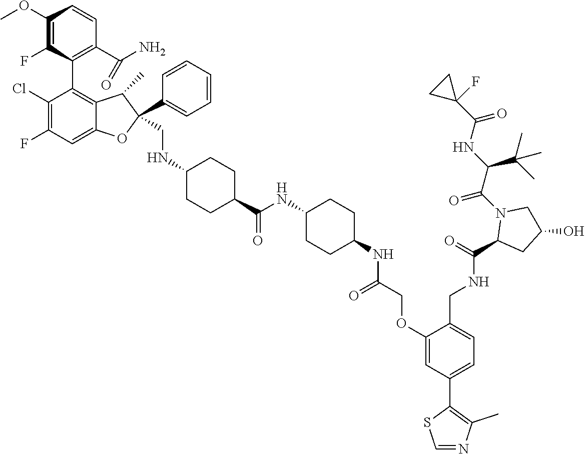 | 627.6 [M + 2H]²⁺/2 0.95 (4) | n.d. |
| | (2S,4R)-N-(2-(2-(((1S,4S)-4-((1S,4R)-4- | | |
| | ((((2S,3S,4S)-4-(6-carbamoyl-2-fluoro-3- | | |
| | methoxyphenyl)-5-chloro-6-fluoro-3- | | |
| | methyl-2-phenyl-2,3-dihydrobenzofuran- | | |
| | 2-yl)methyl)amino)cyclohexane-1- | | |
| | carboxamido)cyclohexyl)amino)-2- | | |
| | oxoethoxy)-4-(4-methylthiazol-5- | | |
| | yl)benzyl)-1-((S)-2-(1- | | |
| | fluorocyclopropane-1-carboxamido)-3,3- | | |
| | dimethylbutanoyl)-4-hydroxypyrrolidine- | | |
| | 2-carboxamide | | |
| | Synthesized from C-I, tert-butyl((1r,4r)-4- | | |
| | aminocyclohexyl)carbamate and L-XIX | | |
| | according to GP 2 | | |
| 40 | 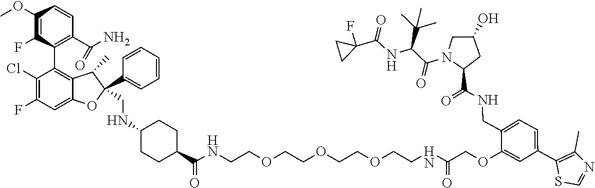 | 666.7 [M + 2H]²⁺/2 0.91 (4) | n.d. |
| | (2S,4R)-N-(2-((1-((1S,4r)-4- | | |
| | ((((2S,3S,4S)-4-(6-carbamoyl-2-fluoro-3- | | |
| | methoxyphenyl)-5-chloro-6-fluoro-3- | | |
| | methyl-2-phenyl-2,3-dihydrobenzofuran- | | |
| | 2-yl)methyl)amino)cyclohexyl)-1,15- | | |
| | dioxo-5,8,11-trioxa-2,14- | | |
| | diazahexadecan-16-yl)oxy)-4-(4- | | |
| | methylthiazol-5-yl)benzyl)-1-((S)-2-(1- | | |
| | fluorocyclopropane-1-carboxamido)-3,3- | | |
| | dimethylbutanoyl)-4-hydroxypyrrolidine- | | |
| | 2-carboxamide | | |
| | Synthesized from C-I, tert-butyl (2-(2-(2- | | |
| | (2- | | |
| | aminoethoxy)ethoxy)ethoxy)ethyl) | | |
| | carbamate and L-XIX according to GP 2 | | |
| 41 | 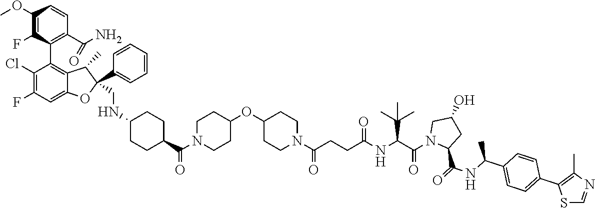 | 639.8 [M + 2H]²⁺/2 0.94 (4) | (400 MHz, DMSO-d₆) δ (ppm) δ 8.98 (s, 1H), 8.37 (d, J = 7.7 Hz, 1H), 7.85 (d, J = 9.2 Hz, 1H), 7.64-7-57 (m, 2H), 7.46- 7.23 (m, 10H), 7.11 (s br, 1H), 7.07 (d, J = 9.7 Hz, 1H), 5.09 (d, J = 3.5 Hz, 1H), 4.98- 4.87 (m, 1H), 4.48 (d, J = 9.3 Hz, 1H), 4.41 (t, J = 8.0 Hz, 1H), 4.30-4.25 (m, 1H), 3.87 (s, 3H), 3.86-3.80 (m, 2H), 3.72-3.54 (m, 6H), 3.28- 3.10 (m, 7H), 3.09-2.96 (m, 2H), 2.45 (s, 3H), 2.42-2.32 |
| | (2S,4R)-1-((S)-2-(4-(4-((1-((1S,4r)-4- | | (m, 2H), 2.24-2.13 (m, 1H), |
| | ((((2S,3S,4S)-4-(6-carbamoyl-2-fluoro-3- | | 2.04-1.98 (m, 1H), 1.83- |
| | methoxyphenyl)-5-chloro-6-fluoro-3- | | 1.69 (m, 7H), 1.62-1.48 (m, |
| | methyl-2-phenyl-2,3-dihydrobenzofuran- | | 2H), 1.37 (d, J = 7.0 Hz, 3H), |
| | 2-yl)methyl)amino)cyclohexane-1- | | 1.34-1.19 (m, 6H), 1.00- |
| | carbonyl)piperidin-4-yl)oxy)piperidin-1- | | 0.80 (m, 15 H) |
| | yl)-4-oxobutanamido)-3,3- | | |
| | dimethylbutanoyl)-4-hydroxy-N-((S)-1-(4- | | |
| | (4-methylthiazol-5- | | |
| | yl)phenyl)ethyl)pyrrolidine-2-carboxamide | | |
| | Synthesized from C-I, | | |
| | tert-butyl 4-(piperidin-4-yloxy)piperidine- | | |
| | 1-carboxylate and 4-(((S)-1-((2S,4R)-4- | | |
| | hydroxy-2-(((S)-1-(4-(4-methylthiazol-5- | | |
| | yl)phenyl)ethyl)carbamoyl)pyrrolidin-1- | | |
| | yl)-3,3-dimethyl-1-oxobutan-2-yl)amino)- | | |
| | 4-oxobutanoic acid (synthesized | | |
| | according to Eur. J. Med. Chem. 2020, | | |
| | 192, 112186) according to GP 2 | | |
| 42 | 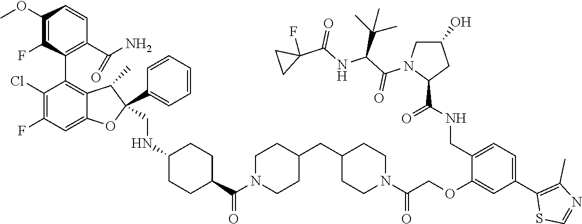 | 662.2 [M + 2H]²⁺/2 0.98 (4) | (400 MHz, DMSO-d₆) δ (ppm) δ 9.00 (s, 1H), 8.60 (s br, 1H), 7.69-7.57 (m, 2H), 7.46- 7.40 (m, 3H), 7.38-7.25 (m, 5H), 7.14 (s br, 1H), 7.10 (d, J = 9.5 Hz, 1H), 7.01-6.96 (m, 2H), 5.19 (d, J = 3.5 Hz, 1H), 4.97 (q, J = 16.5 Hz, 2H), 4.62 (d, J = 9.2 Hz, 1H), 4.52 (t, J = 8.2 Hz, 1H), 4.42-4.23 (m, 5H), 3.93-3.83 (m, 2H), 3.90 (s, 3H), 3.73-3.59 (m, 2H), 3.29-3.22 (m, 1H), 3.21- 3.11 (m, 2H), 3.10-2.90 (m, 2H), 2.65-2.46 (m, 1H), 2.45 |
| | (2S,4R)-N-(2-(2-(4-((1-((1S,4r)-4- | | (s, 3H), 2.26-2.16 (m, 1H), |
| | ((((2S,3S,4S)-4-(6-carbamoyl-2-fluoro-3- | | 2.14-2.07 (m, 1H), 1.98- |
| | methoxyphenyl)-5-chloro-6-fluoro-3- | | 1.89 (m, 1H), 1.84-1.50 (m, |
| | methyl-2-phenyl-2,3-dihydrobenzofuran- | | 10H), 1.44-1.21 (m, 6H), |
| | 2-yl)methyl)amino)cyclohexane-1- | | 1.15-1.07 (m, 2H), 1.02- |
| | carbonyl)piperidin-4-yl)methyl)piperidin- | | 0.81 (m, 19H) |
| | 1-yl)-2-oxoethoxy)-4-(4-methylthiazol-5- | | |
| | yl)benzyl)-1-((S)-2-(1- | | |
| | fluorocyclopropane-1-carboxamido)-3,3- | | |
| | dimethylbutanoyl)-4-hydroxypyrrolidine- | | |
| | 2-carboxamide | | |
| | Synthesized from C-I, tert-butyl 4- | | |
| | (piperidin-4-ylmethyl)piperidine-1- | | |
| | carboxylate and L-XIX according to GP 2 | | |
| 43 | 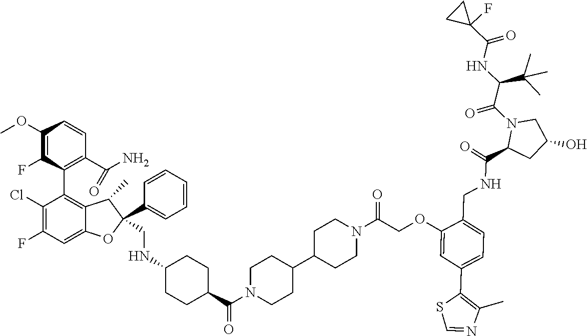 | 654.8 [M + 2H]²⁺/2 0.97 (4) | (400 MHz, DMSO-d₆) δ (ppm) 8.91 (s, 1H), 8.51 (s br, 1H), 7.56-7.50 (m, 2H), 7.37- 7.31 (m, 3H), 7.29-7.16 (m, 5H), 7.05 (s br, 1H), 7.00 (d, J = 9.5 Hz, 1H), 6.91-6.87 (m, 2H), 5.10 (d, J = 3.5 Hz, 1H), 4.98-4.80 (m, 2H), 4.53 (d, J = 9.2 Hz, 1H), 4.43 (t, J = 8.2 Hz, 1H), 4.36-4.14 (m, 5H), 3.86-3.83 (m, 2H), 3.82 (s, 3H), 3.62-3.50 (m, 2H), 3.16 (q, J = 7.1 Hz, 1H), 3.10-3.05 (m, 2H), 2.85 (dt, J = 32.4, 12.6 Hz, 2H), 2.95-2.76 (m, 2H), 2.95-2.76 (m, 2H), 2.42- 2.26 (m, 1H), 2.36 (s, 3H), 2.16-2.07 (m, 1H), 2.06- 1.98 (m, 1H), 1.89-1.80 (m, 1H), 1.74-1.52 (m, 5H), 1.52- 1.44 (m, 2H), 1.35-1.13 (m, 7H), 1.08-1.03 (m, 1H), 0.93- |
| | (2S,4R)-N-(2-(2-(1′-((1S,4r)-4- | | 0.75 (m, 16H) |
| | ((((2S,3S,4S)-4-(6-carbamoyl-2-fluoro-3- | | |
| | methoxyphenyl)-5-chloro-6-fluoro-3- | | |
| | methyl-2-phenyl-2,3-dihydrobenzofuran- | | |
| | 2-yl)methyl)amino)cyclohexane-1- | | |
| | carbonyl)-[4,4′-bipiperidin]-1-yl)-2- | | |
| | oxoethoxy)-4-(4-methylthiazol-5- | | |
| | yl)benzyl)-1-((S)-2-(1- | | |
| | fluorocyclopropane-1-carboxamido)-3,3- | | |
| | dimethylbutanoyl)-4-hydroxypyrrolidine- | | |
| | 2-carboxamide | | |
| | Synthesized from C-I, tert-butyl [4,4′- | | |
| | bipiperidine]-1-carboxylate and L-XIX | | |
| | according to GP 2 | | |
| 44 | 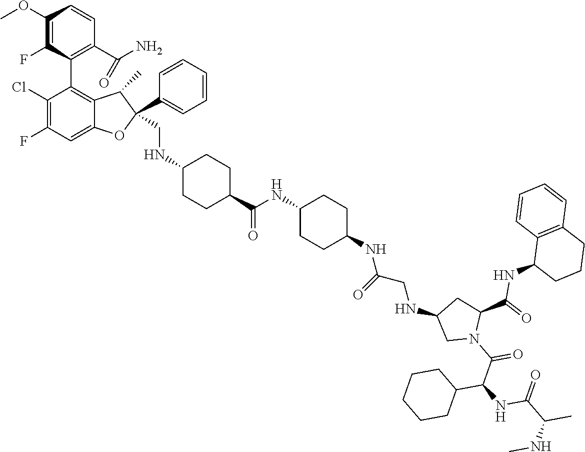 | 603.0 [M + 2H]²⁺/2 0.75 (4) | n.d. |
| | (2S,4S)-4-((2-(((1S,4S)-4-((1S,4S)-4- | | |
| | ((((2S,3S,4S)-4-(6-carbamoyl-2-fluoro-3- | | |
| | methoxyphenyl)-5-chloro-6-fluoro-3- | | |
| | methyl-2-phenyl-2,3-dihydrobenzofuran- | | |
| | 2-yl)methyl)amino)cyclohexane-1- | | |
| | carboxamido)cyclohexyl)amino)-2- | | |
| | oxoethyl)amino)-1-((S)-2-cyclohexyl-2- | | |
| | ((S)-2- | | |
| | (methylamino)propanamido)acetyl)-N- | | |
| | ((R)-1,2,3,4-tetrahydronaphthalen-1- | | |
| | yl)pyrrolidine-2-carboxamide | | |
| | Synthesized from C-I, tert-butyl((1r,4r)-4- | | |
| | aminocyclohexyl)carbamate and L-XX | | |
| | according to GP 2, followed by Boc | | |
| | deprotection | | |

International application PCT/IB2021/052089 (WO2021/186324) is hereby incorporated by reference. PCT/IB2021/052089 is directed to TEAD inhibitors corresponding to formula (Ia) and their synthesis.

1. A conjugate of Formula (I): 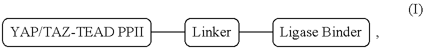
or a pharmaceutically acceptable salt thereof,
wherein:
the YAP/TAZ-TEAD PPI inhibitor is a group that is capable of binding to TEAD;
the Linker is a group that covalently links the YAP/TAZ-TEAD PPI inhibitor to the Ligase Binder; and
the Ligase Binder is a group that is capable of binding to a ligase (e.g., Cereblon E3 Ubiquitin ligase).

2. The conjugate or pharmaceutically acceptable salt thereof according to claim 1, wherein the YAP/TAZ-TEAD PPI inhibitor has a formula YTT-1, YTT-2 or YTT-3: 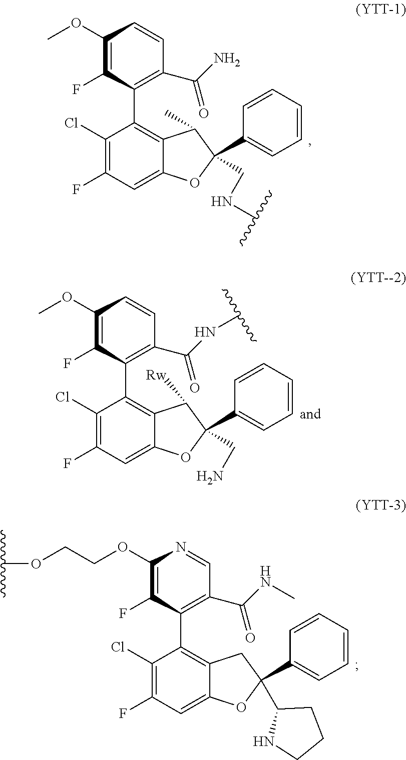
wherein R_{w} is H or CH₃ and [symbol] indicates the point of attachment to the linker.

3. The conjugate or pharmaceutically acceptable salt thereof according to claim 2, wherein the YAP/TAZ-TEAD PPI inhibitor has a formula YTT-1.

4. The conjugate or pharmaceutically acceptable salt thereof according to any one of the preceding claims, wherein the linker has a formula LIN-1:
-L¹-X¹-L²-X²-L³-(LIN-1), and wherein the conjugate has a formula (II): 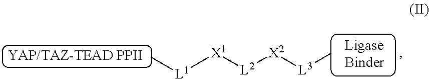
wherein:
the YAP/TAZ-TEAD PPI inhibitor is a group that is capable of binding to TEAD;
the Ligase Binder is a group that is capable of binding to a ligase (e.g., Cereblon E3 Ubiquitin ligase);
L¹ is selected from the group consisting of a bond, —O—, —NR′—, —C(O)—, C₁-C₉alkylene, C₁-C₉heteroalkylene, *C(O)—C₁-C₆alkylene, *C(O)—C₁-C₆heteroalkylene, *C₁-C₆alkylene-C(O), *C₁-C₆ heteroalkylene-C(O), and *L^{1a}-C₄-C₇cycloalkylene, wherein * denotes the point of attachment of L¹ to X¹;
wherein L^{1a} is selected from the group consisting of C(O), *NH—C(O) and *C₁-C₃alkylene-NH—C(O) wherein * denotes the point of attachment of L^{1a} to X¹;
X¹ and X² are each independently selected from the group consisting of a bond, C₄-C₇cycloalkylene, 4 to 7 membered heterocyclylene comprising 1 to 3 heteroatoms independently selected from the group consisting of N, O and S, and 5 or 6 membered heteroarylene comprising 1 to 3 heteroatoms independently selected from the group consisting of N, O and S;
L² is selected from the group consisting of a bond, —O—, —NR′—, —C(O)—, C₁-C₃alkylene, —NR′—C₁-C₉alkylene-NR′—, *C₁-C₉alkylene-NR′—,*NR′—C₁-C₉alkylene, *C(O)NR′—C₁-C₆alkylene, polyethylene glycol, —NR′-polyethylene glycol-NR′—, polyethylene glycol, *NR′-polyethylene glycol and *polyethylene glycol-NR′— wherein * denotes the point of attachment of L² to X²; or
X¹-L²-X² form a 7 to 13 membered spiroheterocyclylene comprising 1 to 4 heteroatoms independently selected from the group consisting of N, O and S;
L³ is selected from the group consisting of a bond, C₁-C₃alkylene, C₂-C₆alkenylene, C₂-C₆alkynylene, C₁-C₆heteroalkylene, —C(O)—, —S(O)₂—, —O—, *C(O)—C₁-C₉alkylene, *C(O)—C₁-C₆alkylene-O, *C(O)—C₁-C₉heteroalkylene, *C₁-C₆alkylene-NH and *NH—C₁-C₃alkylene wherein * denotes the point of attachment of L³ to X²;
and wherein each R′ is independently hydrogen or C₁-C₆alkyl (preferably wherein each R′ is hydrogen).

5. The conjugate or pharmaceutically acceptable salt thereof according to any one of claims 3 to 4 wherein L¹ is *L^{1a}-C₄-C₇cycloalkylene **, wherein L^{1a} is selected from the group consisting of a bond, C(O), *NH—C(O) and *C₁-C₃alkylene-NH—C(O), * denotes the point of attachment to X¹, and ** denotes the point of attachment to the YAP/TAZ-TEAD PPI inhibitor.

6. The conjugate or pharmaceutically acceptable salt thereof according to claim 5 wherein L¹ is *L^{1a}-C₆cycloalkylene ** wherein L^{1a} is selected from the group consisting of C(O), *NH—C(O) and *C₁-C₃alkylene-NH—C(O) (e.g. *C₂alkylene-NH—C(O)).

7. The conjugate or pharmaceutically acceptable salt, thereof according to claim 6 wherein L¹ is

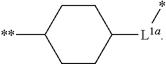

8. The conjugate or pharmaceutically acceptable salt thereof according to any one of claims 3 to 7, wherein X¹-L²-X² is selected from the group consisting of:
a) 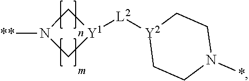
b) 7 to 11 membered spiroheterocyclylene comprising 1 to 3 heteroatoms independently selected from the group consisting of N and O (e.g. 11 membered spiroheterocyclylene comprising 2 heteroatoms which are both N);
c) 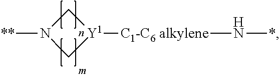
d) **NH—C₁-C₉alkylene-NH—* (e.g. **NH—C₇alkylene-NH—*),
e) NH,
f) C₁-C₆alkylene (e.g. C₅alkylene),
g) **C₁-C₃alkylene-5 or 6 membered heteroarylene comprising 1 to 3 heteroatoms independently selected from the group consisting of N and O—* (e.g. C₅alkylene-5 membered heteroarylene comprising 3 heteroatoms which are all N—*),
h) 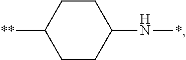
and
i) 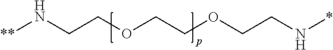
wherein:
Y¹ and Y² are each independently selected from the group consisting of CH and N, n and m are independently 1 or 2, * denotes the point of attachment to L³, ** denotes the point of attachment to L¹, and p is 0 to 23, e.g. p is 0 to 5, e.g. p is 1 to 4, e.g. p is 2 or 3, e.g. p is 2.

9. The conjugate or pharmaceutically acceptable salt hereof according to claim 8, wherein X¹-L²-X² is 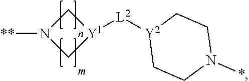
wherein:
Y¹ and Y² are each independently selected from the group consisting of CH and N, n and m are independently 1 or 2, * denotes the point of attachment to L³, and ** denotes the point of attachment to L¹.

10. The conjugate or pharmaceutically acceptable salt thereof according to claim 8 or claim 9, wherein n and m are both 1 or both 2.

11. The conjugate or pharmaceutically acceptable salt thereof according to claim 10,
wherein n and m are both 2.

12. The conjugate or pharmaceutically acceptable salt thereof according to any one of claims 9 to 11, wherein L² is selected from the group consisting of: a bond, C₁-C₃alkylene, —O— and —C(O)—.

13. The conjugate or pharmaceutically acceptable salt thereof according to any one of claims 3 to 12, wherein L³ is selected from the group consisting of: bond, —C(O)—, *C(O)—C₁-C₃alkylene-O, C₁-C₃alkylene (e.g. C₂-C₅alkylene) and *C(O)—C₁-C₆alkylene wherein * denotes the point of attachment to X².

14. The conjugate or pharmaceutically acceptable salt thereof according to any one of the preceding claims wherein the ligase binder is of the formula LIG-1: 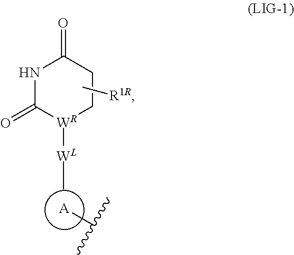
wherein: [symbol]
indicates the point of attachment to the linker,
R^{1R} is selected from the group consisting of H, C₁-C₃alkyl, O—C₁-C₃alkyl, halo (e.g. fluoro or chloro), C₁-C₃haloalkyl, and C₁-C₃hydroxyalkyl,
W^{R} is selected from the group consisting of N and CH,
W^{L} is selected from the group consisting of bond and CH₂ (preferably W is a bond), and 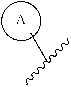
is selected from the group consisting of: 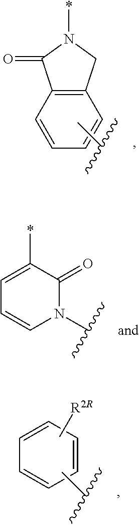
wherein * indicates the point of attachment to W,
R^{2R} is selected from the group consisting of H, C₁-C₃alkyl, O—C₁-C₃alkyl and halo
(e.g. fluoro or chloro), and with the caveat that when 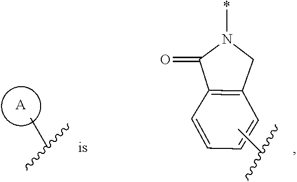
W^{R} is CH.

15. The conjugate or pharmaceutically acceptable salt thereof according to claim 14 wherein R^{1R} is H.

16. The conjugate or pharmaceutically acceptable salt thereof according to claim 14 or claim 15 wherein the ligase binder is selected from the group consisting of:

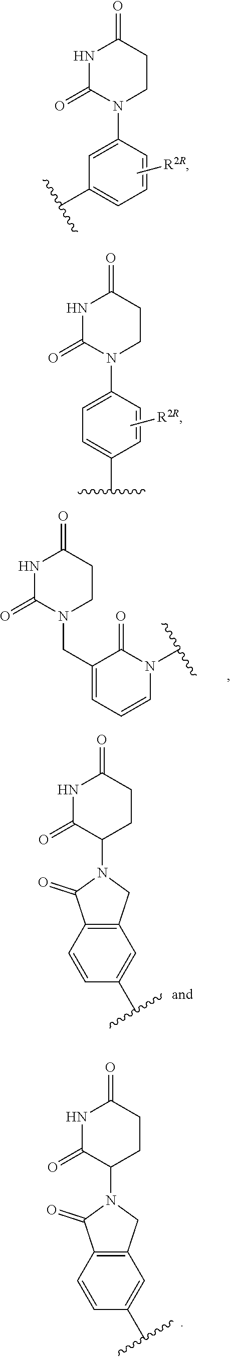

17. The conjugate or pharmaceutically acceptable salt thereof according to claim 16 wherein the ligase binder is selected from the group consisting of:

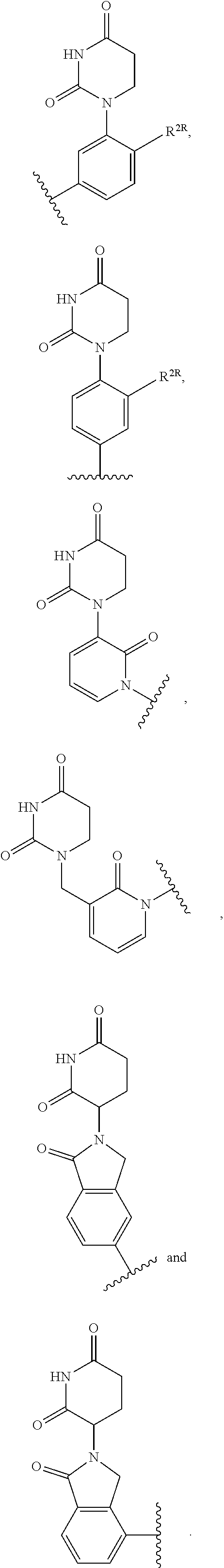

18. The conjugate or pharmaceutically acceptable salt thereof according to claim 17 wherein the ligase binder is selected from the group consisting of:

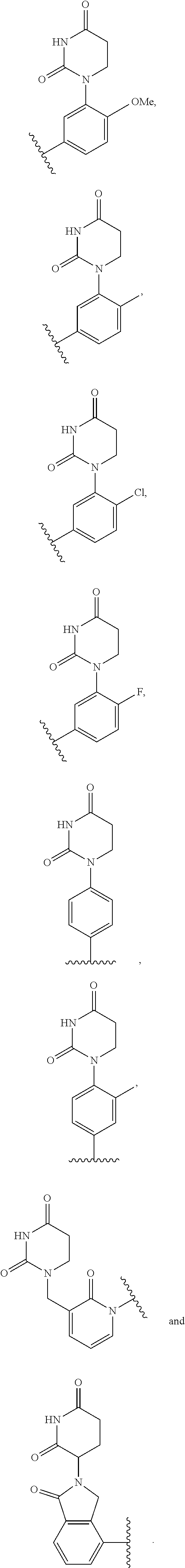

19. The conjugate or pharmaceutically acceptable salt hereof according to any one of claims 1-13 wherein the ligase binder is selected from the group consisting of: 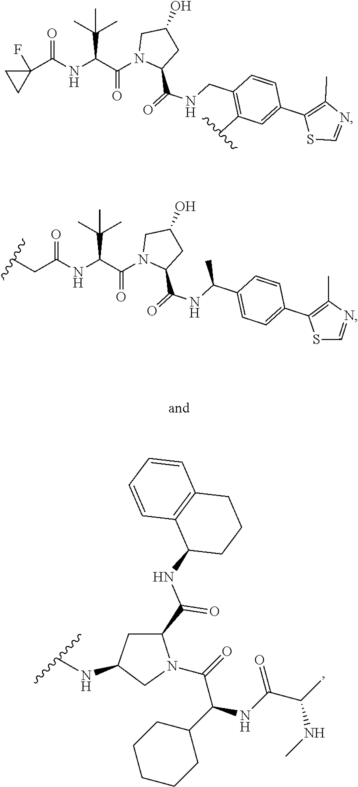
wherein [symbol] indicates the point of attachment to the linker.

20. A conjugate or pharmaceutically acceptable salt thereof selected from the group consisting of:
2-((2S,3S,4S)-5-chloro-2-((((1r,4S)-4-(4-((1-(3-(2,4-dioxotetrahydropyrimidin-1(2H)-yl)-4-methoxybenzoyl)piperidin-4-yl)methyl)piperidine-1-carbonyl)cyclohexyl)amino)methyl)-6-fluoro-3-methyl-2-phenyl-2,3-dihydrobenzofuran-4-yl)-3-fluoro-4-methoxybenzamide;
2-((2S,3S,4S)-5-chloro-2-((((1r,4S)-4-(4-((1-(3-(2,4-dioxotetrahydropyrimidin-1(2H)-yl)-4-methoxybenzoyl)piperidin-4-yl)oxy)piperidine-1-carbonyl)cyclohexyl)amino)methyl)-6-fluoro-3-methyl-2-phenyl-2,3-dihydrobenzofuran-4-yl)-3-fluoro-4-methoxybenzamide;
2-((2S,3S,4S)-5-chloro-2-((((1r,4S)-4-((2-(4-((4-(3-(2,4-dioxotetrahydropyrimidin-1(2H)-yl)-4-methoxybenzoyl) piperazin-1-yl)methyl)piperidin-1-yl)ethyl)carbamoyl)cyclohexyl)amino)methyl)-6-fluoro-3-methyl-2-phenyl-2,3-dihydrobenzofuran-4-yl)-3-fluoro-4-methoxybenzamide;
2-((2S,3S,4S)-5-chloro-2-((((1r,4S)-4-((2-(4-((1-(3-(2,4-dioxotetrahydropyrimidin-1(2H)-yl)-4-methoxybenzoyl)piperidin-4-yl)oxy)piperidin-1-yl)ethyl)carbamoyl)cyclohexyl)amino)methyl)-6-fluoro-3-methyl-2-phenyl-2,3-dihydrobenzofuran-4-yl)-3-fluoro-4-methoxybenzamide;
2-((2S,3S,4S)-5-chloro-2-((((1r,4S)-4-((2-(9-(3-(2,4-dioxotetrahydropyrimidin-1(2H)-yl)-4-methoxybenzoyl)-3,9-diazaspiro [5.5]undecan-3-yl)ethyl)carbamoyl)cyclohexyl)amino)methyl)-6-fluoro-3-methyl-2-phenyl-2,3-dihydrobenzofuran-4-yl)-3-fluoro-4-methoxybenzamide;
2-((2S,3S,4S)-5-chloro-2-((((1r,4S)-4-(4-((4-(3-(2,4-dioxotetrahydropyrimidin-1(2H)-yl)-4-methoxybenzoyl) piperazin-1-yl)methyl)piperidine-1-carbonyl)cyclohexyl)amino)methyl)-6-fluoro-3-methyl-2-phenyl-2,3-dihydrobenzofuran-4-yl)-3-fluoro-4-methoxybenzamide;
2-((2S,3S,4S)-5-chloro-2-((((1r,4S)-4-((2-(4-((1-(3-(2,4-dioxotetrahydropyrimidin-1(2H)-yl)-4-methoxybenzoyl)piperidin-4-yl)methyl)piperidin-1-yl)ethyl)carbamoyl)cyclohexyl)amino)methyl)-6-fluoro-3-methyl-2-phenyl-2,3-dihydrobenzofuran-4-yl)-3-fluoro-4-methoxybenzamide;
2-((2S,3S,4S)-5-chloro-2-((((1r,4S)-4-(3-((1-(3-(2,4-dioxotetrahydropyrimidin-1(2H)-yl)-4-methoxybenzoyl)piperidin-4-yl)oxy) azetidine-1-carbonyl)cyclohexyl)amino)methyl)-6-fluoro-3-methyl-2-phenyl-2,3-dihydrobenzofuran-4-yl)-3-fluoro-4-methoxybenzamide;
2-((2S,3S,4S)-5-chloro-2-((((1r,4S)-4-(3-((4-(3-(2,4-dioxotetrahydropyrimidin-1(2H)-yl)-4-methoxybenzoyl) piperazin-1-yl)methyl) azetidine-1-carbonyl)cyclohexyl)amino)methyl)-6-fluoro-3-methyl-2-phenyl-2,3-dihydrobenzofuran-4-yl)-3-fluoro-4-methoxybenzamide;
2-((2S,3S,4S)-5-chloro-2-((((1r,4S)-4-(1′-(3-(2,4-dioxotetrahydropyrimidin-1(2H)-yl)-4-methoxybenzoyl)-[4,4′-bipiperidine]-1-carbonyl)cyclohexyl)amino)methyl)-6-fluoro-3-methyl-2-phenyl-2,3-dihydrobenzofuran-4-yl)-3-fluoro-4-methoxybenzamide;
2-((2S,3S,4S)-5-chloro-2-((((1r,4S)-4-((2-(1′-(3-(2,4-dioxotetrahydropyrimidin-1(2H)-yl)-4-methoxybenzoyl)-[4,4′-bipiperidin]-1-yl)ethyl)carbamoyl)cyclohexyl)amino)methyl)-6-fluoro-3-methyl-2-phenyl-2,3-dihydrobenzofuran-4-yl)-3-fluoro-4-methoxybenzamide;
2-((2S,3S,4S)-5-chloro-2-((((1r,4S)-4-(4-((4-(2-(3-(2,4-dioxotetrahydropyrimidin-1(2H)-yl)-4-methylphenoxy)acetyl) piperazin-1-yl)methyl)piperidine-1-carbonyl)cyclohexyl)amino)methyl)-6-fluoro-3-methyl-2-phenyl-2,3-dihydrobenzofuran-4-yl)-3-fluoro-4-methoxybenzamide;
2-((2S,3S,4S)-5-chloro-2-((((1r,4S)-4-(4-((1-(4-chloro-3-(2,4-dioxotetrahydropyrimidin-1(2H)-yl)benzoyl)piperidin-4-yl)oxy)piperidine-1-carbonyl)cyclohexyl)amino)methyl)-6-fluoro-3-methyl-2-phenyl-2,3-dihydrobenzofuran-4-yl)-3-fluoro-4-methoxybenzamide;
2-((2S,3S,4S)-5-chloro-2-((((1r,4S)-4-(4-((1-(2-(4-(2,4-dioxotetrahydropyrimidin-1(2H)-yl)-3-methylphenoxy)acetyl)piperidin-4-yl)oxy)piperidine-1-carbonyl)cyclohexyl)amino)methyl)-6-fluoro-3-methyl-2-phenyl-2,3-dihydrobenzofuran-4-yl)-3-fluoro-4-methoxybenzamide;
2-((2S,3S,4S)-5-chloro-2-((((1r,4S)-4-(4-((1-(2-(3-((2,4-dioxotetrahydropyrimidin-1(2H)-yl)methyl)-2-oxopyridin-1(2H)-yl)ethyl)piperidin-4-yl)methyl)piperidine-1-carbonyl)cyclohexyl)amino)methyl)-6-fluoro-3-methyl-2-phenyl-2,3-dihydrobenzofuran-4-yl)-3-fluoro-4-methoxybenzamide;
2-((2S,3S,4S)-5-chloro-2-((((1r,4S)-4-(4-((1-(2-(3-((2,4-dioxotetrahydropyrimidin-1(2H)-yl)methyl)-2-oxopyridin-1(2H)-yl)acetyl)piperidin-4-yl)methyl)piperidine-1-carbonyl)cyclohexyl)amino)methyl)-6-fluoro-3-methyl-2-phenyl-2,3-dihydrobenzofuran-4-yl)-3-fluoro-4-methoxybenzamide;
2-((2S,3S,4S)-5-chloro-2-((((1r,4S)-4-(4-((1-(3-(2,4-dioxotetrahydropyrimidin-1(2H)-yl)-4-methoxybenzoyl)piperidin-4-yl)methyl)piperazine-1-carbonyl)cyclohexyl)amino)methyl)-6-fluoro-3-methyl-2-phenyl-2,3-dihydrobenzofuran-4-yl)-3-fluoro-4-methoxybenzamide;
N-(4-(1-((1S,4r)-4-((((2S,3S,4S)-4-(6-carbamoyl-2-fluoro-3-methoxyphenyl)-5-chloro-6-fluoro-3-methyl-2-phenyl-2,3-dihydrobenzofuran-2-yl)methyl)amino)cyclohexane-1-carbonyl)piperidin-4-yl)butyl)-3-(2,4-dioxotetrahydropyrimidin-1(2H)-yl)-4-methoxybenzamide;
2-((2S,3S,4S)-5-chloro-2-((((1r,4S)-4-(1′-(2-(3-(2,4-dioxotetrahydropyrimidin-1(2H)-yl)-4-methylphenoxy)acetyl)-[4,4′-bipiperidine]-1-carbonyl)cyclohexyl)amino)methyl)-6-fluoro-3-methyl-2-phenyl-2,3-dihydrobenzofuran-4-yl)-3-fluoro-4-methoxybenzamide;
2-((2S,3S,4S)-5-chloro-2-((((1r,4S)-4-(4-((1-(2-(3-(2,4-dioxotetrahydropyrimidin-1(2H)-yl)-4-methylphenoxy)acetyl)piperidin-4-yl)oxy)piperidine-1-carbonyl)cyclohexyl)amino)methyl)-6-fluoro-3-methyl-2-phenyl-2,3-dihydrobenzofuran-4-yl)-3-fluoro-4-methoxybenzamide;
2-((2S,3S,4S)-5-chloro-2-((((1r,4S)-4-(4-((1-(2-(3-(2,4-dioxotetrahydropyrimidin-1(2H)-yl)-4-methylphenoxy)acetyl)piperidin-4-yl)methyl)piperidine-1-carbonyl)cyclohexyl)amino)methyl)-6-fluoro-3-methyl-2-phenyl-2,3-dihydrobenzofuran-4-yl)-3-fluoro-4-methoxybenzamide;
2-((2S,3S,4S)-5-chloro-2-((((1r,4S)-4-(4-((1-(2-(3-(2,4-dioxotetrahydropyrimidin-1(2H)-yl)-4-methylphenoxy)acetyl)piperidin-4-yl)methyl)piperazine-1-carbonyl)cyclohexyl)amino)methyl)-6-fluoro-3-methyl-2-phenyl-2,3-dihydrobenzofuran-4-yl)-3-fluoro-4-methoxybenzamide;
2-((2S,3S,4S)-5-chloro-2-((((1r,4S)-4-((7-(2-(3-(2,4-dioxotetrahydropyrimidin-1(2H)-yl)-4-methylphenoxy) acetamido) heptyl)carbamoyl)cyclohexyl)amino)methyl)-6-fluoro-3-methyl-2-phenyl-2,3-dihydrobenzofuran-4-yl)-3-fluoro-4-methoxybenzamide;
2-((2S,3S,4S)-5-chloro-2-((((1r,4S)-4-(4-((1-(4-chloro-3-(2,4-dioxotetrahydropyrimidin-1(2H)-yl)benzoyl)piperidin-4-yl)methyl)piperidine-1-carbonyl)cyclohexyl)amino)methyl)-6-fluoro-3-methyl-2-phenyl-2,3-dihydrobenzofuran-4-yl)-3-fluoro-4-methoxybenzamide;
2-((2S,3S,4S)-5-chloro-2-((((1r,4S)-4-(4-((1-(2-(4-(2,4-dioxotetrahydropyrimidin-1(2H)-yl) phenoxy)acetyl)piperidin-4-yl)methyl)piperidine-1-carbonyl)cyclohexyl)amino)methyl)-6-fluoro-3-methyl-2-phenyl-2,3-dihydrobenzofuran-4-yl)-3-fluoro-4-methoxybenzamide;
2-((2S,3S,4S)-5-chloro-2-((((1r,4S)-4-(4-((1-(2-(4-(2,4-dioxotetrahydropyrimidin-1(2H)-yl)-3-methylphenoxy)acetyl)piperidin-4-yl)methyl)piperidine-1-carbonyl)cyclohexyl)amino)methyl)-6-fluoro-3-methyl-2-phenyl-2,3-dihydrobenzofuran-4-yl)-3-fluoro-4-methoxybenzamide;
2-((2S,3S,4S)-5-chloro-2-((((1r,4S)-4-(4-(2-(1-(3-(2,4-dioxotetrahydropyrimidin-1(2H)-yl)-4-methoxybenzoyl)piperidin-4-yl)ethyl)piperidine-1-carbonyl)cyclohexyl)amino)methyl)-6-fluoro-3-methyl-2-phenyl-2,3-dihydrobenzofuran-4-yl)-3-fluoro-4-methoxybenzamide;
2-((2S,3S,4S)-5-chloro-2-((((1r,4S)-4-(4-((1-(2-(4-chloro-3-(2,4-dioxotetrahydropyrimidin-1(2H)-yl) phenoxy)acetyl)piperidin-4-yl)methyl)piperidine-1-carbonyl)cyclohexyl)amino)methyl)-6-fluoro-3-methyl-2-phenyl-2,3-dihydrobenzofuran-4-yl)-3-fluoro-4-methoxybenzamide;
2-((2S,3S,4S)-5-chloro-2-((((1r,4S)-4-(4-((1-(2-(3-(2,4-dioxotetrahydropyrimidin-1(2H)-yl)-4-fluorophenoxy)acetyl)piperidin-4-yl)methyl)piperidine-1-carbonyl)cyclohexyl)amino)methyl)-6-fluoro-3-methyl-2-phenyl-2,3-dihydrobenzofuran-4-yl)-3-fluoro-4-methoxybenzamide;
2-((2S,3S,4S)-5-chloro-2-((((1r,4S)-4-(4-((1-(2-(3-(2,4-dioxotetrahydropyrimidin-1(2H)-yl)-4-methoxyphenoxy)acetyl)piperidin-4-yl)methyl)piperidine-1-carbonyl)cyclohexyl)amino)methyl)-6-fluoro-3-methyl-2-phenyl-2,3-dihydrobenzofuran-4-yl)-3-fluoro-4-methoxybenzamide;
2-((2S,3S,4S)-5-chloro-2-((((1r,4S)-4-((5-(2-(2,6-dioxopiperidin-3-yl)-1-oxoisoindolin-4-yl)pentyl)carbamoyl)cyclohexyl)amino)methyl)-6-fluoro-3-methyl-2-phenyl-2,3-dihydrobenzofuran-4-yl)-3-fluoro-4-methoxybenzamide;
4-((2S,4S)-5-Chloro-6-fluoro-2-phenyl-2-((S)-pyrrolidin-2-yl)-2,3-dihydrobenzofuran-4-yl)-6-(2-(2-(4-(2-(2,6-dioxopiperidin-3-yl)-1-oxoisoindolin-5-yl)-1H-1,2,3-triazol-1-yl)ethoxy)ethoxy)-5-fluoro-N-methylnicotinamide;
4-((2S,4S)-5-chloro-6-fluoro-2-phenyl-2-((S)-pyrrolidin-2-yl)-2,3-dihydrobenzofuran-4-yl)-6-(2-(2-(2-(4-(2-(2,6-dioxopiperidin-3-yl)-1-oxoisoindolin-5-yl)-1H-1,2,3-triazol-1-yl)ethoxy)ethoxy)ethoxy)-5-fluoro-N-methylnicotinamide;
4-((2S,4S)-5-chloro-6-fluoro-2-phenyl-2-((S)-pyrrolidin-2-yl)-2,3-dihydrobenzofuran-4-yl)-6-(2-(2-(2-(4-(3-(2,4-dioxotetrahydropyrimidin-1(2H)-yl)phenyl)-1H-1,2,3-triazol-1-yl)ethoxy)ethoxy)ethoxy)-5-fluoro-N-methylnicotinamide;
2-((2S,3S,4S)-5-chloro-2-(((5-(2-(2,6-dioxopiperidin-3-yl)-1-oxoisoindolin-4-yl)pentyl)amino)methyl)-6-fluoro-3-methyl-2-phenyl-2,3-dihydrobenzofuran-4-yl)-3-fluoro-4-methoxybenzamide;
2-((2S,4S)-2-(aminomethyl)-5-chloro-6-fluoro-2-phenyl-2,3-dihydrobenzofuran-4-yl)-N-(5-(2-(2,6-dioxopiperidin-3-yl)-1-oxoisoindolin-4-yl)pentyl)-3-fluoro-4-methoxybenzamide;
2-((2S,4S)-2-(aminomethyl)-5-chloro-6-fluoro-2-phenyl-2,3-dihydrobenzofuran-4-yl)-N-(5-(4-(2-(2,6-dioxopiperidin-3-yl)-1-oxoisoindolin-5-yl)-1H-1,2,3-triazol-1-yl)pentyl)-3-fluoro-4-methoxybenzamide;
(2S,4R)—N-(2-(2-(4-((1-((1S,4r)-4-((((2S,3S,4S)-4-(6-carbamoyl-2-fluoro-3-methoxyphenyl)-5-chloro-6-fluoro-3-methyl-2-phenyl-2,3-dihydrobenzofuran-2-yl)methyl)amino)cyclohexane-1-carbonyl)piperidin-4-yl)oxy)piperidin-1-yl)-2-oxoethoxy)-4-(4-methylthiazol-5-yl)benzyl)-1-((S)-2-(1-fluorocyclopropane-1-carboxamido)-3,3-dimethylbutanoyl)-4-hydroxypyrrolidine-2-carboxamide;
(2S,4R)—N-(2-(2-(((1S,4S)-4-((1S,4R)-4-((((2S,3S,4S)-4-(6-carbamoyl-2-fluoro-3-methoxyphenyl)-5-chloro-6-fluoro-3-methyl-2-phenyl-2,3-dihydrobenzofuran-2-yl)methyl)amino)cyclohexane-1-carboxamido)cyclohexyl)amino)-2-oxoethoxy)-4-(4-methylthiazol-5-yl)benzyl)-1-((S)-2-(1-fluorocyclopropane-1-carboxamido)-3,3-dimethylbutanoyl)-4-hydroxypyrrolidine-2-carboxamide;
(2S,4R)—N-(2-((1-((1S,4r)-4-((((2S,3S,4S)-4-(6-carbamoyl-2-fluoro-3-methoxyphenyl)-5-chloro-6-fluoro-3-methyl-2-phenyl-2,3-dihydrobenzofuran-2-yl)methyl)amino)cyclohexyl)-1,15-dioxo-5,8,11-trioxa-2,14-diazahexadecan-16-yl)oxy)-4-(4-methylthiazol-5-yl)benzyl)-1-((S)-2-(1-fluorocyclopropane-1-carboxamido)-3,3-dimethylbutanoyl)-4-hydroxypyrrolidine-2-carboxamide;
(2S,4R)-1-((S)-2-(4-(4-((1-((1S,4r)-4-((((2S,3S,4S)-4-(6-carbamoyl-2-fluoro-3-methoxyphenyl)-5-chloro-6-fluoro-3-methyl-2-phenyl-2,3-dihydrobenzofuran-2-yl)methyl)amino)cyclohexane-1-carbonyl)piperidin-4-yl)oxy)piperidin-1-yl)-4-oxobutanamido)-3,3-dimethylbutanoyl)-4-hydroxy-N-((S)-1-(4-(4-methylthiazol-5-yl)phenyl)ethyl) pyrrolidine-2-carboxamide;
(2S,4R)—N-(2-(2-(4-((1-((1S,4r)-4-((((2S,3S,4S)-4-(6-carbamoyl-2-fluoro-3-methoxyphenyl)-5-chloro-6-fluoro-3-methyl-2-phenyl-2,3-dihydrobenzofuran-2-yl)methyl)amino)cyclohexane-1-carbonyl)piperidin-4-yl)methyl)piperidin-1-yl)-2-oxoethoxy)-4-(4-methylthiazol-5-yl)benzyl)-1-((S)-2-(1-fluorocyclopropane-1-carboxamido)-3,3-dimethylbutanoyl)-4-hydroxypyrrolidine-2-carboxamide;
(2S,4R)—N-(2-(2-(1′-((1S,4r)-4-((((2S,3S,4S)-4-(6-carbamoyl-2-fluoro-3-methoxyphenyl)-5-chloro-6-fluoro-3-methyl-2-phenyl-2,3-dihydrobenzofuran-2-yl)methyl)amino)cyclohexane-1-carbonyl)-[4,4′-bipiperidin]-1-yl)-2-oxoethoxy)-4-(4-methylthiazol-5-yl)benzyl)-1-((S)-2-(1-fluorocyclopropane-1-carboxamido)-3,3-dimethylbutanoyl)-4-hydroxypyrrolidine-2-carboxamide; and
(2S,4S)-4-((2-(((1S,4S)-4-((1S,4S)-4-((((2S,3S,4S)-4-(6-carbamoyl-2-fluoro-3-methoxyphenyl)-5-chloro-6-fluoro-3-methyl-2-phenyl-2,3-dihydrobenzofuran-2-yl)methyl)amino)cyclohexane-1-carboxamido)cyclohexyl)amino)-2-oxoethyl)amino)-1-((S)-2-cyclohexyl-2-((S)-2-(methylamino) propanamido)acetyl)-N—((R)-1,2,3,4-tetrahydronaphthalen-1-yl) pyrrolidine-2-carboxamide, or a pharmaceutically acceptable salt thereof.

21. A pharmaceutical composition comprising the conjugate or pharmaceutically acceptable salt thereof according to any one of the preceding claims, and one or more pharmaceutically acceptable carriers.

22. A combination comprising the conjugate or pharmaceutically acceptable salt thereof according to any one of claims 1 to 20, and one or more therapeutically active agents.

23. The conjugate or pharmaceutically acceptable salt thereof according to any one of claims 1 to 20 for use as a medicament.

24. The conjugate or pharmaceutically acceptable salt thereof according to any one of claims 1 to 20 for use in the treatment of cancer, preferably wherein the cancer is selected from breast cancer, lung cancer, ovarian cancer, colorectal cancer, malignant pleural mesothelioma, pancreatic cancer, prostate cancer, gastric cancer, esophageal cancer, liver cancer and bone cancer.

25. The conjugate or pharmaceutically acceptable salt thereof according to any one of claims 1 to 20 for use in the treatment of a disease or condition mediated by YAP overexpression and/or YAP amplification and/or YAP/TAZ-TEAD interaction.
